(19) United States
(12) Patent Application Publication
(10) Pub. No.: US 2024/0132484 A1
(43) Pub. Date: Apr. 25, 2024

(54) BROAD SPECTRUM ANTIVIRAL COMPOUNDS TARGETING THE SKI COMPLEX

(71) Applicant: UNIVERSITY OF MARYLAND, BALTIMORE, Baltimore, MD (US)

(72) Inventors: Matthew Frieman, Baltimore, MD (US); Alexander D. Mackerell, JR., Baltimore, MD (US); Stuart WESTON, Baltimore, MD (US)

(21) Appl. No.: 18/265,191

(22) PCT Filed: Dec. 3, 2021
(86) PCT No.: PCT/US2021/061863

§ 371 Date: Jun. 2, 2023

Related U.S. Application Data
(60) Provisional application No. 63/121,120, filed on Dec. 3, 2020.

(51) Int. Cl. C07D 409/12; C07D 211/22; C07D 231/14; C07D 239/40; C07D 307/52; C07D 333/20; C07D 407/12; C07D 413/12; C07D 471/04

(52) U.S. Cl. CPC C07D 409/12; C07D 211/22; C07D 231/14; C07D 239/40; C07D 307/52; C07D 333/20; C07D 407/12; C07D 413/12; C07D 471/04

(57) ABSTRACT

Compounds and methods of using the same for treating conditions alleviated by SKI complex inhibition, viral replication inhibition, or interferon signaling inducement are provided.

### CROSS-REFERENCE TO RELATED APPLICATION

This application claims the benefit of U.S. Provisional Patent Application No. 63/121,120, filed Dec. 3, 2020, which is incorporated by reference herein in its entirety.

### FIELD

The disclosure relates generally to compounds and methods of using the same for treating conditions alleviated by SKI complex inhibition, viral replication inhibition, or interferon signaling inducement.

### BACKGROUND

At the end of 2019 cases of pneumonia of unknown etiology were identified in China. In the first week of January, a novel coronavirus was identified as the cause and was found to be spreading between people. In the months since, that virus has spread around the world leading to the WHO announcing it a pandemic on 11 Mar. 2020 and the milestone of a 3 million confirmed cases was passed on 27 Apr. 2020. Amongst many things that the SARS-CoV-2 (severe acute respiratory syndrome coronavirus-2) outbreak has demonstrated is the need for both specific and broadly acting antiviral therapeutics. There is a need for the development of broad-spectrum antiviral compounds to treat known viruses, and those yet to emerge in the human population. With the emergence of three novel coronaviruses in the past 18 years, there will undoubtedly be more coronaviruses and other viruses that emerge in the future.

### SUMMARY

The disclosure provides a compound of formula (I), or a pharmaceutically acceptable salt, solvate, hydrate, cocrystal, or prodrug thereof:

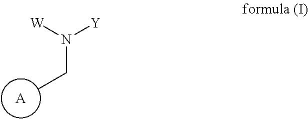

wherein in formula (I):
W is selected from C₁-C₅-alkyl and

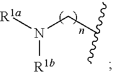

Y is selected from H,

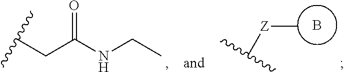

A and B are each independently selected from:

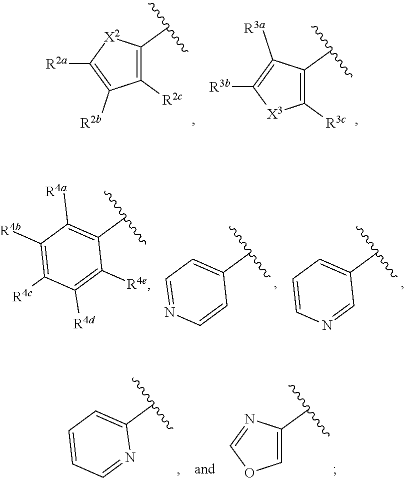

X² and X³ at each occurrence are independently selected from O and S;
Z is selected from —CH₂— and —C(O)—;
R^{1a} and R^{1b} at each occurrence are independently C₁-C₅-alkyl;
R^{2a}, R^{2b}, R^{2c}, R^{3a}, R^{3b}, R^{3c}, R^{4a}, R^{4b}, R^{4c}, R^{4d}, and R^{4e} at each occurrence are independently selected from hydrogen, halo, C₁-C₅-alkyl, trifluoromethyl, —(C₁-C₅-alkyl)-OH, phenyl, thiophenyl, furyl, and —C(O)O—C₁-C₅-alkyl; and
n is an integer selected from 1 to 5.

In some embodiments, W is C₁-C₅-alkyl. In some embodiments, is H or

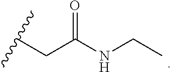 [SMILES: *CC(=O)NCC]

In some embodiments, the compound of formula (I) is a compound of formula (10), or a pharmaceutically acceptable salt, solvate, hydrate, cocrystal, or prodrug thereof:

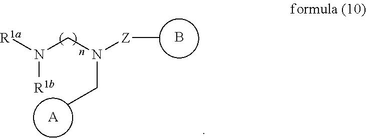

In some embodiments, A and B are each independently selected from:

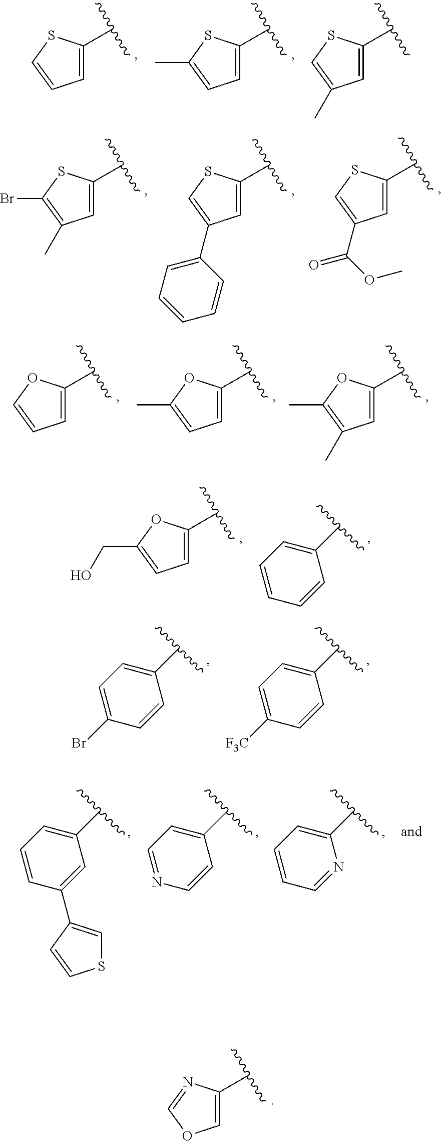

In some embodiments, Z is —CH₂—. In some embodiments,

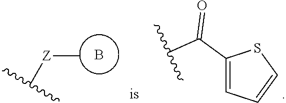

In some embodiments,

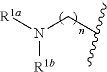

is selected from,

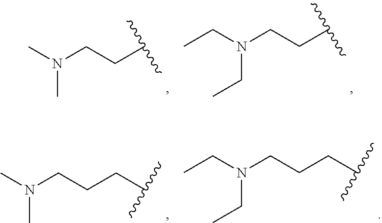

In some embodiments, the compound of formula (I) is a compound having any one of formulas 1001-1038, or a pharmaceutically acceptable salt, solvate, hydrate, cocrystal, or prodrug thereof.

The disclosure provides a compound of formula (II), or a pharmaceutically acceptable salt, solvate, hydrate, cocrystal, or prodrug thereof:

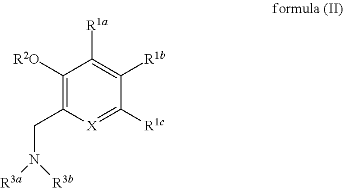

wherein in formula (II):
X is N or CR^{1d};
R^{1a}, R^{1b}, R^{1c}, and R^{1d} are each independently selected from hydrogen, C₁-C₅-alkyl, —OH, C₁-C₅-alkoxy, C₁-C₅-cycloalkyl,

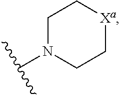

X^{a} is at each occurrence independently selected from —CH₂—, —O—, —NH—, or —N(C₁-C₅-alkyl)-;
R² is selected from hydrogen, C₁-C₅-alkyl, C₁-C₅-alkenyl, —(C₁-C₅-alkyl)-OH,

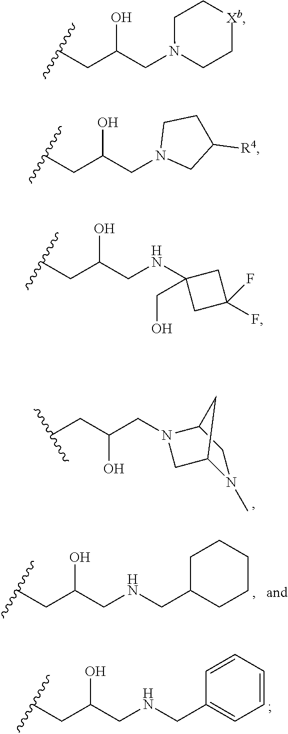

X^{b} is at each occurrence independently selected from —CH₂—, —O—, —NH—, or —N(C₁-C₅-alkyl)-;
R^{3a} and R^{3b} are each independently selected from hydrogen, C₁-C₅-alkyl, —(C₁-C₅-alkyl)-O—(C₁-C₅-alkyl),

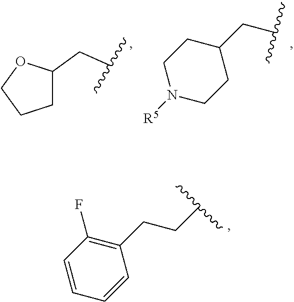

or R^{3a} and R^{3b} are joined together to form

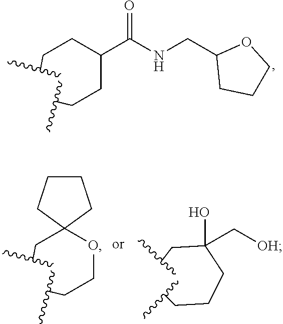 [SMILES: *CCC(CC*)C(=O)NCC1CCCO1 ; *CCOC1(C*)CCCC1 ; "or" ; *CCCC(O)(C*)CO]

R⁴ is at each occurrence independently selected from hydrogen and halo;
R⁵ is at each occurrence independently selected from

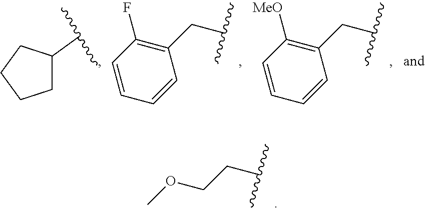

In some embodiments, the compound of formula (II) is a compound of formula 20, or a pharmaceutically acceptable salt, solvate, hydrate, cocrystal, or prodrug thereof:

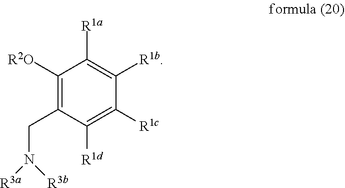

In some embodiments, three of R^{1a}, R^{1b}, R^{1c}, and R^{1d} are hydrogen. In some embodiments, R^{1a} is selected from —OEt,

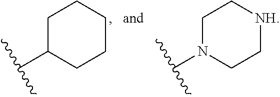 [SMILES: *C1CCCCC1 ; *N1CCNCC1]

In some embodiments, R^{1b} is —OMe. In some embodiments, R^{1d} is selected from —OH, —OMe, and

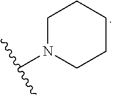 [SMILES: *N1CCCCC1]

In some embodiments, R² is selected from hydrogen,

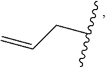 [SMILES: *CC=C]

methyl, ethyl,

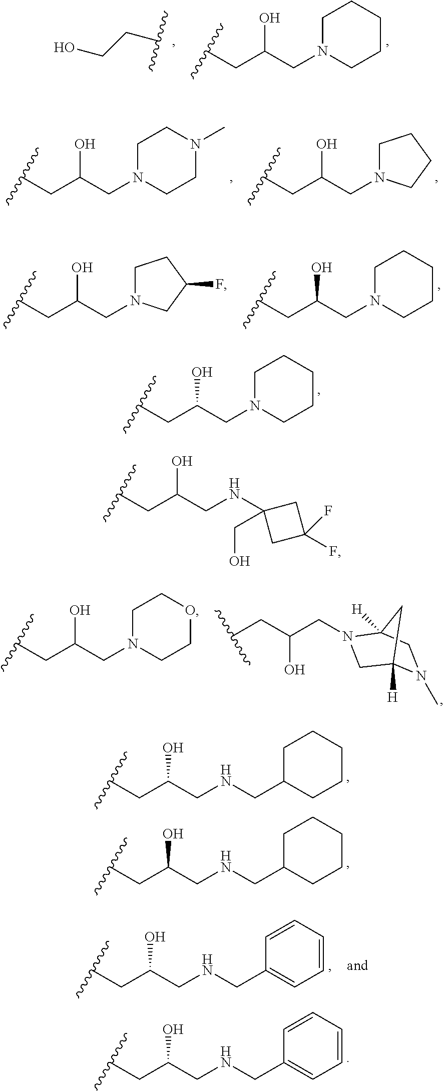

In some embodiments, and R^{3b} are each independently selected from hydrogen,

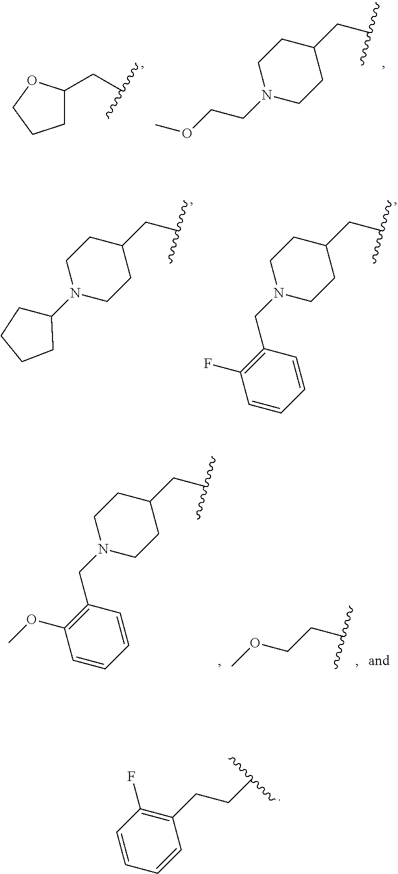

In some embodiments, R^{3a} and R^{3b} join together to form a ring selected from

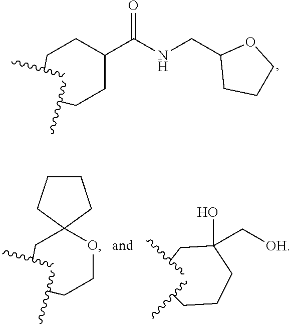 [SMILES: *CCC(CC*)C(=O)NCC1CCCO1 ; *CCOC1(C*)CCCC1 ; "and" ; *CCCC(O)(C*)CO]

In some embodiments, the compound of formula (II) is a compound having any one of formulas 2001-2028, or a pharmaceutically acceptable salt, solvate, hydrate, cocrystal, or prodrug thereof:

The disclosure provides a compound of formula (III), or a pharmaceutically acceptable salt, solvate, hydrate, cocrystal, or prodrug thereof:

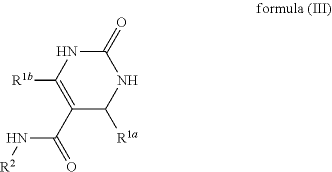

wherein in formula (III):

R^{1a} is selected from

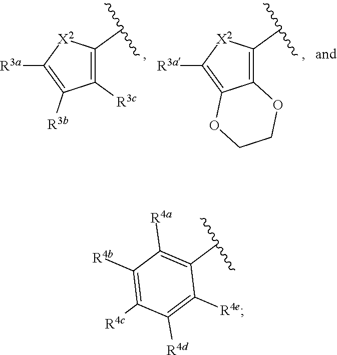

R^{1b} is selected from C₁-C₅-alkyl and —(C₁-C₅-alkyl)-O—(C₁-C₅-alkyl);
R² is selected from C₁-C₁₀-alkyl, C₃-C₁₀-cycloalkyl, —(CH₂)—C₃-C₁₀-cycloalkyl, and —(CH₂)—C₆-C₁₀-aryl;
X² and X^{2′} at each occurrence are independently selected from O and S; and
R^{3a}, R^{3b}, R^{3c}, R^{3a′}, R^{4a}, R^{4b}, R^{4c}, R^{4d}, and R^{4e} at each occurrence are independently selected from hydrogen, halo, C₁-C₅-alkyl, and C₁-C₅-alkoxy.

In some embodiments, R^{1a} is selected from

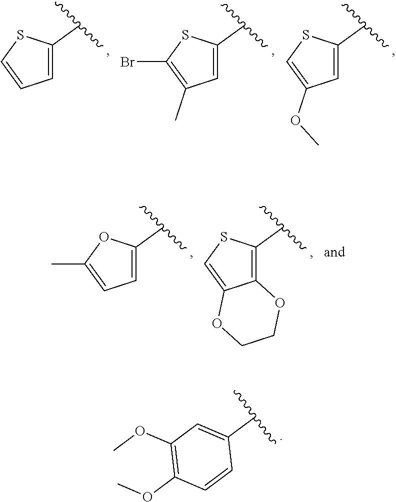

In some embodiments, R^{1b} is selected from methyl and

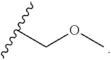 [SMILES: *COC]

In some embodiments, R² is selected from

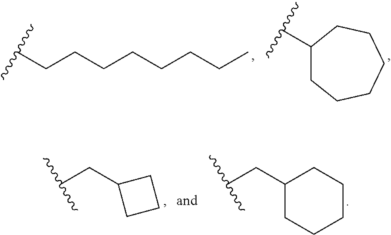

In some embodiments, compound of formula (III) is a compound having any one of formulas 3001-3018, or a pharmaceutically acceptable salt, solvate, hydrate, cocrystal, or prodrug thereof.

The disclosure provides a compound of formula (IV), or a pharmaceutically acceptable salt, solvate, hydrate, cocrystal, or prodrug thereof:

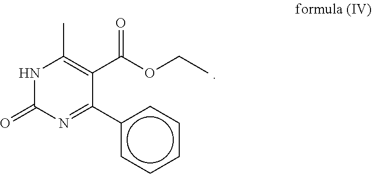

The disclosure provides a compound of formula (V), or a pharmaceutically acceptable salt, solvate, hydrate, cocrystal, or prodrug thereof:

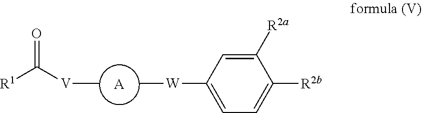

wherein in formula (V).
A is selected from

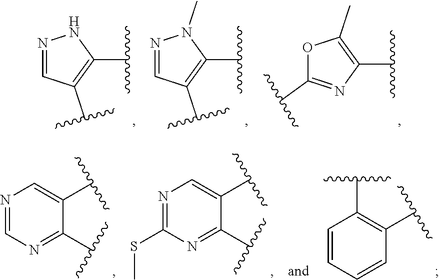

V is selected from a single bond and

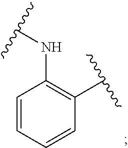 [SMILES: *Nc1ccccc1*]

W is selected from a single bond,

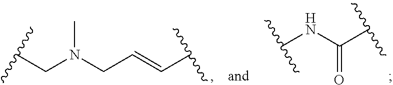

R¹ is selected from

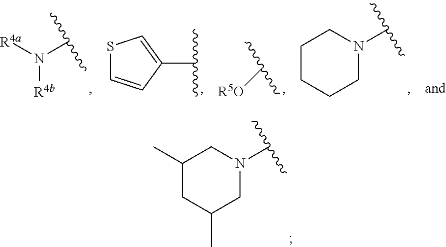

R^{2a} and R^{2b} are each independently selected from hydrogen, —OH, and C₁-C₅-alkoxy, or R^{2a} and R^{2b} are joined to form

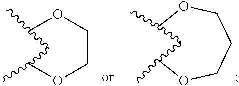

R^{4a} is selected from hydrogen and C₁-C₅-alkyl,
R^{4b} is selected from C₃-C₁₀-cycloalkyl, —(CH₂)—C₃-C₁₀-cycloalkyl,

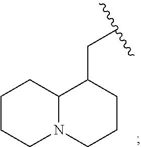 [SMILES: *CC1CCCN2CCCCC12]

and
R⁵ is selected from hydrogen, C₁-C₁₀-alkyl, and

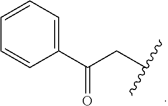 [SMILES: *CC(=O)c1ccccc1]

In some embodiments, the compound of formula (V) is a compound of formula (50), or pharmaceutically acceptable salt, solvate, hydrate, cocrystal, or prodrug thereof:

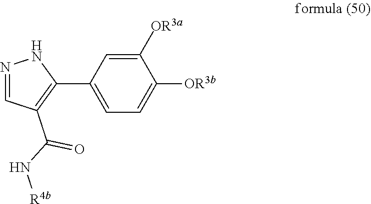

wherein in formula (50):
R^{3a} and R^{3b} are each independently C₁-C₅-alkyl or optionally can be joined to form

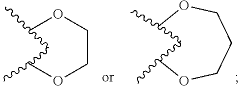

and
R^{4b} is selected from C₃-C₁₀-cycloalkyl and —(CH₂)—C₃-C₁₀-cycloalkyl.

In some embodiments, R^{3a} and R^{3b} are each methyl. In some embodiments, R^{4b} is selected from

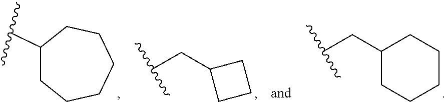

In some embodiments, the compound of formula (V) is a compound having any one of formulas 5001-5013, or a pharmaceutically acceptable salt, solvate, hydrate, cocrystal, or prodrug thereof.

The disclosure provides a compound of formula (VI), or a pharmaceutically acceptable salt, solvate, hydrate, cocrystal, or prodrug thereof:

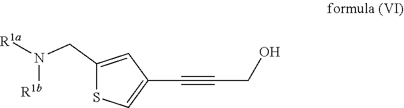

wherein in formula (VI):
R^{1a} and R^{1b} are each independently selected from C₁-C₅-alkyl,

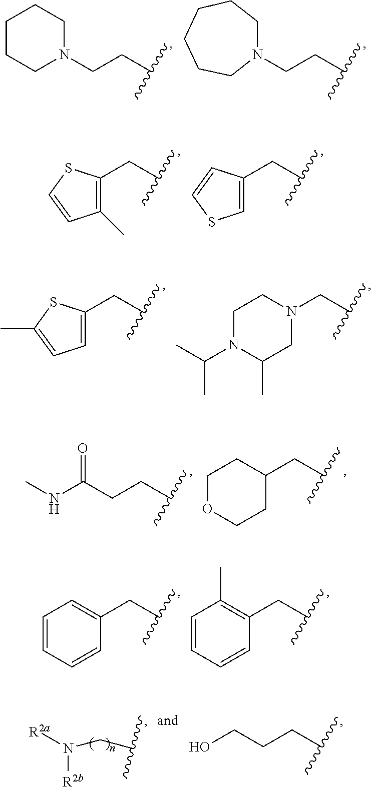

or R^{1a} and R^{1b} are joined together to form

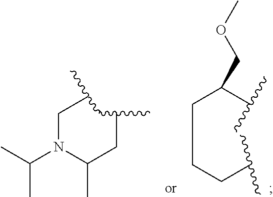

R^{2a} and R^{2b} at each occurrence are independently C₁-C₅-alkyl; and
n is an integer from 1 to 5.

In some embodiments, R^{2a} and R^{2b} are each methyl. In some embodiments, n is 2. In some embodiments, the compound of formula (VI) is a compound having any one of formulas 6001-6013, or a pharmaceutically acceptable salt, solvate, hydrate, cocrystal, or prodrug thereof.

The disclosure provides a compound of formula (VII), or a pharmaceutically acceptable salt, solvate, hydrate, cocrystal, or prodrug thereof:

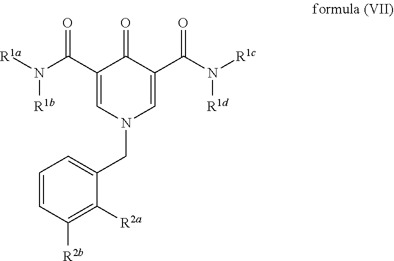

wherein in formula (VII):
R^{1a}, R^{1b}, R^{1c}, and R^{1d} are each independently selected from hydrogen, C₁-C₅-alkyl, C₃-C₁₀-cycloalkyl,

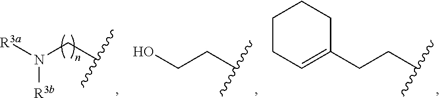

or R^{1a} and R^{1b} and/or R^{1c} and R^{1d} are joined to form,

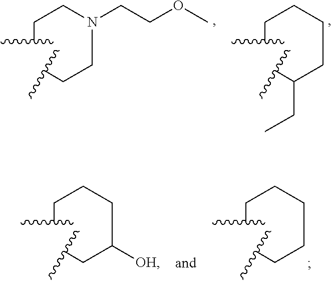

R^{2a} and R^{2b} are each independently selected from hydrogen and C₁-C₅-alkoxy;
R^{3a} and R^{3b} at each occurrence are independently C₁-C₅-alkyl; and
n is an integer selected from 1 to 5.

In some embodiments, R^{1a}, R^{1b}, R^{1c}, and R^{1d} are each independently selected from hydrogen, methyl,

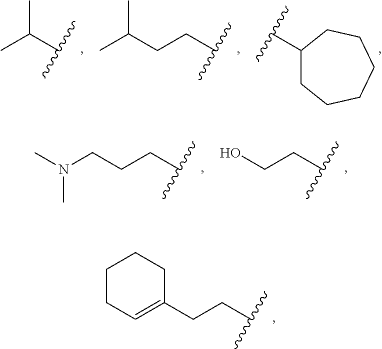

or R^{1a} and R^{1b} and/or R^{1c} and R^{1d} are joined to form

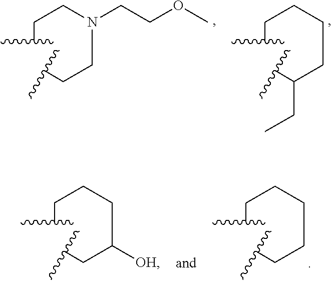

In some embodiments, the compound of formula (VII) is a compound having any one of formulas 7001-7006, or a pharmaceutically acceptable salt, solvate, hydrate, cocrystal, or prodrug thereof.

The disclosure provides a compound of any one of formula (VIIIa)-(VIIIf), or a pharmaceutically acceptable salt, solvate, hydrate, cocrystal, or prodrug thereof:

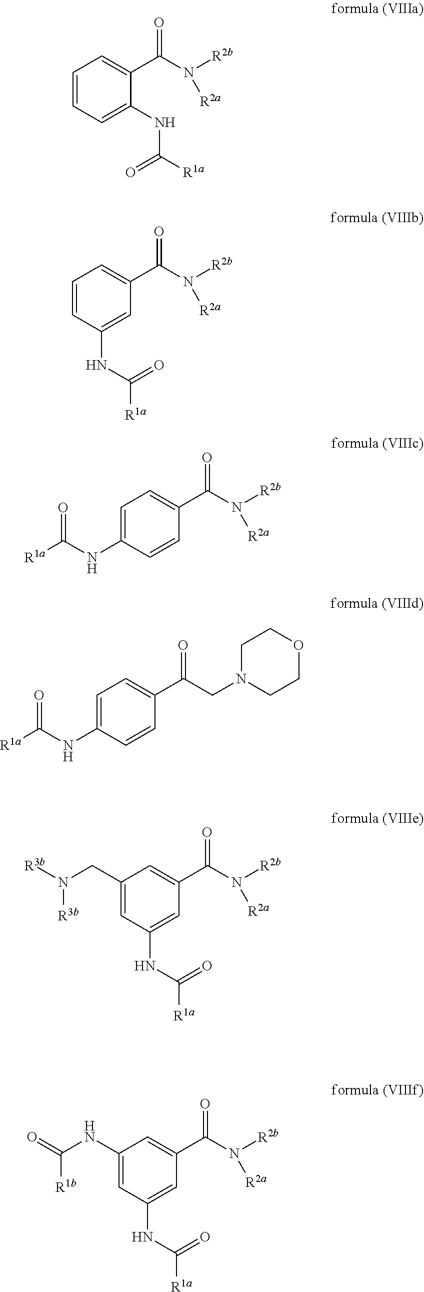

R^{1a} and R^{1b} are each independently selected from C₁-C₅-alkyl,

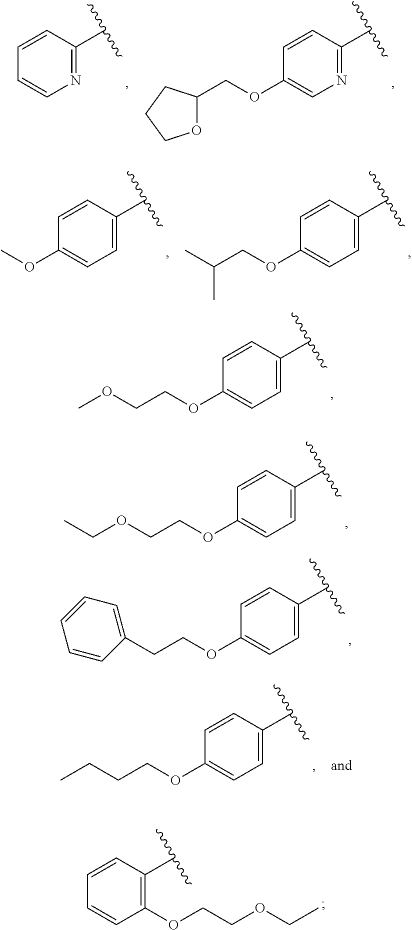

and
R^{2a}, R^{2b}, R^{3a}, and R^{3b} are each independently selected from hydrogen, C₁-C₅-alkyl,

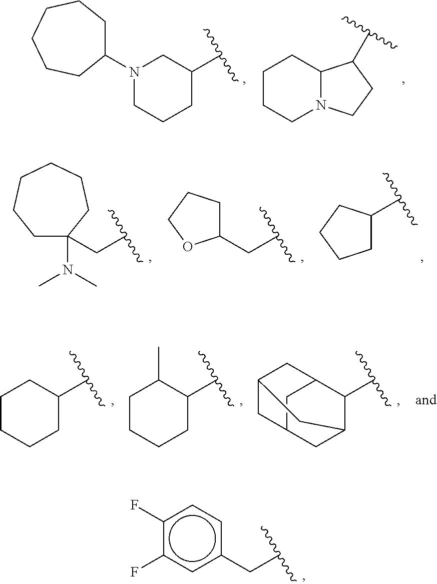

or R^{2a} and R^{2b} and/or R^{3a} and R^{3b} are joined together to form

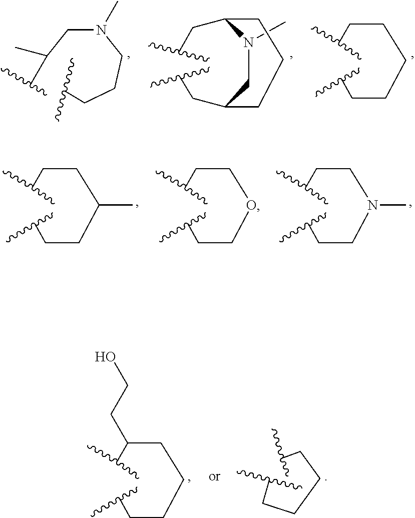

In some embodiments, R^{1a} and R^{1b} are each independently selected from methyl, ethyl,

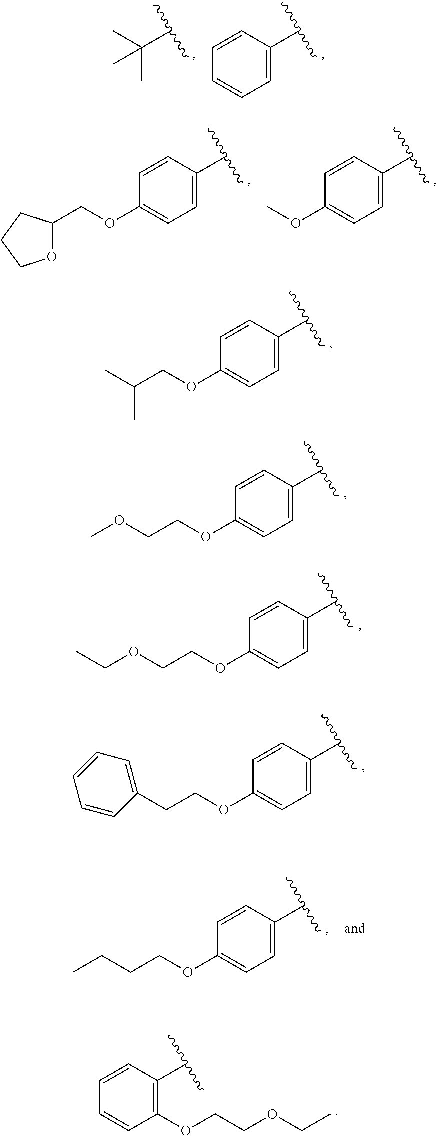

In some embodiments, and R^{2b} are each independently selected from hydrogen, methyl,

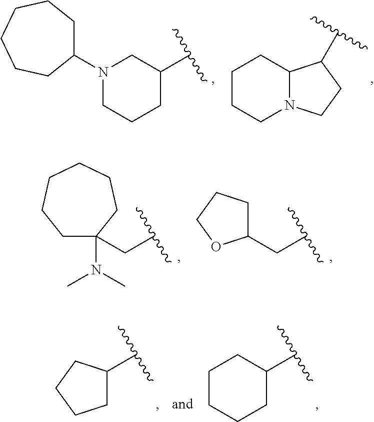

or R^{2a} and R^{2b} are joined together to form

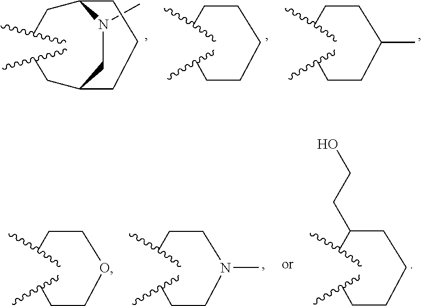

In some embodiments, R^{2a}, R^{2b}, R^{3a}, and R^{3b} are each independently selected from hydrogen,

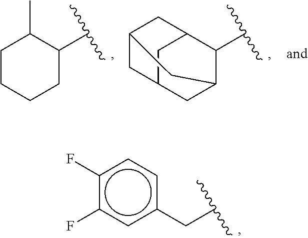

or R^{2a} and R^{2b} and/or R^{3a} and R^{3b} are joined together to form

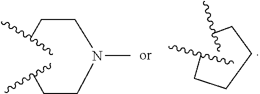

In some embodiments, the compound is selected from:
a) a compound of formula (VIIIa) having any one of formulas 8001-8010, or a pharmaceutically acceptable salt, solvate, hydrate, cocrystal, or prodrug thereof,
b) a compound of formula (VIIIb) having any one of formulas 8101-8105, or a pharmaceutically acceptable salt, solvate, hydrate, cocrystal, or prodrug thereof,
c) a compound of formula (VIIIc) having formula 8201, or a pharmaceutically acceptable salt, solvate, hydrate, cocrystal, or prodrug thereof,
d) a compound of formula (VIIId) having formula 8301, or a pharmaceutically acceptable salt, solvate, hydrate, cocrystal, or prodrug thereof,
e) a compound of formula (VIIIe) having any one of formulas 8401-8403, or a pharmaceutically acceptable salt, solvate, hydrate, cocrystal, or prodrug thereof, and
f) a compound of formula (VIIIf) having formula 8501, or a pharmaceutically acceptable salt, solvate, hydrate, cocrystal, or prodrug thereof.

In some embodiments, the compound of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42 UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72 inhibits SKI complex activity.

In some embodiments, the compound of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42 UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72 inhibits viral replication.

In some embodiments, the compound of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42 UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72 induces interferon signaling.

The disclosure provides a pharmaceutical composition for treating a condition alleviated by inhibiting SKI complex activity, the pharmaceutical composition comprising one or more compounds of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72 or a pharmaceutically acceptable salt, solvate, hydrate, cocrystal, or prodrug thereof, and a pharmaceutically acceptable carrier.

The disclosure provides a pharmaceutical composition for treating a condition alleviated by inhibiting viral replication, the pharmaceutical composition comprising one or more compounds of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18 D49, UMB18 D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72 or a pharmaceutically acceptable salt, solvate, hydrate, cocrystal, or prodrug thereof, and a pharmaceutically acceptable carrier.

The disclosure provides a pharmaceutical composition for treating a condition alleviated by inducing interferon signaling, the pharmaceutical composition comprising one or more compounds of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18 D49, UMB18 D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72 or a pharmaceutically acceptable salt, solvate, hydrate, cocrystal, or prodrug thereof, and a pharmaceutically acceptable carrier.

In some embodiments, the condition is selected from a viral infection, a bacterial infection, and cancer. In some embodiments, the bacterial infection is selected from a lung infection, skin infection, soft tissue infection, gastrointestinal infection, urinary tract infection, meningitis, and sepsis. In some embodiments, the cancer is selected from adrenocortical cancer, hepatocellular cancer, hepatoblastoma, malignant melanoma, ovarian cancer, Wilm's tumor, Barrett's esophageal cancer, prostate cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. In some embodiments, the viral infection is caused by influenza, Middle East respiratory syndrome-related coronavirus (MERS-CoV), rhinovirus, polio, measles, Ebola, Coxsackie, West Nile, yellow fever, Dengue fever, lassa, lymphocytic choriomeningitis, Junin, Machupo, guanarito, hantavirus, Rift Valley Fever, La Crosse, California encephalitis, Crimean-Congo, Marburg, Japanese Encephalitis, Kyasanur Forest, Eastern equine encephalitis, Western equine encephalitis, severe acute respiratory syndrome (SARS), severe acute respiratory syndrome coronavirus 2 (SARS-CoV-2), parainfluenza, Tacaribe, Pichinde viruses, bat coronaviruses, seasonal coronaviruses (229E, OC43, HKU1 and NL63), enterovirus 68, enterovirus 71. In some embodiments, the viral infection is caused by influenza. In some embodiments, the viral infection is caused by severe acute respiratory syndrome coronavirus 2 (SARS-CoV-2).

The disclosure provides a method of treating a condition by inhibiting SKI complex activity in a patient in need of said treatment, the method comprising administering to the patient a therapeutically effective amount of a compound of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22__D48, UMB18_D49, UMB18_D50, UMB22_5_D51, UMB22_16 D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or a pharmaceutically acceptable salt, solvate, hydrate, cocrystal, or prodrug thereof.

The disclosure provides a method of treating a condition by inhibiting viral replication in a patient in need of said treatment, the method comprising administering to the patient a therapeutically effective amount of a compound of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or a pharmaceutically acceptable salt, solvate, hydrate, cocrystal, or prodrug thereof.

The disclosure provides a method of treating a condition alleviated by inducing interferon signaling in a patient in need of said treatment, the method comprising administering to the patient a therapeutically effective amount of a compound of any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or a pharmaceutically acceptable salt, solvate, hydrate, cocrystal, or prodrug thereof.

### BRIEF DESCRIPTION OF THE FIGURES

FIG. 1 illustrates the growth curves of S. cerevisiae expressing individual Influenza virus genes in the presence of 2% galactose to induce viral gene expression. Growth curves over 48 hours and OD600 were measured every 30 minutes.

FIG. 2 illustrates the inhibition of Influenza virus replication when SKIV2L and TTC37 are knocked down by siRNA. A549 cells were transfected with 2 pools of siRNA targeting each of the identified genes. After 3 days of siRNA treatment, Influenza virus was added to cells and then analyzed at 24 hours post infection by plaque assay to monitor virus growth.

FIG. 3 illustrates a model of SILCS mapping. Predicted orientation of compound 96509034 (UMBCADD-0018, CPK) bound to Ski8 (white surface representation) along with the SILCS FragMaps (apolar: green, H-bond acceptor: red, H-bond donor: blue, and positive: cyan at −0.9 kcal/mol). Ligand binding is driven by occupation of aliphatic and aromatic groups in the apolar FragMaps, by the hydroxyl in the H-bond donor and acceptor FragMaps and by the fluorine atom in the H-bond acceptor FragMap. The SILCS methodology accounts for protein flexibility allowing the aromatic ring to penetrate under the protein surface during SILCS-MC docking.

FIG. 4 illustrates the testing of SKI complex targeted compounds against Influenza virus. Compound names are on the Y-axis and PFU/virus/ml is on the X-axis. All results are from plaque assays for Influenza virus infection.

FIG. 5 illustrates the testing of SKI complex targeted compounds against Influenza virus round 2). Compound names are on the Y-axis and PFU/virus/ml is on the X-axis.

FIGS. 6A-6D illustrate yeast suppressor screening identifies the SKI complex as a suppressor NS1 and ORF4a-mediated slow growth; FIG. 6-A: Genes from the IAV genome (CA09 sequences) were cloned into a galactose inducible expression vector and transformed into yeast. Different clones were picked and analyzed for growth rate (as determined by OD600) in galactose containing media. Plotted is the mean OD600 measures over a 48 h growth period looking at 4 independent colonies across 2 independent experiments (error bars are the standard deviation across assessed colonies. FIG. 6-B: Yeast knockouts for each component of the SKI complex were transformed with the NS1 galactose inducible expression plasmid, or empty vector control (EV). Growth rate of these yeast was measured over a 48 h culture period. Mean OD600 between 3 independent colonies in 2 independent experiments is plotted with error bars being the standard deviation. FIG. 6-C: As in B, but with ORF4a expression plasmid. FIG. 6-D: Protein extracts were made from SKI knockout yeast (or wild type control [WT]) as assessed in B and C and samples were western blotted to look for NS1 and ORF4a expression through a C-terminal GFP tag (actin was used as a loading control).

FIG. 7 illustrates that knockdown of the SKI complex by siRNA inhibits replication of IAV and MERS-CoV; FIG. 7-A: A549 cells were transfected with siRNAs targeting the different components of the SKI complex using two unique sequences for each of the three target genes along with scrambled and mock controls. After 3 days of transfection, cells were infected with IAV at MOI 0.01. After 24 h, supernatant was collected, and viral titer assessed by plaque assay. Plotted is the mean PFU/ml from 3 independent experiments with error bars being standard deviation. FIG. 7-B: as in A but with Huh7 cells and MERS-CoV infection at MOI 0.1. Virus titer was determined by TCID50 assay. Plotted is the mean TCID50/ml from 3 independent experiments with error bars being standard deviation. FIG. 7-C: a third siRNA sequence for each of the three SKI genes was transfected into A549 cells for three days, at which point the cells were infected and assessed as in A. Plotted is a representative experiment of two showing the mean PFU/ml from triplicate wells of infection. FIG. 7-D: A549 cells were transfected for three days as described and collected in Trizol for qRT-PCR analysis of each of the SKI genes being targeted by siRNA (all three unique sequences). Data are a representative experiment of 3 performed in triplicate wells. PCR reads were normalized with GAPDH and fold change was set relative to scrambled siRNA transfected cells. FIG. 7-E: A549 and Huh7 cells were transfected with SKIV2L targeting siRNA (sequence 1 and 2) for three days prior to collection in RIPA lysis buffer. Samples were western blotted for SKIV2L or tubulin as a loading control. Data is representative of 2 independent repeats. FIG. 7-F: A549 and FIG. 7-G: Huh7 cells were transfected with siRNAs targeting the SKI complex and cell viability was assessed over the three-day period by CellTiter-Glo assay. Data are the mean relative luminescence set relative to scrambled control from a representative experiment performed in quadruplicate of 3 (A549) or 2 (Huh7) independent experiments.

FIG. 8 illustrates modelling compounds to bind to the SKI complex and screening for antiviral activity. The 3D structure of SKI8 was subjected to SILCS simulations from which the FIG. 8-A: FragMaps (Mesh representations for apolar [green, −0.9 kcal/mol], hydrogen-bond donor [blue, −0.6 kcal/mol], hydrogen bond acceptor [red, −0.6 kcal/mol], positive [cyan, −1.2 kcal/mol] and negative −1.2 kcal/mol] functional groups) were calculated from which fragment-binding Hotspots were determined (all Hotspots as blue spheres with those defining the identified binding site as larger spheres colored by ranking (low to high as blue to red). FIG. 8-B: expanded view of putative binding site with the FragMaps and the Hotspots defining that site. FIG. 8-C: putative binding site with the FragMaps, the pharmacophore features (spheres, aromatic [cyan] and hydrogen-bond donor [blue]) and the SILCS-MC docked orientation of the ligand. Compounds predicted to bind from the SILCS simulations were purchased and screened for antiviral activity. A549 cells were infected with IAV at MOI 0.01 for 24 h and treated at 50 μM or 10 μM. Virus was collected, and PFU/ml determined by plaque assay. FIG. 8D: compounds 1-20 were tested at each concentration with single wells of infection. 50 μM data is from one experiment, 10 μM data is from two independent experiments with error bars being standard deviation. Dotted line to denote the DMSO control PFU/ml for ease of visualization. FIG. 8-E: compounds 21-40 were tested at each concentration. Data from two independent experiments in both cases with error bars being standard deviation. UMB18 included as a positive control in both repeats since it appeared to be a good candidate hit in D. FIG. 8-F: structural variants of UMB18 were investigated to test if any had greater antiviral activity than the lead compound. Data from two independent experiments in both cases with error bars being standard deviation. UMB18-2 was also listed as UMB40 which was only realized after experiments were performed. FIGS. 8-G-J: chemical structures of the four compounds considered as hits to follow up on.

FIG. 9 illustrates SKI targeting compounds have antiviral activity against IAV and MERS-CoV. FIG. 9-A: A549 cells were infected with IAV at MOI 0.01 and treated with UMB18 for 24 h. Drug was added at the indicated range of concentrations. Based on the stock of compound, 0.5% DMSO acted as the vehicle control for 50 μM and 25 μM while 0.1% acts as the control for all other concentrations. Virus was collected after 24 h, and PFU/ml determined by plaque assay. Data are from 3 independent experiments performed in triplicate with mean PFU/ml displayed and error bars representing standard deviation. FIG. 9-B: Huh7 cells were infected with MERS-CoV at MOI 0.1 and treated with UMB18 for 24 h. Drug was added with comparable controls to A. Virus was collected and titer determined by TCID50 assay. Data are from 3 independent experiments as in A. FIG. 9-C: A549 cells were treated with the displayed concentrations of UMB18 for 24 h, after which CellTiter-Glo assays were performed to assess cell viability. Data are from a representative experiment of three independent experiments all performed in quadruplicate. Plotted are the mean relative luminescence to matched DMSO vehicle control with error bars displaying the standard deviation. FIG. 9-D: As in C but for Huh7 cells. FIGS. 9E and 9F: As A and B but for UMB18-2. FIG. 9-G: A549 cells were treated with UMB28 or UMB36 and compared with UMB18 at either 50 μM or 10 μM (with 0.5% or 0.1% DMSO being the appropriate negative controls) and infected with IAV at MOI 0.01. Virus was collected after 24 h, and PFU/ml determined by plaque assay. Data are from 3 independent experiments (one of a single well and two of triplicate wells) with the mean PFU/ml displayed and error bars being the standard deviation. FIG. 9-H: As in G but Huh7 cells were treated and infected with MERS-CoV at MOI 0.1 for 24 h. Virus titer was determined by TCID50 assay. Data are from 3 independent experiments all of triplicate wells with the mean TCID50/ml displayed and error bars being the standard deviation.

FIG. 10 illustrates that UMB18 inhibits filovirus infection. Huh7 cells were treated with UMB18 for test, toremifene citrate (TOMF) as a positive control and DMSO as a negative control. Treatments were over an 8-point dose curve with 3-fold dilutions, each in triplicate. Cells were infected with FIG. 10-A: Ebola virus Makona strain (EBOV) or FIG. 10-B: Marburg virus Angola strain (MARV) for 48 h. Cells were fixed and labelled with antibodies to VP40 for each virus. Infected cells were detected by peroxidase secondary labeling to determine the percentage inhibition of infection by each treatment. Cytotoxicity is also displayed which was determined by CellTiter-Glo assay on uninfected samples. Data are from one representative of two independent experiments. Dotted line is at 50% inhibition for determining IC50 values.

FIG. 11 illustrates that SKI targeting lead compounds inhibit viral mRNA and protein production. A time of addition experiment was performed to investigate what stages of IAV infection UMB18 (FIG. 11-A) and UMB18-2 (FIG. 11-B) inhibit. A549 cells were plated and treated with drug 2 h prior to infection (−2 h), at the time of infection (0 h) or 2 h after virus was added to cells (+2 h). Cells were infected at MOI 0.01 for 24 h, at which point supernatant was collected and used to determine viral production by plaque assay. Mean PFU/ml and standard deviation are displayed from 2 independent experiments performed in triplicate for each compound with error bars being standard deviation. FIG. 11-C: A549 cells were infected with IAV at MOI 3 for 8 h with UMB18 treatment. Cells were collected in Trizol and NS1 mRNA transcript analyzed by qRT-PCR. Input levels were normalized to GAPDH and fold change of transcript levels were determined relative to DMSO control for each concentration of compound. Data are from 3 independent experiments performed on triplicate wells. FIG. 11-D: As in C, but with UMB18-2 treatment. FIG. 11-E: Using the same extracted RNA as in the qRT-PCR experiments for D, an M-RTPCR protocol was used to amplify all IAV segments. These were then run on an agarose gel and imaged. Displayed are the amplifications from two independent wells of treatment and infection for UMB18-2 at 50 μM and 10 μM and three wells for DMSO controls. FIG. 11-F: In parallel with collecting cells in Trizol for qRT-PCR analysis in C and D, a separate well of cells were also collected in RIPA lysis buffer and used for western blotting of NS1 (or tubulin for loading control) to corroborate the mRNA data. Displayed is a representative blot of the three independent repeats for each compound.

FIG. 12 illustrates that UMB18-2 inhibits SARS-CoV and SARS-CoV-2. FIG. 12-A: Huh7-ACE2 cells were infected with SARS-CoV and treated with UMB18-2 at 50 μM or 10 μM (with 0.5% or 0.1% DMSO being the appropriate negative controls) for 24 h. Supernatant was collected and used for TCID50 assay to determine viral titer. Mean TCID50/ml and standard deviation are displayed from 3 independent experiments performed in triplicate. FIG. 12-B: As in A but using Vero cells and infection with SARS-CoV-2 (it was found that Huh7-ACE2 did not release virus particles and therefore had to use a different cell line). FIG. 12-C: As in B but infection at MOI 0.01. Cells that were infected in B (MOI 0.1) were collected in Trizol after 24 h infection and used for qRT-PCR analysis. Primers targeting N (FIG. 12-D) or RdRp (FIG. 12-E) were used. Input levels were normalized to 18S RNA and fold change of transcript levels were determined relative to DMSO control for each concentration of compound. Data are from the same 3 independent experiments as B. Individual data points are displayed because of one experiment having much higher reads with 10 M treatment, the two points are the same samples with the two different primers.

FIGS. 13-A and 13-B illustrate the structure of SKI Complex used to model compounds that bind to pocket at interface of subunits; docking of compound on SKI8 on face that binds to SKI3.

FIGS. 14-A and 14-B illustrate the validation of large colony suppressors from yeast knockout screen. Genetic suppressors were revalidated by transforming known knockout yeast collected from an arrayed library. Proteins in a variety of pathways were identified including the SKI complex protein, SKI2.

FIG. 15 illustrates the siRNA knockdown of SKI complex proteins in influenza virus infection (A549 cells, 2 siRNAs tested per gene, 72 hour transfection before infection with NL09 (H1N1), MOI 0.01 for 24 hrs).

FIG. 16 illustrates the siRNA knockdown of SKI complex proteins in MERS Coronavirus infection.

FIG. 17 illustrates the initial compound series modeled to bind to SKI8/SKI3 interface (influenza infection, MOI 0.01, 24 hr time point).

FIG. 18 illustrates results from lead hit 96509034, initial SAR performed from available compounds (influenza infection, MOI 0.01, 24 hr time point).

FIG. 19 illustrates additional results from lead hit 96509034.

FIG. 20 illustrates the determination of broader concentration curve on SKI targeted compounds. Reduction of virus growth was found via readout of fluorescence in infected cells.

FIGS. 21-A and 21-B illustrate inhibition of SARS-CoV and MERS-CoV replication with 96509034 treatment.

FIG. 22 illustrates the structures of several identified compounds.

FIGS. 23-A-23-C illustrate the similarity search of analogs of UMB18/96509034 to identify if either of the two-ring substructures of the lead have activity. Two compounds were obtained and tested for each two-ring substructure.

FIGS. 24-A-24-R illustrate the chemical structures, log P values, and molecular weights of compounds UMB28-1 to UMB28_18, UMB5_1 to UMB57, UMB10_1 to UMB10_7, UMB22_1 to UMB22_20, UMB40_1 to UMB40_10, UMB42_1 to UMB42_12, and UMB23_1 to UMB23_14.

FIGS. 25-A-25-S depict the structures of compounds SILCS 1-72.

FIG. 26 illustrates IAV data for compounds UMB 41-116.

FIG. 27 illustrates IAV data by scaffold for compounds UMB 41-116.

FIG. 28 illustrates a similarity search of MolPort database undertaken on UMB22_5 and 16 to develop initial structure-activity relationship. SARS2 data. Based on average over all concentrations tested only a few compounds show activity with the best being 4- to 5-fold worse than UMB18G as well as worse than the leads UMB22_5 and UMB22_16. Subsequent development of these leads was undertaken in the optimization efforts with SilcsBio LLC and Dalriada.

FIG. 29 illustrates SILCS Hotspots binding site identification on WDR61.

FIG. 30 illustrates lead compounds.

FIGS. 31-A-31-C illustrate SILCS FragMaps used to guide ligand design overlaid on structure of WDR61 and lead compound UMB18. FIG. 31-A) SILCS FragMaps overlaid on the structure of WDR61 (cyan cartoon) including UMB18 (spheres, atom type colored). FIG. 31-B and FIG. 31-C) SILCS B) standard and C) halogen FragMaps overlaid on WDR61 binding site including UMB18 (sticks, atom colored). UMB18 is shown in the SILCS-MC docked orientation. Standard SILCS Fragmaps in mesh include generic apolar (green), generic hydrogen-bond donor (blue) and acceptor (red), negatively charged (orange) and positively charged (cyan) functional groups and the SILCS halogen FragMaps include fluoro- (magenta), chloro- (pink) and bromo- (brown) benzene FragMaps. FragMaps shown at a GFE cutoff of −1.2 kcal/mol or −0.9 kcal/mol for the hydrogen bond donor and acceptor FragMaps.

FIG. 32 illustrates UMB18 in it SILCS-MC docked conformation (sticks, atom colored) in two orientations to aid visualization along with the atom-based GFE scores in green.

FIG. 33-A and FIG. 33-B) illustrate SILCS A) standard and B) halogen FragMaps overlaid on WDR61 binding site including UMB28 (sticks, atom colored). UMB28 is shown in the SILCS-MC docked orientation. FIG. 33-C and FIG. 33-D) Atom-based GFE scores for UMB28 in two orientations to facilitate visualization. Standard SILCS Fragmaps in mesh include generic apolar (green), generic hydrogen-bond donor (blue) and acceptor (red), negatively charged (orange) and positively charged (cyan) functional groups and the SILCS halogen FragMaps include fluoro- (magenta), chloro- (pink) and bromo- (brown) benzene FragMaps. FragMaps shown at a GFE cutoff of −1.2 kcal/mol or −0.9 kcal/mol for the hydrogen bond donor and acceptor FragMaps.

FIG. 34-A and FIG. 34-B illustrate SILCS A) standard and B) halogen FragMaps overlaid on WDR61 binding site including UMB22_5 (sticks, atom colored). UMB22_5 is shown in the SILCS-MC docked orientation. FIG. 34-C and FIG. 34-D) Atom-based GFE scores for UMB22_5 in two orientations to facilitate visualization. Standard SILCS Fragmaps in mesh include generic apolar (green), generic hydrogen-bond donor (blue) and acceptor (red), negatively charged (orange) and positively charged (cyan) functional groups and the SILCS halogen FragMaps include fluoro- (magenta), chloro- (pink) and bromo- (brown) benzene FragMaps. FragMaps shown at a GFE cutoff of −1.2 kcal/mol or −0.9 kcal/mol for the hydrogen bond donor and acceptor FragMaps.

FIG. 35-A and FIG. 35-B illustrate SILCS A) standard and B) halogen FragMaps overlaid on WDR61 binding site including UMB22_16 (sticks, atom colored). UMB22_16 is shown in the SILCS-MC docked orientation. FIG. 35-C and FIG. 35-D) Atom-based GFE scores for UMB22_16 in two orientations to facilitate visualization. Standard SILCS Fragmaps in mesh include generic apolar (green), generic hydrogen-bond donor (blue) and acceptor (red), negatively charged (orange) and positively charged (cyan) functional groups and the SILCS halogen FragMaps include fluoro- (magenta), chloro- (pink) and bromo- (brown) benzene FragMaps. FragMaps shown at a GFE cutoff of −1.2 kcal/mol or −0.9 kcal/mol for the hydrogen bond donor and acceptor FragMaps.

FIG. 36 illustrates SARS2 infection data for SILCS compounds 1-72 including UMB18, UMB 28 and UMB 22 scaffolds.

FIG. 37 illustrates Cellular toxicity data for SILCS compounds 1-72 including UMB18, 28 and 22 scaffolds.

FIG. 38 illustrates SILCS predicted binding orientations to WDR61.

FIG. 39 illustrates the structures of compounds UMB18_D43 (SICLS43), UMB.28_D39 (SILCS39), and UMB22_D62 (SICLS62).

FIG. 40 illustrates non-limiting examples of UMB18 analogs.

FIG. 41 illustrates non-limiting examples of UMB18 analogs with a modified central ring.

FIG. 42 illustrates non-limiting examples of UMB18 analogs.

FIG. 43 illustrates non-limiting examples of UMB22 analogs.

FIG. 44 illustrates non-limiting examples of UMB22 analogs.

FIG. 45 illustrates Surface Plasmon Resonance (SPR) experiments of UMB18, SILCS4, SILCS32 and SILCS35.

FIG. 46 illustrates SPR experiments which provide a kinetic analysis of SILCS4 and SILCS32.

FIG. 47 illustrates SILCS modeling, 72 SilcsBio analogs.

FIG. 48 illustrates SILCS modeling, 72 SilcsBio analogs following BML optimization of the SILCS FragMaps.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include, but are not limited to, the compounds described herein and, more specifically, compounds of any of one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, and their features and limitations as described herein.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., increased sensitivity to apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. In some embodiments, the biological molecules modulated by the methods and compounds of the disclosure to effect treatment may include the SKI complex.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a compound of any of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone.

Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of any of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16 D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72) is a prodrug within the scope of the disclosure. Various forms of prodrugs are well known in the art. A comprehensive description of pro drugs and prodrug derivatives are described in. (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R)C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)— radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R² of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R)C(O)OR^{a}, —N(R^{a})C(O)R^{a}, —N(R^{a})C(O)N(R^{a})₂, N(R^{a})C(NR^{a})N(R^{a})₂, —N(R^{a})S(O)R^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the disclosure. All embodiments of the disclosure can, in the alternative, be more specifically defined by any of the transitional terms “comprising,” “consisting essentially of,” and “consisting of.”

### SKI Complex Modulators

Novel antivirals are needed that are effective against a wide range of viruses for both current and future viral strains. Targeting of host pathways that are responsible for controlling viral replication are a novel way of inhibiting a range of viruses. The SKI complex controls the level of RNA in a cell as well as impacting the Interferon signaling pathway. Modulation of the SKI complex by small molecules affects both its RNA degradation capacity and also affects Interferon levels in the cell, of which both can reduce viral replication. The disclosure demonstrates that modulation of the SKI complex by small molecules is effective against both the Influenza virus and MERS Coronavirus. The disclosure also includes compounds useful as broad spectrum antiviral that inhibit the SKI complex.

The SKI complex is an RNA helicase complex involved with various aspects of RNA metabolism (K. Januszyk, C. D. Lima, The eukaryotic RNA exosome. (Current opinion in structural biology 24, 132-140, 2014). There have been some suggestions that the complex is involved with regulation of the interferon response (S. C. Eckard et al., The SKIV2L RNA exosome limits activation of the RIG-I-like receptors. Nature immunology 15, 839-845, 2014), and there is a link to cap-snatching by influenza virus (ncbi.nlm.nih.gov/pmc/articles/PMC6217988/), but beyond this, it is a protein complex that has not been heavily linked to viral replication. Using a yeast suppressor screen, genetic interaction between proteins of IAV and MERS-CoV and the SKI complex, which developed into our identification of the SKI complex was identified as being a potential antiviral target. Three chemical structures were identified that display broad-spectrum antiviral activity, with the lead compounds inhibiting influenza, all three pathogenic human coronaviruses and filoviruses, all of which cause significant human morbidity and mortality. Without any particular limitation, it is suggested that the mechanism of antiviral action is an inhibition of viral mRNA production.

The role of the SKI complex in viral replication was investigated because work in yeast suggested a genetic interaction between viral proteins and the yeast protein complex. Suppressor screening was previously used to identify SIRT1 as a proviral factor for MERS-CoV replication (Weston2019). Here there was added in screening data for IAV NS1 to find that this protein and ORF4a of MERS-CoV may interact with the SKI complex.

Viral infection can have a huge burden on human health. Influenza has historically caused numerous large epidemics and pandemics such as 1918 Spanish 'flu and 2009 Swine 'flu. Ebola has causes sporadic outbreaks since the 1970s, but in recent years these have been growing in scale. The 2014 West Africa Ebola outbreak saw over 28,000 people contract the disease and an ongoing outbreak has close to 4,000 cases. Coronaviruses have always posed a threat of mass spread because of their respiratory transmission. 2002-2003 saw the emergence of SARS-CoV which infected over 8,000 people, killing roughly 10%, in a matter of months, while MERS-CoV has sporadically spread since 2012, causing around 2,500 infections with a case fatality rate of around 35%.

The year 2020 has seen the rapid emergence of a novel human coronavirus, SARS-CoV-2, which in a matter of months spread from China, became a pandemic and has infected over 3 million people and counting. These outbreaks highlight the huge lack of antiviral therapeutic options available for treatment. Two complimentary approaches for this are to find antivirals that target multiple viruses, such as nucleotide analogues (e.g. remdesivir, ncbi.nlm.nih.gov/pmc/articles/PMC5844999/), and antivirals that target the host. Combination therapy is a highly effective strategy to limit viral resistance as clearly demonstrated for HIV (ncbi.nlm.nih.gov/pmc/articles/PMC3088245) and having multiple broad-spectrum approaches will be a powerful way to combat viral infection in the future. Herein it is demonstrated that the SKI complex is a potential host-directed broad-spectrum antiviral target.

The disclosure provides a compound of formula (I), or a pharmaceutically acceptable salt, solvate, hydrate, cocrystal, or prodrug thereof:

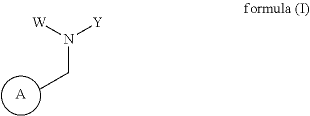

wherein in formula (I):
W is selected from C₁-C₅-alkyl and

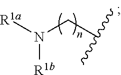

Y is selected from H,

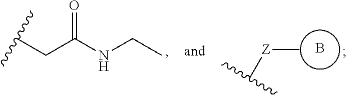

A and B are each independently selected from:

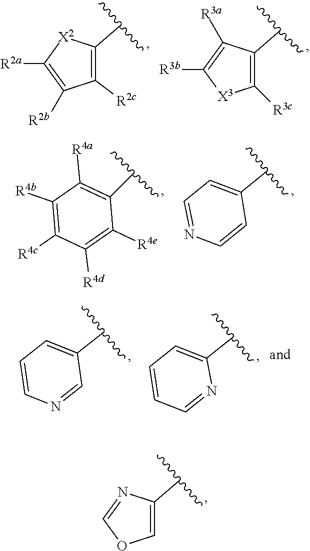

X² and X³ at each occurrence are independently selected from O and S;
Z is selected from —CH₂— and —C(O)—;
R^{1a} and R^{1b} at each occurrence are independently C₁-C₅-alkyl;
R^{2a}, R^{2b}, R^{2c}, R^{3a}, R^{3b}, R^{3c}, R^{4a}, R^{4b}, R^{4c}, R^{4d}, and R^{4e} at each occurrence are independently selected from hydrogen, halo, C₁-C₅-alkyl, trifluoromethyl, —(C₁-C₅-alkyl)-OH, phenyl, thiophenyl, furyl, and —C(O)O—C₁-C₅-alkyl; and
n is an integer selected from 1 to 5.

In some embodiments, W is C₁-C₅-alkyl.

In some embodiments, is H or

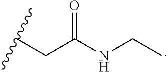 [SMILES: *CC(=O)NCC]

In some embodiments, the compound of formula (I) is a compound of formula (10), or a pharmaceutically acceptable salt, solvate, hydrate, cocrystal, or prodrug thereof:

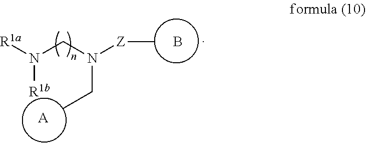

In some embodiments, A and B are each independently selected from:

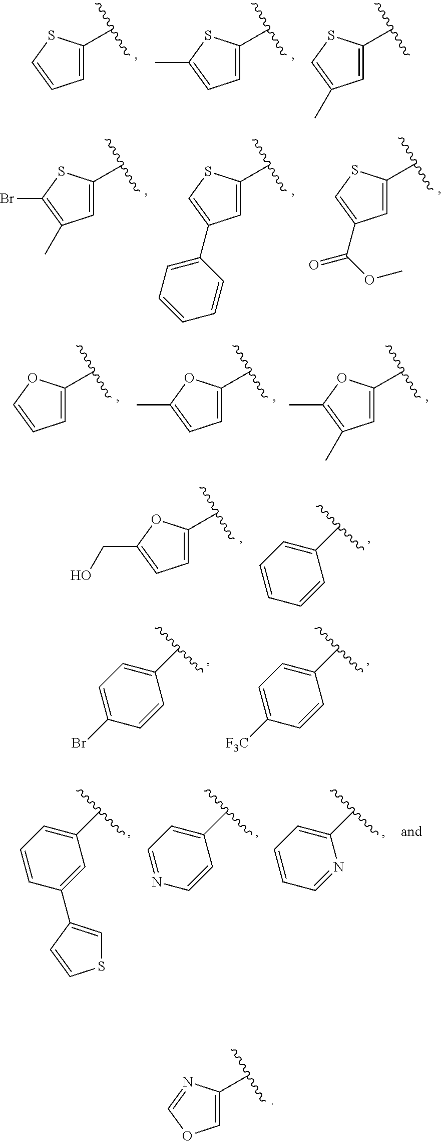

In some embodiments, Z is —CH₂—.

In some embodiments,

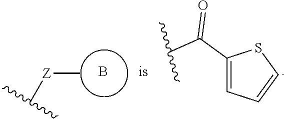

In some embodiments,

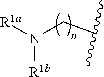

is selected from

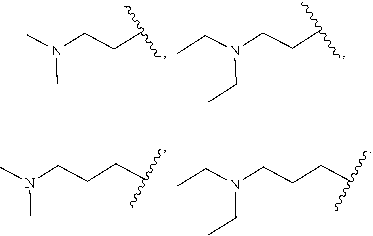

In some embodiments, the compound of formula (I) is a compound having any one of formulas 1001-1038, or a pharmaceutically acceptable salt, solvate, hydrate, cocrystal, or prodrug thereof:

| Cpd. # | Structure |
|---|---|
| 1001 | 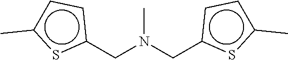 |
| 1002 | 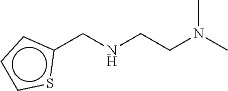 |
| 1003 | 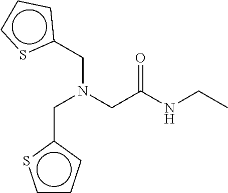 |
| 1004 | 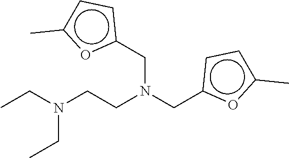 |
| 1005 | 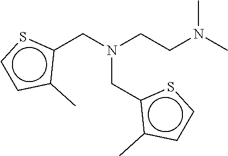 |
| 1006 | 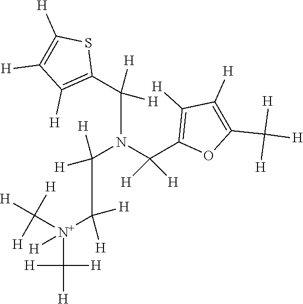 [SMILES: Cc1ccc(CN(CC[NH+](C)C)Cc2cccs2)o1] |
| 1007 | 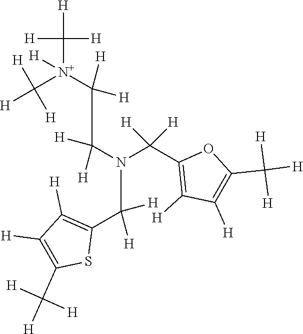 [SMILES: Cc1ccc(CN(CC[NH+](C)C)Cc2ccc(C)s2)o1] |
| 1008 | 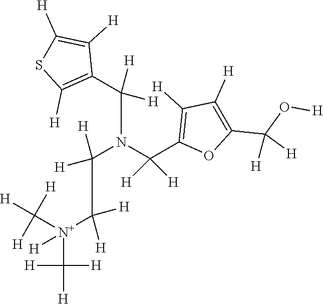 [SMILES: C[NH+](C)CCN(Cc1ccsc1)Cc1ccc(CO)o1] |
| 1009 | 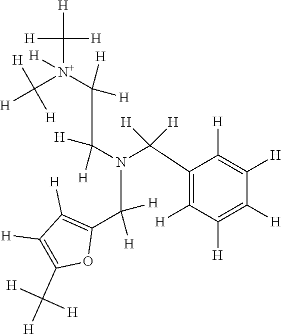 [SMILES: Cc1ccc(CN(CC[NH+](C)C)Cc2ccccc2)o1] |
| 1010 | 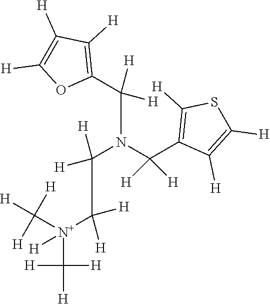 [SMILES: C[NH+](C)CCN(Cc1ccsc1)Cc1ccco1] |
| 1011 | 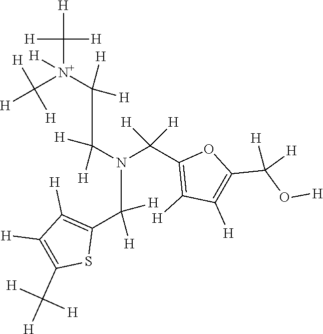 [SMILES: Cc1ccc(CN(CC[NH+](C)C)Cc2ccc(CO)o2)s1] |
| 1012 | 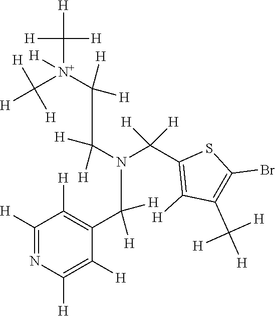 [SMILES: Cc1cc(CN(CC[NH+](C)C)Cc2ccncc2)sc1Br] |
| 1013 | 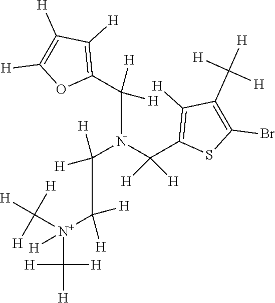 [SMILES: Cc1cc(CN(CC[NH+](C)C)Cc2ccco2)sc1Br] |
| 1014 | 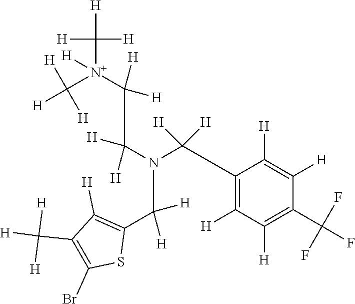 [SMILES: Cc1cc(CN(CC[NH+](C)C)Cc2ccc(C(F)(F)F)cc2)sc1Br] |
| 1015 | 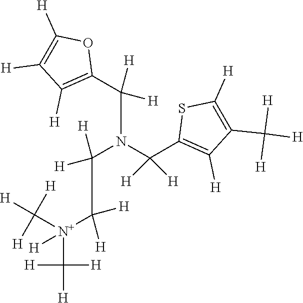 [SMILES: Cc1csc(CN(CC[NH+](C)C)Cc2ccco2)c1] |
| 1016 | 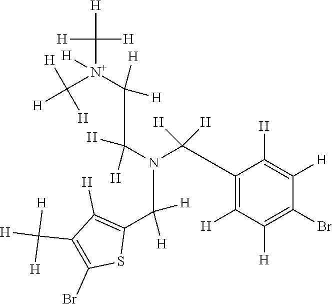 [SMILES: Cc1cc(CN(CC[NH+](C)C)Cc2ccc(Br)cc2)sc1Br] |
| 1017 | 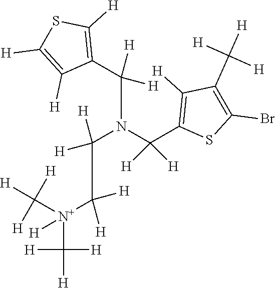 [SMILES: Cc1cc(CN(CC[NH+](C)C)Cc2ccsc2)sc1Br] |
| 1018 | 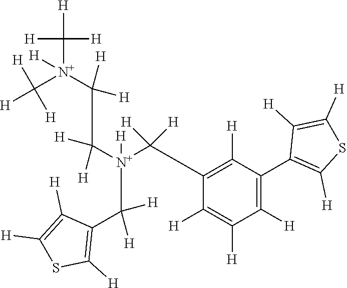 [SMILES: C[NH+](C)CC[NH+](Cc1ccsc1)Cc1cccc(-c2ccsc2)c1] |
| 1019 | 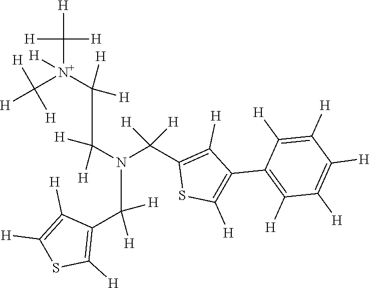 [SMILES: C[NH+](C)CCN(Cc1ccsc1)Cc1cc(-c2ccccc2)cs1] |
| 1020 | 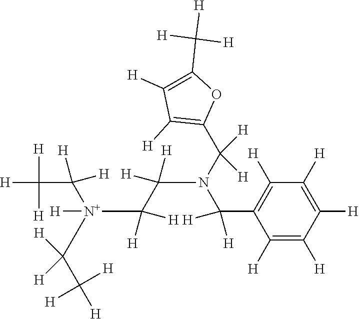 [SMILES: CC[NH+](CC)CCN(Cc1ccccc1)Cc1ccc(C)o1] |
| 1021 | 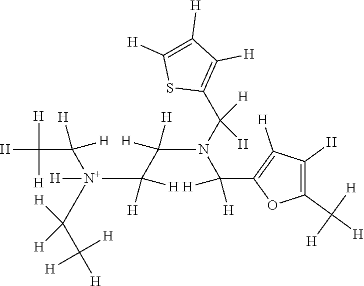 [SMILES: CC[NH+](CC)CCN(Cc1ccc(C)o1)Cc1cccs1] |
| 1022 | 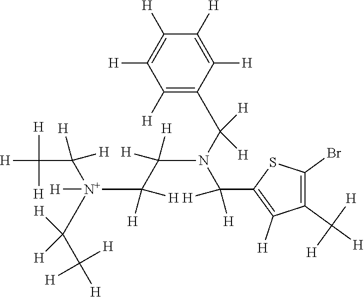 [SMILES: CC[NH+](CC)CCN(Cc1ccccc1)Cc1cc(C)c(Br)s1] |
| 1023 | 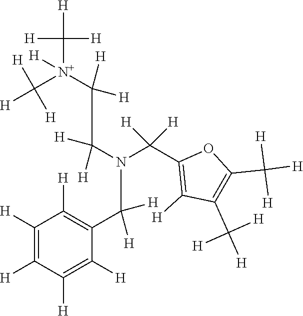 [SMILES: Cc1cc(CN(CC[NH+](C)C)Cc2ccccc2)oc1C] |
| 1024 | 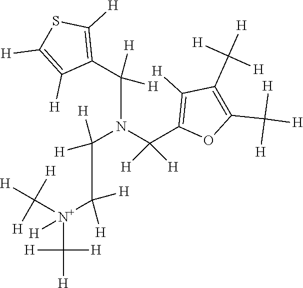 [SMILES: Cc1cc(CN(CC[NH+](C)C)Cc2ccsc2)oc1C] |
| 1025 | 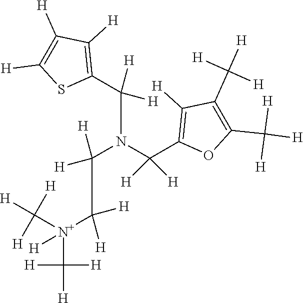 [SMILES: Cc1cc(CN(CC[NH+](C)C)Cc2cccs2)oc1C] |
| 1026 | 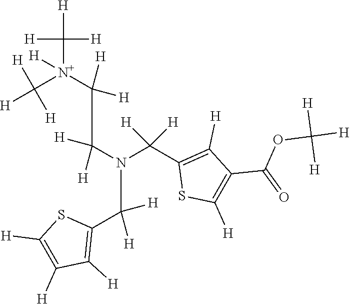 [SMILES: COC(=O)c1csc(CN(CC[NH+](C)C)Cc2cccs2)c1] |
| 1027 | 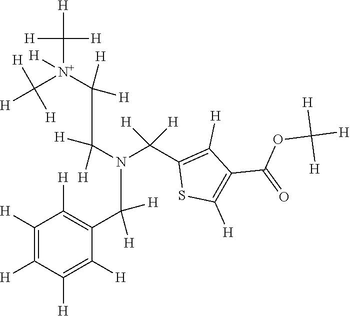 [SMILES: COC(=O)c1csc(CN(CC[NH+](C)C)Cc2ccccc2)c1] |
| 1028 | 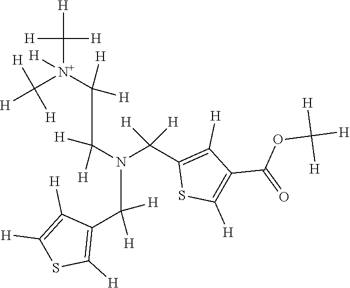 [SMILES: COC(=O)c1csc(CN(CC[NH+](C)C)Cc2ccsc2)c1] |
| 1029 | 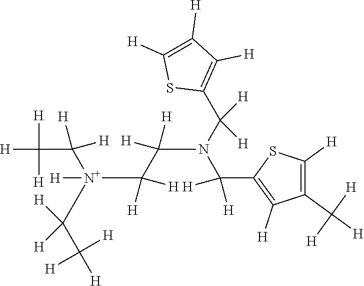 [SMILES: CC[NH+](CC)CCN(Cc1cccs1)Cc1cc(C)cs1] |
| 1030 | 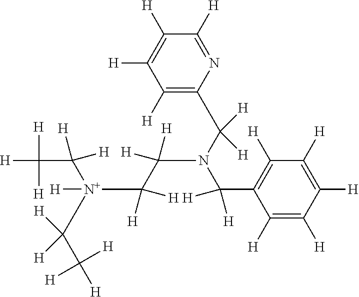 [SMILES: CC[NH+](CC)CCN(Cc1ccccc1)Cc1ccccn1] |
| 1031 | 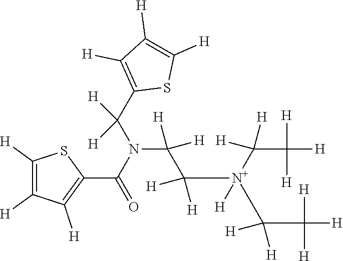 [SMILES: CC[NH+](CC)CCN(Cc1cccs1)C(=O)c1cccs1] |
| 1032 | 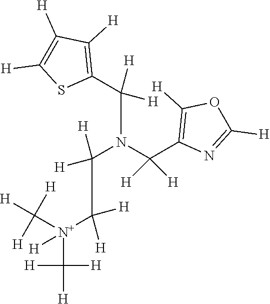 [SMILES: C[NH+](C)CCN(Cc1cocn1)Cc1cccs1] |
| 1033 | 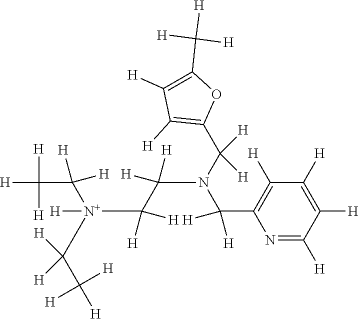 [SMILES: CC[NH+](CC)CCN(Cc1ccccn1)Cc1ccc(C)o1] |
| 1034 | 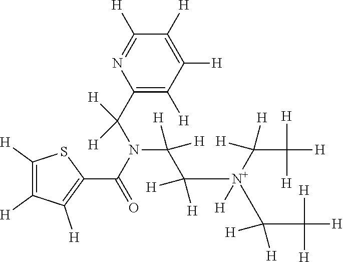 [SMILES: CC[NH+](CC)CCN(Cc1ccccn1)C(=O)c1cccs1] |
| 1035 | 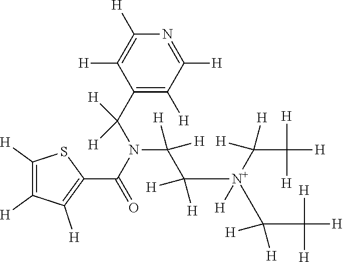 [SMILES: CC[NH+](CC)CCN(Cc1ccncc1)C(=O)c1cccs1] |
| 1036 | 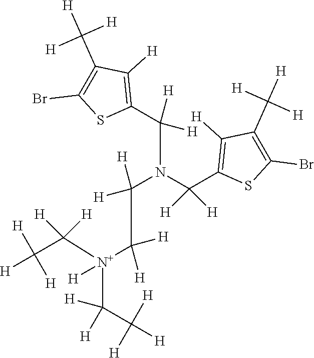 [SMILES: CC[NH+](CC)CCN(Cc1cc(C)c(Br)s1)Cc1cc(C)c(Br)s1] |
| 1037 | 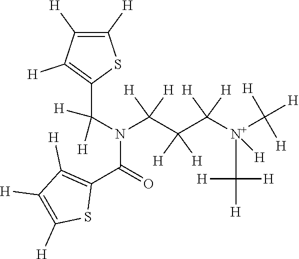 [SMILES: C[NH+](C)CCCN(Cc1cccs1)C(=O)c1cccs1] |
| 1038 | 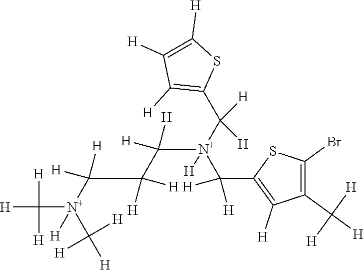 [SMILES: Cc1cc(C[NH+](CCC[NH+](C)C)Cc2cccs2)sc1Br] |

The disclosure provides a compound of formula (II), or a pharmaceutically acceptable salt, solvate, hydrate, cocrystal, or prodrug thereof:

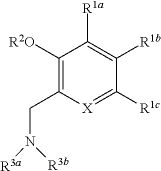

wherein in formula (II):
X is N or CR^{1d};
R^{1a}, R^{1b}, R^{1c}, and R^{1d} are each independently selected from hydrogen, C₁-C₅-alkyl, —OH, C₁-C₅-alkoxy, C₁-C₅-cycloalkyl,

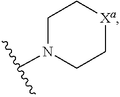

X^{a} is at each occurrence independently selected from —CH₂—, —O—, —NH—, or —N(C₁-C₅-alkyl)-;
R² is selected from hydrogen, C₁-C₅-alkyl, C₁-C₅-alkenyl, —(C₁-C₅-alkyl)-OH,

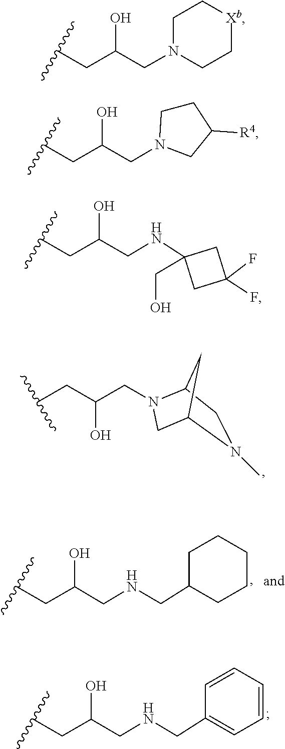

X^{b} is at each occurrence independently selected from —CH₂—, —O—, —NH—, or —N(C₁-C₅-alkyl)-;
R^{3a} and R^{3b} are each independently selected from hydrogen, C₁-C₅-alkyl, —(C₁-C₅-alkyl)-O—(C₁-C₅-alkyl),

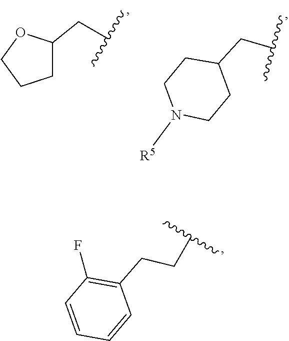

or R^{3a} and R^{3b} are joined together to form

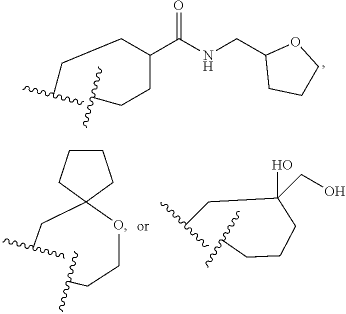 [SMILES: *CCC(CC*)C(=O)NCC1CCCO1 ; *CCOC1(C*)CCCC1 ; *CCCC(O)(C*)CO]

R⁴ is at each occurrence independently selected from hydrogen and halo;
R⁵ is at each occurrence independently selected from

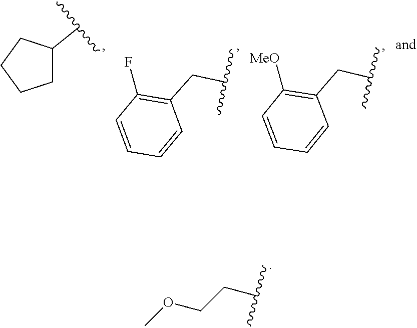

In some embodiments, the compound of formula (II) is a compound of formula 20, or a pharmaceutically acceptable salt, solvate, hydrate, cocrystal, or prodrug thereof:

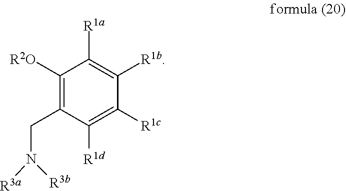

In some embodiments, three of R^{1a}, R^{1b}, Rio, and R^{1d} are hydrogen.

In some embodiments, R^{1a} is selected from —OEt,

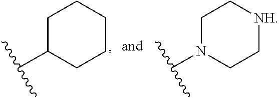 [SMILES: *C1CCCCC1 ; *N1CCNCC1]

In some embodiments, R^{1b} is —OMe.

In some embodiments, R^{1d} is selected from —OH, —OMe, and

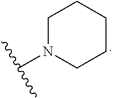 [SMILES: *N1CCCCC1]

In some embodiments, R² is selected from hydrogen,

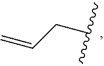 [SMILES: *CC=C]

methyl, ethyl,

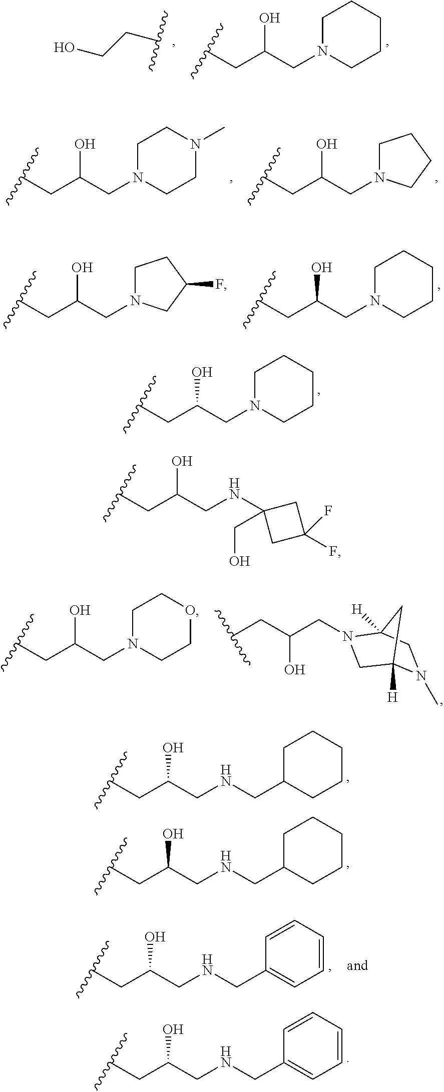

In some embodiments, and R^{3b} are each independently selected from hydrogen,

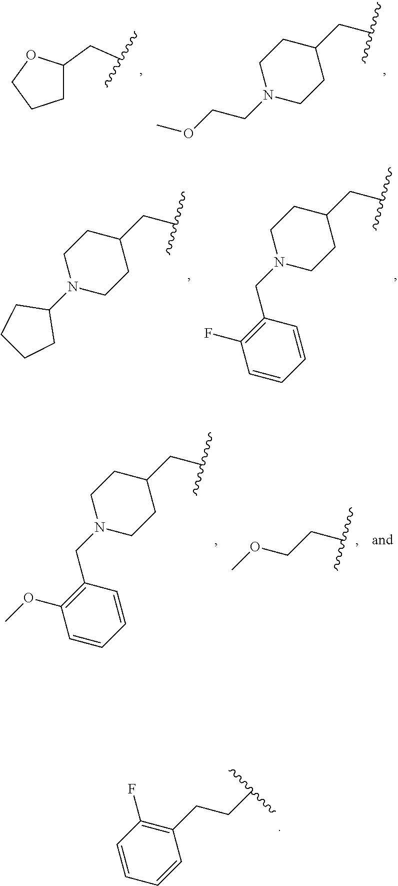

In some embodiments, R^{3a} and R^{3b} join together to form a ring selected from

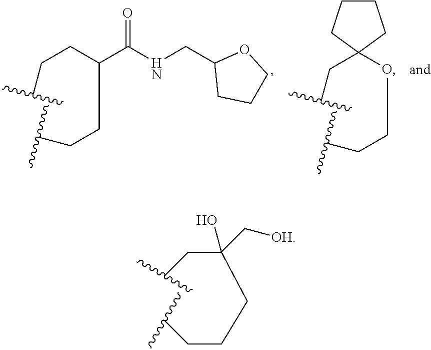 [SMILES: *CCC(CC*)C(=O)NCC1CCCO1 ; *CCOC1(C*)CCCC1 ; *CCCC(O)(C*)CO]

In some embodiments, the compound of formula (II) is a compound having any one of formulas 2001-2028, or a pharmaceutically acceptable salt, solvate, hydrate, cocrystal, or prodrug thereof:

| Cpd. # | Structure |
|---|---|
| 2001 | 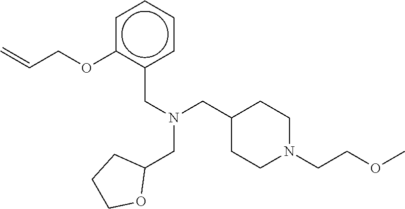 |
| 2002 | 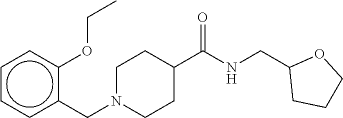 |
| 2003 | 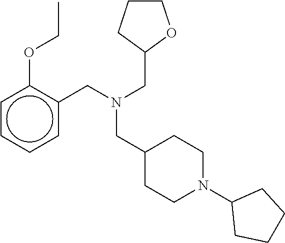 |
| 2004 | 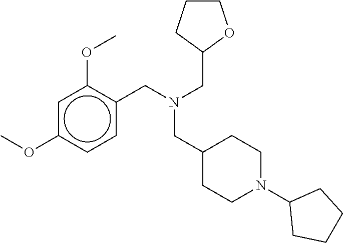 |
| 2005 | 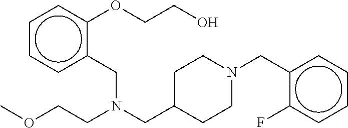 |
| 2006 | 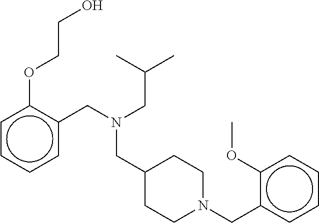 |
| 2007 | 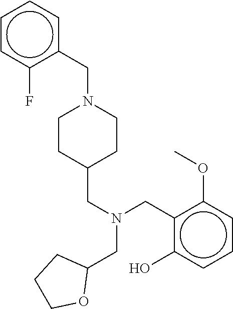 |
| 2008 | 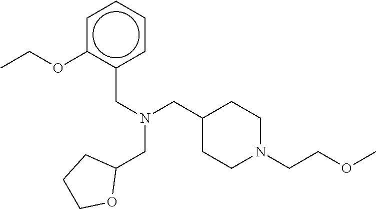 |
| 2009 | 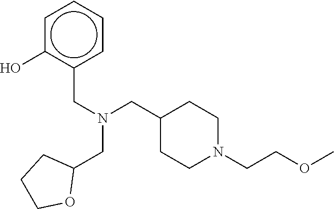 |
| 2010 | 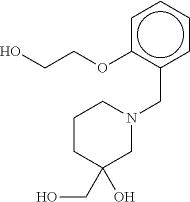 |
| 2011 | 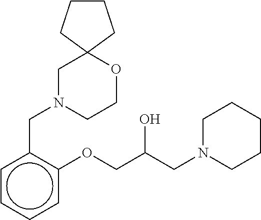 |
| 2012 | 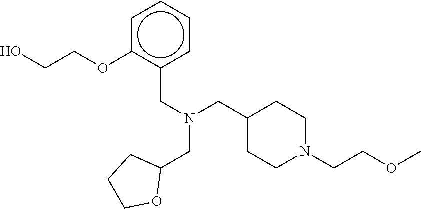 |
| 2013 | 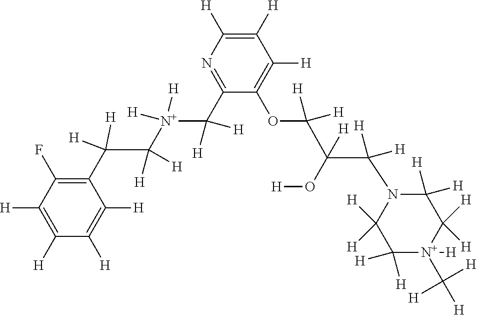 [SMILES: C[NH+]1CCN(CC(O)COc2cccnc2C[NH2+]CCc2ccccc2F)CC1] |
| 2014 | 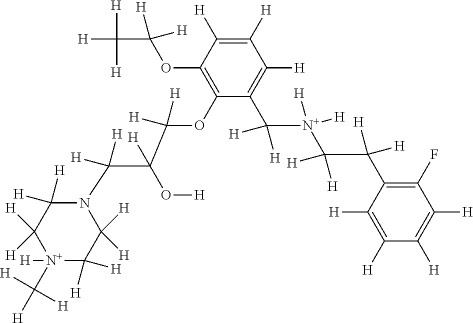 [SMILES: CCOc1cccc(C[NH2+]CCc2ccccc2F)c1OCC(O)CN1CC[NH+](C)CC1] |
| 2015 | 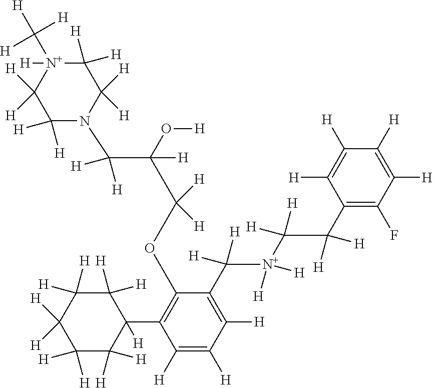 [SMILES: C[NH+]1CCN(CC(O)COc2c(C[NH2+]CCc3ccccc3F)cccc2C2CCCCC2)CC1] |
| 2016 | 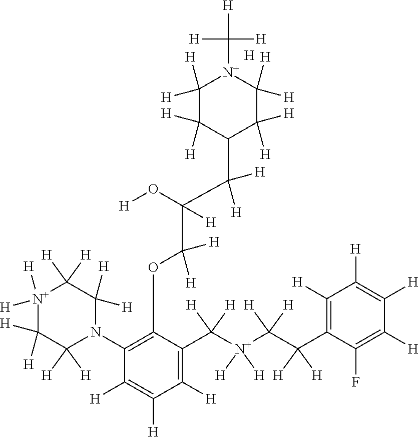 |
| 2017 | 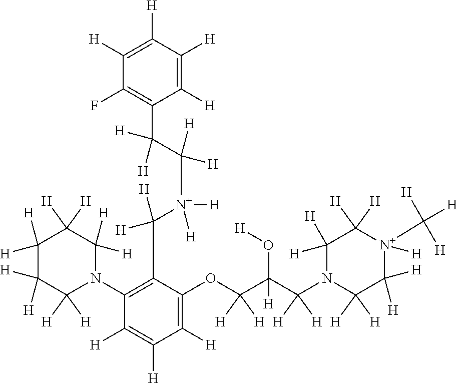 [SMILES: C[NH+]1CCN(CC(O)COc2cccc(N3CCCCC3)c2C[NH2+]CCc2ccccc2F)CC1] |
| 2018 | 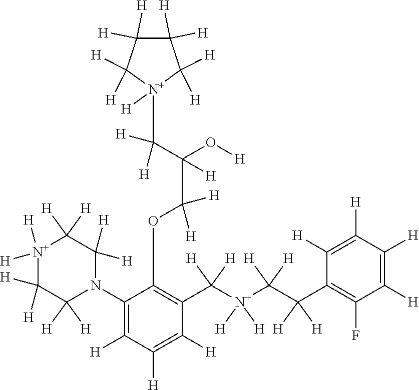 [SMILES: OC(COc1c(C[NH2+]CCc2ccccc2F)cccc1N1CC[NH2+]CC1)C[NH+]1CCCC1] |
| 2019 | 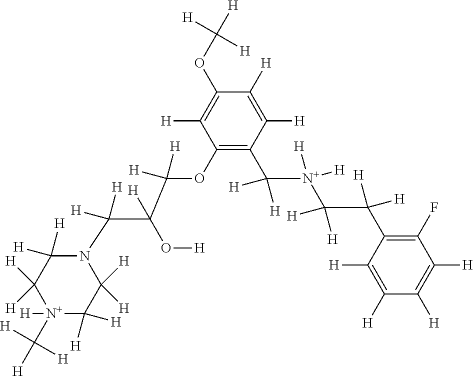 [SMILES: COc1ccc(C[NH2+]CCc2ccccc2F)c(OCC(O)CN2CC[NH+](C)CC2)c1] |
| 2020 | 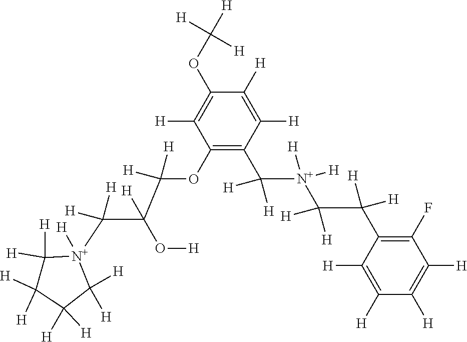 [SMILES: COc1ccc(C[NH2+]CCc2ccccc2F)c(OCC(O)C[NH+]2CCCC2)c1] |
| 2021 | 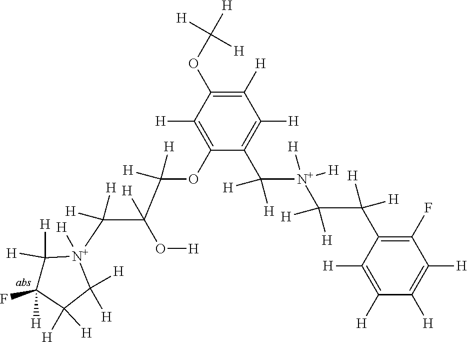 [SMILES: COc1ccc(C[NH2+]CCc2ccccc2F)c(OCC(O)C[NH+]2CC[C@@H](F)C2)c1] |
| 2022 | 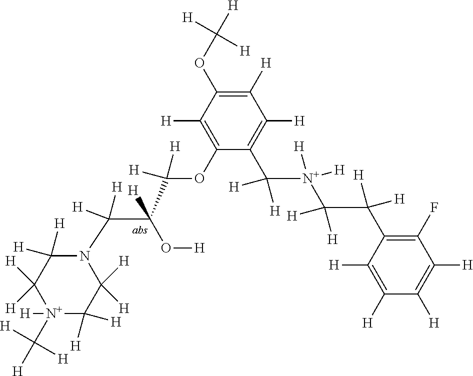 [SMILES: COc1ccc(C[NH2+]CCc2ccccc2F)c(OC[C@H](O)CN2CC[NH+](C)CC2)c1] |
| 2023 | 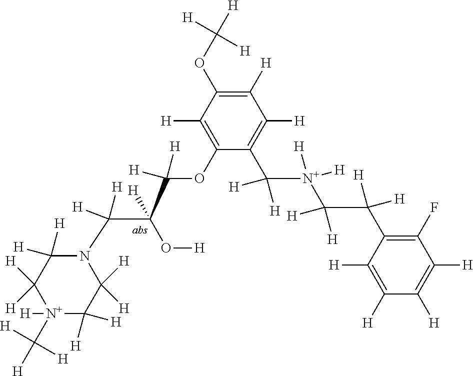 [SMILES: COc1ccc(C[NH2+]CCc2ccccc2F)c(OC[C@@H](O)CN2CC[NH+](C)CC2)c1] |
| 2024 | 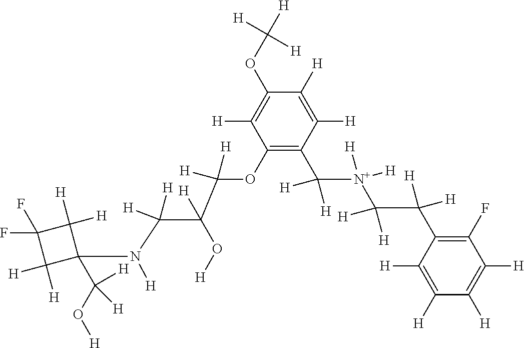 [SMILES: COc1ccc(C[NH2+]CCc2ccccc2F)c(OCC(O)CNC2(CO)CC(F)(F)C2)c1] |
| 2025 | 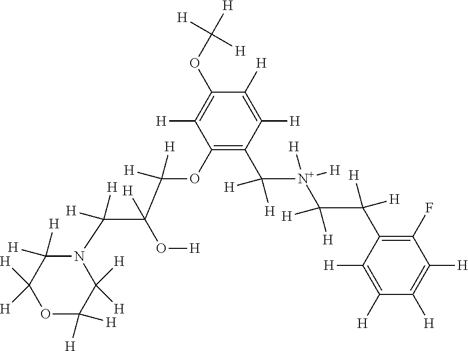 [SMILES: COc1ccc(C[NH2+]CCc2ccccc2F)c(OCC(O)CN2CCOCC2)c1] |
| 2026 | 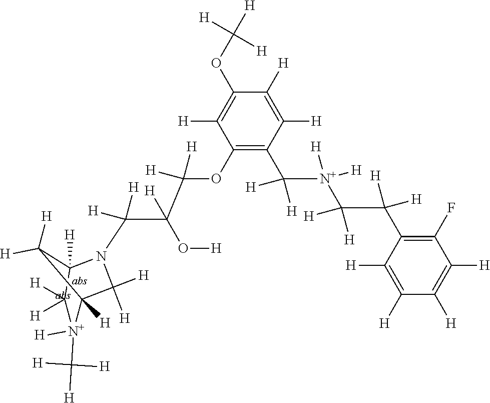 [SMILES: COc1ccc(C[NH2+]CCc2ccccc2F)c(OCC(O)CN2C[C@H]3C[C@@H]2C[NH+]3C)c1] |
| 2027 | 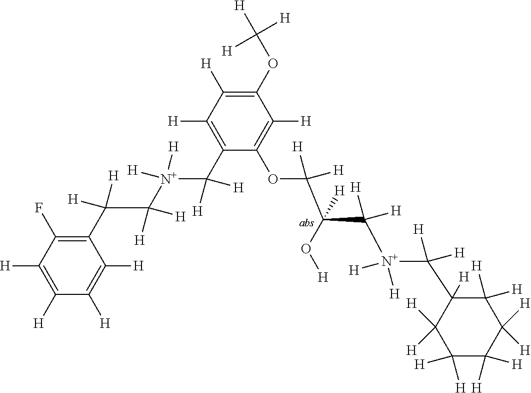 [SMILES: COc1ccc(C[NH2+]CCc2ccccc2F)c(OC[C@H](O)C[NH2+]CC2CCCCC2)c1] |
| 2028 | 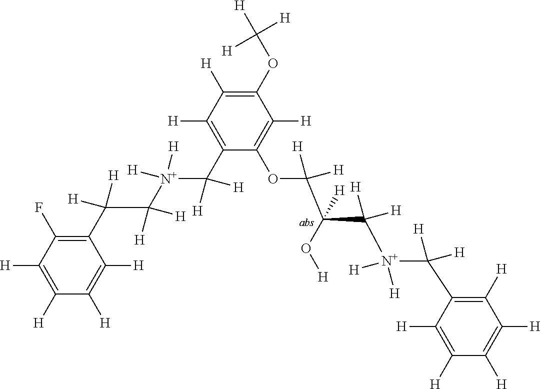 [SMILES: COc1ccc(C[NH2+]CCc2ccccc2F)c(OC[C@H](O)C[NH2+]Cc2ccccc2)c1] |

The disclosure provides a compound of formula (III), or a pharmaceutically acceptable salt, solvate, hydrate, cocrystal, or prodrug thereof:

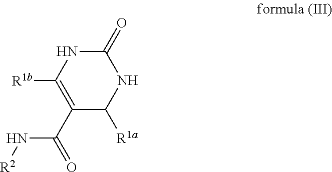

wherein in formula (III):
R^{1a} is selected from

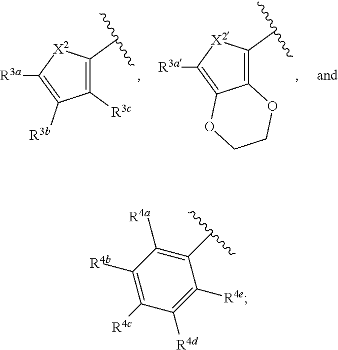

R^{1b} is selected from C₁-C₅-alkyl and —(C₁-C₅-alkyl)-O—(C₁-C₅-alkyl);
R² is selected from C₁-C₁₀-alkyl, C₃-C₁₀-cycloalkyl, —(CH₂)—C₃-C₁₀-cycloalkyl, and —(CH₂)—C₆-C₁₀-aryl;
X² and X^{2′} at each occurrence are independently selected from O and S; and
R^{3a}, R^{3b}, R^{3c}, R^{3a′}, R^{4a}, R^{4b}, R^{4c}, R^{4d}, and R^{4e} at each occurrence are independently selected from hydrogen, halo, C₁-C₅-alkyl, and C₁-C₅-alkoxy.

In some embodiments, R^{1a} is selected from

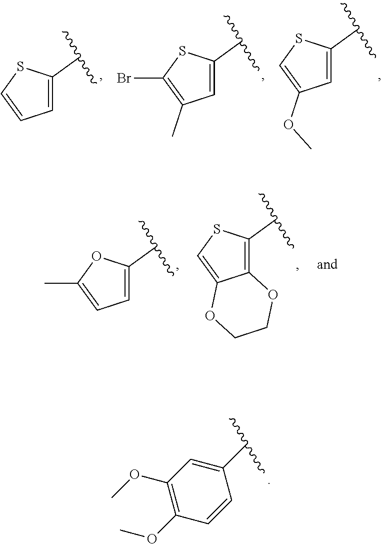

In some embodiments, R^{1b} is selected from methyl and

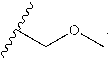 [SMILES: *COC]

In some embodiments, R² is selected from

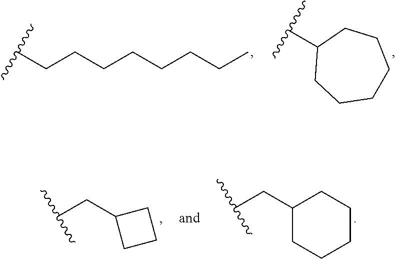

In some embodiments, compound of formula (III) is a compound having any one of formulas 3001-3018, or a pharmaceutically acceptable salt, solvate, hydrate, cocrystal, or prodrug thereof:

| Cpd. # | Structure |
|---|---|
| 3001 | 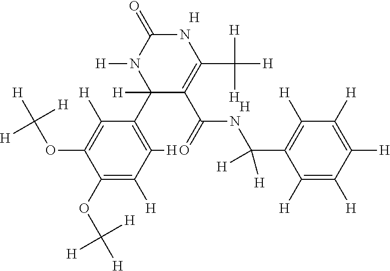 |
| 3002 | 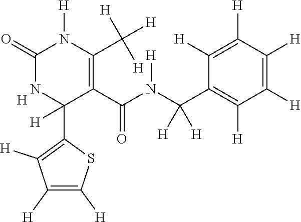 |
| 3003 | 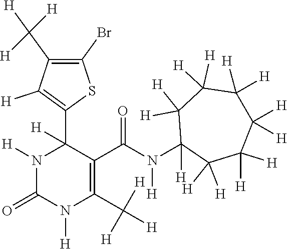 [SMILES: CC1=C(C(=O)NC2CCCCCC2)C(c2cc(C)c(Br)s2)NC(=O)N1] |
| 3004 | 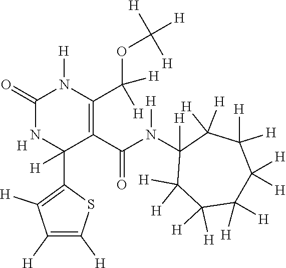 |
| 3005 | 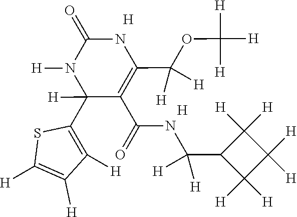 |
| 3006 | 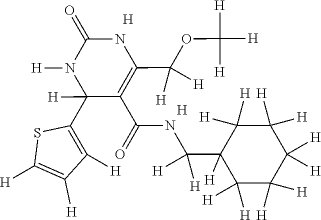 |
| 3007 | 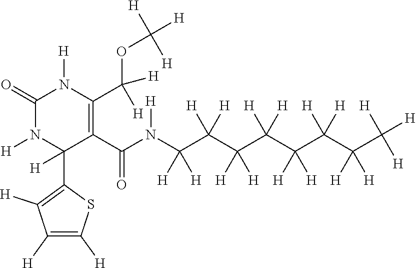 |
| 3008 | 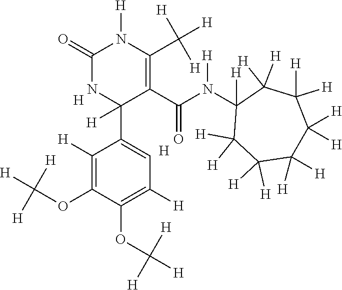 |
| 3009 | 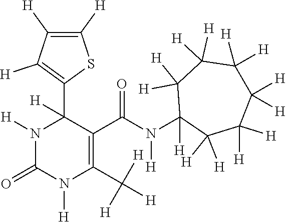 [SMILES: CC1=C(C(=O)NC2CCCCCC2)C(c2cccs2)NC(=O)N1] |
| 3010 | 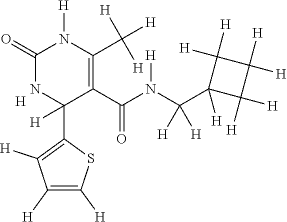 |
| 3011 | 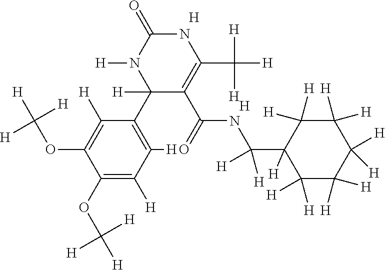 |
| 3012 | 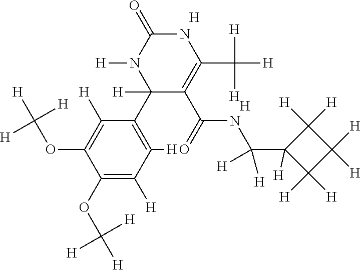 |
| 3013 | 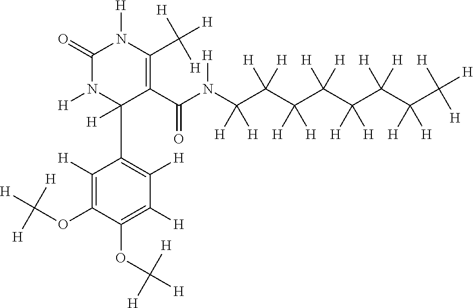 |
| 3014 | 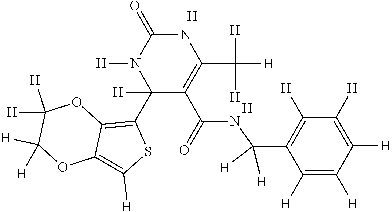 |
| 3015 | 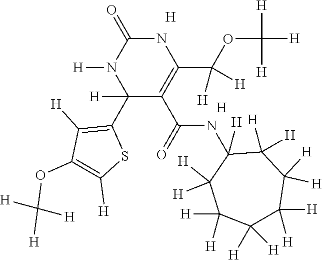 |
| 3016 | 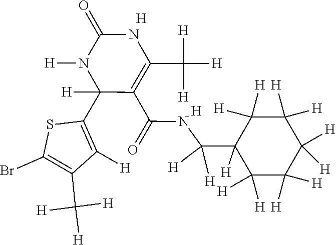 |
| 3017 | 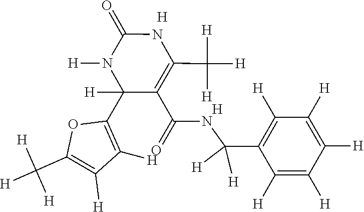 |
| 3018 | 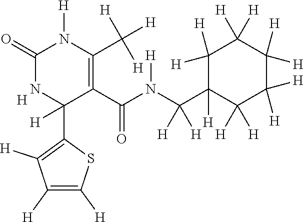 |

The disclosure provides a compound of formula (IV), or a pharmaceutically acceptable salt, solvate, hydrate, cocrystal, or prodrug thereof:

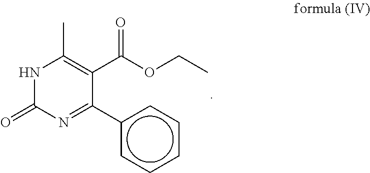

The disclosure provides a compound of formula (V), or a pharmaceutically acceptable salt, solvate, hydrate, cocrystal, or prodrug thereof:

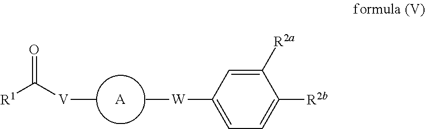

wherein in formula (V):
A is selected from

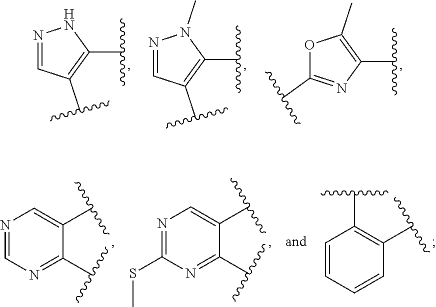

V is selected from a single bond and

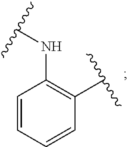 [SMILES: *Nc1ccccc1*]

W is selected from a single bond,

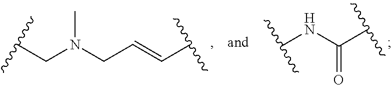

R¹ is selected from

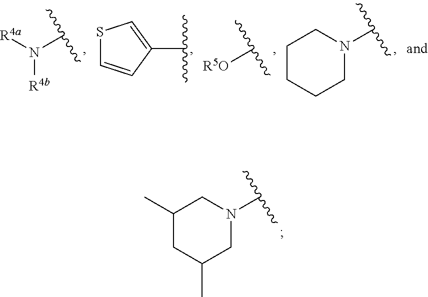

R^{2a} and R^{2b} are each independently selected from hydrogen, —OH, and C₁-C₅-alkoxy, or R^{2a} and R^{2b} are joined to form

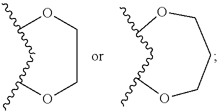

R^{4a} is selected from hydrogen and C₁-C₅-alkyl,
R^{4b} is selected from C₃-C₁₀-cycloalkyl, —(CH₂)—C₃-C₁₀-cycloalkyl,

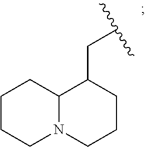 [SMILES: *CC1CCCN2CCCCC12]

and
R⁵ is selected from hydrogen, C₁-C₁₀-alkyl, and

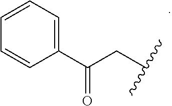 [SMILES: *CC(=O)c1ccccc1]

In some embodiments, the compound of formula (V) is a compound of formula (50), or pharmaceutically acceptable salt, solvate, hydrate, cocrystal, or prodrug thereof:

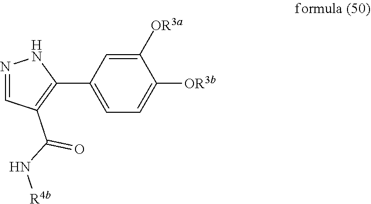

wherein in formula (50):
R^{3a} and R^{3b} are each independently C₁-C₅-alkyl or optionally can be joined to form

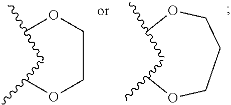

and
R^{4b} is selected from C₃-C₁₀-cycloalkyl and —(CH₂)—C₃-C₁₀-cycloalkyl.

In some embodiments, R^{3a} and R^{3b} are each methyl.

In some embodiments, R^{4b} is selected from

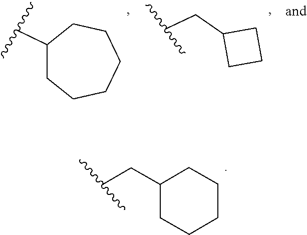 [SMILES: *C1CCCCCC1 ; *CC1CCC1 ; *CC1CCCCC1]

In some embodiments, the compound of formula (V) is a compound having any one of formulas 5001-5013, or a pharmaceutically acceptable salt, solvate, hydrate, cocrystal, or prodrug thereof:

| Cpd. | |
|---|---|
| # | Structure |
| 5001 | 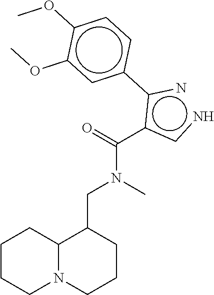 |
| 5002 | 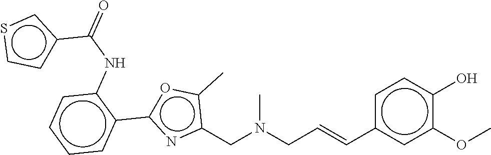 |
| 5003 | 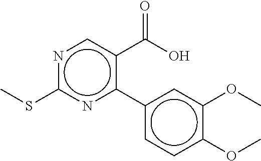 |
| 5004 | 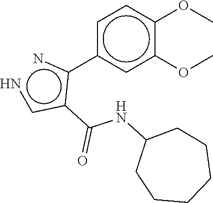 |
| 5005 | 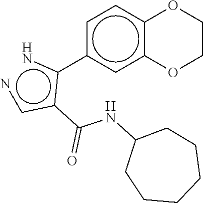 |
| 5006 | 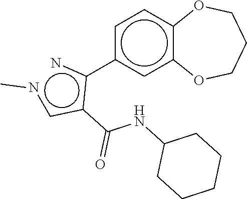 |
| 5007 | 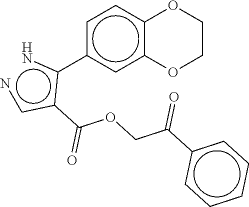 |
| 5008 | 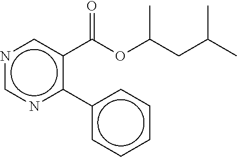 |
| 5009 | 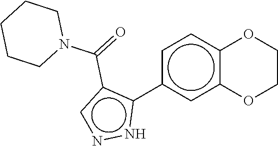 |
| 5010 | 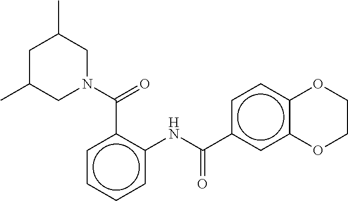 |
| 5011 | 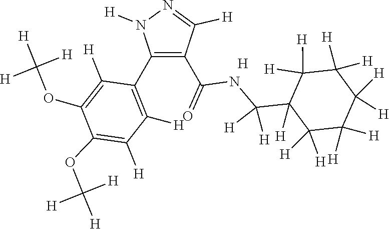 |
| 5012 | 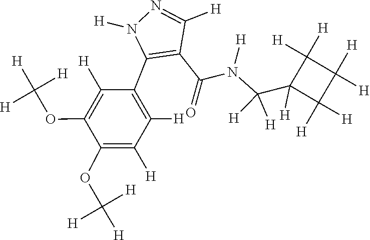 |
| 5013 | 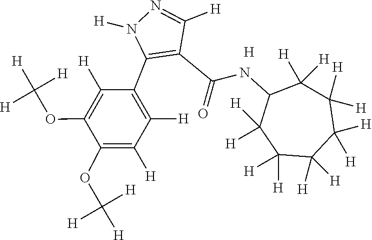 |

The disclosure provides a compound of formula (VI), or a pharmaceutically acceptable salt, solvate, hydrate, cocrystal, or prodrug thereof:

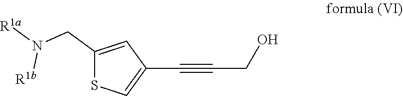

wherein in formula (VI):
R^{1a} and R^{1b} are each independently selected from C₁-C₅-alkyl,

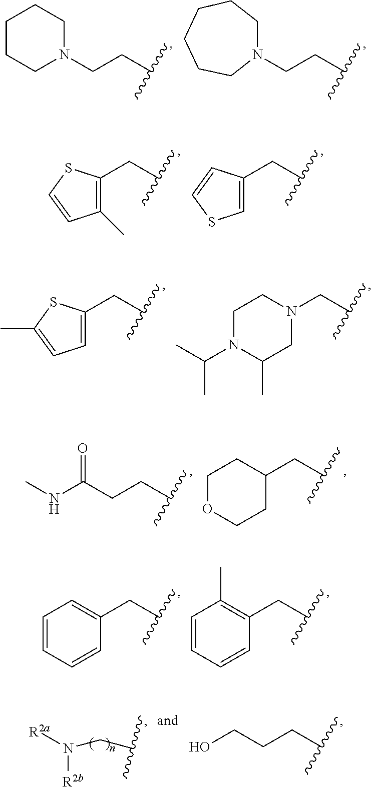

or R^{1a} and R^{1b} are joined together to form

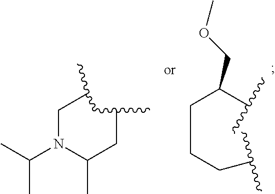

R^{2a} and R^{2b} at each occurrence are independently C₁-C₅-alkyl; and
n is an integer from 1 to 5.

In some embodiments, R^{2a} and R^{2b} are each methyl.

In some embodiments, n is 2.

In some embodiments, the compound of formula (VI) is a compound having any one of formulas 6001-6013, or a pharmaceutically acceptable salt, solvate, hydrate, cocrystal, or prodrug thereof:

| Cpd. | |
|---|---|
| # | Structure |
| 6001 | 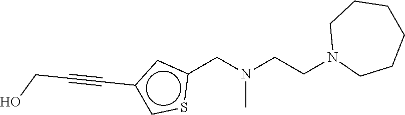 |
| 6002 | 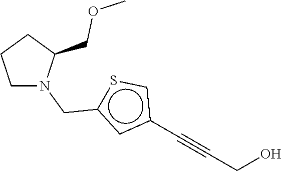 |
| 6003 | 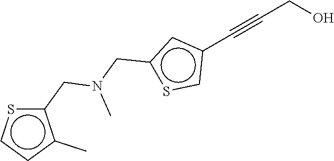 |
| 6004 | 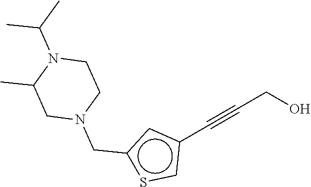 |
| 6005 | 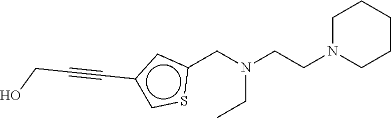 |
| 6006 | 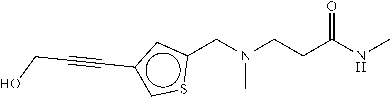 |
| 6007 | 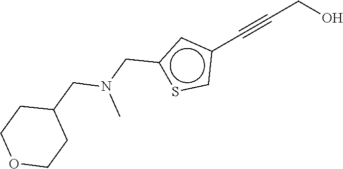 |
| 6008 | 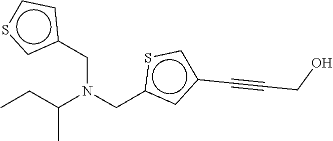 |
| 6009 | 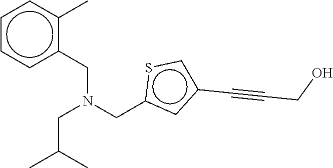 |
| 6010 | 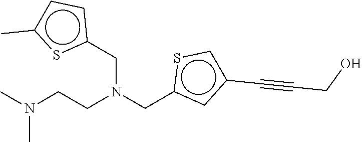 |
| 6011 | 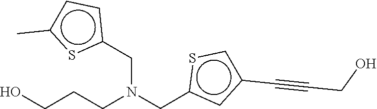 |
| 6012 | 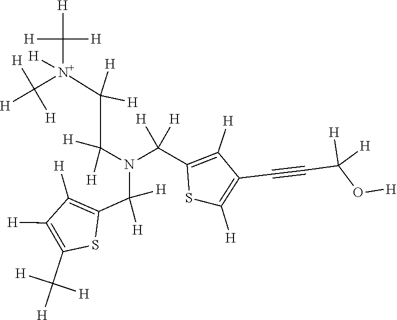 [SMILES: Cc1ccc(CN(CC[NH+](C)C)Cc2cc(C#CCO)cs2)s1] |
| 6013 | 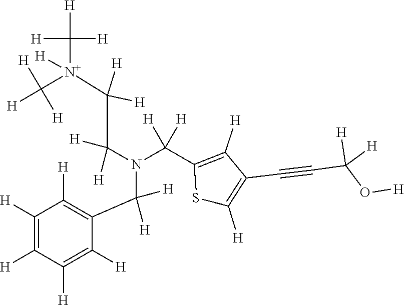 [SMILES: C[NH+](C)CCN(Cc1ccccc1)Cc1cc(C#CCO)cs1] |

The disclosure provides a compound of formula (VII), or a pharmaceutically acceptable salt, solvate, hydrate, cocrystal, or prodrug thereof:

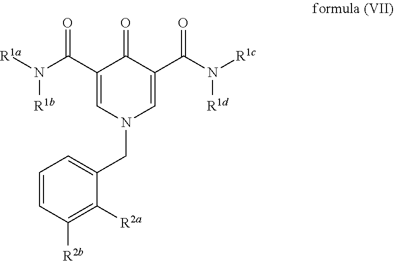

wherein in formula (VII):
R^{1a}, R^{1b}, R^{1c}, and R^{Id} are each independently selected from hydrogen, C₁-C₅-alkyl, C₃-C₁₀-cycloalkyl,

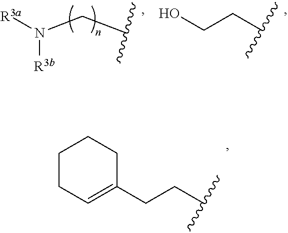

or R^{1a} and R^{1b} and/or R^{1c} and R^{1d} are joined to form

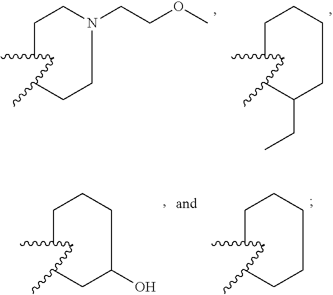

R^{2a} and R^{2b} are each independently selected from hydrogen and C₁-C₅-alkoxy;
R^{3a} and R^{3b} at each occurrence are independently C₁-C₅-alkyl; and
n is an integer selected from 1 to 5.

In some embodiments, R^{1a}, R^{1b}, R^{1c}, and R^{1d} are each independently selected from hydrogen, methyl,

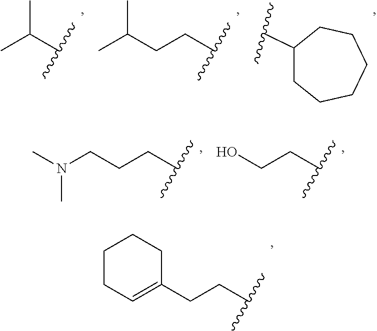

or R^{1a} and R^{1b} and/or R^{1c} and R^{1d} are joined to form

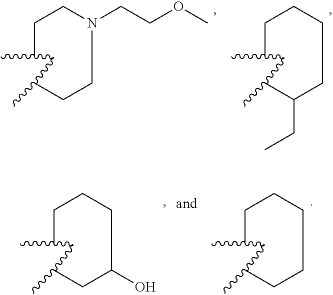

In some embodiments, the compound of formula (VII) is a compound having any one of formulas 7001-7006, or a pharmaceutically acceptable salt, solvate, hydrate, cocrystal, or prodrug thereof:

| Cpd. | |
|---|---|
| # | Structure |
| 7001 | 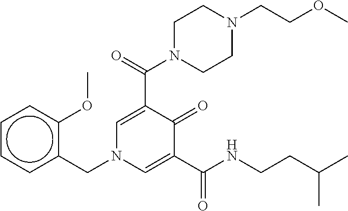 |
| 7002 | 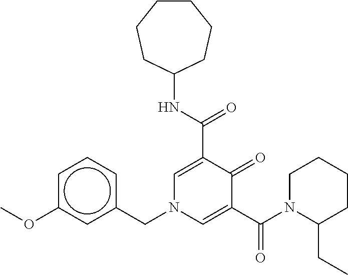 |
| 7003 | 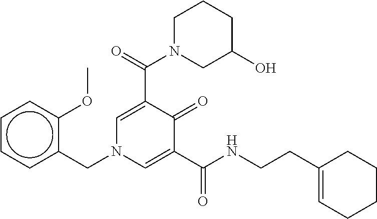 |
| 7004 | 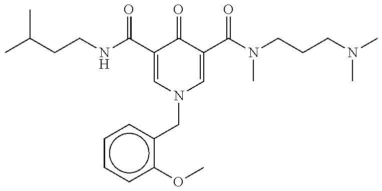 |
| 7005 | 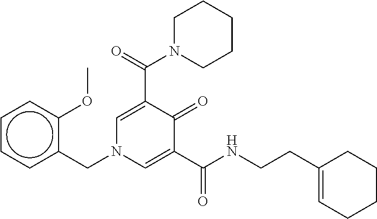 |
| 7006 | 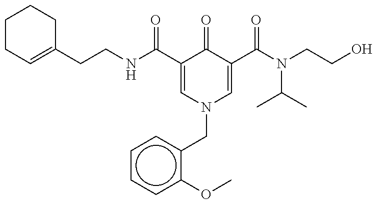 |

The disclosure provides a compound of any one of formula (VIIIa)-(VIIIf), or a pharmaceutically acceptable salt, solvate, hydrate, cocrystal, or prodrug thereof:

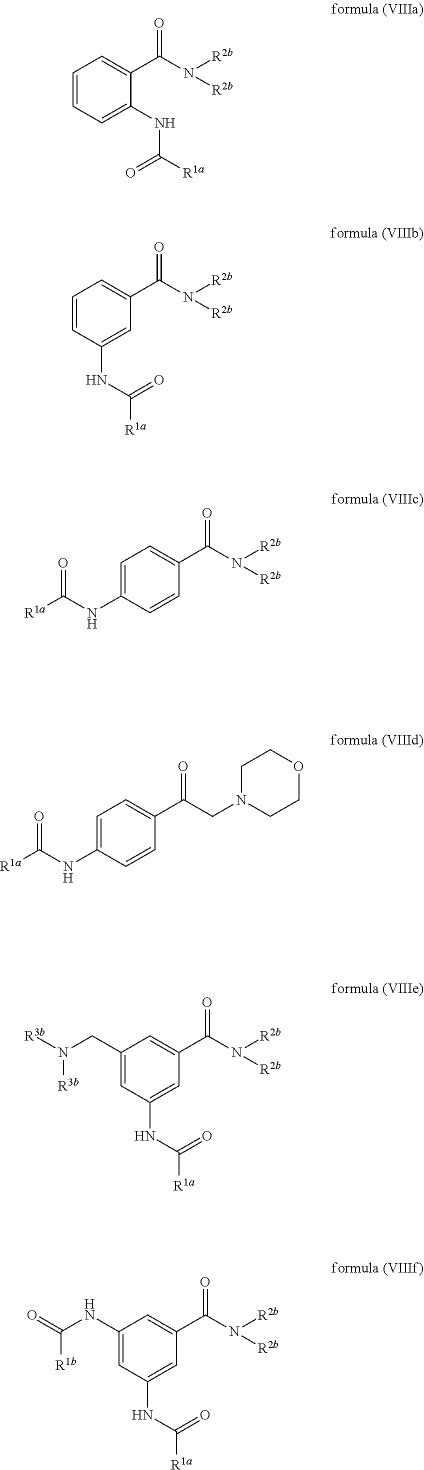

wherein in formula (VIIIa)-(VIIIf):

R^{1a} and R^{1b} are each independently selected from C₁-C₅-alkyl,

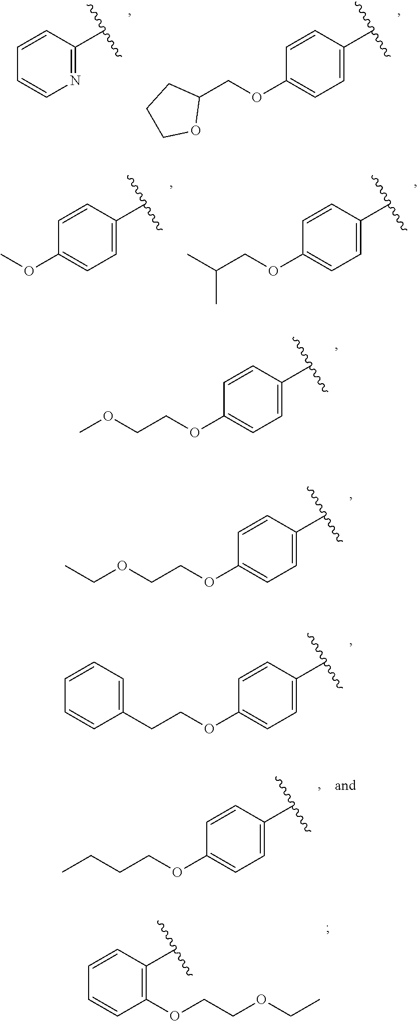

R^{2a}, R^{2b}, R^{3a}, and R^{3b} are each independently selected from hydrogen, C₁-C₅-alkyl,

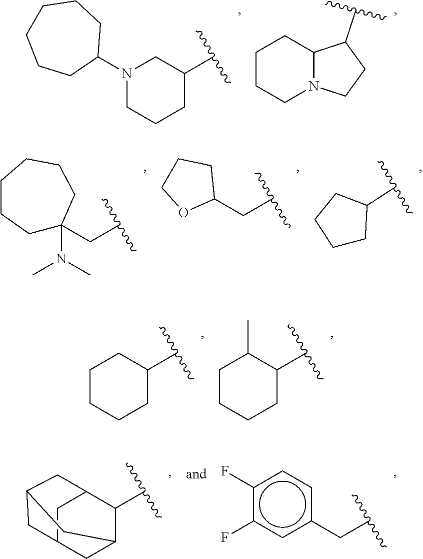

or R^{2a} and R^{2b} and/or R^{3a} and R^{3b} are joined together to form

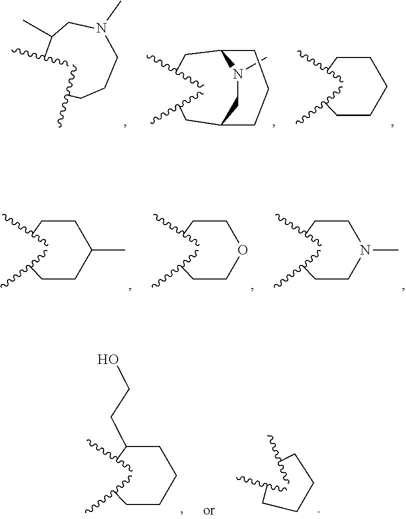

In some embodiments, R^{1a} and R^{1b} are each independently selected from methyl, ethyl,

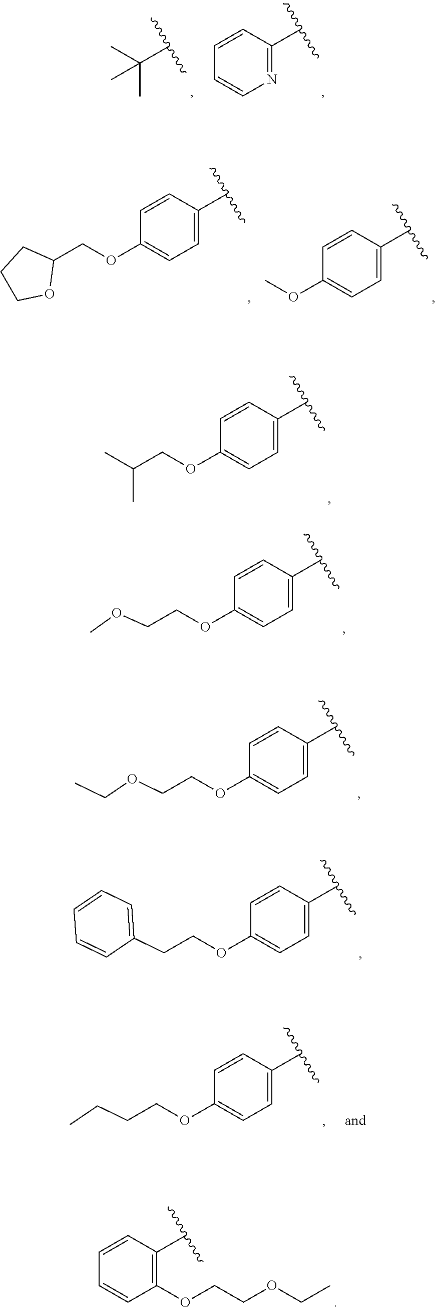

In some embodiments, and R^{2b} are each independently selected from hydrogen, methyl,

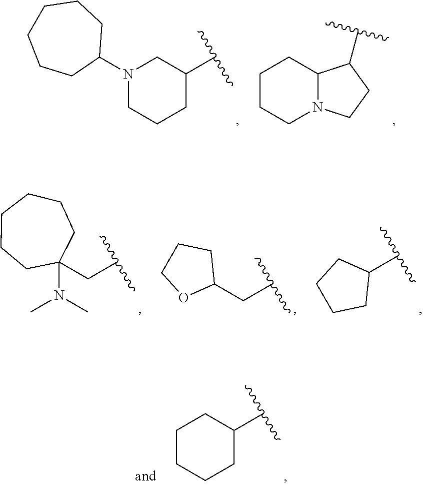

or R^{2a} and R^{2b} are joined together to form

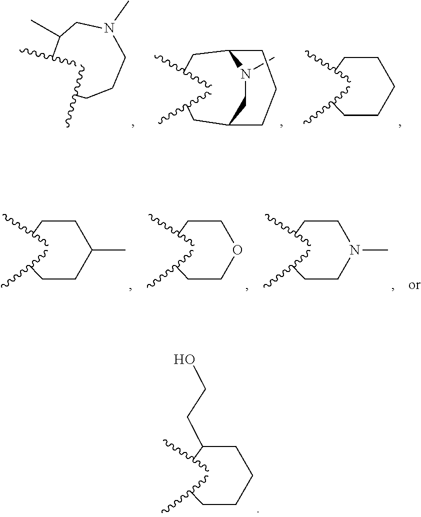

In some embodiments, R^{2a}, R^{2b}, R^{3a} and R^{3b} are each independently selected from hydrogen,

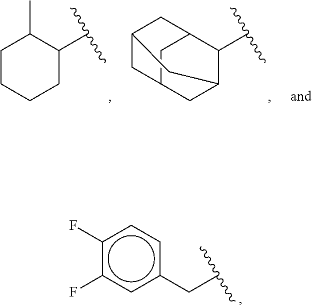

or R^{2a} and R^{2b} and/or R^{3a} and R^{3b} are joined together to form

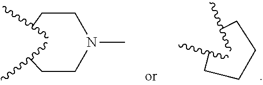

In some embodiments, the compound is selected from:
a) a compound of formula (VIIIa) having any one of formulas 8001-8010, or a pharmaceutically acceptable salt, solvate, hydrate, cocrystal, or prodrug thereof:

| Cpd. | |
|---|---|
| # | Structure |
| 8001 | 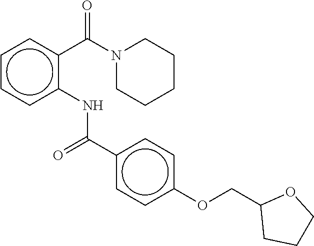 |
| 8002 | 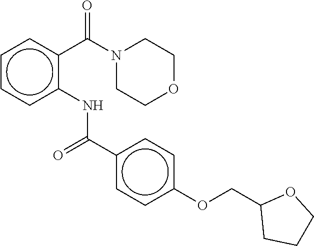 |
| 8003 | 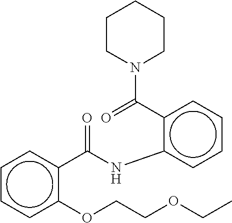 |
| 8004 | 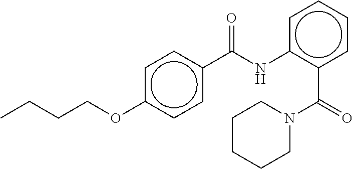 |
| 8005 | 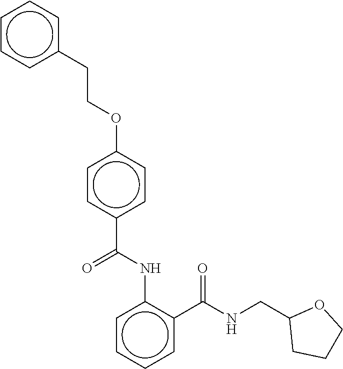 |
| 8006 | 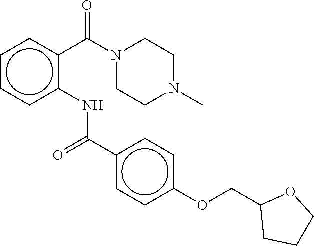 |
| 8007 | 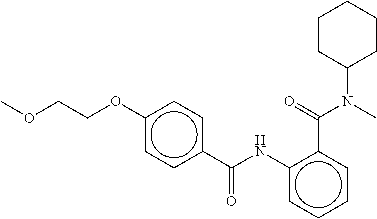 |
| 8008 | 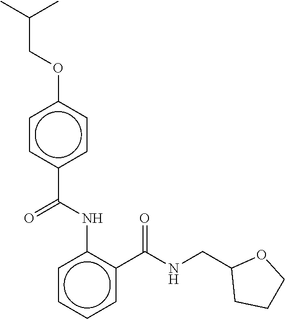 |
| 8009 | 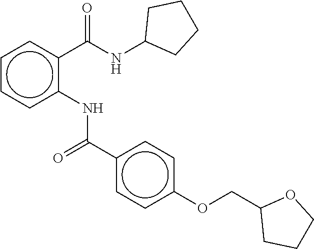 |
| 8010 | 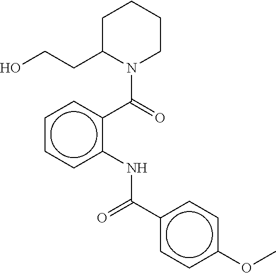 |

b) a compound of formula (VIIIb) having any one of formulas 8101-8105, or a pharmaceutically acceptable salt, solvate, hydrate, cocrystal, or prodrug thereof:

| Cpd. | |
|---|---|
| # | Structure |
| 8101 | 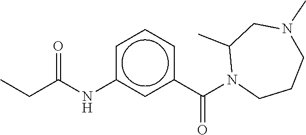 |
| 8102 | 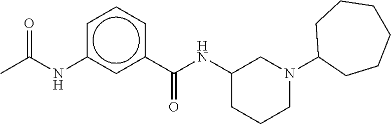 |
| 8103 | 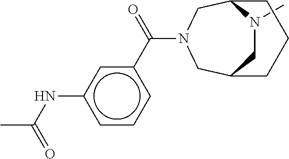 |
| 8104 | 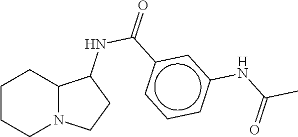 |
| 8105 | 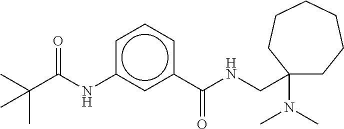 |

c) a compound of formula (VIIIc) having formula 8201, or a pharmaceutically acceptable salt, solvate, hydrate, cocrystal, or prodrug thereof:

| Cpd. | |
|---|---|
| # | Structure |
| 8201 | 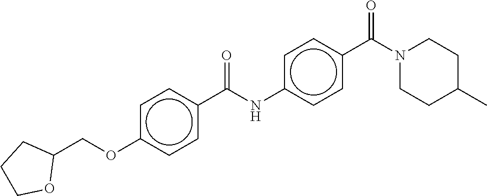 |

d) a compound of formula (VIIId) having formula 8301, or a pharmaceutically acceptable salt, solvate, hydrate, cocrystal, or prodrug thereof:

| Cpd. | |
|---|---|
| # | Structure |
| 8301 | 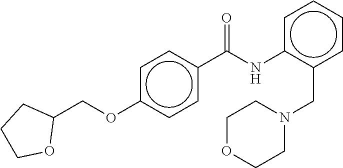 |

e) a compound of formula (VIIIe) having any one of formulas 8401-8403, or a pharmaceutically acceptable salt, solvate, hydrate, cocrystal, or prodrug thereof:

| Cpd. | |
|---|---|
| # | Structure |
| 8401 | 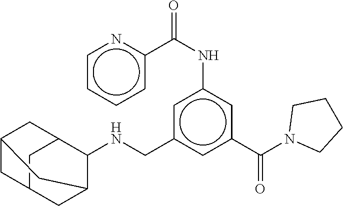 |
| 8402 | 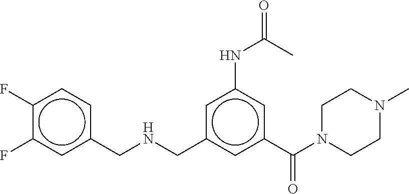 |
| 8403 | 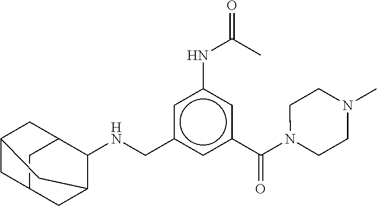 |

f) a compound of formula (VIIIf) having formula 8501, or a pharmaceutically acceptable salt, solvate, hydrate, cocrystal, or prodrug thereof:

| Cpd. | |
|---|---|
| # | Structure |
| 8501 | 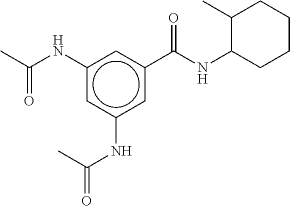 |

The disclosure also provides compounds, or pharmaceutically acceptable salts, solvates, hydrates, cocrystals, or prodrugs thereof,

The disclosure also provides compounds as described herein, wherein the compounds inhibit SKI complex activity. The disclosure also provides compounds as described herein, wherein the compounds inhibit viral replication. The disclosure also provides compounds as described herein, wherein the compounds induce interferon signaling.

In some embodiments, the disclosure provides a compound having any of formula (I), formula (10), formula (II), formula (20), formula (III), formula (IV), formula (V), formula (50), formula (VI), formula (VII), formula (VIIIa), formula (VIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5 D61 to UMB22_5_D70, UMB18_D71, or UMB18_D72, but excluding one or more compounds having the following formulas:

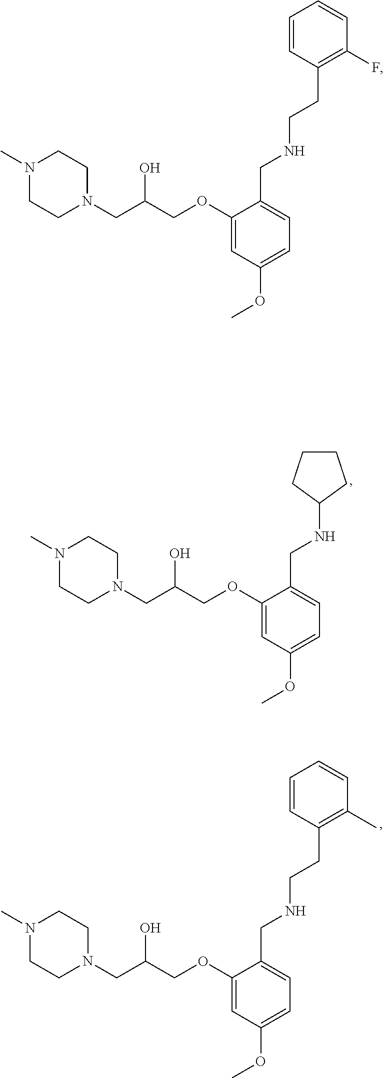

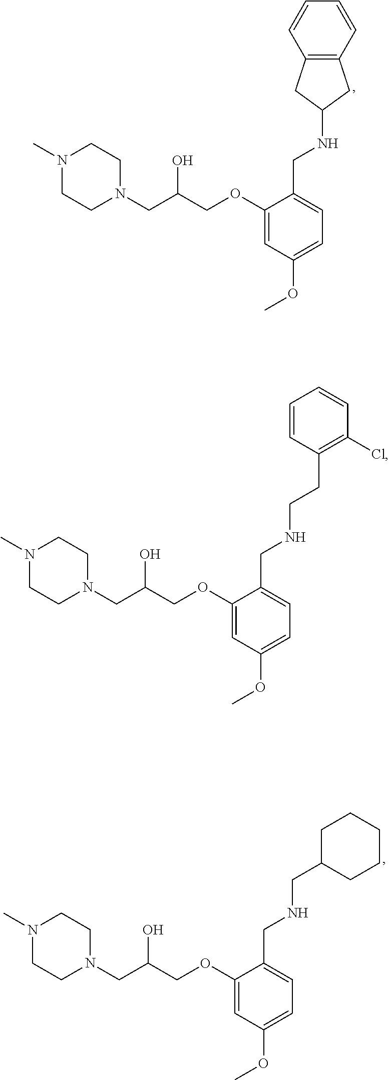 [SMILES: COc1ccc(CNC2Cc3ccccc3C2)c(OCC(O)CN2CCN(C)CC2)c1 ; COc1ccc(CNCCc2ccccc2Cl)c(OCC(O)CN2CCN(C)CC2)c1 ; COc1ccc(CNCC2CCCCC2)c(OCC(O)CN2CCN(C)CC2)c1]

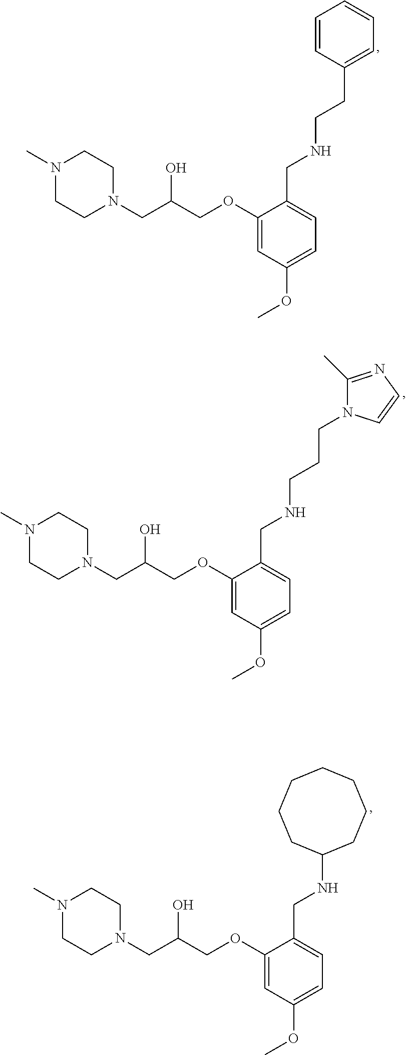 [SMILES: COc1ccc(CNCCc2ccccc2)c(OCC(O)CN2CCN(C)CC2)c1 ; COc1ccc(CNCCCn2ccnc2C)c(OCC(O)CN2CCN(C)CC2)c1 ; COc1ccc(CNC2CCCCCCC2)c(OCC(O)CN2CCN(C)CC2)c1]

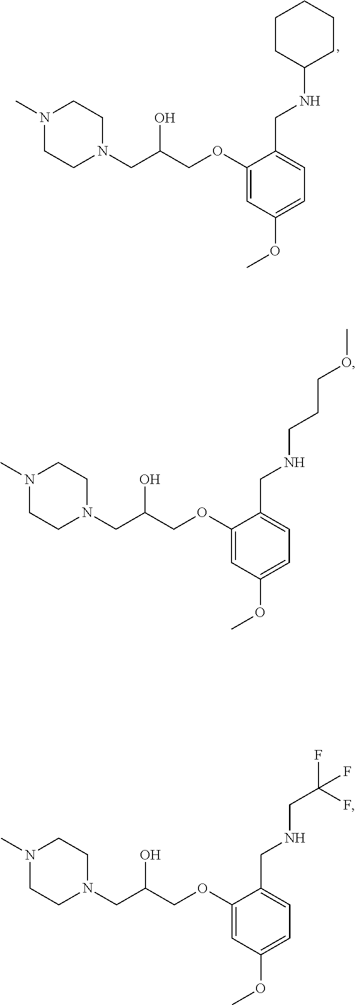

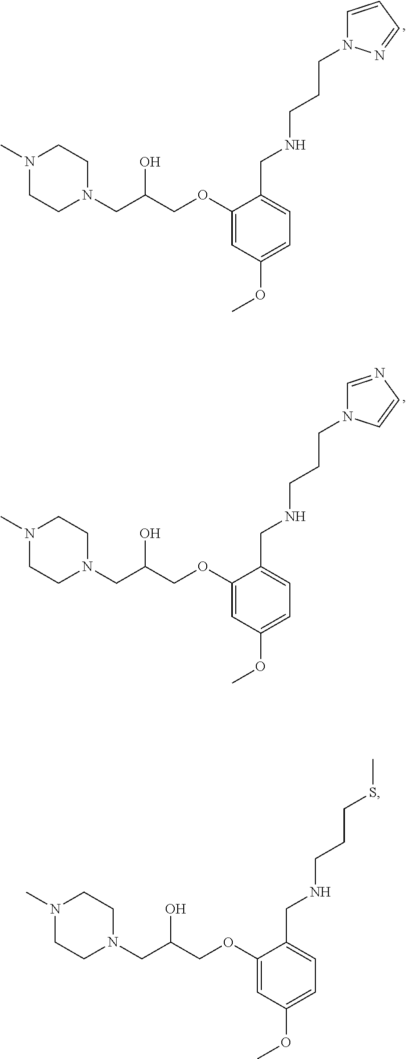

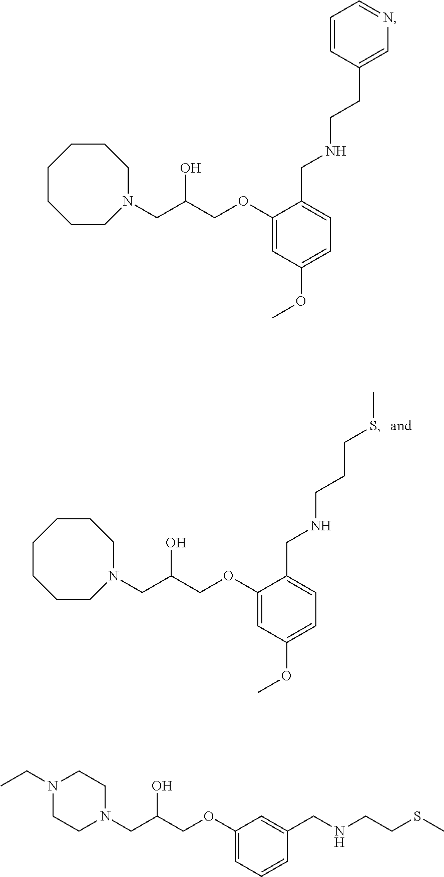

In some embodiments, the disclosure provides a compound having any of formula (I), formula (10), formula (II), formula (20), formula (III), formula (IV), formula (V), formula (50), formula (VI), formula (VII), formula (VIIIa), formula (VIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18 D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, but excluding one or more compounds having any one of formulas 2001-a to 2234-a as described herein, or any one of formulas 2001-b to 2234-b as described herein, or any one of formulas 3001 to 3234 as described herein, wherein the substitution patterns of compounds 2001-a to 2234-a are as defined by formula (II-a), the substitution patterns of compounds 2001-b to 2234-b are as defined by formula (II-b), and the substitution patterns of compounds 3001 to 3234 are as defined by formula (III):

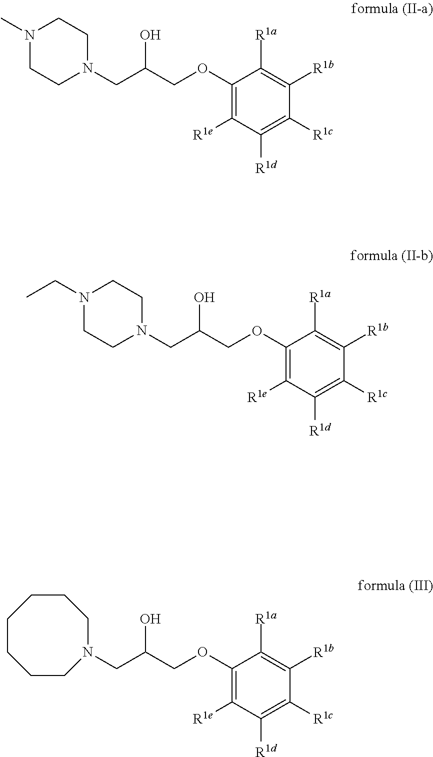

| Cpd. | | | | | |
|---|---|---|---|---|---|
| # | R^{1a} | R^{1b} | R^{1c} | R^{1d} | R^{1e} |
| 2001-a 2001-b 3001 | 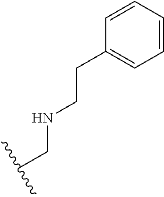 [SMILES: *CNCCc1ccccc1] | H | H | H | H |
| 2002-a 2002-b 3002 | 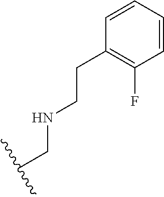 [SMILES: *CNCCc1ccccc1F] | H | H | H | H |
| 2003-a 2003-b 3003 | 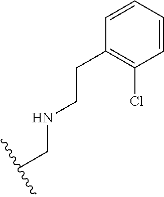 [SMILES: *CNCCc1ccccc1Cl] | H | H | H | H |
| 2004-a 2004-b 3004 | 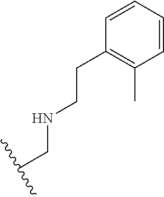 [SMILES: *CNCCc1ccccc1C] | H | H | H | H |
| 2005-a 2005-b 3005 | 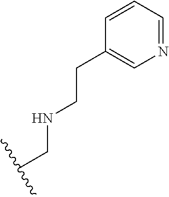 [SMILES: *CNCCc1cccnc1] | H | H | H | H |
| 2006-a 2006-b 3006 | 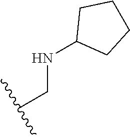 [SMILES: *CNC1CCCC1] | H | H | H | H |
| 2007-a 2007-b 3007 | 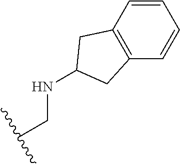 [SMILES: *CNC1Cc2ccccc2C1] | H | H | H | H |
| 2008-a 2008-b 3008 | 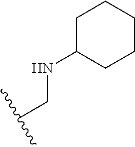 [SMILES: *CNC1CCCCC1] | H | H | H | H |
| 2009-a 2009-b 3009 | 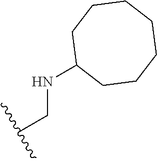 [SMILES: *CNC1CCCCCCC1] | H | H | H | H |
| 2010-a 2010-b 3010 | 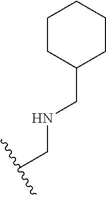 [SMILES: *CNCC1CCCCC1] | H | H | H | H |
| 2011-a 2011-b 3011 | 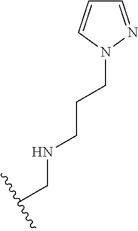 [SMILES: *CNCCCn1cccn1] | H | H | H | H |
| 2012-a 2012-b 3012 | 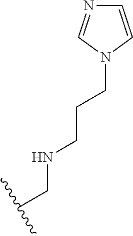 [SMILES: *CNCCCn1ccnc1] | H | H | H | H |
| 2013-a 2013-b 3013 | 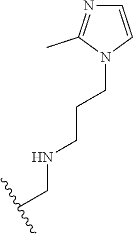 [SMILES: *CNCCCn1ccnc1C] | H | H | H | H |
| 2014-a 2014-b 3014 | 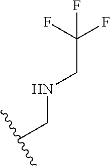 [SMILES: *CNCC(F)(F)F] | H | H | H | H |
| 2015-a 2015-b 3015 | 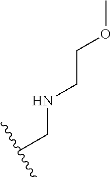 [SMILES: *CNCCOC] | H | H | H | H |
| 2016-a 2016-b 3016 | 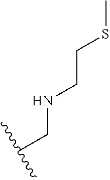 [SMILES: *CNCCSC] | H | H | H | H |
| 2017-a 2017-b 3017 | 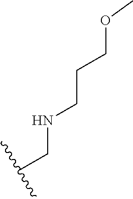 [SMILES: *CNCCCOC] | H | H | H | H |
| 2018-a 2018-b 3018 | 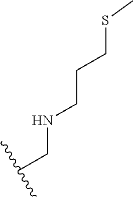 [SMILES: *CNCCCSC] | H | H | H | H |
| 2019-a 2019-b 3019 | H | 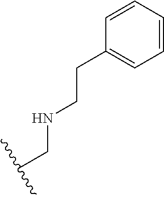 [SMILES: *CNCCc1ccccc1] | H | H | H |
| 2020-a 2020-b 3020 | H | 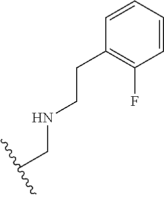 [SMILES: *CNCCc1ccccc1F] | H | H | H |
| 2021-a 2021-b 3021 | H | 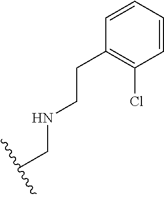 [SMILES: *CNCCc1ccccc1Cl] | H | H | H |
| 2022-a 2022-b 3022 | H | 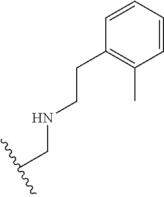 [SMILES: *CNCCc1ccccc1C] | H | H | H |
| 2023-a 2023-b 3023 | H | 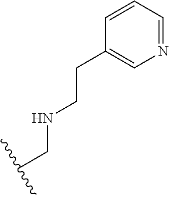 [SMILES: *CNCCc1cccnc1] | H | H | H |
| 2024-a 2024-b 3024 | H | 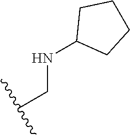 [SMILES: *CNC1CCCC1] | H | H | H |
| 2025-a 2025-b 3025 | H | 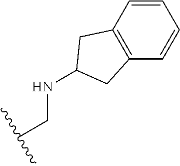 [SMILES: *CNC1Cc2ccccc2C1] | H | H | H |
| 2026-a 2026-b 3026 | H | 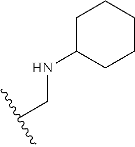 [SMILES: *CNC1CCCCC1] | H | H | H |
| 2027-a 2027-b 3027 | H | 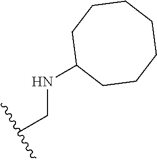 [SMILES: *CNC1CCCCCCC1] | H | H | H |
| 2028-a 2028-b 3028 | H | 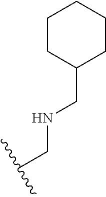 [SMILES: *CNCC1CCCCC1] | H | H | H |
| 2029-a 2029-b 3029 | H | 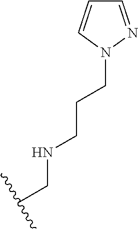 [SMILES: *CNCCCn1cccn1] | H | H | H |
| 2030-a 2030-b 3030 | H | 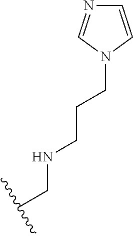 [SMILES: *CNCCCn1ccnc1] | H | H | H |
| 2031-a 2031-b 3031 | H | 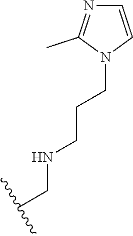 [SMILES: *CNCCCn1ccnc1C] | H | H | H |
| 2032-a 2032-b 3032 | H | 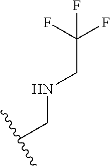 [SMILES: *CNCC(F)(F)F] | H | H | H |
| 2033-a 2033-b 3033 | H | 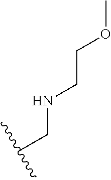 [SMILES: *CNCCOC] | H | H | H |
| 2034-a 2034-b 3034 | H | 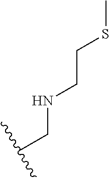 [SMILES: *CNCCSC] | H | H | H |
| 2035-a 2035-b 3035 | H | 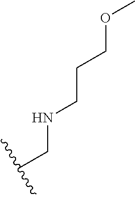 [SMILES: *CNCCCOC] | H | H | H |
| 2036-a 2036-b 3036 | H | 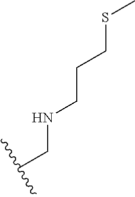 [SMILES: *CNCCCSC] | H | H | H |
| 2037-a 2037-b 3037 | H | H | 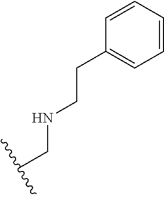 [SMILES: *CNCCc1ccccc1] | H | H |
| 2038-a 2038-b 3038 | H | H | 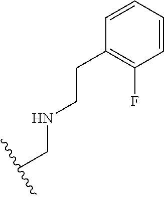 [SMILES: *CNCCc1ccccc1F] | H | H |
| 2039-a 2039-b 3039 | H | H | 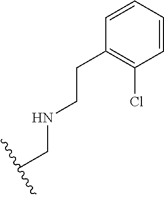 [SMILES: *CNCCc1ccccc1Cl] | H | H |
| 2040-a 2040-b 3040 | H | H | 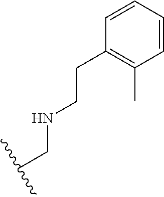 [SMILES: *CNCCc1ccccc1C] | H | H |
| 2041-a 2041-b 3041 | H | H | 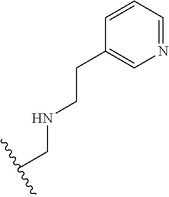 [SMILES: *CNCCc1cccnc1] | H | H |
| 2042-a 2042-b 3042 | H | H | 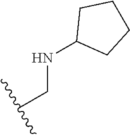 [SMILES: *CNC1CCCC1] | H | H |
| 2043-a 2043-b 3043 | H | H | 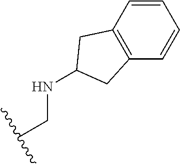 [SMILES: *CNC1Cc2ccccc2C1] | H | H |
| 2044-a 2044-b 3044 | H | H | 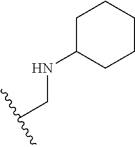 [SMILES: *CNC1CCCCC1] | H | H |
| 2045-a 2045-b 3045 | H | H | 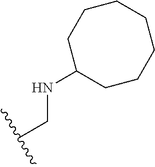 [SMILES: *CNC1CCCCCCC1] | H | H |
| 2046-a 2046-b 3046 | H | H | 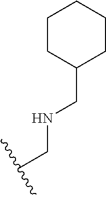 [SMILES: *CNCC1CCCCC1] | H | H |
| 2047-a 2047-b 3047 | H | H | 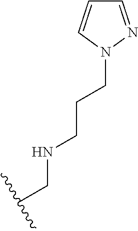 [SMILES: *CNCCCn1cccn1] | H | H |
| 2048-a 2048-b 3048 | H | H | 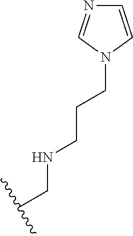 [SMILES: *CNCCCn1ccnc1] | H | H |
| 2049-a 2049-b 3049 | H | H | 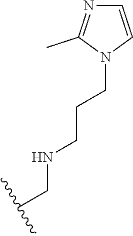 [SMILES: *CNCCCn1ccnc1C] | H | H |
| 2050-a 2050-b 3050 | H | H | 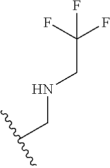 [SMILES: *CNCC(F)(F)F] | H | H |
| 2051-a 2051-b 3051 | H | H | 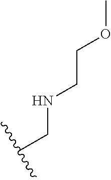 [SMILES: *CNCCOC] | H | H |
| 2052-a 2052-b 3052 | H | H | 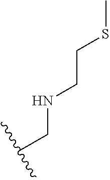 [SMILES: *CNCCSC] | H | H |
| 2053-a 2053-b 3053 | H | H | 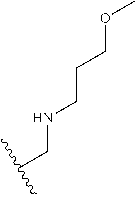 [SMILES: *CNCCCOC] | H | H |
| 2054-a 2054-b 3054 | H | H | 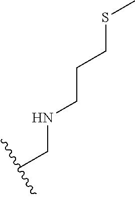 [SMILES: *CNCCCSC] | H | H |
| 2055-a 2055-b 3055 | 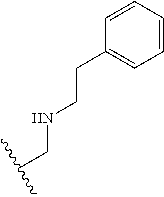 [SMILES: *CNCCc1ccccc1] | —OMe | H | H | H |
| 2056-a 2056-b 3056 | 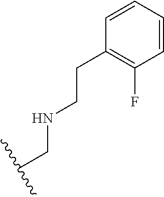 [SMILES: *CNCCc1ccccc1F] | —OMe | H | H | H |
| 2057-a 2057-b 3057 | 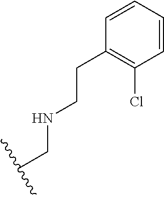 [SMILES: *CNCCc1ccccc1Cl] | —OMe | H | H | H |
| 2058-a 2058-b 3058 | 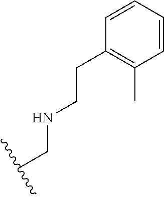 [SMILES: *CNCCc1ccccc1C] | —OMe | H | H | H |
| 2059-a 2059-b 3059 | 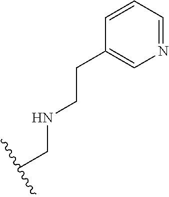 [SMILES: *CNCCc1cccnc1] | —OMe | H | H | H |
| 2060-a 2060-b 3060 | 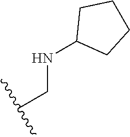 [SMILES: *CNC1CCCC1] | —OMe | H | H | H |
| 2061-a 2061-b 3061 | 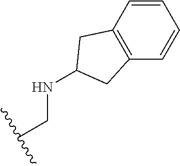 [SMILES: *CNC1Cc2ccccc2C1] | —OMe | H | H | H |
| 2062-a 2062-b 3062 | 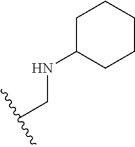 [SMILES: *CNC1CCCCC1] | —OMe | H | H | H |
| 2063-a 2063-b 3063 | 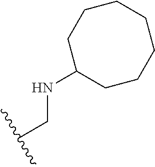 [SMILES: *CNC1CCCCCCC1] | —OMe | H | H | H |
| 2064-a 2064-b 3064 | 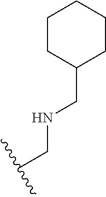 [SMILES: *CNCC1CCCCC1] | —OMe | H | H | H |
| 2065-a 2065-b 3065 | 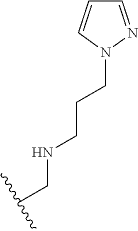 [SMILES: *CNCCCn1cccn1] | —OMe | H | H | H |
| 2066-a 2066-b 3066 | 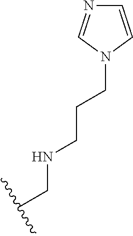 [SMILES: *CNCCCn1ccnc1] | —OMe | H | H | H |
| 2067-a 2067-b 3067 | 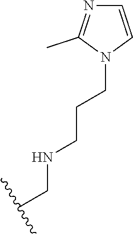 [SMILES: *CNCCCn1ccnc1C] | —OMe | H | H | H |
| 2068-a 2068-b 3068 | 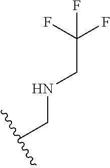 [SMILES: *CNCC(F)(F)F] | —OMe | H | H | H |
| 2069-a 2069-b 3069 | 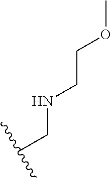 [SMILES: *CNCCOC] | —OMe | H | H | H |
| 2070-a 2070-b 3070 | 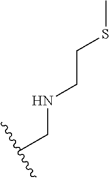 [SMILES: *CNCCSC] | —OMe | H | H | H |
| 2071-a 2071-b 3071 | 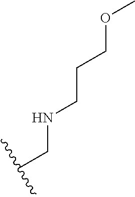 [SMILES: *CNCCCOC] | —OMe | H | H | H |
| 2072-a 2072-b 3072 | 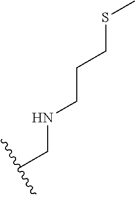 [SMILES: *CNCCCSC] | —OMe | H | H | H |
| 2073-a 2073-b 3073 | 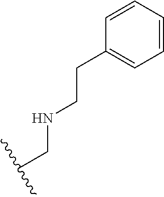 [SMILES: *CNCCc1ccccc1] | H | —OMe | H | H |
| 2074-a 2074-b 3074 | 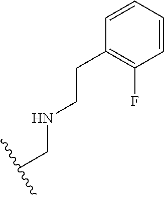 [SMILES: *CNCCc1ccccc1F] | H | —OMe | H | H |
| 2075-a 2075-b 3075 | 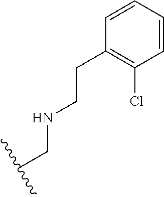 [SMILES: *CNCCc1ccccc1Cl] | H | —OMe | H | H |

In some embodiments, the disclosure provides a compound having any of any of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, but excluding one or more compounds selected from UMB28-1 to UMB28_18, UMB5_1 to UMB5_7, UMB10_1 to UMB_107, UMB22_1 to UMB22_20, UMB40_1 to UMB40_10, UMB42_1 to UMB42_12, and UMB23_1 to UMB23_14.

### Methods of Treatment

The compounds and compositions described herein can be used in methods for treating diseases, including but not limited to: a method of treating a condition by inhibiting SKI complex activity in a patient in need of said treatment, the method comprising administering to the patient a therapeutically effective amount of a compound of any of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salts, solvates, hydrates, cocrystals, or prodrugs thereof; a method of treating a condition by inhibiting viral replication in a patient in need of said treatment, the method comprising administering to the patient a therapeutically effective amount of a compound of any of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB_5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28 D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salts, solvates, hydrates, cocrystals, or prodrugs thereof; or a method of treating a condition by inducing interferon signaling in a patient in need of said treatment, the method comprising administering to the patient a therapeutically effective amount of a compound of any of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salts, solvates, hydrates, cocrystals, or prodrugs thereof.

In some embodiments, a condition is selected from a viral infection, a bacterial infection, and cancer. In some embodiments, a bacterial infection is selected from a lung infection, skin infection, soft tissue infection, gastrointestinal infection, urinary tract infection, meningitis, and sepsis. In some embodiments, a cancer is selected from adrenocortical cancer, hepatocellular cancer, hepatoblastoma, malignant melanoma, ovarian cancer, Wilm's tumor, Barrett's esophageal cancer, prostate cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. In some embodiments, a viral infection is caused by influenza, Middle East respiratory syndrome-related coronavirus (MERS-CoV), rhinovirus, polio, measles, Ebola, Coxsackie, West Nile, yellow fever, Dengue fever, lassa, lymphocytic choriomeningitis, Junin, Machupo, guanarito, hantavirus, Rift Valley Fever, La Crosse, California encephalitis, Crimean-Congo, Marburg, Japanese Encephalitis, Kyasanur Forest, Eastern equine encephalitis, Western equine encephalitis, severe acute respiratory syndrome (SARS), severe acute respiratory syndrome coronavirus 2 (SARS-CoV-2), parainfluenza, Tacaribe, or Pichinde viruses. In some embodiments, the viral infection is caused by influenza. In some embodiments, the viral infection is caused by severe acute respiratory syndrome coronavirus 2 (SARS-CoV-2).

In some embodiments, the methods for treating diseases described herein include the use of a compound selected from a compound of any of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf, or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salts, solvates, hydrates, cocrystals, or prodrugs thereof.

Efficacy of the methods, compounds, and combinations of compounds described herein in treating, preventing and/or managing the indicated diseases or disorders can be tested using various animal models known in the art. For example, methods for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy of treatments for colorectal cancer, including the CT26 model, are described in Castle, et al., BMC Genomics, 2013, 15, 190; Endo, et al., Cancer Gene Therapy, 2002, 9, 142-148; Roth et al., Adv. Immunol. 1994, 57, 281-351; Fearon, et al., Cancer Res. 1988, 48, 2975-2980.

### Pharmaceutical Compositions

In an embodiment, the disclosure provides a pharmaceutical composition for use in the treatment of the diseases and conditions described herein.

The pharmaceutical compositions are typically formulated to provide a therapeutically effective amount of a compound of any of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16 D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salts, solvates, hydrates, cocrystals, or prodrugs thereof, as described herein, as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are for use in the treatment of, without limitation, a condition selected from a viral infection, a bacterial infection, and cancer, the pharmaceutical composition comprising one or more compounds, or pharmaceutically acceptable salts, solvates, hydrates, cocrystals, or prodrugs thereof, having any one of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, and a pharmaceutically acceptable carrier. In some embodiments, a bacterial infection is selected from a lung infection, skin infection, soft tissue infection, gastrointestinal infection, urinary tract infection, meningitis, and sepsis. In some embodiments, a cancer is selected from adrenocortical cancer, hepatocellular cancer, hepatoblastoma, malignant melanoma, ovarian cancer, Wilm's tumor, Barrett's esophageal cancer, prostate cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. In some embodiments, a viral infection is caused by influenza, Middle East respiratory syndrome-related coronavirus (MERS-CoV), rhinovirus, polio, measles, Ebola, Coxsackie, West Nile, yellow fever, Dengue fever, lassa, lymphocytic choriomeningitis, Junin, Machupo, guanarito, hantavirus, Rift Valley Fever, La Crosse, California encephalitis, Crimean-Congo, Marburg, Japanese Encephalitis, Kyasanur Forest, Eastern equine encephalitis, Western equine encephalitis, severe acute respiratory syndrome (SARS), severe acute respiratory syndrome coronavirus 2 (SARS-CoV-2), parainfluenza, Tacaribe, or Pichinde viruses. In some embodiments, the viral infection is caused by influenza. In some embodiments, the viral infection is caused by severe acute respiratory syndrome coronavirus 2 (SARS-CoV-2), formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42 UMB18_D43 to UMB18_D47, UMB22_5_D48, UMB18_D49, UMB18_D50, UMB22_5_D51, UMB22_16 D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%1, 1.75%, 1.50%, 125%1, 1%, 0.5%, 0.4%, 0.3% 0.2%, 0.1%, 0.09% 0.08%, 0.07%, 0.06%, 0.05%, 0.04%, 0.03% 0.02% 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5 7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.10% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB_57, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42. UMB18_D43 to UMB18_D47, UMB22_5_D48, UMB18 D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42 UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf, or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42 UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, provided in the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing: a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, and a pharmaceutical excipient suitable for administration.

In preferred embodiments, the disclosure provides a solid pharmaceutical composition for oral administration containing: (i) an effective amount of: a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, and (ii) a pharmaceutical excipient suitable for administration. In some embodiments, the composition further contains (iii) an effective amount of an additional active pharmaceutical ingredient. For example, additional active pharmaceutical ingredients, as used herein, may include one or more compounds that induce SKI complex inhibition, viral replication inhibition, or interferon signaling. Such additional active pharmaceutical ingredients may also include those compounds used for sensitizing cells to additional agent(s), such as inducers of apoptosis and/or cell cycle arrest, and chemoprotection of normal cells through the induction of cell cycle arrest prior to treatment with chemotherapeutic agents.

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, 6-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing: a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, and a pharmaceutical excipient suitable for injection. Components and amounts of compounds in the compositions are as described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating a compound of formula (I), formula (10), formula (II), formula (20), formula (III), formula (IV), formula (V), formula (50), formula (VI), formula (VII), formula (VIIIa), formula (VIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing: a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (Ville), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of: a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16 D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, or a pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, can also be administered intraadiposally or intrathecally.

The compositions of the disclosure may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the disclosure may slow or inhibit the migration and proliferation of smooth muscle cells in the arterial wall which contribute to restenosis. A compound of the disclosure may be administered, for example, by local delivery from the struts of a stent, from a stent graft, from grafts, or from the cover or sheath of a stent. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. A compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the disclosure in a suitable solvent, followed by evaporation of the solvent. Excess drug on the surface of the stent may be removed via an additional brief solvent wash. In yet other embodiments, compounds of the disclosure may be covalently linked to a stent or graft. A covalent linker may be used which degrades in vivo, leading to the release of the compound of the disclosure. Any bio-labile linkage may be used for such a purpose, such as ester, amide or anhydride linkages. A compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, may additionally be administered intravascularly from a balloon used during angioplasty. Extravascular administration of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, via the pericard or via advential application of formulations of the disclosure may also be performed to decrease restenosis.

Exemplary parenteral administration forms include solutions or suspensions of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, in sterile aqueous solutions, for example, aqueous propylene glycol or dextrose solutions. Such dosage forms can be suitably buffered, if desired.

The disclosure also provides kits. The kits include a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, the compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, and another active pharmaceutical ingredient are provided as separate compositions in separate containers within the kit. In some embodiments, the compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In some embodiments, the kits described herein are for use in the treatment of a condition selected from a viral infection, a bacterial infection, and cancer. In some embodiments, a bacterial infection is selected from a lung infection, skin infection, soft tissue infection, gastrointestinal infection, urinary tract infection, meningitis, and sepsis. In some embodiments, a cancer is selected from adrenocortical cancer, hepatocellular cancer, hepatoblastoma, malignant melanoma, ovarian cancer, Wilm's tumor, Barrett's esophageal cancer, prostate cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. In some embodiments, a viral infection is caused by influenza, Middle East respiratory syndrome-related coronavirus (MERS-CoV), rhinovirus, polio, measles, Ebola, Coxsackie, West Nile, yellow fever, Dengue fever, lassa, lymphocytic choriomeningitis, Junin, Machupo, guanarito, hantavirus, Rift Valley Fever, La Crosse, California encephalitis, Crimean-Congo, Marburg, Japanese Encephalitis, Kyasanur Forest, Eastern equine encephalitis, Western equine encephalitis, severe acute respiratory syndrome (SARS), severe acute respiratory syndrome coronavirus 2 (SARS-CoV-2), parainfluenza, Tacaribe, or Pichinde viruses. In some embodiments, the viral infection is caused by influenza. In some embodiments, the viral infection is caused by severe acute respiratory syndrome coronavirus 2 (SARS-CoV-2).

### Dosages and Dosing Regimens

The amounts of: a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42 UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, may be provided in units of mg/kg of body mass or in mg/m² of body surface area.

In some embodiments, a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein is administered in multiple doses. In a preferred embodiment, a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB51 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein is administered in multiple doses. Dosing may be once, twice, three times, four times, five times, six times, or more than six times per day. Dosing may be once a month, once every two weeks, once a week, or once every other day. In other embodiments, a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, is administered about once per day to about 6 times per day. In some embodiments, a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf, or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, is administered once daily, while in other embodiments, a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein is administered twice daily, and in other embodiments a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, is administered three times daily.

Administration a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB51 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, may continue as long as necessary. In some embodiments, a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB15_to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, is administered for more than 1, 2, 3, 4, 5, 6, 7, 14, or 28 days. In some embodiments, a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein is administered for less than 28, 14, 7, 6, 5, 4, 3, 2, or 1 day. In some embodiments, a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42 UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein is administered chronically on an ongoing basis—e.g., for the treatment of chronic effects. In another embodiment, the administration of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB5_7, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB_57, UMB10_1 to UMB10_7, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of a compound of formula (I), formula (10), formula (II), formula (20), formula (III), formula (IV), formula (V), formula (50), formula (VI), formula (VII), formula (VIIIa), formula (VIIIb), formula (VIIIc), formula (VIIId), formula (VIIIe), formula (VIIIf), or formula 1001-1038, 2001-2028, 3001-3018, 5001-5013, 6001-6013, 7001-7006, 8001-8010, 8101-8105, 8201, 8301, 8401-8403, or 8501, UMB21_1 to UMB_18, UMB5_1 to UMB57, UMB10_1 to UMB_107, UMB22_1 to UMB22_20, UMB40_1 to UMB40_10, UMB42_1 to UMB42_12, UMB23_1 to UMB23_14, UMB28_D1 to UMB28_D42, UMB18_D43 to UMB18_D47, UMB22_5_D48, UMB18_D49, UMB18_D50, UMB22_5_D51, UMB22_16_D52, UMB22_5_D53, UMB22_16_D54, UMB22_5_D55 to UMB22_5_D59, UMB22_16_D60, UMB22_5_D61 to UMB22_5_D70, UMB18_D71, or UMB18_D72, or pharmaceutically acceptable salt thereof, described herein, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Targeting the SKI Complex for Anti-Viral Therapeutic Development

This example describes the use of the yeast Saccharomyces cerevisiae as a system to identify novel functional interactions between viral proteins and eukaryotic cells. Viral proteins must intimately interact with the host cell machinery during virus replication. These results demonstrate that when the Influenza virus NS1 gene is expressed in yeast it causes a slow growth phenotype. NS1 has been characterized as an interferon antagonist in mammalian cells, yet yeast lack an interferon system, suggesting further interactions between NS1 and eukaryotic cells. Using the slow growth phenotype as a reporter of NS1 function, the yeast knockout library collection was utilized to perform a suppressor screen where several genes in the SKI complex were identified as hits. The SKI complex in yeast, (consisting of SKI2/SKI3/SKI8) through humans (SKIV2L, TTC37 and WDR61, respectively) is part of the RNA exosome complex which degrades RNA. It was found that when SKIV2L, the human homologue of SKI2, and TTC37, the human homologue of SKI3, were knocked down by siRNAs, there was reduced Influenza virus and MERS-CoV replication. While not wishing to be bound by any particular theory, these results suggest that SKIV2L and TTC37 are proviral factors for multiple viruses. Moreover, compounds selected to bind to the interface between SKIV2L and WDR61 (the human homologue of SKI8), inhibit Influenza virus replication demonstrating that the SKI complex is a viable anti-viral target for further development

Influenza Virus Genes Expressed in Yeast Produce a Slow Growth Phenotype

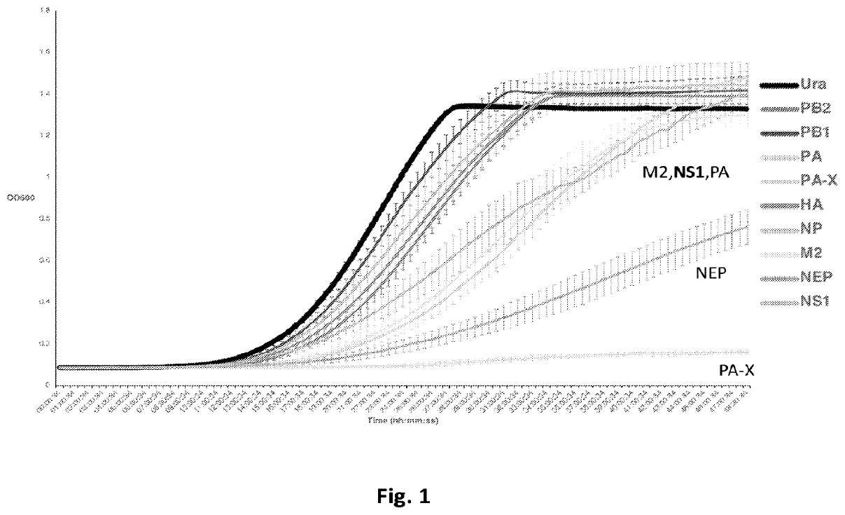

Genes from the Influenza virus genome were cloned into a galactose inducible (GAL1) yeast expression vector with a C-terminal GFP tag and transformed into yeast. When grown in the presence of 2% glucose (Glu), the expression of viral genes was inhibited and the yeast containing this plasmid could grow similarly to yeast transformed with a vector control. However, when grown in the presence of 2% galactose (Gal), viral genes were expressed. Whether any of the Influenza encoded proteins could inhibit yeast growth was analyzed by performing 48 hour (h) growth curves, measuring the OD600 as the readout for growth on an automated plate reader. It was found that five Influenza virus proteins inhibited growth of yeast (FIG. 1).

It was hypothesized that the slow growth phenotype induced by NS1 was a result of the viral protein disrupting normal cellular function, allowing a suppressor screen to be performed. It was also hypothesized that knockout of yeast genes involved in the NS1-mediated slow growth may reduce the level of inhibition and therefore increase growth rate. To test this hypothesis, the inducible NS1 plasmid was transformed into a pooled collection of the yeast knockout library. This library consisted of ˜4600 non-essential gene knockouts. When this transformed library was plated onto Glu plates, the yeast colonies that formed were of similar size. However, when plated to Gal plates to induce NS1 expression, abroad range of colony sizes were observed. Large colonies were picked, validated and sequenced to identify what deletion the yeast had in their genome which made them resistant to NS1 expression. Several SKI gene family members were identified in this screen.

Inhibition of the SKIV2L and TTC37 Genes Reduce Influenza Virus Replication.

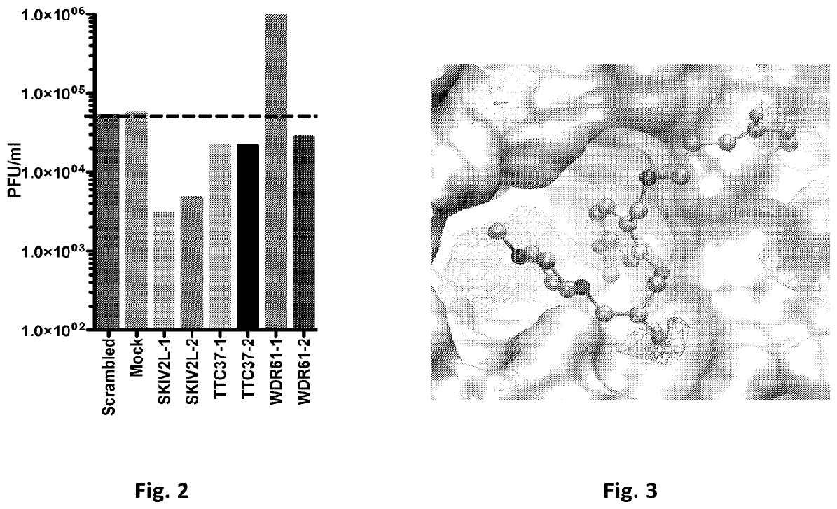

SKIV2L (human homologue of SKIV2L), TTC37 (human homologue of SKI3) and WDR61 (human homologue of SKI8) were knocked down in human A549 cells with siRNAs and the effect of Influenza virus replication was assessed (FIG. 2). It was found that knockdown of SKIV2L and TTC37 resulted in reduced virus replication while knockdown of WDR61 resulted in either no effect or an increase in replication.

Compounds Targeting the Interface Between SKI3 and SKI8 Inhibit Influenza Virus Replication.

The data demonstrates that there could be an effect of the SKI complex on Influenza virus replication and that structure of this complex is important for its proper function. A computer aided drug design algorithm called SILCS (Site Identification by Ligand Competitive Saturation) was next utilized to identify putative drug binding sites on target proteins or enzymes of interest, in this case the interface between SKIV2L, TTC37 and WDR61 based on the use of the three-dimensional structures of SKI2, SKI3 and SKI8, respectively. From SILCS, 3D functional group probability distribution maps, termed FragMaps, were obtained from the PDB coordinates of the available structures of the SKI2/3/8 complex (FIG. 3). The FragMaps are used to identify regions on the enzyme's surface for which different types of functional groups may have favorable interactions and can be used to identify putative ligand binding pockets and subsequently to direct small molecule inhibitor design. Notably, SILCS allows for 1) qualitative, visual analysis of the protein-binding pockets to drive the design of synthetically accessible modifications and for 2) quantitative predictions of changes in binding affinity associated with the designed modifications. As the SILCS FragMaps encompass the entire protein, they allow for identification of multiple putative pockets followed by database screening. This allowed for chosen binding pockets to be targeted for selection and docking.

The site targeted on ySki8 was selected based on it being in contact with residues on ySki3 in the Ski complex crystallographic structure (PDB ID: 4BUJ) and on the pattern of SILCS FragMaps showing the presence of adjacent apolar regions along with local polar regions that would potentially allow for the binding of drug like molecules that contain multiple ring systems with polar characteristics. Twenty top scoring compounds were selected and tested at 10 μM concentration for their ability to block Influenza virus infection in A549 cells. Of these 20 compounds, one compound was found to inhibit Influenza virus replication over 20 fold. A broader concentration range was tested for all 20 compounds and the same compound proved effective at 50 μM, 10 μM and 1 μM compared to control treated cells. This compound, called UMBCADD-0018, was identified for further anti-Influenza virus inhibition development. Of the 20 compounds tested, multiple compounds with a similar structure to UMBCADD-0018 also showed effects against Influenza virus in A549 cells, while those with divergent structures had no effect, suggesting the use of the UMBCADD-0018 (#96509034) scaffold as an Influenza antiviral.

As described herein, compounds have been identified which have broadly acting antiviral activity. The compounds have been modeled to target the SKI complex (part of the RNA exosome) via in silico modeling of a library of compounds on the yeast SKI complex crystal structure. The SKI complex is made up of protein components which are called yeast SKI2/human SKIV2L, yeast SKI3/human TTC37 and yeast SKIS/human WDR61. The function of the SKI complex in yeast and mammalian cells is to unwind RNA, the ySKI2/hSKIV2L protein has helicase activity, and feed RNA into the degradation machinery of the RNA exosome for degradation. The compounds are predicted to bind in a pocket at the interface of SKI8/WDR61 and SKI3/TTC37. Treating cells with these compounds induced basal Interferon induced gene expression, and hypersensitized the cell to further Interferon induction. After treatment with the identified compounds, infection of cells with the viruses tested lead to induction of interferon and anti-viral proteins, resulting in reduced virus replication. Exemplary compounds with anti-viral activity include #96509034, 5612793 and 10253964, which were purchased from Chembridge Corp. Additionally, an analogue of #96509034 was also identified to have increased activity (catalog #27092311). In view of the identification of the target and compounds that bind, novel analogues are developed that exhibit similar or improved binding activity.

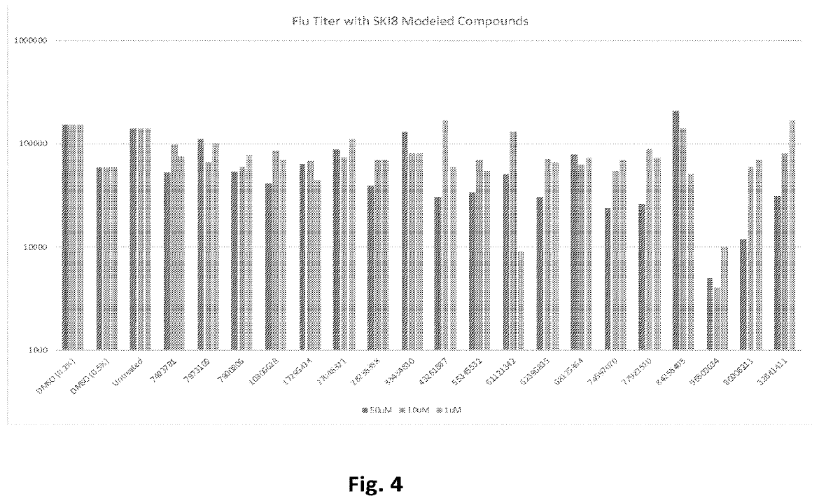

FIG. 4 illustrates the testing of SKI complex targeted compounds against Influenza virus. Compound names are on the X-axis and PFU/virus/ml is on the Y-axis. All results are from plaque assays for Influenza virus infection. The data and experimental protocols on which FIG. 4 is based is described in the following tables:

| | Mol | |
|---|---|---|
| ID | Weight | Name/Structure |
| 7493781 | 493.6542 | 2-({5-[1-(4-ethylphenoxy)ethyl]-4-methyl-4H- |
| | | 1,2,4-triazol-3-yl}thio)-N-(5-methyl-4-phenyl-1, |
| | | 3-thiazol-2-yl)acetamide |
| 7973169 | 408.5454 | N-(3-{[2-(4-cyclohexylphenoxy)acetyl]amino} |
| | | phenyl)pentanamide |
| 7900806 | 427.4611 | 2-[2-(cyclohexylamino)-2-oxoethoxy]-N-(4- |
| | | methoxy-2-nitrophenyl)benzamide |
| 10306628 | 398.553 | 1-(4-ethyl-1-piperazinyl)-3-(3-{[methyl(2- |
| | | pyridinylmethyl)amino]methyl}phenoxy)- |
| | | 2-propanol |
| 17295424 | 417.4686 | N-[(5-methyl-2-{2-[(3- |
| | | phenoxypropanoyl)amino]phenyl}-1,3-oxazol-4- |
| | | yl)methyl]-2-butynamide |
| 23566722 | 392.5897 | 1-(3-{[[3-(dimethylamino)propyl](methyl)amino] |
| | | methyl}phenoxy)-3-(4-ethyl-1-piperazinyl)-2- |
| | | propanol |
| 27046321 | 482.6283 | N-[(2R*,3R*)-1′-[(3,5-dimethyl-1H-pyrazol-1- |
| | | yl)acetyl]-2-(2-methoxyethoxy)-2,3- |
| | | dihydrospiro[indene-1,4′-piperidin]-3-yl]-2- |
| | | methylpropanamide |
| 28236358 | 447.6669 | 1-[cyclohexyl(methyl)amino]-3-[2-methoxy-4- |
| | | ({[2-(1-methyl-4-piperidinyl)ethyl]amino}methyl) |
| | | phenoxy]-2-propanol |
| 35434810 | 429.5643 | 3-[5-(2,5-dimethoxybenzyl)-1,3,4-oxadiazol-2-yl]- |
| | | N-(3,3,5,5-tetramethylcyclohexyl)propanamide |
| 43261887 | 427.567 | 4-(cyclopropylcarbonyl)-7-(5-methyl-2-thienyl)-9- |
| | | (tetrahydro-2H-pyran-2-ylmethoxy)-2,3,4,5- |
| | | tetrahydro-1,4-benzoxazepine |
| 55345532 | 495.6242 | N′-[2-(1-cyclohexen-1-yl)ethyl]-N-(2-hydroxyethyl)- |
| | | N-isopropyl-1-(2-methoxybenzyl)-4-oxo-1,4- |
| | | dihydro-3,5-pyridinedicarboxamide |
| 61121342 | 449.5208 | 2-[(1-adamantylmethyl)({3-[3-(trifluoromethyl) |
| | | benzyl]-1,2,4-oxadiazol-5-yl}methyl)amino]ethanol |
| 62396835 | 431.5366 | methyl 1-{2-hydroxy-3-[3-({methyl[(3-methyl-5- |
| | | isoxazolyl)methyl]amino}methyl)phenoxy]propyl}- |
| | | 4-piperidinecarboxylate |
| 68125364 | 521.6619 | N-{[5-[(cyclohexylmethyl)thio]-4-(4-fluorophenyl)- |
| | | 4H-1,2,4-triazol-3-yl]methyl}-2-(2-oxo-4-phenyl-1- |
| | | pyrrolidinyl)acetamide |
| 74597070 | 389.5862 | 1-[cyclohexyl(methyl)amino]-3-[2-({[2-(1- |
| | | pyrrolidinyl)ethyl]amino}methyl)phenoxy]-2- |
| | | propanol |
| 77921510 | 402.5413 | 1-(4-ethyl-1-piperazinyl)-3-[3-({methyl[(3-methyl-5- |
| | | isoxazolyl)methyl]amino}methyl)phenoxy]-2- |
| | | propanol |
| 84256405 | 496.661 | N-[3-(4-{[3-(1H-1,2,3-benzotriazol-1-yl)propyl] |
| | | amino}-1-piperidinyl)phenyl]-4-phenylbutanamide |
| 96509034 | 431.5553 | 1-[2-({[2-(2-fluorophenyl)ethyl]amino}methyl)-5- |
| | | methoxyphenoxy]-3-(4-methyl-1-piperazinyl)-2- |
| | | propanol |
| 86006211 | 381.5849 | 1-[2-({[2-(diethylamino)ethyl]amino}methyl) |
| | | phenoxy]-3-(4-thiomorpholinyl)-2-propanol |
| 32841411 | 420.5998 | 1-(2-{[(2,3-dihydroimidazo[2,1-b][1,3]thiazol-6- |
| | | ylmethyl)amino]methyl}phenoxy)-3-(4- |
| | | thiomorpholinyl)-2-propanol |

| | | | Mol | | ml |
|---|---|---|---|---|---|
| Tuve | ID | mg | Weight | g in 1 = 10 mM | DMSO |
| 1 | 7493781 | 1 | 493.6542 | 4.936542 | 0.20 |
| 2 | 7973169 | 1 | 408.5454 | 4.085454 | 0.24 |
| 3 | 7900806 | 1 | 427.4611 | 4.274611 | 0.23 |
| 4 | 10306628 | 1 | 398.553 | 3.98553 | 0.25 |
| 5 | 17295424 | 1 | 417.4686 | 4.174686 | 0.24 |
| 6 | 23566722 | | | | |
| 7 | 27046321 | 1 | 482.6283 | 4.826283 | 0.21 |
| 8 | 28236358 | 1 | 447.6669 | 4.476669 | 0.22 |
| 9 | 35434810 | 1 | 429.5643 | 4.295643 | 0.23 |
| 10 | 43261887 | 1 | 427.567 | 4.27567 | 0.23 |
| 11 | 55345532 | 1 | 495.6242 | 4.956242 | 0.20 |
| 12 | 61121342 | 1 | 449.5208 | 4.495208 | 0.22 |
| 13 | 62396835 | 1 | 431.5366 | 4.315366 | 0.23 |
| 14 | 68125364 | 1 | 521.6619 | 5.216619 | 0.19 |
| 15 | 74597070 | 1 | 389.5862 | 3.895862 | 0.26 |
| 16 | 77921510 | 1 | 402.5413 | 4.025413 | 0.25 |
| 17 | 84256405 | 1 | 496.661 | 4.96661 | 0.20 |
| 18 | 96509034 | 1 | 431.5553 | 4.315553 | 0.23 |
| 19 | 86006211 | 1 | 381.5849 | 3.815849 | 0.26 |
| 20 | 32841411 | 1 | 420.5998 | 4.205998 | 0.24 |
| Flu titer | | | Flu titer | | |
| (pfu/ml) | | | (pfu/ml) | | |
| 153000 | DMSO | 10 | 153000 | DMSO | 50 |
| | (0.1%) | μM | | (0.1%) | μM |
| 58700 | DMSO | | | DMSO | |
| | (0.5%) | | 58700 | (0.5%) | |
| 139000 | Untreated | | 139000 | Untreated | |
| 98000 | 7493781 | | 53000 | 7493781 | |
| 66000 | 7973169 | | 110000 | 7973169 | |
| 60000 | 7900806 | | 54000 | 7900806 | |
| 85000 | 10306628 | | 42000 | 10306628 | |
| 68000 | 17295424 | | 64000 | 17295424 | |
| 74000 | 27046321 | | 87000 | 27046321 | |
| 70000 | 28236358 | | 39000 | 28236358 | |
| 80000 | 35434810 | | 130000 | 35434810 | |
| 170000 | 43261887 | | 30000 | 43261887 | |
| 70000 | 55345532 | | 34000 | 55345532 | |
| 130000 | 61121342 | | 50000 | 61121342 | |
| 71000 | 62396835 | | 30000 | 62396835 | |
| 62000 | 68125364 | | 79000 | 68125364 | |
| 55000 | 74597070 | | 24000 | 74597070 | |
| 90000 | 77921510 | | 26000 | 77921510 | |
| 140000 | 84256405 | | 210000 | 84256405 | |
| 4000 | 96509034 | | 5000 | 96509034 | |
| 60000 | 86006211 | | 12000 | 86006211 | |
| 80000 | 32841411 | | 31000 | 32841411 | |
| | | | Flu titer | | |
| | | | (pfu/ml) | | |
| | | | 153000 | DMSO | 1 μM |
| | | | | (0.1%) | |
| | | | 58700 | DMSO | |
| | | | | (0.5%) | |
| | | | 139000 | Untreated | |
| | | | 75000 | 7493781 | |
| | | | 101000 | 7973169 | |
| | | | 77000 | 7900806 | |
| | | | 70000 | 10306628 | |
| | | | 44000 | 17295424 | |
| | | | 110000 | 27046321 | |
| | | | 70000 | 28236358 | |
| | | | 80000 | 35434810 | |
| | | | 60000 | 43261887 | |
| | | | 55000 | 55345532 | |
| | | | 9000 | 61121342 | |
| | | | 66000 | 62396835 | |
| | | | 72000 | 68125364 | |
| | | | 70000 | 74597070 | |
| | | | 72000 | 77921510 | |
| | | | 50000 | 84256405 | |
| | | | 10000 | 96509034 | |
| | | | 70000 | 86006211 | |
| | | | 170000 | 32841411 | |

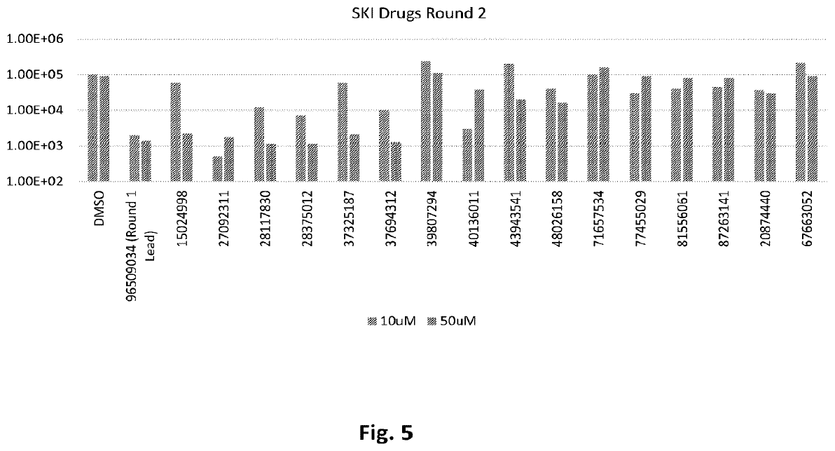

FIG. 5 illustrates the testing of subsequent round SKI complex targeted compounds against Influenza virus. Compound names are on the X-axis and PFU/virus/ml is on the Y-axis. All results are from plaque assays for Influenza virus infection. The data and experimental protocols on which FIG. 5 is based is described in the following table:

| | | Compound | | |
|---|---|---|---|---|
| | | # | 10 μM | 50 μM |
| | DMSO | DMSO | 100000 | 90000 |
| 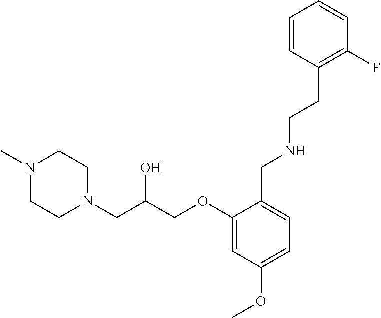 [SMILES: COc1ccc(CNCCc2ccccc2F)c(OCC(O)CN2CCN(C)CC2)c1] | 18-lead | 96509034 (Round 1 Lead) | 2000 | 1400 |
| 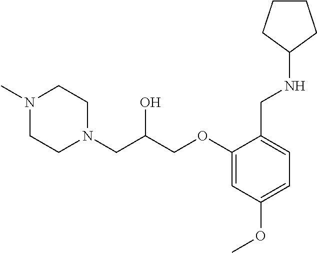 [SMILES: COc1ccc(CNC2CCCC2)c(OCC(O)CN2CCN(C)CC2)c1] | 18-1 | 15024998 | 60000 | 2200 |
| 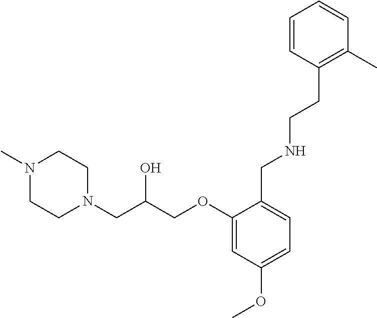 | 18-2 | 27092311 | 500 | 1700 |
| 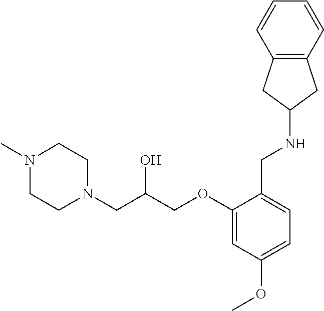 [SMILES: COc1ccc(CNC2Cc3ccccc3C2)c(OCC(O)CN2CCN(C)CC2)c1] | 18-3 | 28117830 | 12000 | 1100 |
| 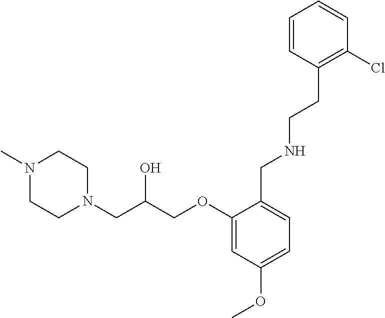 [SMILES: COc1ccc(CNCCc2ccccc2Cl)c(OCC(O)CN2CCN(C)CC2)c1] | 18-4 | 28375012 | 7000 | 1100 |
| 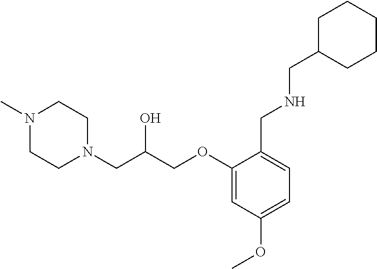 [SMILES: COc1ccc(CNCC2CCCCC2)c(OCC(O)CN2CCN(C)CC2)c1] | 18-5 | 37325187 | 60000 | 2100 |
| 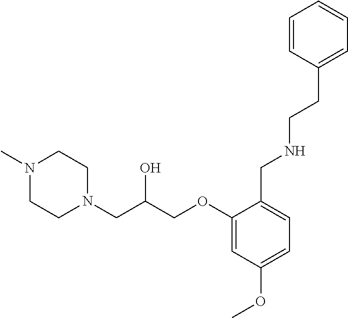 [SMILES: COc1ccc(CNCCc2ccccc2)c(OCC(O)CN2CCN(C)CC2)c1] | 18-6 | 37694312 | 10000 | 1300 |
| 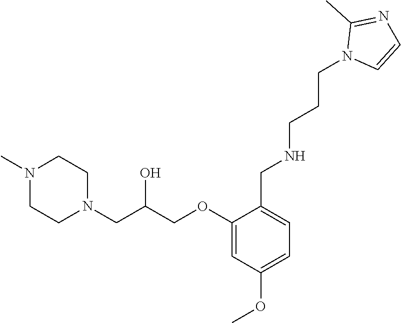 [SMILES: COc1ccc(CNCCCn2ccnc2C)c(OCC(O)CN2CCN(C)CC2)c1] | 18-7 | 39807294 | 240000 | 110000 |
| 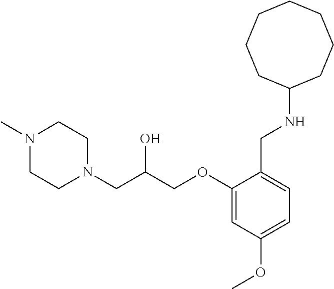 [SMILES: COc1ccc(CNC2CCCCCCC2)c(OCC(O)CN2CCN(C)CC2)c1] | 18-8 | 40136011 | 3000 | 38000 |
| 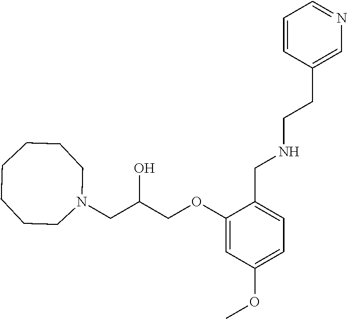 [SMILES: COc1ccc(CNCCc2cccnc2)c(OCC(O)CN2CCCCCCC2)c1] | 18-9 | 48943541 | 200000 | 20000 |
| 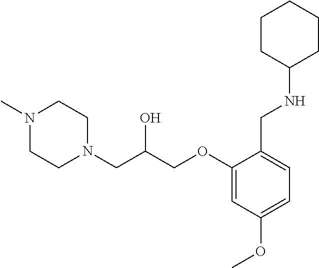 [SMILES: COc1ccc(CNC2CCCCC2)c(OCC(O)CN2CCN(C)CC2)c1] | 18-10 | 48026158 | 40000 | 16000 |
| 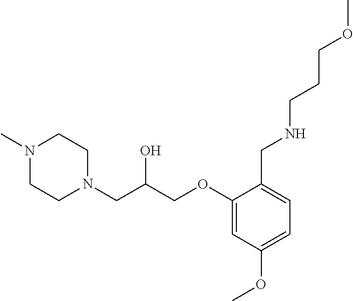 | 18-11 | 71657534 | 100000 | 160000 |
| 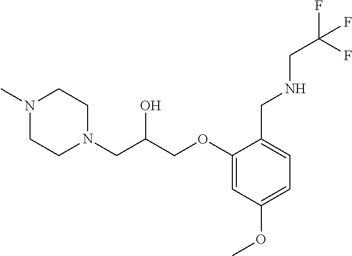 [SMILES: COc1ccc(CNCC(F)(F)F)c(OCC(O)CN2CCN(C)CC2)c1] | 18-12 | 77455029 | 30000 | 90000 |
| 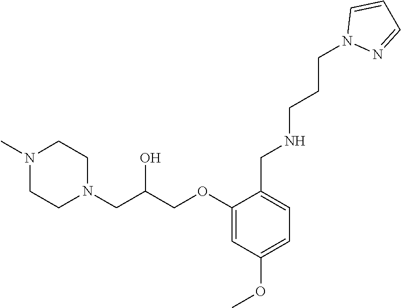 [SMILES: COc1ccc(CNCCCn2cccn2)c(OCC(O)CN2CCN(C)CC2)c1] | 18-13 | 81556061 | 40000 | 80000 |
| 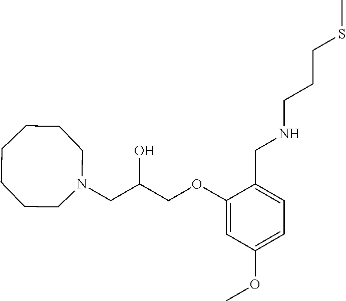 [SMILES: COc1ccc(CNCCCSC)c(OCC(O)CN2CCCCCCC2)c1] | 18-14 | 87263141 | 4400 | 80000 |
| 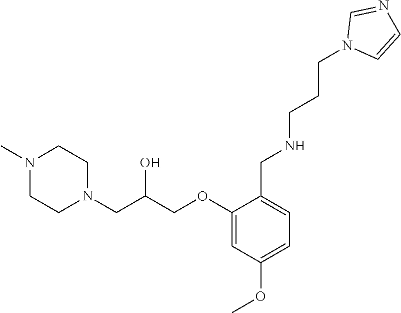 [SMILES: COc1ccc(CNCCCn2ccnc2)c(OCC(O)CN2CCN(C)CC2)c1] | 18-15 | 20874440 | 36000 | 30000 |
| 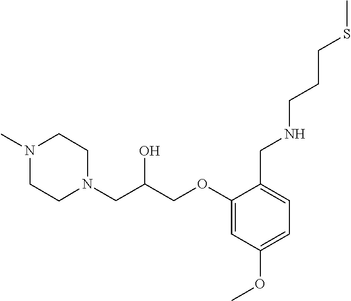 | 18-16 | 67663052 | 21000 | 90000 |

| 50 μM | | | | |
|---|---|---|---|---|
| screen | | first | second | |
| Compound | | experiment | experiment | Avg |
| 0.5% | | PFU/ml | PFU/ml | PFU/ml |
| DMSO | ID | 90000 | 34000 | 62000 |
| 18 | 96509034 | 1400 | 300 | 850 |
| 18-1 | 15024998 | 2200 | 1400 | 1800 |
| 18-2 | 27092311 | 1700 | 100 | 900 |
| 18-3 | 28117830 | 1100 | 1700 | 1400 |
| 18-4 | 28375012 | 1100 | 300 | 700 |
| 18-5 | 3735187 | 2100 | 400 | 1250 |
| 18-6 | 37694312 | 1300 | 300 | 800 |
| 18-7 | 39807294 | 110000 | 31000 | 70500 |
| 18-8 | 40136011 | 3800 | 200 | 2000 |
| 18-9 | 43943541 | 20000 | 2600 | 11300 |
| 18-10 | 48026158 | 16000 | 1900 | 8950 |
| 18-11 | 71657534 | 160000 | 31000 | 95500 |
| 18-12 | 77455029 | 90000 | 26000 | 58000 |
| 18-13 | 81556061 | 80000 | 110000 | 95000 |
| 18-14 | 87263141 | 80000 | 1100 | 40550 |
| 18-15 | 20874440 | 70000 | 1000 | 35500 |
| 18-16 | 67663052 | 30000 | 2300 | 16150 |
| 10 μM | | | | |
| screen | | | | |
| Compound | | first | second | Avg |
| 0.1% | | experiment | experiment | PFU/ml |
| DMSO | ID | 100000 | 42000 | 71000 |
| 18 | 96509034 | 2000 | 1100 | 1550 |
| 18-1 | 15024998 | 60000 | 19000 | 39500 |
| 18-2 | 27092311 | 500 | 1900 | 1200 |
| 18-3 | 28117830 | 12000 | 1000 | 6500 |
| 18-4 | 28375012 | 7000 | 800 | 3900 |
| 18-5 | 37325187 | 60000 | 500 | 30250 |
| 18-6 | 37694312 | 10000 | 500 | 5250 |
| 18-7 | 39807294 | 240000 | 44000 | 142000 |
| 18-8 | 40136011 | 3000 | 700 | 1850 |
| 18-9 | 43943541 | 200000 | 9000 | 104500 |
| 18-10 | 48026158 | 40000 | 2600 | 21300 |
| 18-11 | 71657534 | 100000 | 39000 | 69500 |
| 18-12 | 77455029 | 30000 | 28000 | 29000 |
| 18-13 | 81556061 | 40000 | 20000 | 30000 |
| 18-14 | 87263141 | 44000 | 12000 | 28000 |
| 18-15 | 20874440 | 16000 | 8000 | 12000 |
| 18-16 | 67663052 | 210000 | 18000 | 114000 |

The reduction of the SKI complex activity through the disclosed compounds reduces the RNA degradation activity of the RNA Exosome, leading to increased cytoplasmic RNA levels and triggering an induction of interferon signaling. The induction of interferon signaling provides a broad-spectrum anti-viral response, leading to protection from a wide array of viral pathogens. The disclosed compounds can also be used to treat other diseases where interferon induction is protective, including cancer and bacterial infections.

### Example 2: The SKI Complex is a Broad-Spectrum, Host-Directed, Antiviral Drug Target for Coronaviruses, Influenza and Filoviruses

The severe acute respiratory syndrome coronavirus-2 (SARS-CoV-2) pandemic of 2020 has made it clear that there is a need for better antiviral countermeasures. Here presented is work that defines the mammalian SKI complex, an RNA helicase that links to the RNA exosome, as a broad-spectrum, host-directed, antiviral drug target. A yeast suppressor screening was used to find a functional genetic interaction between proteins from influenza A virus (IAV) and Middle East respiratory syndrome coronavirus (MERS-CoV). Subsequent siRNA-mediated knockdown of the mammalian SKI complex showed that this group of protein is important for replication of both of IAV and MERS-CoV. Using in silico modeling approaches it was found a potential binding pocket on one of the SKI complex subunits and screened compounds predicted to bind for potential antiviral activity. This screening found three distinct chemical structures that all displayed antiviral activity against IAV and MERS-CoV. The lead compound was termed UMB18 and have a very similarly structured chemical termed UMB18-2. These chemicals were additionally found to inhibit replication of the filoviruses Ebola and Marburg along with the other pathogenic human coronaviruses, SARS-CoV and SARS-CoV-2. Treatment of cells with the lead compounds and infection with IAV was used to determine that the mechanism of inhibition is through inhibition of viral RNA production. This work defines the mammalian SKI complex as a potential broad-spectrum antiviral drug target and identifies potential lead compounds for further development.

At the end of 2019 cases of pneumonia of unknown etiology were identified in China. In the first week of January, a novel coronavirus was identified as the cause and was found to be spreading between people. In the months since, that virus has spread around the world leading to the WHO announcing it a pandemic on 11 Mar. 2020 and the milestone of a 3 million confirmed cases was passed on 27 Apr. 2020. Amongst many things that the SARS-CoV-2 (severe acute respiratory syndrome coronavirus-2) outbreak has demonstrated is the need for both specific and broadly acting antiviral therapeutics. There is a need for the development of broad-spectrum antiviral compounds to treat viruses we know about, and those yet to emerge in the human population. With the emergence of three novel coronaviruses in the past 18 years, there will undoubtedly be more coronaviruses and other viruses that emerge in the future.

Here, it is detailed work identifying the SKI complex as a potential host-directed broad-spectrum antiviral target. The SKI complex is an RNA helicase involved in several aspects of RNA metabolism. It has previously been suggested that the SKI complex can regulate the IFN response and has a link to influenza cap-snatching, but beyond this has not been associated with viral replication. Using yeast suppressor screening we identified that influenza A virus (IAV) NS1 and Middle East respiratory syndrome coronavirus (MERS-CoV) ORF4a proteins have a genetic interaction with the SKI complex. It was a previously used yeast suppressor screening approach to identify SIRT1 as a proviral factor for MERS-CoV in mammalian cells (Weston2019). Finding that both viral proteins have a genetic interaction with the yeast SKI complex we subsequently determined that siRNA knockdown of the human SKI complex resulted in significant reduction in replication of these two distinct viruses, suggesting the complex may be a potential broad-spectrum antiviral target. Using an in silico modeling approach it was identified a binding pocket on one of the subunits of the SKI complex and screened compounds predicted to bind for antiviral activity. This work identified three chemical backbone structures that were capable of inhibiting both IAV and MERS-CoV. Data is presented to suggest that the mechanism of antiviral action is through inhibition of viral mRNA production. Moreover, the lead compound was found to inhibit replication of the filoviruses Ebola and Marburg, extending the broad-spectrum activity to a third viral family that causes significant human morbidity and mortality. Finally, it was found that the lead compound has broad anti-coronavirus activity, being capable of inhibiting SARS-CoV and SARS-CoV-2 replication.

This work identifies a potential novel host factor involved in the replication of coronaviruses, influenza and filoviruses. Multiple chemical structures were identified that are modeled to interact with the SKI complex, and all show broad-spectrum antiviral activity. These chemical structures will act as the basis of structure-activity relationship studies in the search for more potent chemicals. Developing broad-spectrum antivirals that target both the viruses themselves and the host they infect that can be used in combination may be the best approach to prepare for the next viral disease outbreak we have to deal with as a population. This work details a novel host target and identifies promising lead compounds with broad-spectrum antiviral activity.

Materials and Methods

Plasmids and compounds: genes from influenza were synthesized by Biobasic Inc. using sequence information for the CAL09 strain. Genes were cloned into a modified pRS413 plasmid containing a GAL1 promoter. See Weston2019 for further detail on the yeast plasmid and cloning. All SKI targeting compounds were purchased from the ChemBridge Hit2Lead library.

Yeast: see Weston2019 for experimental details on yeast. In brief, plasmids were transformed into the PDR1/PDR3 knockout strain derived from BY4742 (Matα, his3Δ1, leu2Δ0, lys2Δ0, ura3Δ0) as described (Basu2009&Weston2019). For growth experiments, single colonies of yeast were picked from plates and grown for two days at 30° C. to reach stationary phase in CAA media, containing 2% raffinose. Cultures were subsequently diluted in CAA media containing 2% galactose to induce gene expression. Optical density (OD600) of the cultures were analyzed using a Synergy HTX Multi-Mode plate reader.

Mammalian cell culture: A549 and Huh7 cells were cultured in DMEM (Quality Biological), supplemented with 10% (v/v) fetal calf serum (FCS; Sigma) and 1% (v/v) penicillin/streptomycin (pen/strep, 10,000 U/ml/10 mg/ml; Gemini Bio-Products). Vero E6 cells were cultured in DMEM supplemented with FCS and pen/strep, as A549 and Huh7, but additionally supplemented with 1% (v/v) L-glutamine (2 mM final concentration, Gibco). Cells were maintained at 37° C. and 5% CO₂.

Viruses: influenza A virus NL09 strain was a kind gift from Florian Kramer (Icahn School of Medicine, Mt. Sinai). MERS-CoV (Jordan strain—GenBank accession no. KC776174.1, MERS-CoV-Hu/Jordan-N3/2012) stocks were prepared by infection of Vero E6 cells and titer determined by plaque assay using these cells (as described previously Coleman2015). SARS-CoV MA15 has been described previously (Roberts2007). Stocks were produced as for MERS-CoV. Samples of SARS-CoV-2 were obtained from the CDC following isolation from a patient in Washington State, USA (WA-1 strain—BEI #NR-52281). Stocks were prepared by infection of Vero E6 cells for two days when CPE was starting to be visible. Media were collected and clarified by centrifugation prior to being aliquoted for storage at −80° C. Titer of stock was determined by plaque assay using Vero E6 cells as described previously (Coleman2015). All coronavirus work was performed in a Biosafety Level 3 laboratory and approved by our Institutional Biosafety Committee. Influenza work was performed at Biosafety Level 2.

Filovirus

Virus infections: in all instances, cells were plated one day prior to infection with the indicated viruses at the MOI indicated in the text. For influenza infections, virus was diluted to the appropriate level in DMEM with 4% (w/v) bovine serum albumin (Sigma), 1% pen/strep and 1 μg/ml TPCK-treated trypsin (Sigma): “infection media”. Cells were inoculated with virus for 1 hour (h) at 37° C./5% CO₂ (100 μl inoculum in a 24 well plate with rocking of the plate every 10 minutes (min) to avoid drying out of cells). After that incubation, media were removed and replaced with fresh infection media and returned to the incubator. For coronavirus infections, virus was diluted to the appropriate MOI in culture media and added to cells.

siRNA knockdown: Cells were transfected with indicated siRNA purchased from Sigma using their Rosetta prediction algorithm and purchasing the top three ranked siRNA sequences. Scrambled siRNA was used as a control (MISSION siRNA Universal Negative Control #1 [Sigma]). For transfection of cells in a 24 well dish, 4.4 μl Opti-MEM (Gibco) was mixed with 2.2 μl Oligofectamine (Thermo Scientific) and incubated for 5 min at room temperature (RT) and then mixed with 35.5 μl Opti-MEM and 0.8 μl of 50 μM siRNA. This mix was incubated for 20 min at RT. A further 177 μl of Opti-MEM was added to the transfection mixture, media were removed from cells and 200 μl of transfection mixture was added. After a 4 h incubation at 37° C./5% CO₂, 200 μl of 20% FCS DMEM was added to the cells resulting in a final concentration of 10%. Cells were then incubated at 37° C./5% CO₂, for 3 days prior to experimental use.

RNA extraction and qRT-PCR: cells were collected in TRIzol and RNA was extracted using Direct-zol RNA miniprep kit (Zymo Research) as per the manufacturer's instructions. RNA was converted to cDNA using RevertAid RT Kit (Thermo Scientific), with 12 μl of extracted RNA per reaction. For qRT-PCR, 2 μl of cDNA reaction product was mixed with PowerUp SYBR Green Master Mix (Applied Biosystems) and gene specific primers as listed in the table. To normalize loading, GAPDH or 18S were used as housekeeping genes (18S was analysed by TaqMan Gene Expression Assays (Applied Biosystems) and TaqMan Fast Advanced Master Mix). Fold change between drug treated and vehicle control was determined by calculating ΔΔCT after normalization to the housekeeper gene.

| Gene target | FWD (5′ to 3′) | REV (5′ to 3′) |
|---|---|---|
| SKIV2L | TGCAAGACCTA | GAATGAGGCGA |
| | GTGTTGAAG | TAGAAATCAC |
| TTC37 | TTATTGACGTG | GTTATTGTGAG |
| | CTGGTAAAC | GACAATCTCTG |
| WDR61 | ATTCTGTCCTG | AAAGAAGGTGT |
| | ATGACACTC | GAACACAAG |
| IAV NS1 | GACCRATCCTG | AGGGCATTYTG |
| | TCACCTCTGAC | GACAAAKCGTC |
| | | TA |
| SARS-CoV- | CACATTGGCAC | GAGGAACGAGA |
| 2 N | CCGCAATC | AGAGGCTTG |
| SARS-CoV- | GTGARATGGTC | CARATGTTAAAS |
| 2 RdRp | ATGTGTGGCGG | ACACTATTAGCA |
| | | TA |

Multi-segment RT-PCR (M-RTPCR): See (Zhou2012) for full detail of M-RTPCR protocol. Briefly, A549 cells were infected with IAV at MOI 3 for 8 h and treated with UMB18-2 or DMSO control. Cells were collected in TRIzol and RNA was extracted and converted to cDNA as detailed above. From this reaction, 2 μl of cDNA was used in a PCR reaction using Phusion Flash PCR Master Mix (Thermo Scientific) and M-RTPCR primers, MBTuni-12 (5′-ACGCGTGATCAGCAAAAGCAGG-3′) and MBTuni-13 (5′-ACGCGTGATCAGTAGAAACAAGG-3′). The reaction product was then separated on an agarose gel and imaged with a BioRad ChemiDoc system.

Western blotting: Western blots were performed as described in Weston2019. Primary antibodies used as follows: rabbit anti-SKIV2L (61 μg/150 μl, Proteintech), rabbit anti-H1N1 NS1 (0.5 mg/ml, Genscript) and mouse anti-tubulin (clone DMA1A, Sigma). SKIV2L and tubulin targeting antibodies were diluted 1:1000 and NS1 targeting antibodies were diluted 1:300 for use. Secondary antibodies were used as follows: goat anti-rabbit HRP (0.8 mg/ml, Thermo Scientific) and goat anti-mouse Alexa Fluor 546 (2 mg/ml, Life Technologies). HRP conjugated secondary antibodies were diluted 1:10,000 and fluorescent secondary antibodies were diluted 1:2000.

CellTiter-Glo assays: cells were plated in opaque 96 well plates one day prior to siRNA transfection. Plates were collected on days 1, 2 and 3 post-transfection and used for CellTiter-Glo Luminescent Cell Viability Assay (Promega) as per the manufacturer's instruction. Luminescence was read using a Synergy HTX Multi-Mode plate reader. For assessing viability of cells treated with compounds, cells were plated one day prior to use and treated for 24 h prior to being used in CellTiter-Glo assays.

Computational modeling: computer-aided drug design based on the Site-Identification by Ligand Competitive Saturation (SILCS) technology was applied to identify compounds that bind to SKI8 thereby perturbing the SKI complex such that compounds may potentially either inhibit or enhance complex formation. The asymmetric unit of the SKI complex X-ray crystallographic structure, PDB ID: 4BUJ, (Halbach, 2013 #10480) was prepared for analysis in the program CHARMM{Brooks, 2009 #9380} using the CHARMM-GUI. (Jo, 2008 #9540) The crystallographic asymmetric unit includes two monomers of SKI2, two monomers of SKI3 and four monomers of SKI8. Protein-protein interaction sites were defined as those residues on SKI8, chain C, with non-hydrogen atoms within 3.5 Å of non-hydrogen atom on all surrounding protein monomers. The resulting residues are listed in Table. The SKI8 monomer, PDB ID: 1S4U, (Cheng, 2004 #10481) was then subjected to SILCS Grand-Canonical Monte Carlo/Molecular Dynamics (GCMC/MD) simulations. {Lakkaraju, 2015 #9858} The simulations involve sampling of the distribution of a collection of eight solutes (benzene, propane, methanol, formamide, imidazole, acetaldehyde, methylammonium and acetate) at approximately 0.25 M in explicit water GCMC/MD simulations from which functional group affinity patterns normalized for the solutes in solution are obtained and converted to free energy patterns using a Boltzmann transformation, yielding grid free energies (GFE) mapped on a 1×1×1 Å grid encompassing the protein. Thus, the GFE FragMaps include contributions from protein flexibility, protein and functional group desolvation and protein-functional group interactions and, as they are precomputed, may be used for rapid estimation of ligand affinities and pharmacophore development. SILCS simulations were performed with the SilcsBio software suite (SilcsBio LLC).

Putative binding site identification involved the SILCS Hotspots approach where the binding affinity pattern of fragment molecules over the entire protein is calculated. Following two rounds clustering sites on the protein to which the fragments bind are identified for consideration as putative binding sites for drug-like molecules. This process involved visual inspection of the pattern of Hotspots on the protein in conjunction with analysis of the SILCS GFE FragMaps, the protein structure and the SILCS exclusion maps that identify regions of the protein structure that can relax to accommodate ligand binding, information not available through analysis of a protein crystal structure. From this analysis, a putative binding pocket in the central region of the protein as defined by the 7-strand beta propeller motif of SKI8 was identified. In preparation for in silico database screening pharmacophores, which represent the types of functional groups and their spatial relationships required on ligands that bind the site, were generated based on the SILCS-Pharm approach. This approach generated a collection of 11 pharmacophore features in the binding region along with the SILCS exclusion map which defines the extent of the binding region to which ligands can bind. Inspection of the pharmacophore features was undertaken using the SilcsBio Gui (SilcsBio LLC) from which a total of 3 pharmacophores, each with three aromatic features and one polar feature were generated. The two pharmacophores with the polar feature being a hydrogen-bond donor or acceptor were selected for screening with the third with a cationic feature was not used. In silico screening based on those pharmacophores was performed against a virtual database of approximately 780,000 compounds available from the vendors Chembridge or Maybridge. The database was generated in house and includes all accessible protonation and tautomeric states yielding a total of approximately 1.8 million species. In addition, the database includes up to 100 conformations of each ligand thereby accounting for ligand flexibility in the pharmacophore screen. Screening against the two pharmacophores was performed using the program Pharmer with the default settings. In the screen the root-mean square distance difference between the location of pharmacophore features and the corresponding functional groups on the ligands are determined (RMSDpharm) for all states and conformations of each ligand with the lowest RMSDpharm for each ligand used for compound ranking. From this procedure, the top 10,000 compounds were selected from each screen. Subsequent compound selection was based on energy criteria in the context of the SILCS GFE FragMaps. This procedure involves Monte Carlo conformational sampling in field of the FragMaps termed SILCS MC. MC sampling involves “local” sampling of translational, rotational and dihedral degrees of freedom as previously described (Ustach, 2019 #10245). This procedure, which is initiated from the position and conformation of each ligand from the pharmacophore screen allows each compounds to relax in the field of the FragMaps from which the ligand GFE (LGFE) for each ligand is scored. The LGFE, which is a sum of the GFE contribution of selected atoms in the molecule, is an approximate metric of the binding free energy of each molecule. As this stage the two searches were combined, and the top 1000 compounds selected based on the ratio of the LGFE/RMSDpharm metrics. The use of the LGFE/RMSDpharm ratio is designed to allow for the estimated binding affinity and agreement with the targeted ligand to be taken into account during ranking. Further screening of top 1000 compounds was performed to select compounds with drug-like or lead-like properties based on the 4-dimensional bioavailability indicator being >−4.5, the log of the partition coefficient calculated using MOE (Chemical Computing Group) less than 5 and the molecular weight being below 500 daltons. This yielded a total of 202 compounds that were subjected to chemical fingerprint clustering using MOE BIT-MACCS fingerprints and the Tanimoto Index (Chemical Computing Group). This led to final compounds being selected.

Once active, hit compounds were identified lead expansion was undertaken. Inspection of the compounds showing some level of activity showed several of them to have chemical structures similar to UMB18. Accordingly, additional compounds with a similar chemical scaffold were identified via chemical fingerprint similarity searching. This search used both chemical fingerprint (BIT-MACCS) and physiochemical fingerprint (MPMFP) in two separate screens. Screening was against the full University of Maryland CADD Center database of 5.04 million compounds. From these searches 253 compounds with a Tanimoto index >0.85 were selected from the chemical BIT-MACCS search and 246 compounds with a Tanimoto index >0.91 based on the physiochemical MPMFP search.

Results

The SKI complex has a genetic interaction with IAV NS1 and MERS-CoV ORF4a

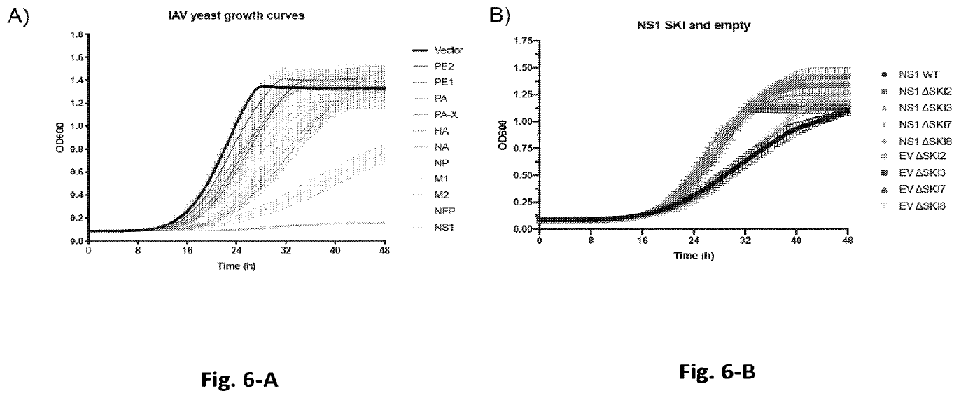

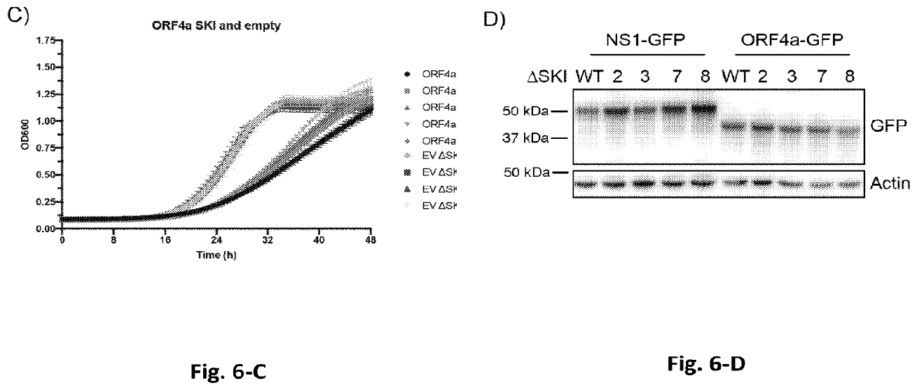

It was previously demonstrated that certain MERS-CoV proteins are capable of causing slow growth when expressed in the yeast S. cerevisiae (Weston2019). The slow growth phenotype induced by MERS-CoV ORF4a was used to perform suppressor screening to identify genetic interactors of this protein in yeast; from this work we found the mammalian homologue of the yeast gene SIR2 (mammalian SIRT1) is a proviral factor in MERS-CoV replication (Weston2019). In addition to MERS-CoV, proteins encoded by influenza A virus (IAV) are capable of causing a slow growth phenotype in S. cerevisiae (Basu2009 and FIG. 6-A). We focused our attention on the NS1 protein as we had previously validated its slow growing phenotype in this yeast system (Basu2009) and it has similarity to MERS-CoV ORF4a in that it is a double stranded RNA binding protein that can inhibit the IFN response in mammalian cells. We performed suppressor screening in the yeast knockout library for IAV NS1 (see Weston2019 for details on suppressor screening). From this screening, we found 101 yeast colonies that suppressed the IAV NS1 phenotype, representing 69 unique genes. In follow up validation experiments, 14 of these genes were determined to be bona fide suppressors (Table 1).

TABLE 1
Table of the yeast genes that were found in suppressor screening of the NS1
YKO library; the “hit times” column refers to the number of individual colonies that were found
to have the same gene deletion. Key terms taken from the Saccharomyces Genome Database
(https://www.yeastgenome.org/).

| Hit times | Yeast gene | Yeast protein | Human homolog | Key terms |
|---|---|---|---|---|
| 9 | YPR189W | SKI3 | TTC37 | RNA exosome, RNA processing |
| 4 | YJL172W | CPS1 | PM20D1 | Vacuolar carboxypeptidase S |
| 3 | YNL058C | YNL058C | | Vacuole |
| 3 | YNL058C | YNL058C | unknown | Vacuole localization |
| 3 | YPL171C | OYE3 | | NADPH oxidoreductase |
| 2 | YGL213C | SKI8 | WDR61 | RNA exosome, RNA processing |
| 2 | YLR180W | SAM1 | MAT1A | S-AdoMet production |
| 2 | YOL020W | TAT2 | | Tryptophan/tyrosine permease |
| 2 | YIL122W | POG1 | | Mitochondrial DNA polymerase |
| 1 | YHR049W | FSH1 | OVCA2 | Serine hydrolase |
| 1 | YDR146C | SWI5 | SWIS | Transcription factor in mitosis |
| 1 | YBL013W | FMT1 | MTFMT | Methionyl-tRNA formyltransferase |
| 1 | YJL208C | NUC1 | ENDOG | Mitochondrial nuclear endonuclease |
| 1 | YBR233W | PBP2 | PCBP2/4 | RNA binding protein, similar to mammalian nuclear RNP K protein |

The most frequent hit from the NS1 screening was the yeast gene YPR189W/SKI3. SKI3 is a member of the yeast SKI complex comprised of SKI2, SKI3, SKI7 and SKI8. Interestingly, the gene YGL213C/SKI8 was also a validated suppressor for IAV NS1 (Table 1) and SKI7 was a validated hit from our previous suppressor screen using MERS-CoV ORF4a (Weston2019). It was therefore decided to directly investigate whether all of the yeast SKI complex genes would act as suppressors for each of the viral proteins. Yeast knocked out for each of the four SKI genes were collected from an arrayed knockout library and transformed with expression vectors for IAV NS1 or MERS-CoV ORF4a and analyzed for their growth rate. All of SKI2, SKI3 and SKI8 were potent suppressors for the NS1 slow growth phenotype, while SKI7 had only minimal effect (FIG. 6-B). The suppressor phenotypes for ORF4a were milder than those seen for NS1, however all of the SKI knockout strains gave an increase in growth rate compared to wild type cells, with loss of SK17 giving the largest increase (FIG. 6-C). These alterations to growth rate were not the consequence of a loss of viral protein expression (FIG. 6-D). Overall, these data demonstrate that in S. cerevisiae, there is a functional genetic interaction between IAV NS1 and MERS-CoV ORF4a with the yeast SKI complex.

The SKI Complex is Required for IAV and MERS-CoV Replication

The yeast SKI complex has a functional interaction with IAV NS1 and MERS-CoV ORF4a (FIG. 6), suggesting that this protein complex may be involved with replication of these two viruses. The SKI complex is well conserved between yeast and mammalian cells. The mammalian homologues of SKI2, SKI3 and SKI8 are SKIV2L, TTC37 and WDR61, respectively (hereafter, the yeast genes and human genes will be denoted by these different names). The mammalian homologue of SKI7 is poorly defined and we have excluded that from further study here. To investigate whether there is a role of the mammalian SKI complex in replication of IAV or MERS-CoV multiple siRNA sequences were analyzed targeting each of the three genes (SKIV2L, TTC37 and WDR61). A549 (IAV) or Huh7 (MERS-CoV) cells were transfected with these six individual siRNA sequences, a scrambled control or mock transfected for three days, prior to being infected with each virus for 24 h (MOI 0.01 and MOI 0.1 for IAV and MERS-CoV, respectively). After the infection, virus was collected and titered.

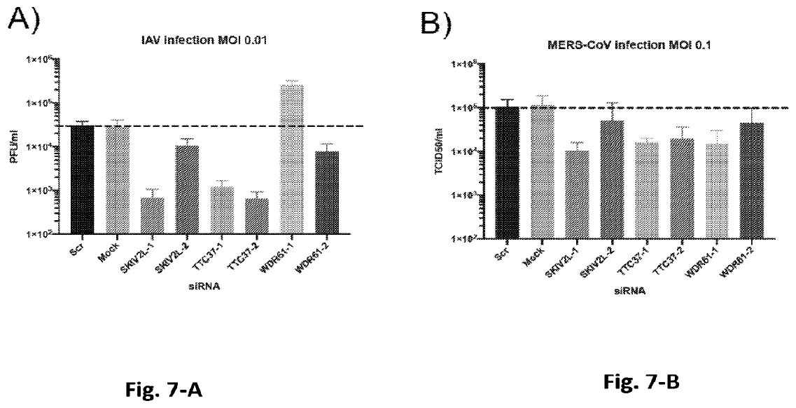

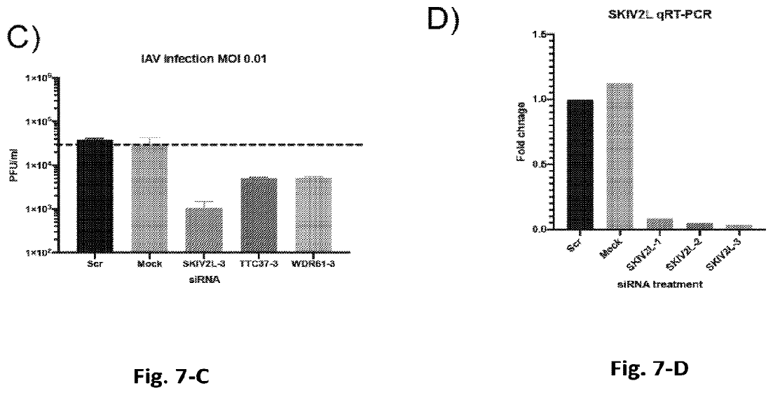

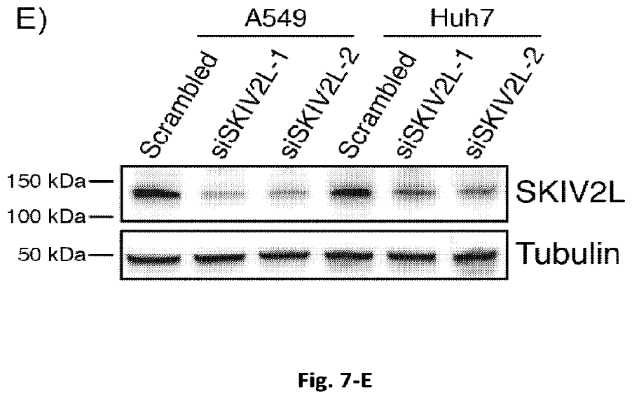

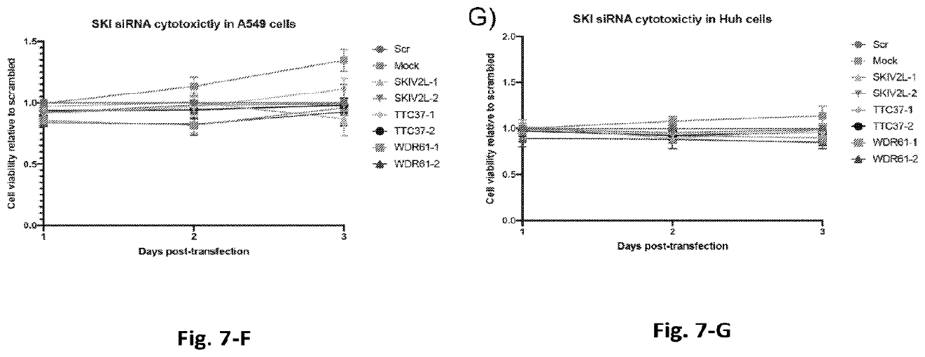

For IAV infection, both siRNA sequences for SKIV2L and TTC37 gave a significant reduction in viral replication (FIG. 7-A). The knockdown of WDR61 with one sequence also gave a significant reduction, while the other gave an enhancement in replication of IAV (FIG. 7A). For MERS-CoV, all of the siRNA sequences gave a reduction in replication to varying levels (FIG. 7-B). Owing to the discrepancy in the results of the two WDR61 sequences for IAV infection, a third siRNA sequence was tested for each of the SKI genes and found that all three gave an inhibition of IAV replication (FIG. 7-C). Knockdown of each of the SKI genes in A549 cells was confirmed by qRT-PCR for each of the different sequences (FIG. 7-D), and at the protein level for SKIV2L (FIG. 7-E) (unable to find usable antibodies for TTC37 and WDR61). Importantly, over the three-day transfection time course, none of the siRNA sequences resulted in a significant reduction in cell viability as assessed by CellTiter-Glo assay (FIGS. 7-F and 7-G). Overall, siRNA mediated knockdown of each of the different components of the mammalian SKI complex result in a reduced replication of IAV and MERS-CoV, suggesting the SKI complex may be a conserved proviral factor for these two very different viruses.

The SKI Complex is a Potential Antiviral Target

The data suggest a genetic interaction between viral proteins and the SKI complex in yeast and that the mammalian SKI complex may be important for replication of two very different viruses. It was therefore speculated that the SKI complex may be a potential broad-spectrum antiviral target. No compounds targeting the SKI complex have been developed, as such we took a computational modeling approach using the yeast SKI complex for which there are published structural data (Halbach2013).

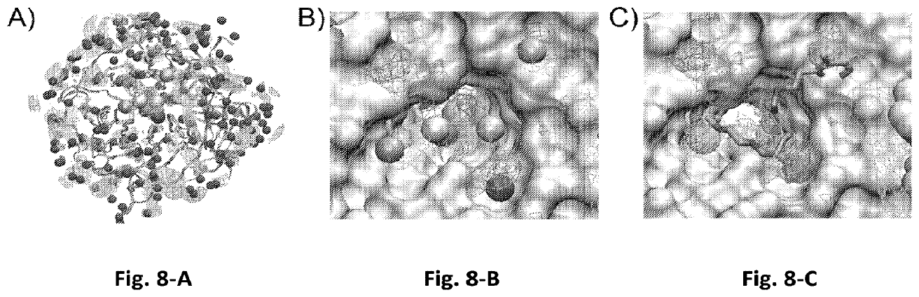

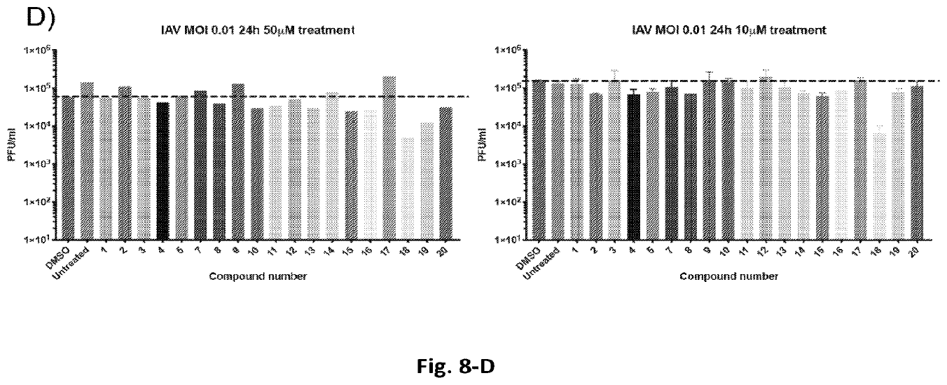

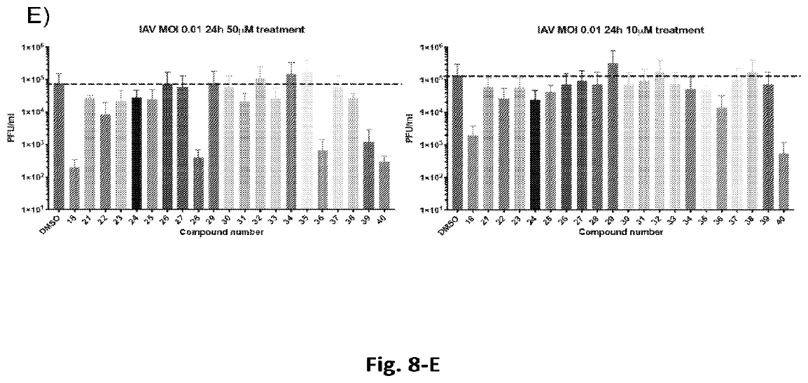

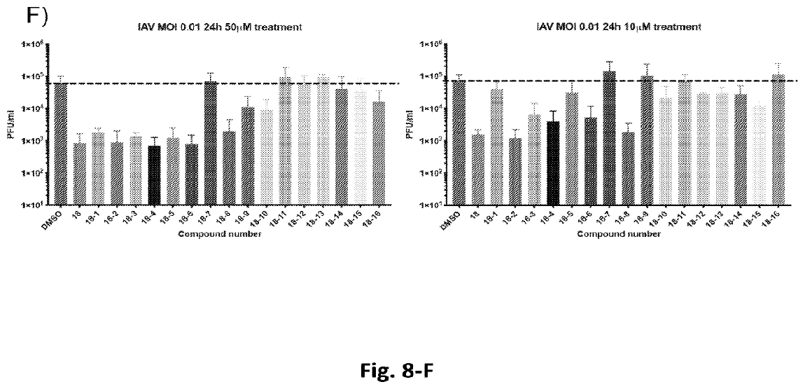

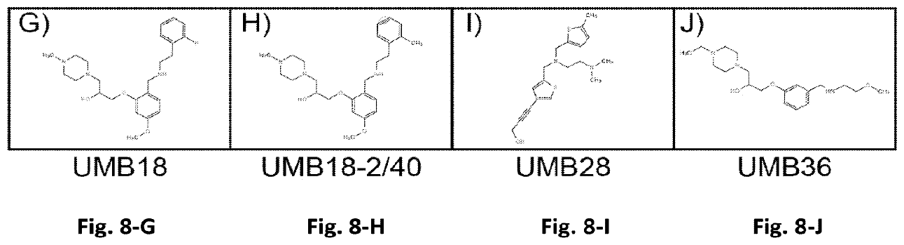

Ligand design efforts targeted the identification of compounds that would perturb the SKI complex, focusing on SKI8. The process involved the identification of regions on SKI8 in contact with other protein monomers in the complex (Halbach2013) along with the identification of putative ligand binding sites using the Site Identification by Ligand Competitive Saturation (SILCS) approach (Guvench2009). Based on SILCS Hotspots and FragMap analysis (MacKerell2020) a putative binding site on the edge of the central region of the beta propeller of the SKI8 monomer was identified (FIGS. 8-A and 8-B). The region includes residues 20, 125, 188, 205 and 237 of SKI8. To initiate the screening of ligands targeting the binding pocket the SILCS-Pharm approach (Yu2015) was applied to develop multiple pharmacophores for in silico screening of a database of ˜780,000 commercially available compounds. Upon visual inspection, two pharmacophores that each include 4 features (one of these shown in FIG. 8-C) were selected for further in silico screening using the program Pharmer (Koes2011). For each screen the top 10,000 compounds were selected based on the root mean square spatial difference between the pharmacophore features and the respective functional groups on the ligands (RMSDpharm). Each set of 10,000 compounds was then subjected to SILCS-MC docking initiated from the pharmacophore screen orientations. From this, the ligand grid free energy (LGFE), a metric of the binding affinity, was calculated (Raman2013). Results from these screens were then combined and ranked based on the LGFE/RMSDpharm score that biases compounds towards those with the highest predicted binding affinity and good agreement with the pharmacophores used to initially select those compounds. From this, the top 1000 compounds were selected and screened for drug and lead-like characteristics based on 4-dimensional bioavailability (Oashi2011) >−4.5, log P<5 and molecular weight <500 daltons. This yielded 202 compounds that were subject to chemical fingerprint clustering from which a number of compounds were purchases and assessed.

Having mapped a potential compound docking site at the interface of WDR61 and TTC37, we purchased 39 compounds (in two sets) that were predicted to bind and tested for antiviral activity. In the first batch of 20 compounds tested, one of these showed antiviral activity against influenza, hereafter referred to as UMB18 (FIG. 8-D). The second set had three further compounds that showed a degree of antiviral activity (referred to as UMB28, UMB36 and UMB40 in FIG. 8-E). While screening the second set of compounds, also screened was a set of chemical analogues to initial hit UMB18 (FIG. 8-F). Of these 20 further compounds, none showed any greater antiviral activity against IAV infection, but the compound 18-2 showed a similar level of inhibition. As part of the predictive process, structurally related compounds were not excluded. As such, the compound coded UMB40 was also in the SAR set as UMB18-2, blindly re-validating this initial hit. Referring to this compound as UMB18-2 for the remainder of the disclosure. Overall, we modelled a library of compounds that may potentially target the SKI complex and found four capable of inhibiting IAV infection: UMB18 (FIG. 8-G), UMB18-2 (FIG. 8-H), UMB28 (FIG. 8-I), and UMB36 (FIG. 8-J).

Investigation of Lead SKI Targeting Compounds for Antiviral Activity

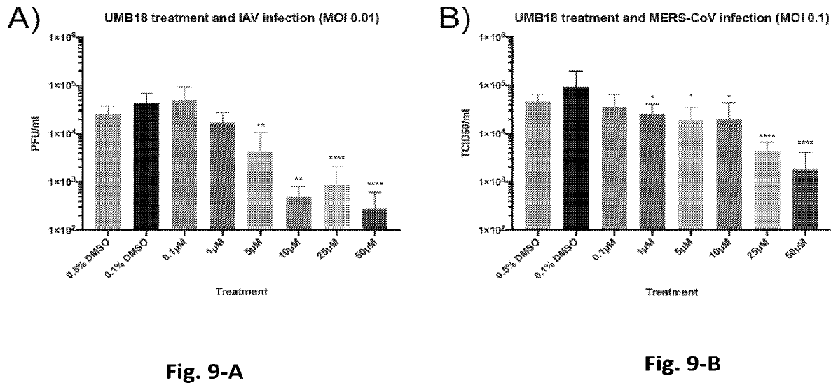

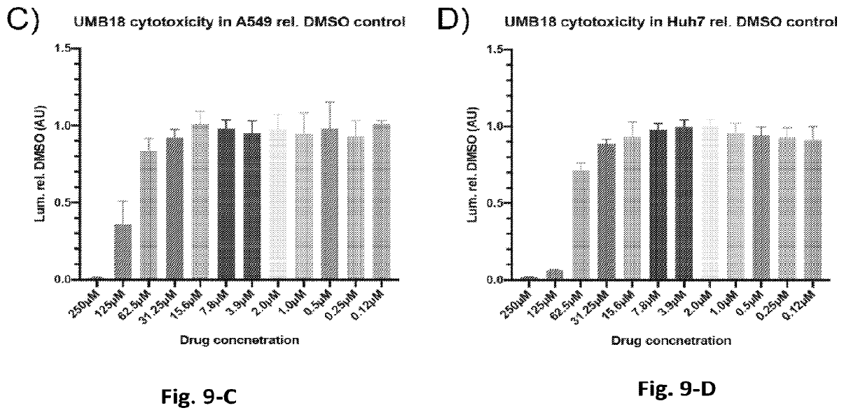

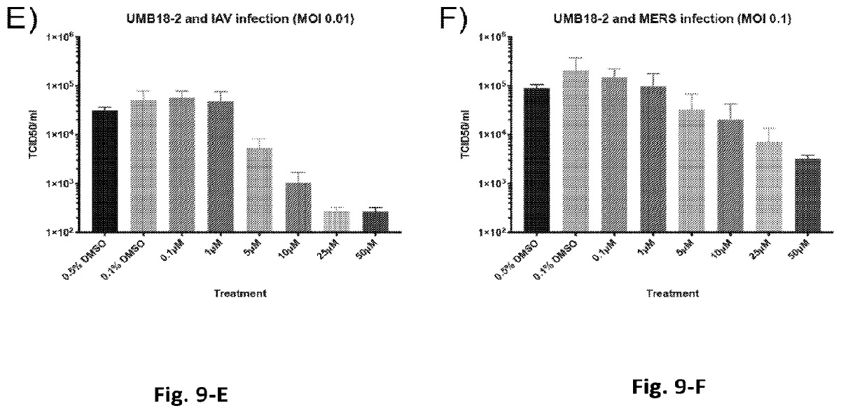

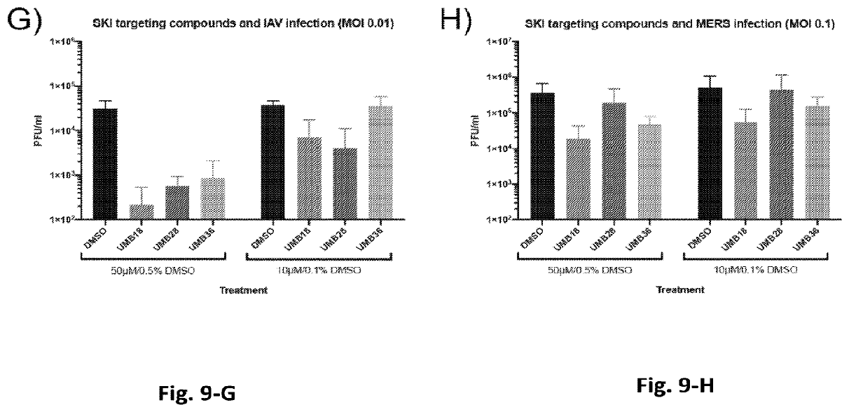

From the initial screening experiments, UMB18 and UMB18-2 appeared to show the greatest antiviral activity against IAV infection. These two compounds differ by only a hydroxyl and fluoride group (FIGS. 8G and 8H), so they were considered them largely similar and it was decided to approach these as lead compounds. Having displayed inhibition of IAV infection at concentrations of 50 μM and 10 μM treatments in screening, the dose dependency of the compounds were investigated. Cells were treated with UMB18 across a boarder range of concentrations and infected with IAV (FIG. 9-A). These data demonstrate dose dependent inhibition of IAV by UMB18 and an IC50 value of ˜5 μM. Having seen that the SKI complex is also required for MERS-CoV infection (FIG. 7-B), UMB18 was tested against this virus and found it capable of inhibiting infection with a similar IC50 as for IAV (FIG. 9-B). These data suggest that UMB18 may have potential as a broad-spectrum antiviral compound. The antiviral activity is not a result of cell cytotoxicity; antiviral concentrations caused minimal toxicity as assessed by CellTiter-Glo assay in both A549 cells (IAV infection) and Huh7 cells (MERS-CoV) (FIGS. 9-C and 9-D). UMB18-2 also showed similar inhibitory profiles against both viruses, suggesting the small difference between these two compounds does not influence the antiviral activity (FIGS. 9-E and 9-F). Overall, UMB18 and UMB18-2 both appear to have antiviral against IAV and MERS-CoV with IC50 values around ˜5 μM.

Assessment of Other Chemical Compounds Targeting the SKI Complex for Antiviral Activity

In addition to the UMB18 and UMB18-2 compounds, two further chemical structures were identified that inhibited IAV infection (FIGS. 8-I and 8-J). While both had antiviral activity, neither appeared to be as potent as UMB18 in the initial tests (FIG. 8-E). We further investigated this with more direct comparisons. At 50 μM, both UMB28 and UMB36 showed similar inhibition of IAV as UMB18, but both had reduced inhibition at 10 μM (FIG. 9-G). Similar results were seen for antiviral activity against MERS-CoV (FIG. 9-H). These data from FIG. 9 demonstrate UMB18 and UMB18-2 as our most potent antiviral compounds, but that different chemical structures that are modeled to target the SKI complex also display broad-spectrum antiviral activity.

UMB18 Inhibits Filovirus Infection

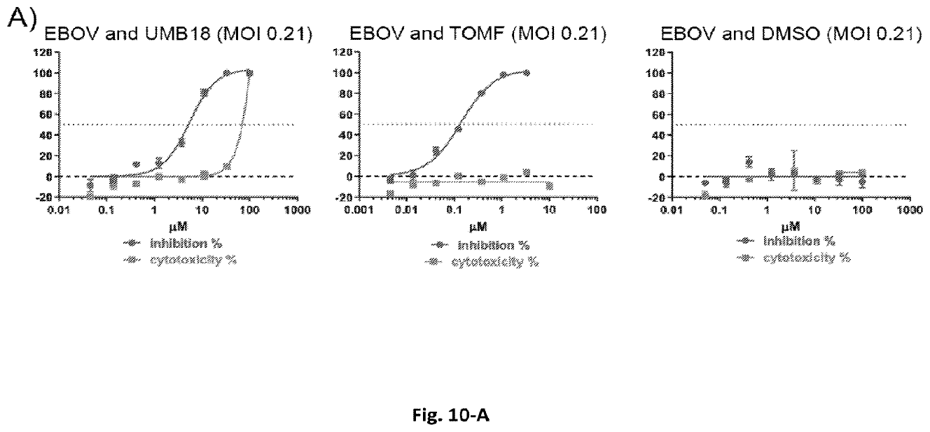

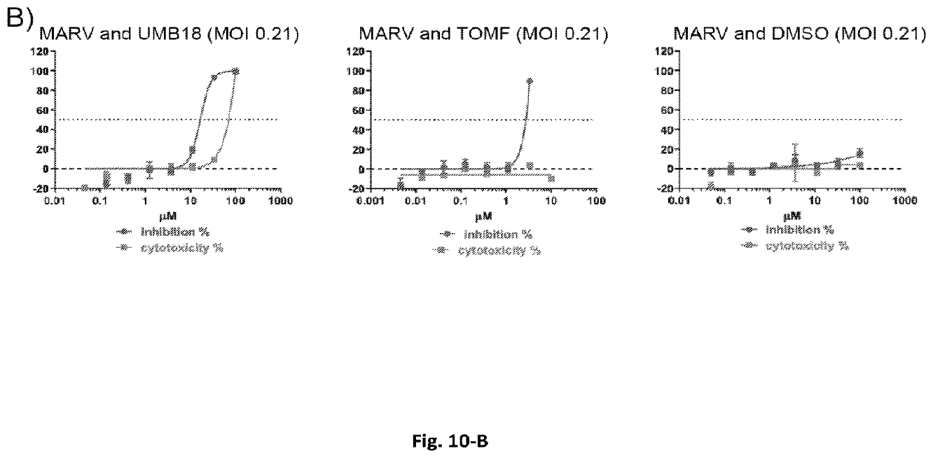

The breadth of antiviral activity of lead compound UMB18 was further investigated. For this, another family of viruses was tested that cause severe human mortality, the filoviruses, specifically Ebolavirus (Makona strain, EBOV) and Marburg virus (Angola stain, MARV). Huh7 cells were treated with UMB18 across an 8-point dose curve and infected with EBOV (FIG. 10-A-10-C) or MARV (FIG. 10-B) at MOI 0.21 and 0.5 for 48 h. The percentage of inhibition was plotted along with the assessment of cytotoxicity at each concentration in the absence of infection. Toremifene citrate was used as a positive control for inhibition, and both were compared to DMSO as the negative control. Both EBOV and MARV were found to be inhibited by UMB18 at non-cytotoxic concentrations. EBOV appeared to be more sensitive to UMB18 with an IC50 calculated as ˜5 μM, a very similar value to that seen for IAV and MERS-CoV (FIG. 4). MARV was comparably less sensitive to UMB18 with an IC50 around 16 μM. These data further extend the notion that UMB18 has broad-spectrum antiviral activity.

SKI Targeting Compounds Inhibit Production of Viral RNA

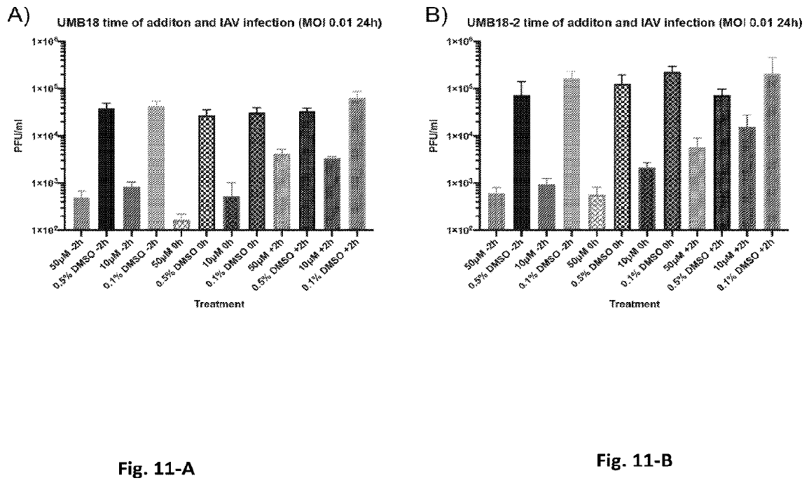

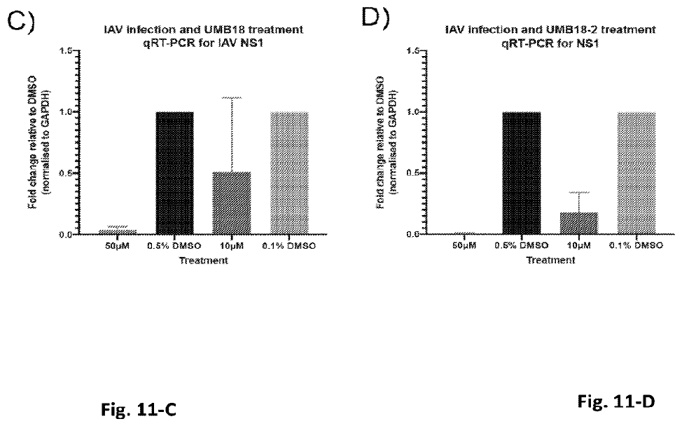

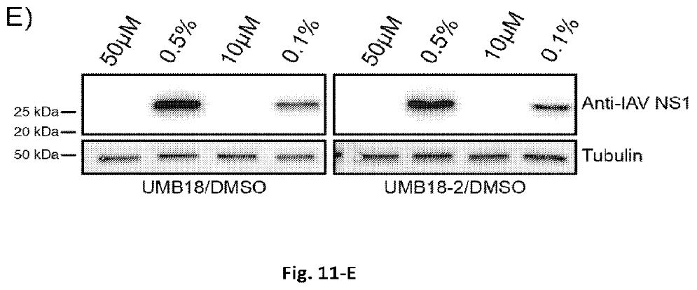

To better understand the mechanism of action of lead SKI targeting compounds a time of addition assay was used. In all experiments previously discussed, virus and compound were added to cells at the same time. In the time of addition assays, cells were either pre-treated with compound for 2 h (−2 h), had the compound added with IAV as before (0 h) or compound was added 2 h after cells were infected (+2 h). Pre-treatment of cells and addition of drug at the same time as virus lead to a similar level of inhibition with both UMB18 and UMB18-2 (FIGS. 11-A and 11-B). Addition of drug at 2 h after infection was started showed a marked reduction in the level of inhibition. However, the compounds were still able to inhibit infection compared to DMSO control. Since the SKI complex is involved in RNA metabolism, next it was investigated whether treatment with lead compounds had an impact on viral mRNA production, and by extension, viral protein production. IAV mRNA is seen to peak at around 4-5 h post-infection in A549 cells (Laske2019), which may explain reduced activity of our compounds when added at 2 h post-infection since mRNA production would have begun. Cells were treated with UMB18 or UMB18-2 and infected with IAV at MOI 3, to ensure all cells in the plate would be infected. After 8 hours, cells were collected in TRIzol or RIPA lysis buffer for analysis of viral mRNA production and protein production. Using NS1 as the reporter gene, treatment with either compound lead to a marked reduction in mRNA at both 50 μM and 10 μM, with a lower level of inhibition seen at 10 μM, suggesting dose dependency (FIGS. 11-C and 11-D). To further assess loss of IAV RNA multi-segment RT-PCR approach was used (M-RTPCR Zhou2012) to amplify all segments of the IAV genome. It was found that from cells treated with UMB18-2 there was a total loss of IAV RNA that could be amplified by this protocol compared to DMSO controls (FIG. 11-E), further suggesting that the compounds inhibit viral replication by inhibiting RNA production. In agreement with the lack of mRNA, a lack of NS1 protein was also observed when cells were treated with UMB18 or UMB18-2 (FIG. 11-F). Overall, these data suggest that lead compounds are capable of inhibiting viral mRNA production.

SKI Complex Targeting Compounds have Broad Anti-Coronavirus Activity

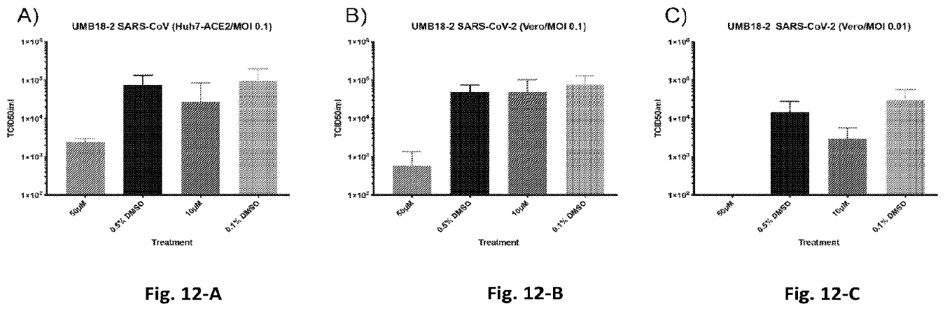

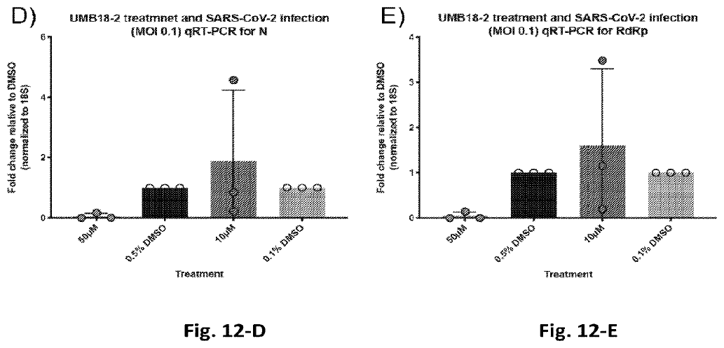

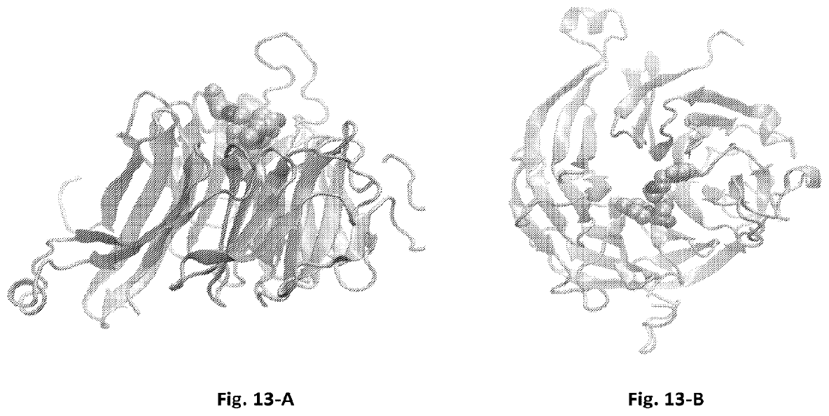

Coronaviruses SARS-CoV-1 and SARS-CoV-2 utilize a difference cell surface receptor to MERS-CoV and there is therefore different cell line permissivity. Huh7 cells were used for the MERS-CoV work described here, but neither SARS-CoV nor SARS-CoV-2 infect these cells. The receptor for both of these additional coronaviruses is ACE2 (Li2003, Zhou2020, Wan2020, Hoffmann2020). SARS-CoV has previously been shown to infect Huh7 cells overexpressing the ACE2 receptor, and thus, Huh7 cells stably expressing ACE2 was used for SARS-CoV work. Huh7-ACE2 cells were infected with SARS-CoV and treated with UMB18-2. Virus was collected after 24 h and titered by TCID50 assay, to match the work with MERS-CoV. SARS-CoV was found to be sensitive to UMB18-2 similarly to MERS-CoV, with 50 μM treatments showing a 1 log reduction in virus production and 10 μM showing inhibition, but to a lesser extent (FIG. 12-A).

Even though SARS-CoV-2 utilizes the same cell surface receptor as SARS-CoV, no infectious virus particles were released from the Huh7-ACE2 cells and therefore Vero E6 cells were used instead. In the filovirus experiments, it was found that Vero E6 cells had lower IC50 values than Huh7 cells (data not shown), suggesting that SKI targeting compounds may be less efficacious in a monkey cell line. It was found that when Vero cells were infected with SARS-CoV-2 at either MOI 0.1 (FIG. 12-B) or MOI 0.01 (FIG. 12-C) and treated with UMB18-2 this compound was capable of inhibiting virus production as measured by TCID50 at 50 μM, but less so at 10 μM. Treatment with UMB18-2 was also capable of inhibiting SARS-CoV-2 mRNA production (using two gene targets [FIGS. 12-D and 12-E]), suggesting a similar mechanism of inhibition as demonstrated for IAV. These data suggest that SKI targeting compounds may have broad antiviral activity against coronaviruses, targeting the three human pathogenic viruses of the family along with having antiviral activity against influenza and filoviruses.

### Example 3: Broad Spectrum Antiviral Develop by Inhibition of the SKI Complex

Compounds which have broadly acting antiviral activity were identified. The compounds are modeled to target the SKI complex (part of the RNA exosome) via in silico modeling of a library of compounds on the SKI complex crystal structure. The SKI complex is made up of protein components which are called yeast SKI2/human SKIV2L, yeast SKI3/human TTC37 and yeast SKI8/human WDR61. The function of the SKI complex in yeast and mammalian cells is to unwind RNA, the ySKI2/hSKIV2L protein has helicase activity, and feed RNA into the degradation machinery of the RNA exosome for degradation. The compounds are predicted to bind in a pocket at the interface of SKI8/WDR61 and SKI3/TTC37. The findings herein indicate that treating cells with these compounds induces basal Interferon induced gene expression and hypersensitizes the cell to further Interferon induction. After treatment with the identified compounds, infection of cells with the viruses tested leads to induction of interferon and anti-viral proteins resulting in reduced virus replication. The compounds with anti-viral activity were purchased from Chembridge Corp, and are catalog #96509034, 5612793 and 10253964. Additionally, an analogue of #96509034 was also identified to have increased activity, its catalog # is 27092311.

Yeast Expression Identifies Viral Proteins that Effect Yeast Growth.

It was demonstrated that expression of the viral proteins in yeast, including the SARS-CoV PLpro, CHIKV nsP2, and IAV NS1 proteins cause an inhibition of growth when expressed in the yeast S. cerevisiae. For the investigation of host genes that effect growth of Influenza virus, it was initiated with expression of Influenza virus NS1 gene due to its role in Influenza virus replication and innate immune antagonism. In yeast, the viral proteins are under the control of a GAL1 promoter such that when grown in glucose, gene expression is inhibited, and the yeast grow similarly to wildtype or vector control. When grown in the presence of 2% galactose, the viral gene is expressed and growth is assayed on both agar plates and liquid media.

Growth curves were performed for yeast expressing either an empty plasmid or Influenza virus NS1. Over the 48-hour time course, empty vector yeast reaching saturation ˜24 hours from the start of the experiment. Comparatively, yeast expressing NS1 grew significantly slower, only catching up to the control yeast at the 48 hour timepoint.

Yeast Knockout Screen for Suppressors of Influenza NS1 Slow Growth Phenotype Using Pooled Yeast Deletions.

A library of yeast knockouts has been previously created where every non-essential gene is deleted individually and replaced with a 60 nucleotide DNA barcode sequence. These barcodes contain 20 common nucleotides either side of a 20 nucleotide unique sequence that is specific to each gene knockout. Sequencing of these unique 20 nucleotides can therefore identify the gene that has been knocked out from an individual yeast cell. A pool of this knockout library was transformed with a plasmid to express NS1 and selected for transformants on URA-media containing 2% glucose such that all viral gene expression would be repressed. These transformants were collected and plated on URA-media containing 2% galactose. Yeast cells within the library knocked out for a gene involved in NS1-mediated slow growth could grow faster, and form larger colonies, identifying genetic suppressors.

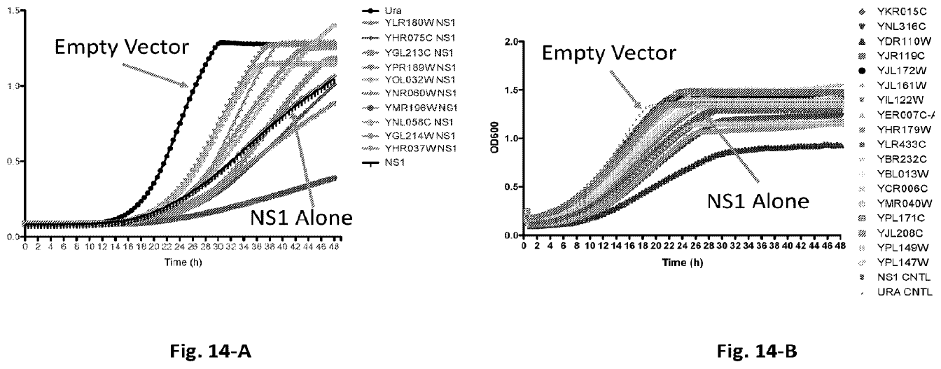

Identification of genetic suppressors of NS1. The large colonies were picked and grown in liquid media to validate the suppressor phenotype. Expression of the viral protein was confirmed by western blot, and validated hits were then sequenced to determine which gene was knocked out. Genetic suppressors were revalidated by transforming known knockout yeast collected from an arrayed library. Proteins in a variety of pathways were identified including the SKI complex protein, SKI2 (FIGS. 14A and 14B).

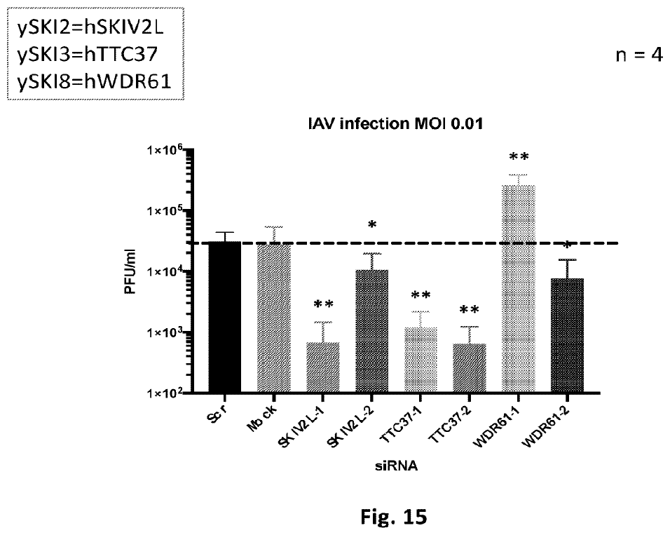

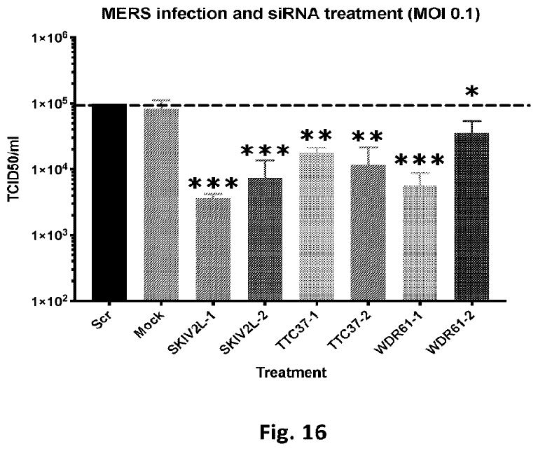

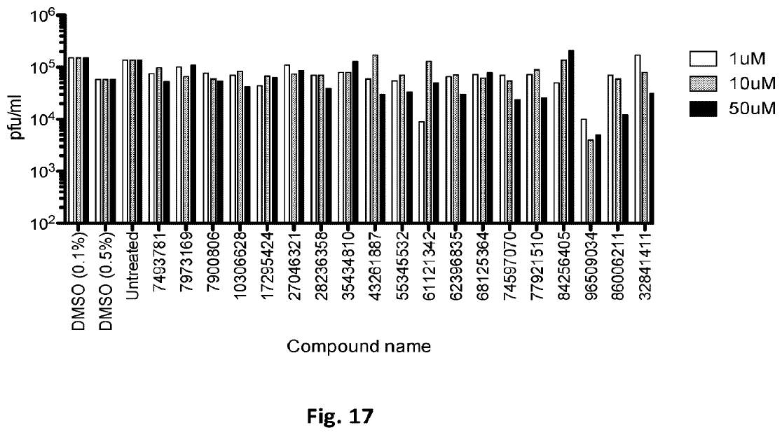

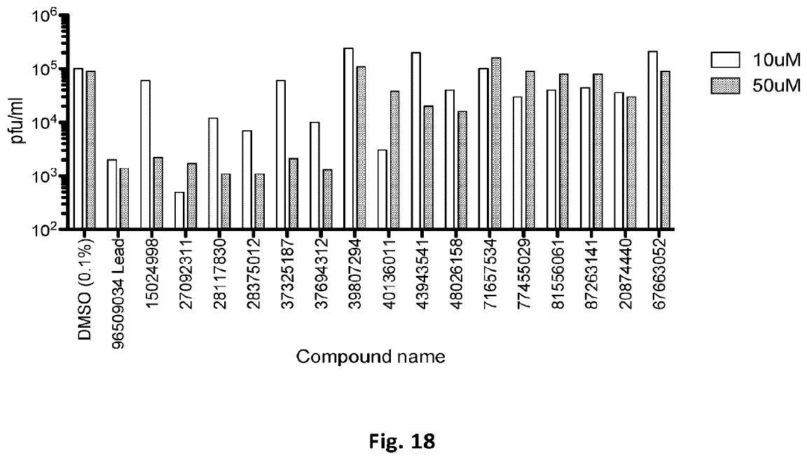

Validation of SKI complex effect on Influenza virus replication. The SKI complex proteins including SKI2, SKI3, SKI7 and SKI8 was the chosen hits for investigation from the pooled screen due to their potential role in viral replication and because SKI complex genes were found in multiple yeast screens for viral genes. It was therefore investigated whether SKI complex genes had a role in Influenza virus replication in cell culture. Knockdown experiments with siRNAs targeting SKIV2L, TTC37 and WDR61 (the human homologues of yeast genes SKI2, SKI3 and SKI8, respectively) were performed (FIG. 15). A549 cells were transfected with 2 different siRNAs targeting each of the genes and knockdown was confirmed by quantitative RT-PCR. Cells were infected with Influenza virus and media assayed for virus levels (FIG. 16). We found that Influenza virus replication was reduced by ˜2 logs in SKIV2L and TTC37 siRNA transfected cells compared to scrambled control siRNA. Huh7 cells were also transfected with the same siRNAs and infected with MERS-CoV (FIG. 16). Similar to the Influenza virus infection experiments, reduction of MERS-CoV replication between 1-2 logs after knockdown of SKIV2L and TTC37 was found. Without wishing to be bound by any particular theory, it is believed that this demonstrates that the SKI complex may be involved in regulating virus replication for multiple virus families.

In Silico Modeling of Compounds to Affect SKI Complex Function.

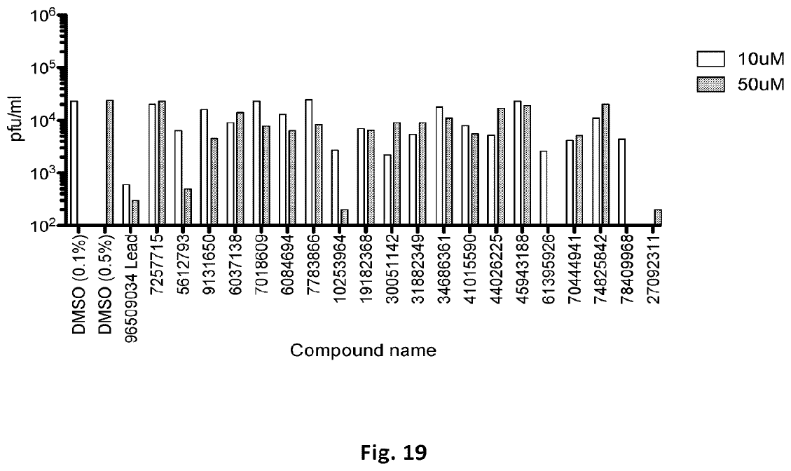

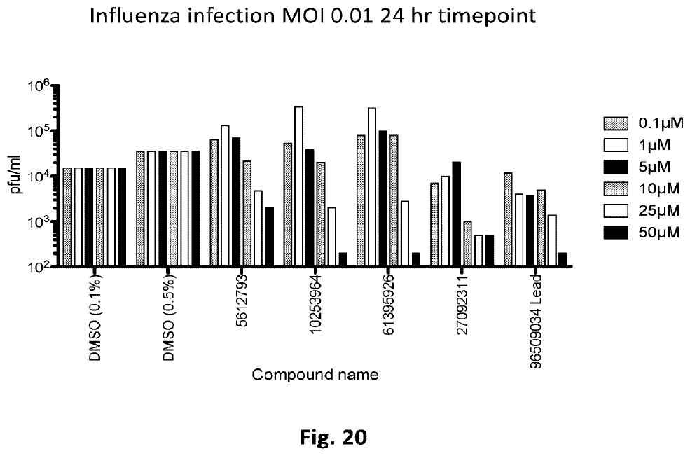

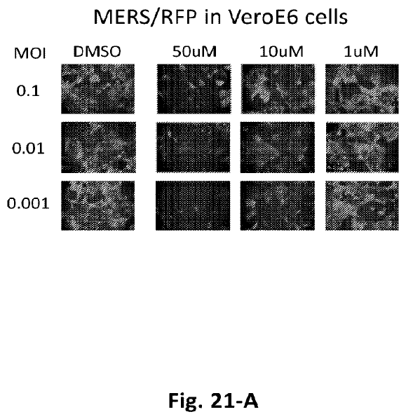

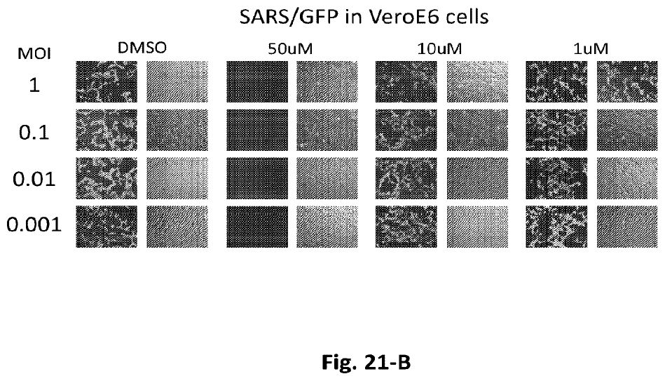

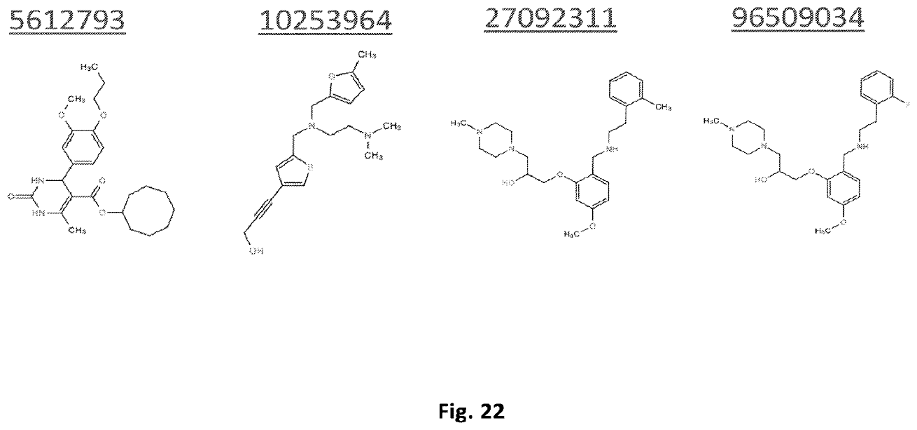

The structure of the yeast SKI complex (SKI2,3,8) has been previously published. The yeast SKI complex structure and modeled compounds that bind at the of SKI8.SKI3 interaction face were identified. Using this computer aided drug design approach, an initial 40 compounds were purchased from Chembridge and tested for their ability to inhibit Influenza virus replication. For the testing, A549 cells were treated with each compound at 50 μM, 10 μM and 1 μM concentrations and then infected with Influenza virus. At 24 hours post infection, supernatant was assayed for the level of Influenza virus replication by plaque assay. Through these experiments 4 compounds that inhibit Influenza virus replication greater than 1 log were identified (FIG. 17, FIG. 18 and FIG. 19). Broader concentration curve on SKI targeted compounds were determined (FIG. 20). Reduction of virus growth was found via readout of fluorescence in infected cells. Additionally, compound 96509034 was tested against Ebola virus and Marburg virus (FIGS. 21A and 21B). In all viruses tested, 96509034 has inhibited virus replication with an IC50 of ˜5 μM. Structures of Identified Compounds are depicted in FIG. 22.

Potential Mechanism of Action.

Without wishing to be bound by any particular theory, it is hypothesized that mechanism of action for the antiviral properties of the SKI complex and alterations in its activity are through effects on the innate immune response. Induction of the innate immune response is critical to the protection of the host from pathogens. Proper regulation of this response is modulated by host proteins to confer anti-viral induction while limiting the pathologic effects of constitutive interferon expression. The RIGI and MDA5 proteins are cytoplasmic sensors that distinguish between viral RNA and host RNA to confer proper induction if the interferon pathway. Specific host RNA degradation complexes regulate the controlled turnover of cellular RNAs and are required for proper cellular metabolism and function. Inhibition of these complexes could lead to increased host RNA levels and altering of the sensitivity of the RNA sensors leading to induction of the IFN pathway irrespective of viral infection. Without wishing to be bound by any particular theory, it is hypothesized that reduction of the SKI complex activity either via siRNA knockdown experiments or through the compounds identified herein, reduce the RNA degradation activity of the RNA Exosome leading to increased cytoplasmic RNA levels, triggering an induction of interferon signaling. The induction of interferon signaling would provide a broad spectrum anti-viral response leading to protection from a wide array of viral pathogens. This may also effect other diseases where interferon induction is protective including cancer and bacterial infection.

### Example 4

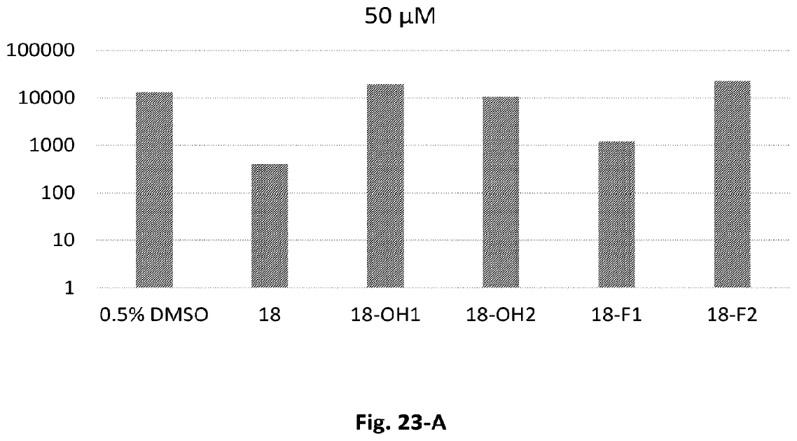

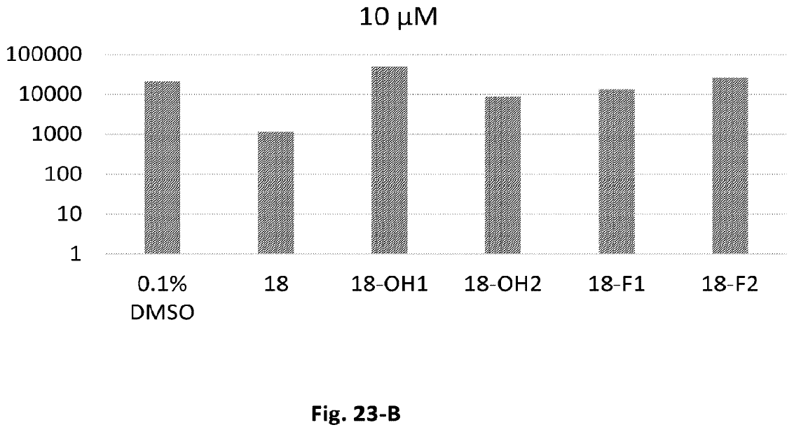

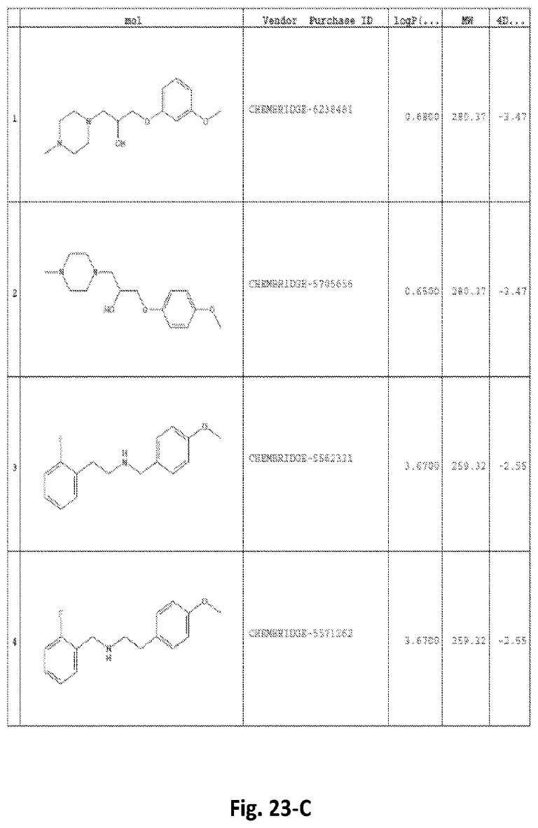

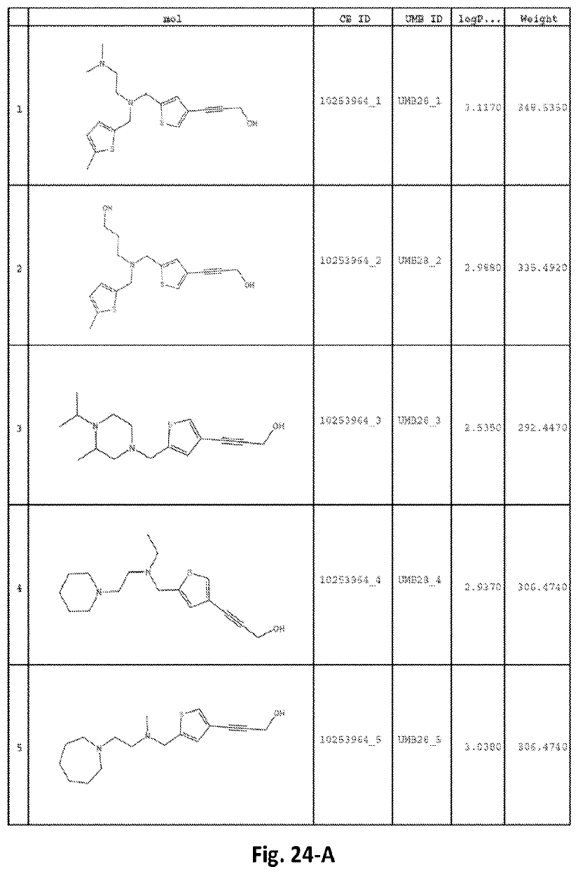

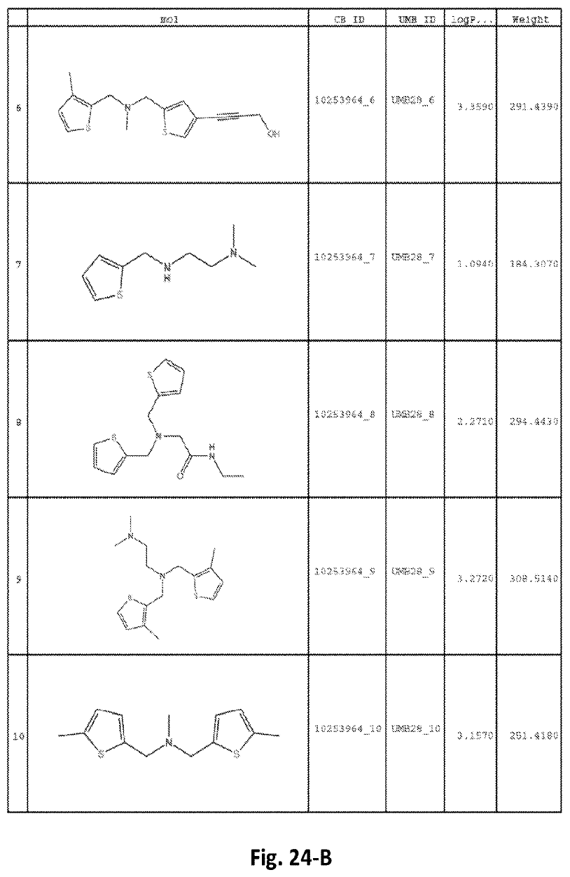

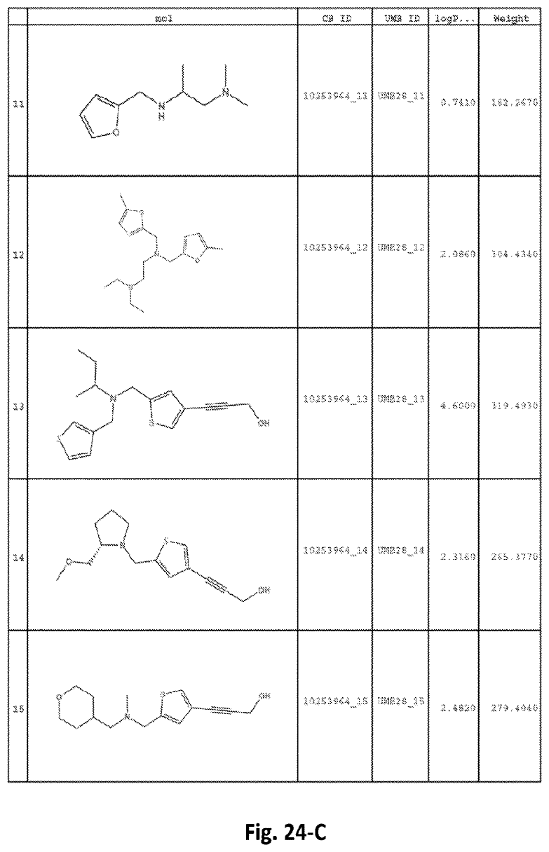

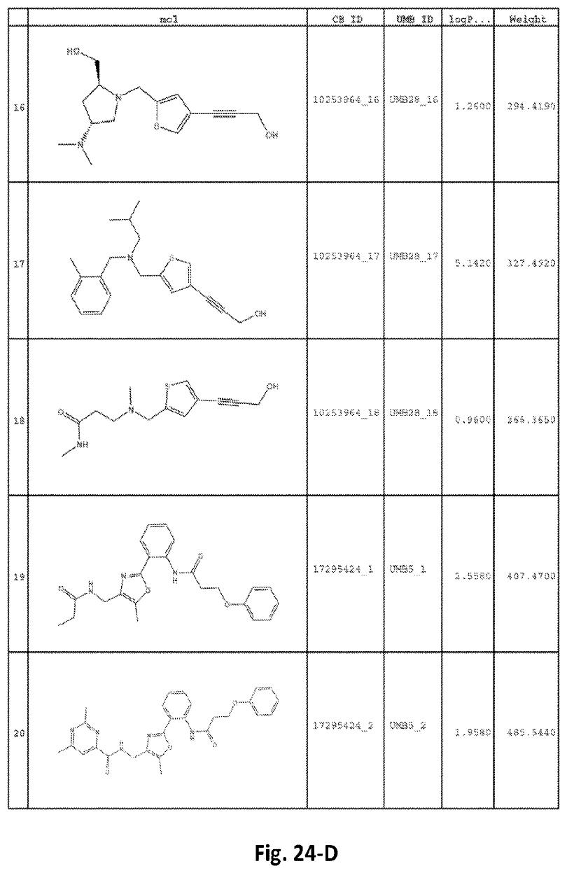

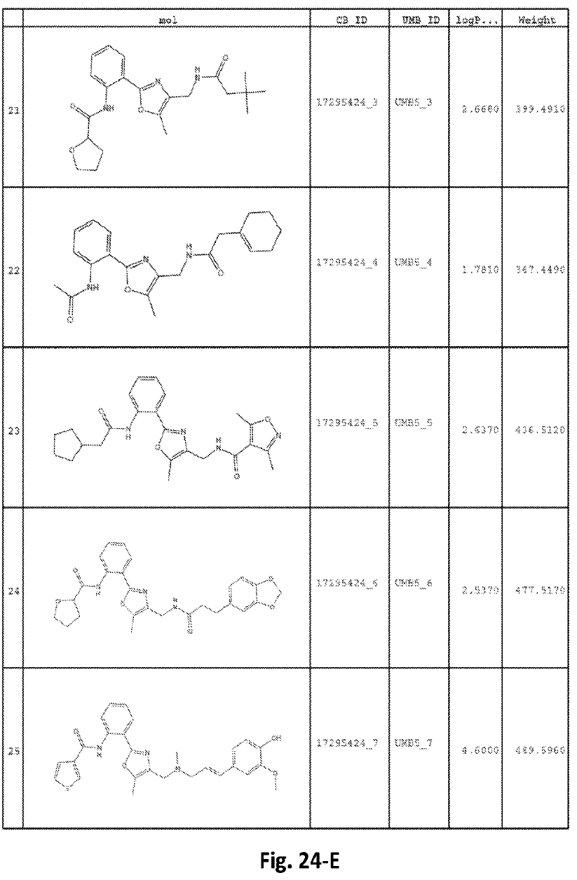

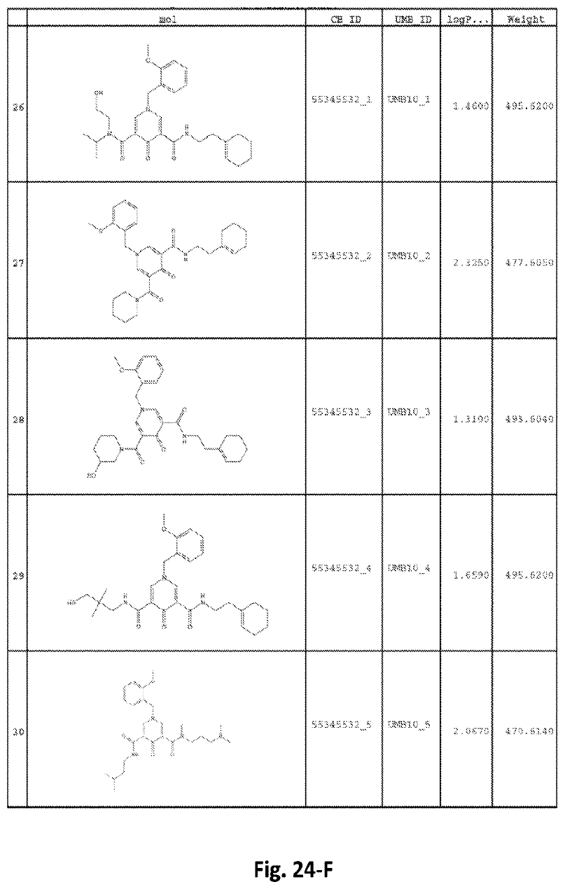

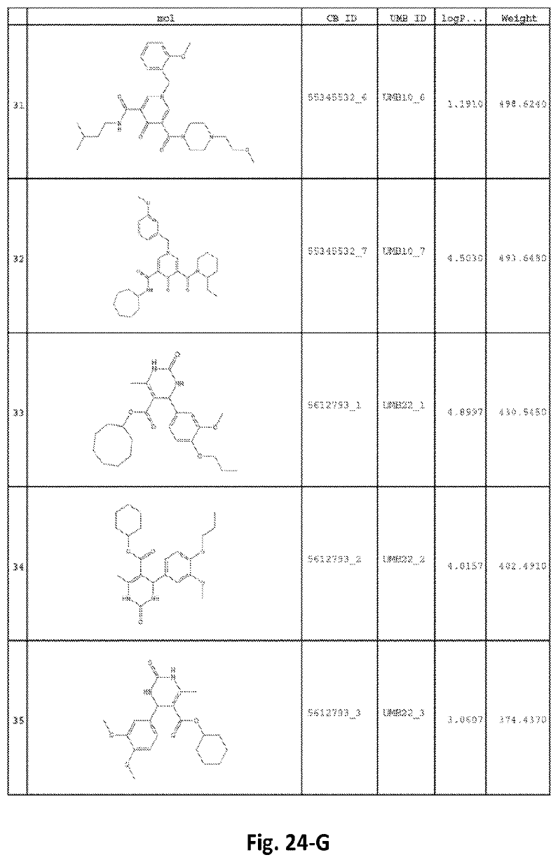

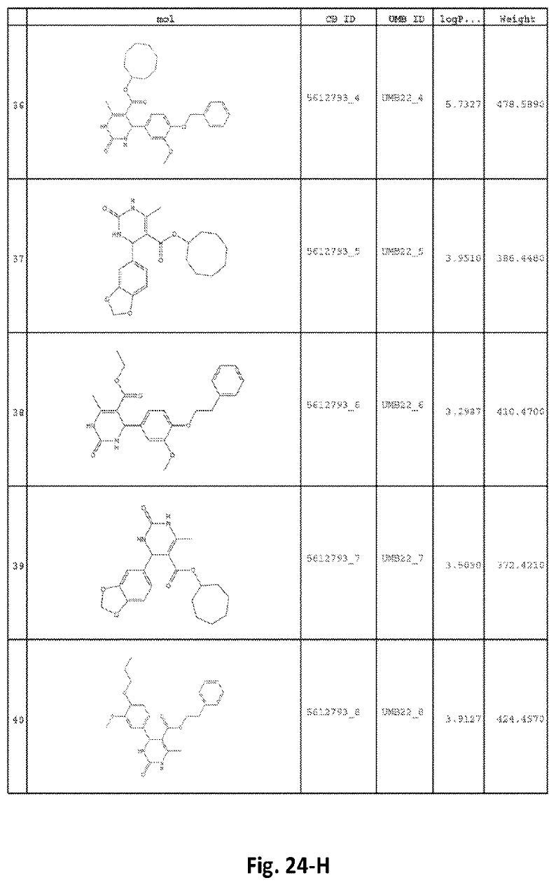

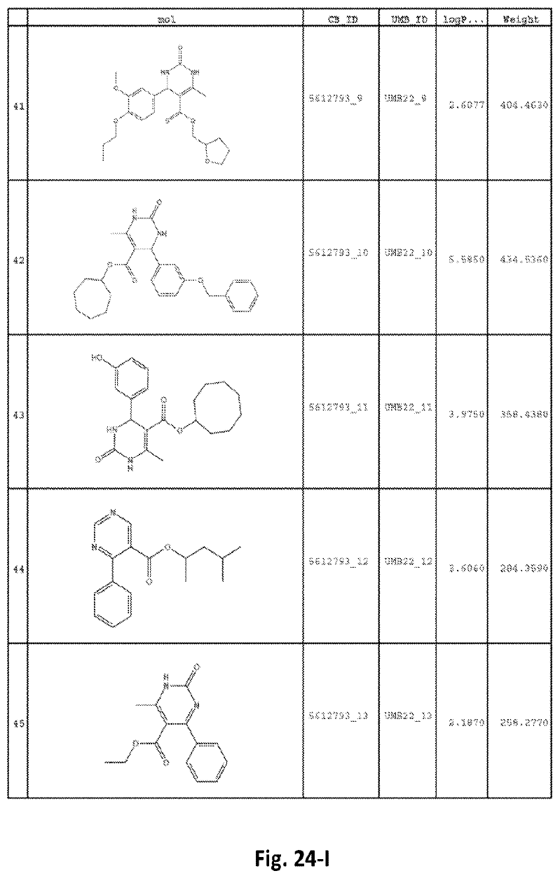

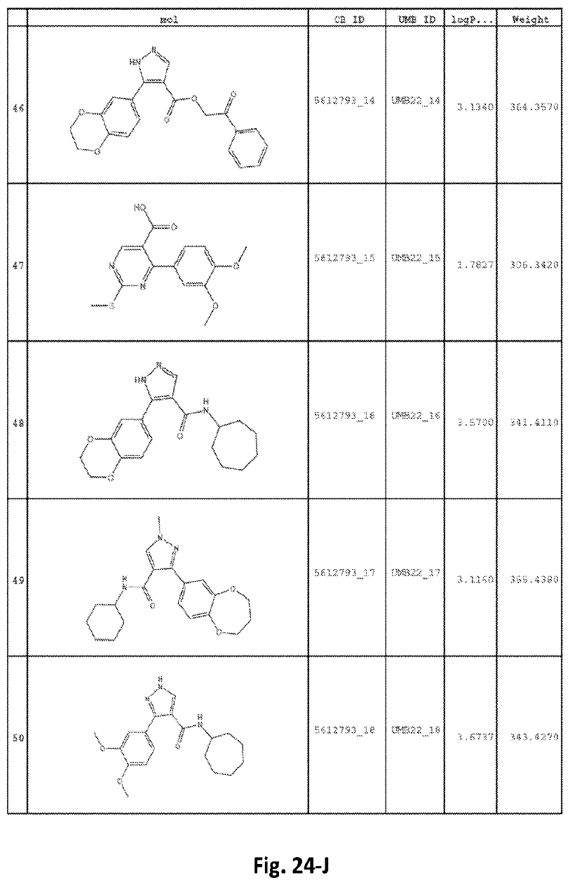

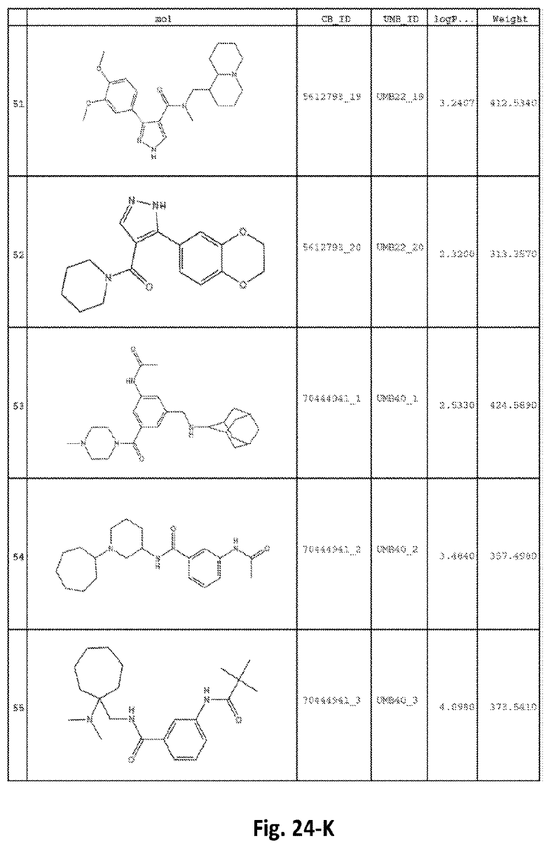

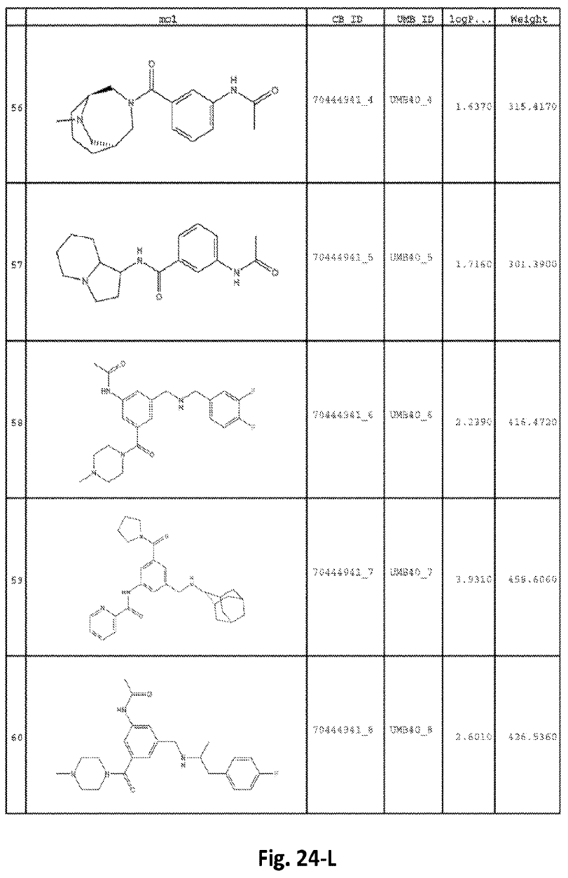

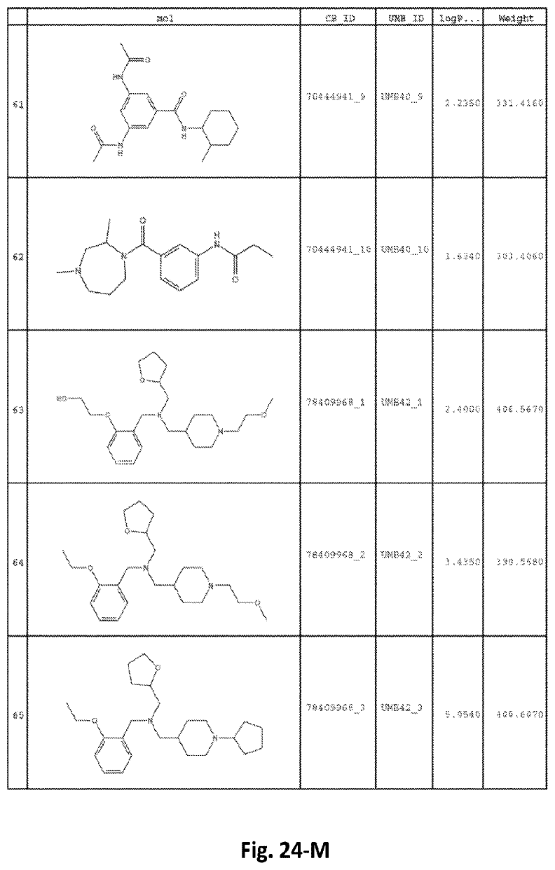

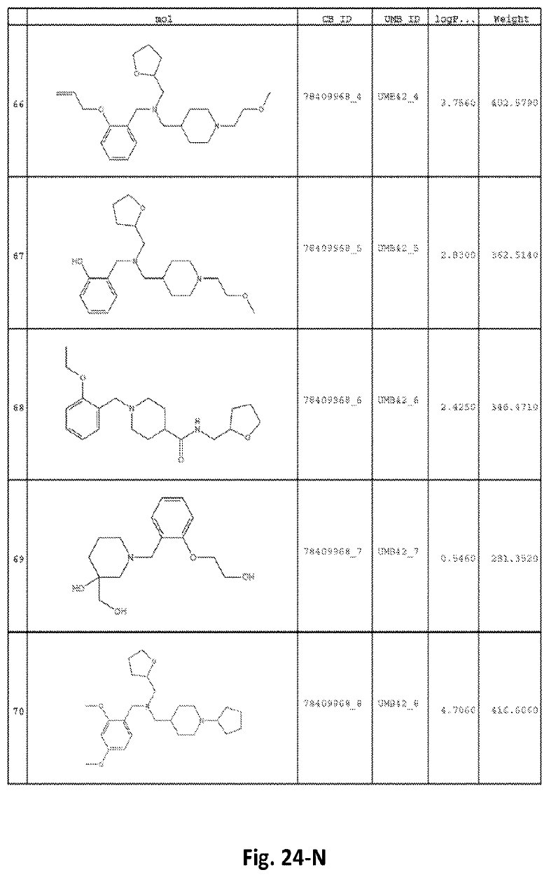

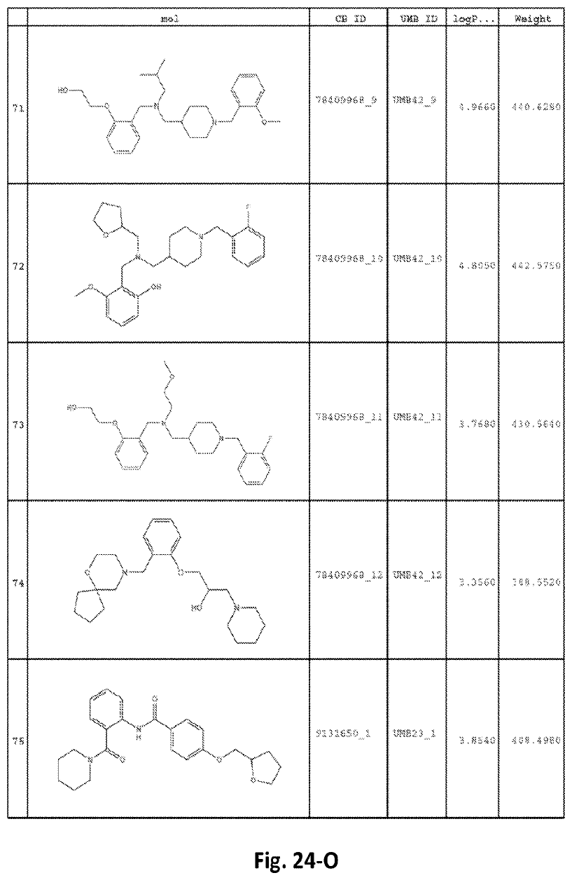

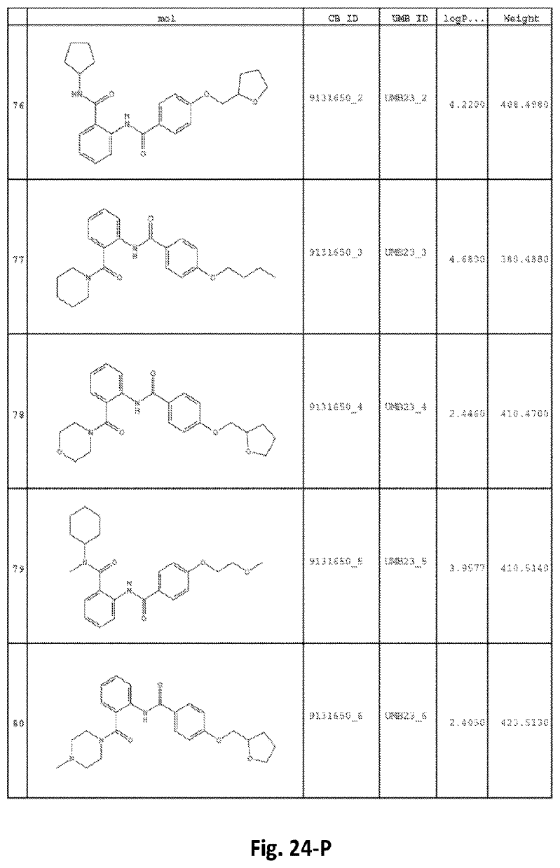

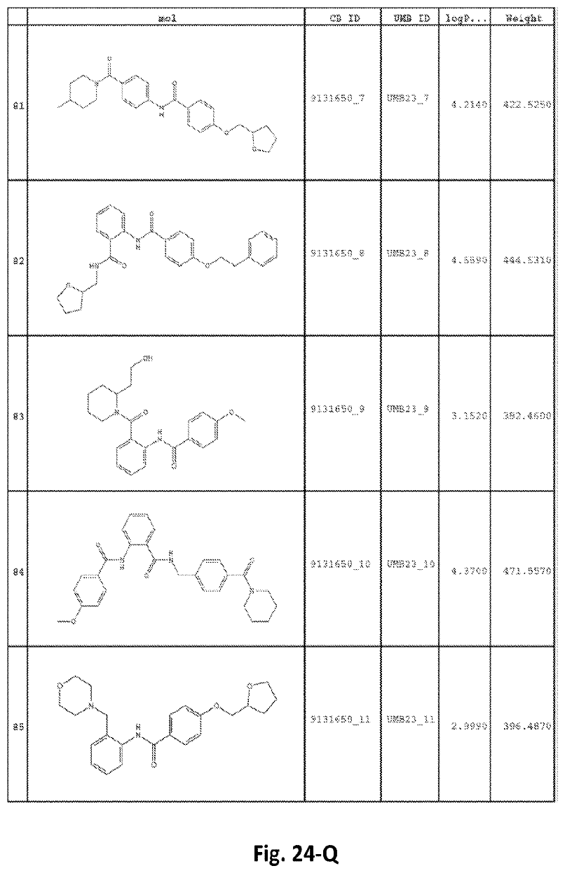

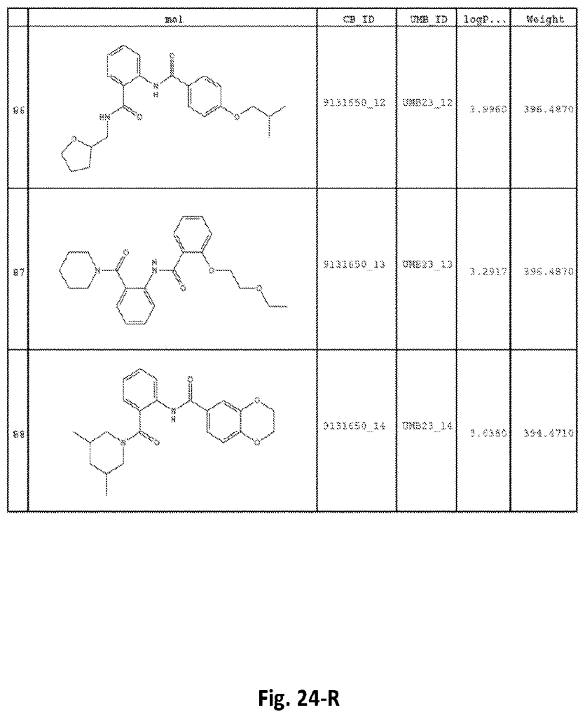

Similarity search of analogs of UMB18/96509034 to identify if either of the two-ring substructures of the lead have activity. Two compounds were obtained and tested for each two-ring substructure. Some activity was obtained for one of the substructures, see FIGS. 23A-23C, and the following table:

| 50 μM | | | | | |
|---|---|---|---|---|---|
| screen | | | | | |
| Compound ID | | 1-24-20 (triplicate wells) | | Avg | |
| 0.5% | | PFU/ml | PFU/ml | PFU/ml | |
| DMSO | | 21000 | 5300 | 15000 | 13150 |
| 18 | 96509034 | 600 | 200 | 300 | 400 |
| 18-OH1 | 6238481 | 26000 | 12000 | 30000 | 19000 |
| 18-OH2 | 5705656 | 8000 | 13000 | 16000 | 10500 |
| 18-F1 | 5562331 | 1100 | 6700 | 1400 | 1200 |
| 18-F2 | 5571262 | 23000 | 22000 | 20000 | 22500 |
| 10 μM | | | | | |
| screen | | | | | |
| Compound ID | | 1-24-20 (triplicate wells) | | Avg | |
| 0.1% | | PFU/ml | PFU/ml | PFU/ml | |
| DMSO | | 23000 | 19000 | 29000 | 21000 |
| 18 | 96509034 | 1300 | 1000 | 4000 | 1150 |
| 18-OH1 | 6238481 | 27000 | 70000 | 37000 | 48500 |
| 18-OH2 | 5705656 | 14000 | 3300 | 25000 | 8650 |
| 18-F1 | 5562331 | 15000 | 12000 | 12000 | 13500 |
| 18-F2 | 5571262 | 23000 | 29000 | 60000 | 26000 |

| | | | | Mol | |
|---|---|---|---|---|---|
| Molecule | Supplier | ID | Alt ID | Wt | CB ID |
| O═C(OCC)C1═C(C)NC(═O)NC1c1cc(OC)c(OCCc2ccccc2)cc1 | Hit2Lead | 7679642 | | 410 | 5612793_6 |
| O═C(Nc1c(-c2oc(C)c(CN(C/C═C/c3cc(OC)c(O)cc3)C)n2)cccc1)c1cscc1 | Hit2Lead | 48325627 | | 490 | 17295424_7 |
| O═C(NCCC1═CCCCC1)C═1C(═O)C(C(═O)N2CCCCC2)═CN | Hit2Lead | 93701800 | | 478 | 55345532_2 |
| (Cc2c(OC)cccc2)C = 1 | | | | | |
| OCC#Cc1cc(CN(Cc2c(C)ccs2)C)sc1 | Hit2Lead | 91240494 | | 291 | 10253964_6 |
| O═C(Nc1cc(C(═O)N2C[C@H]3N(C)C[C@@H] | Hit2Lead | 49602830 | | 315 | 70444941_4 |
| (C2)CCC3)ccc1)C | | | | | |
| O(C[C@H]1N(Cc2scc(C#CCO)c2)CCC1)C | Hit2Lead | 10434393 | | 265 | 10253964_14 |
| OCC#Cc1cc(CN2CC(C)N(C(C)C)CC2)sc1 | Hit2Lead | 30933538 | | 292 | 10253964_3 |
| OCC#Cc1cc(CN(CCN2CCCCCC2)C)sc1 | Hit2Lead | 37497358 | | 306 | 10253964_5 |
| OCC#Cc1cc(CN(CC2CCOCC2)C)sc1 | Hit2Lead | 37644792 | | 279 | 10253964_15 |
| O(CC(O)CN1CCCCC1)c1c(CN2CC3(OCC2)CCCC3)cccc1 | Hit2Lcad | 43103865 | | 389 | 78409968_12 |
| S(C)c1nc(c(C(═O)O)cn1)-c1cc(OC)c(OC)cc1 | Hit2Lead | 15578104 | | 306 | 5612793_15 |
| O(CCO)c1c(CN2CC(O)(CO)CCC2)cccc1 | Hit2Lead | 75846711 | | 281 | 78409968_7 |
| O═C(NC)CCN(Cc1scc(C#CCO)c1)C | Hit2Lead | 41503365 | | 266 | 10253964_18 |
| OCC#Cc1cc(CN(CCN2CCCCC2)CC)sc1 | Hit2Lead | 68133153 | | 306 | 10253964_4 |
| O(CCN1CCC(CN(Cc2c(OCCO)cccc2)CC2OCCC2)CC1)C | Hit2Lead | 78409968 | | 407 | 78409968_1 |
| O(CCN1CCC(CN(Cc2c(O)cccc2)CC2OCCC2)CC1)C | Hit2Lead | 49010376 | | 363 | 78409968_5 |
| O(CCN1CCC(CN(Cc2c(OCC)cccc2)CC2OCCC2)CC1)C | Hit2Lead | 35161547 | | 391 | 78409968_2 |
| Fc1c(CN2CCC(CN(Cc3c(OC)cccc3O)CC3OCCC3)CC2)cccc1 | Hit2Lead | 66893200 | | 443 | 78409968_10 |
| O(CCO)c1c(CN(CC(C)C)CC2CCN(Cc3c(OC)cccc3)CC2)cccc1 | Hit2Lead | 97913321 | | 441 | 78409968_9 |
| Fc1c(CN2CCC(CN(CCOC)Cc3c(OCCO)cccc3)CC2)cccc1 | Hit2Lead | 66918704 | | 431 | 78409968_11 |
| O═C(Nc1cc(C(═O)NC2CN(C3CCCCCC3)CCC2)ccc1)C | Hit2Lead | 38740381 | | 357 | 70444941_2 |
| O(CCN1CCC(CN(Cc2c(OCC═C)cccc2)CC2OCCC2)CC1)C | Hit2Lead | 97880594 | | 403 | 78409968_4 |
| O(C)c1c(CN(CC2OCCC2)CC2CCN(C3CCCC3)CC2)ccc(OC)c1 | Hit2Lead | 58156196 | | 417 | 78409968_8 |
| O(CC)c1c(CN(CC2OCCC2)CC2CCN(C3CCCC3)CC2)cccc1 | Hit2Lead | 38521191 | | 401 | 78409968_3 |
| O═C(Nc1cc(C(═O)N2CCN(C)CC2)cc(CNC2C3CC4CC2CC(C3)C4)c1)C | Hit2Lead | 70444941 | | 425 | 70444941_1 |
| O═C(N(C(C)C)CCO)C═1C(═O)C(C(═O)NCCC2═CCCCC2)═CN | Hit2Lead | 55345532 | | 496 | 55345532_1 |
| (Cc2c(OC)cccc2)C = 1 | | | | | |
| O═C(N(CCCN(C)C)C)C═1C(═O)C(C(═O)NCCC(C)C)═CN | Hit2Lead | 23614835 | | 471 | 55345532_5 |
| (Cc2c(OC)cccc2)C = 1 | | | | | |
| Fc1c(F)ccc(CNCc2cc(NC(═O)C)cc(C(═O)N3CCN(C)CC3)c2)c1 | Hit2Lead | 73759021 | | 416 | 70444941_6 |
| O═C(NCCC1═CCCCC1)C═1C(═O)C(C(═O)N2CC(O)CCC2)═CN | Hit2Lead | 53145516 | | 494 | 55345532_3 |
| (Cc2c(OC)cccc2)C = 1 | | | | | |
| O═C(NC1CCCCCC1)C═1C(═O)C(C(═O)N2C(CC)CCCC2)═CN | Hit2Lead | 47976750 | | 494 | 55345532_7 |
| (Cc2cc(OC)ccc2)C = 1 | | | | | |
| O═C(NCCC(C)C)C═1C(═O)C(C(═O)N2CCN(CCOC)CC2)═CN | Hit2Lead | 24668741 | | 499 | 55345532_6 |
| (Cc2c(OC)cccc2)C = 1 | | | | | |
| O═C(Nc1cc(C(═O)N2CCCC2)cc(CNC2C3CC4CC2CC(C3)C4)c1)c1ncccc1 | Hit2Lead | 92881721 | | 459 | 70444941_7 |
| O═C(Nc1cc(C(═O)N2C(C)CN(C)CCC2)ccc1)CC | Hit2Lead | 41610622 | | 303 | 70444941_10 |
| OCC#Cc1cc(CN(CCN(C)C)Cc2sc(C)cc2)sc1 | Hit2Lead | 10253964 | | 349 | 10253964_1 |
| OCC#Cc1cc(CN(CCCO)Cc2sc(C)cc2)sc1 | Hit2Lead | 24200140 | | 335 | 10253964_2 |
| OCC#Cc1cc(CN(CC(C)C)Cc2c(C)cccc2)sc1 | Hit2Lead | 18052219 | | 327 | 10253964_17 |
| N(CCN(C)C)(Cc1c(C)ccs1)Cc1c(C)ccs1 | Hit2Lead | 10355732 | | 309 | 10253964_9 |
| OCC#Cc1cc(CN(C(CC)C)Cc2cscc2)sc1 | Hit2Lead | 26918198 | | 319 | 10253964_13 |
| O═C(OC1CCCCC1)C1═C(C)NC(═O)NC1c1cc(OC)c(OC)cc1 | Vitas-M | STK364138 | | 374.44 | 5612793_3 |
| | Labs | | | | |
| O═C(NC1CCCCCC1)c1c(-c2cc(OC)c(OC)cc2)n[nH]c1 | Vitas-M | STL184833 | | 343.43 | 5612793_18 |
| | Labs | | | | |
| O═C(Nc1c(C(═O)N2CCCCC2)cccc1)c1ccc(OCCCC)cc1 | Vitas-M | STL071122 | | 380.49 | 9131650_3 |
| | Labs | | | | |
| O═C(Nc1c(C(═O)N2CCCCC2)cccc1)c1c(OCCOCC)cccc1 | Vitas-M | STL066877 | | 396.49 | 9131650_13 |
| | Labs | | | | |
| O═C(Nc1c(C(═O)N2CCOCC2)cccc1)c1ccc(OCC2OCCC2)cc1 | Vitas-M | STL068656 | | 410.47 | 9131650_4 |
| | Labs | | | | |
| O═C(Nc1ccc(C(═O)N2CCC(C)CC2)cc1)c1ccc(OCC2OCCC2)cc1 | Vitas-M | STL068664 | | 422.52 | 9131650_7 |
| | Labs | | | | |
| O═C(Nc1c(C(═O)N2CCCCC2)cccc1)c1ccc(OCC2OCCC2)cc1 | Vitas-M | STL068657 | | 408.5 | 9131650_1 |
| | Labs | | | | |
| O═C(Nc1c(C(═O)N2CCN(C)CC2)cccc1)c1ccc(OCC2OCCC2)cc1 | Vitas-M | STL068659 | | 423.51 | 9131650_6 |
| | Labs | | | | |
| N(Cc1sc(C)cc1)(Cc1sc(C)cc1)C | Vitas-M | STL069512 | | 251.42 | 10253964_10 |
| | Labs | | | | |
| O═C(OC1CCCCCCC1)C1═C(C)NC(═O)NC1c1cc(O)ccc1 | Vitas-M | STK062421 | | 358.44 | 5612793_11 |
| | Labs | | | | |
| O═C(NCC1OCCC1)C1CCN(Cc2c(OCC)cccc2)CC1 | Vitas-M | STK017119 | | 346.47 | 78409968_6 |
| | Labs | | | | |
| N(CCN(Cc1oc(C)cc1)Cc1oc(C)cc1)(CC)CC | Vitas-M | STK161925 | | 304.43 | 10253964_12 |
| | Labs | | | | |
| O═C(N(CC1C2N(CCC1)CCCC2)C)c1c(-c2cc(OC)c(OC)cc2)n[nH]c1 | Vitas-M | STL546978 | | 412.53 | 5612793_19 |
| | Labs | | | | |
| O═C(OCCc1ccccc1)C1═C(C)NC(═O)NC1c1cc(OC)c(OCCC)cc1 | Vitas-M | STK083274 | | 424.5 | 5612793_8 |
| | Labs | | | | |
| O═C(OC1CCCCCCC1)C1═C(C)NC(═O)NC1c1cc(OC)c(OCc2ccccc2)cc1 | Vitas-M | STK075753 | | 478.59 | 5612793_4 |
| | Labs | | | | |
| O═C(OCC1OCCC1)C1═C(C)NC(═O)NC1c1cc(OC)c(OCCC)cc1 | Vitas-M | STK054478 | | 404.46 | 5612793_9 |
| | Labs | | | | |
| O═C(OC(CC(C)C)C)c1c(-c2ccccc2)ncnc1 | Vitas-M | STK244118 | | 396.49 | 5612793_12 |
| | Labs | | | | |
| O═C(NCC1OCCC1)c1c(NC(═O)c2ccc(OCCc3ccccc3)cc2)cccc1 | Vitas-M | STK276413 | | 444.53 | 9131650_8 |
| | Labs | | | | |
| O═C(N(C)C1CCCCC1)c1c(NC(═O)c2ccc(OCCOC)cc2)cccc1 | Vitas-M | STK345854 | | 410.51 | 9131650_5 |
| | Labs | | | | |
| O═C(NC1CCCCCC1)c1c(-c2cc3OCCOc3cc2)[nH]nc1 | Enamine | Z224428262 | MolPort- | 341.4 | 5612793_16 |
| | Ltd | | 009- | | |
| | | | 439-585 | | |
| O═C(Nc1cc(C(═O)NCC2(N(C)C)CCCCCC2)ccc1)C(C)(C)C | Enamine | Z253654054 | MolPort- | 373.5 | 70444941_3 |
| | Ltd | | 009- | | |
| | | | 471-445 | | |
| O═C(NC1CCCCC1)c1c(-c2cc3OCCCOc3cc2)nn(C)c1 | Enamine | Z79410098 | MolPort- | 355.4 | 5612793_17 |
| | Ltd | | 009- | | |
| | | | 228-990 | | |
| O═C(NC1CCCC1)c1c(NC(═O)c2ccc(OCC3OCCC3)cc2)cccc1 | Enamine | Z28085941 | MolPort- | 408.5 | 9131650_2 |
| | Ltd | | 009- | | |
| | | | 496-837 | | |
| O═C(Nc1c(CN2CCOCC2)cccc1)c1ccc(OCC2OCCC2)cc1 | UkrOrgSyn- | PB146722878 | MolPort- | 471.6 | 9131650_11 |
| | thesis | | 005- | | |
| | Ltd. Stock | | 781-557 | | |
| O═C(OCC(═O)c1ccccc1)c1c(-c2cc3OCCOc3cc2)[nH]nc1 | Enamine | Z217871108 | MolPort- | 364.4 | 5612793_14 |
| | Ltd | | 005- | | |
| | | | 524-491 | | |
| O═C(Nc1cc(NC(═O)C)cc(C(═O)NC2C(C)CCCC2)c1)C | Enamine | Z27075709 | MolPort- | 331.4 | 70444941_9 |
| | Ltd | | 004- | | |
| | | | 209-658 | | |
| O═C(Nc1cc(C(═O)NC2C3N(CC2)CCCC3)ccc1)C | Enamine | Z767436704 | MolPort- | 301.4 | 70444941_5 |
| | Ltd | | 020- | | |
| | | | 114-021 | | |
| O═C(OCC)C═1C(c2ccccc2)═NC(═O)NC═1C | Enamine | Z1665450493 | MolPort- | 284.4 | 5612793_13 |
| | Ltd | | 028- | | |
| | | | 802-725 | | |
| O═C(NCC1OCCC1)c1c(NC(═O)c2ccc(OCC(C)C)cc2)cccc1 | Enamine | Z739206878 | MolPort- | 396.5 | 9131650_12 |
| | Ltd | | 009- | | |
| | | | 088-584 | | |
| O═C(N1CCCCC1)c1c(-c2cc3OCCOc3cc2)[nH]nc1 | Enamine | Z224448688 | MolPort- | 313.4 | 5612793_20 |
| | Ltd | | 009- | | |
| | | | 527-391 | | |
| O═C(Nc1c(C(═O)N2CC(C)CC(C)C2)cccc1)c1cc2OCCOc2cc1 | Enamine | Z394427336 | MolPort- | 394.5 | 9131650_14 |
| | Ltd | | 009- | | |
| | | | 306-253 | | |
| O═C(NCC)CN(Cc1sccc1)Cc1sccc1 | Enamine | Z162917606 | MolPort- | 294.4 | 10253964_8 |
| | Ltd | | 009- | | |
| | | | 133-665 | | |
| N(CCNCc1sccc1)(C)C | Enamine | Z90512807 | MolPort- | 184.3 | 10253964_7 |
| | Ltd | | 000- | | |
| | | | 938-899 | | |
| O═C(OC1CCCCCCC1)C1═C(C)NC(═O)NC1c1cc(OC)c(OCCC)cc1 | Specs | AG-205/ | MolPort- | 430.5 | 5612793_1 |
| | | 11867180 | 000- | | |
| | | | 907-180 | | |
| O═C(Nc1c(C(═O)N2C(CCO)CCCC2)cccc1)c1ccc(OC)cc1 | Specs | AP-064/ | MolPort- | 382.5 | 9131650_9 |
| | | 41684584 | 002- | | |
| | | | 090-971 | | |
| O═C(OC1CCCCCC1)C1═C(C)NC(═O)NC1c1cc(OCc2ccccc2)ccc1 | Specs | AG-205/ | MolPort- | 434.5 | 5612793_10 |
| | | 11867172 | 000- | | |
| | | | 911-785 | | |
| O═C(OC1CCCCCCC1)C1═C(C)NC(═O)NC1c1cc2OCOc2cc1 | Specs | AG-690/ | MolPort- | 386.4 | 5612793_5 |
| | | 33475017 | 000- | | |
| | | | 906-361 | | |
| O═C(OC1CCCCCC1)C1═C(C)NC(═O)NC1c1cc2OCOc2cc1 | ChemDiv, | 1308- | MolPort- | 372.4 | 5612793_7 |
| | Inc | 0451 | 001- | | |
| | | | 928-269 | | |

| | | | 50 μM treatment | |
|---|---|---|---|---|
| | | UMB ID | DMSO | |
| Molecule | UMB ID | new | (0.5%) | 33000 |
| | | | UMB18 | 0 |
| O═C(OCC)C1═C(C)NC(═O)NC1c1cc(OC)c(OCCc2ccccc2)cc1 | UMB22_6 | 41 | UMB41 | 33000 |
| O═C(Nc1c(-c2oc(C)c(CN(C/C═C/c3cc(OC)c(O)cc3)C)n2)cccc1)c1cscc1 | UMB5_7 | 42 | UMB42 | 10000 |
| O═C(NCCC1═CCCCC1)C═1C(═O)C(C(═O)N2CCCCC2)═CN | UMB10_2 | 43 | UMB43 | 35000 |
| (Cc2c(OC)cccc2)C = 1 | | | | |
| OCC#Cc1cc(CN(Cc2c(C)ccs2)C)sc1 | UMB28_6 | 44 | UMB44 | 33000 |
| O═C(Nc1cc(C(═O)N2C[C@H]3N(C)C[C@@H] | UMB40_4 | 45 | UMB45 | 32000 |
| (C2)CCC3)ccc1)C | | | | |
| O(C[C@H]1N(Cc2scc(C#CCO)c2)CCC1)C | UMB28_14 | 46 | UMB46 | 34000 |
| OCC#Cc1cc(CN2CC(C)N(C(C)C)CC2)sc1 | UMB28_3 | 47 | UMB47 | 190000 |
| OCC#Cc1cc(CN(CCN2CCCCCC2)C)sc1 | UMB28_5 | 48 | UMB48 | 23000 |
| OCC#Cc1cc(CN(CC2CCOCC2)C)sc1 | UMB28_15 | 49 | UMB49 | 46000 |
| O(CC(O)CN1CCCCC1)c1c(CN2CC3(OCC2)CCCC3)cccc1 | UMB42_12 | 50 | UMB50 | 0 |
| S(C)c1nc(c(C(═O)O)cn1)-c1cc(OC)c(OC)cc1 | UMB22_15 | 51 | UMB51 | 19000 |
| O(CCO)c1c(CN2CC(O)(CO)CCC2)cccc1 | UMB42_7 | 52 | UMB52 | 16000 |
| O═C(NC)CCN(Cc1scc(C#CCO)c1)C | UMB28_18 | 53 | UMB53 | 110000 |
| OCC#Cc1cc(CN(CCN2CCCCC2)CC)sc1 | UMB28_4 | 54 | UMB54 | 2000 |
| O(CCN1CCC(CN(Cc2c(OCCO)cccc2)CC2OCCC2)CC1)C | UMB42_1 | 55 | UMB55 | 8000 |
| O(CCN1CCC(CN(Cc2c(O)cccc2)CC2OCCC2)CC1)C | UMB42_5 | 56 | UMB56 | 11000 |
| O(CCN1CCC(CN(Cc2c(OCC)cccc2)CC2OCCC2)CC1)C | UMB42_2 | 57 | UMB57 | 3000 |
| Fc1c(CN2CCC(CN(Cc3c(OC)cccc3O)CC3OCCC3)CC2)cccc1 | UMB42_10 | 58 | UMB58 | 0 |
| O(CCO)c1c(CN(CC(C)C)CC2CCN(Cc3c(OC)cccc3)CC2)cccc1 | UMB42_9 | 59 | UMB59 | 1000 |
| Fc1c(CN2CCC(CN(CCOC)Cc3c(OCCO)cccc3)CC2)cccc1 | UMB42_11 | 60 | UMB60 | 3000 |
| | | | DMSO | 18500 |
| | | | (0.5%) | |
| | | | UMB18 | 0 |
| | | | UMB18 | 0 |
| | | | (gel) | |
| O═C(Nc1cc(C(═O)NC2CN(C3CCCCCC3)CCC2)ccc1)C | UMB40_2 | 61 | UMB61 | 1000 |
| O(CCN1CCC(CN(Cc2c(OCC═C)cccc2)CC2OCCC2)CC1)C | UMB42_4 | 62 | UMB62 | 0 |
| O(C)c1c(CN(CC2OCCC2)CC2CCN(C3CCCC3)CC2)ccc(OC)c1 | UMB42_8 | 63 | UMB63 | 1000 |
| O(CC)c1c(CN(CC2OCCC2)CC2CCN(C3CCCC3)CC2)cccc1 | UMB42_3 | 64 | UMB64 | 0 |
| O═C(Nc1cc(C(═O)N2CCN(C)CC2)cc(CNC2C3CC4CC2CC(C3)C4)c1)C | UMB40_1 | 65 | UMB65 | 2000 |
| O═C(N(C(C)C)CCO)C═1C(═O)C(C(═O)NCCC2═CCCCC2)═CN | UMB10_1 | 66 | UMB66 | 0 |
| (Cc2c(OC)cccc2)C = 1 | | | | |
| O═C(N(CCCN(C)C)C)C═1C(═O)C(C(═O)NCCC(C)C)═CN | UMB10_5 | 67 | UMB67 | 1000 |
| (Cc2c(OC)cccc2)C = 1 | | | | |
| Fc1c(F)ccc(CNCc2cc(NC(═O)C)cc(C(═O)N3CCN(C)CC3)c2)c1 | UMB40_6 | 68 | UMB68 | 6000 |
| O═C(NCCC1═CCCCC1)C═1C(═O)C(C(═O)N2CC(O)CCC2)═CN | UMB10_3 | 69 | UMB69 | 8500 |
| (Cc2c(OC)cccc2)C = 1 | | | | |
| O═C(NC1CCCCCC1)C═1C(═O)C(C(═O)N2C(CC)CCCC2)═CN | UMB10_7 | 70 | UMB70 | 7000 |
| (Cc2cc(OC)ccc2)C = 1 | | | | |
| O═C(NCCC(C)C)C═1C(═O)C(C(═O)N2CCN(CCOC)CC2)═CN | UMB10_6 | 71 | UMB71 | 3000 |
| (Cc2c(OC)cccc2)C = 1 | | | | |
| O═C(Nc1cc(C(═O)N2CCCC2)cc(CNC2C3CC4CC2CC(C3)C4)c1)c1ncccc1 | UMB40_7 | 72 | UMB72 | 0 |
| O═C(Nc1cc(C(═O)N2C(C)CN(C)CCC2)ccc1)CC | UMB40_10 | 73 | UMB73 | 2000 |
| OCC#Cc1cc(CN(CCN(C)C)Cc2sc(C)cc2)sc1 | UMB28_1 | 74 | UMB74 | 0 |
| OCC#Cc1cc(CN(CCCO)Cc2sc(C)cc2)sc1 | UMB28_2 | 75 | UMB75 | 12000 |
| OCC#Cc1cc(CN(CC(C)C)Cc2c(C)cccc2)sc1 | UMB28_17 | 76 | UMB76 | 5000 |
| N(CCN(C)C)(Cc1c(C)ccs1)Cc1c(C)ccs1 | UMB28_9 | 77 | UMB77 | 0 |
| OCC#Cc1cc(CN(C(CC)C)Cc2cscc2)sc1 | UMB28_13 | 78 | UMB78 | 3000 |
| | | | DMSO | 7000 |
| | | | (0.5%) | |
| | | | UMB18 | 0 |
| | | | UMB18 | |
| | | | (gel) | |
| O═C(OC1CCCCC1)C1═C(C)NC(═O)NC1c1cc(OC)c(OC)cc1 | UMB22_3 | 79 | UMB79 | 15500 |
| O═C(NC1CCCCCC1)c1c(-c2cc(OC)c(OC)cc2)n[nH]c1 | UMB22_18 | 80 | UMB80 | 8500 |
| O═C(Nc1c(C(═O)N2CCCCC2)cccc1)c1ccc(OCCCC)cc1 | UMB23_3 | 81 | UMB81 | 9000 |
| O═C(Nc1c(C(═O)N2CCCCC2)cccc1)c1c(OCCOCC)cccc1 | UMB23_13 | 82 | UMB82 | 0 |
| O═C(Nc1c(C(═O)N2CCOCC2)cccc1)c1ccc(OCC2OCCC2)cc1 | UMB23_4 | 83 | UMB83 | 0 |
| O═C(Nc1ccc(C(═O)N2CCC(C)CC2)cc1)c1ccc(OCC2OCCC2)cc1 | UMB23_7 | 84 | UMB84 | 2000 |
| O═C(Nc1c(C(═O)N2CCCCC2)cccc1)c1ccc(OCC2OCCC2)cc1 | UMB23_1 | 85 | UMB85 | 5000 |
| O═C(Nc1c(C(═O)N2CCN(C)CC2)cccc1)c1ccc(OCC2OCCC2)cc1 | UMB23_6 | 86 | UMB86 | 5000 |
| N(Cc1sc(C)cc1)(Cc1sc(C)cc1)C | UMB28_10 | 87 | UMB87 | 2000 |
| O═C(OC1CCCCCCC1)C1═C(C)NC(═O)NC1c1cc(O)ccc1 | UMB22_11 | 88 | UMB88 | 5000 |
| O═C(NCC1OCCC1)C1CCN(Cc2c(OCC)cccc2)CC1 | UMB42_6 | 89 | UMB89 | 7000 |
| N(CCN(Cc1oc(C)cc1)Cc1oc(C)cc1)(CC)CC | UMB28_12 | 90 | UMB90 | 0 |
| O═C(N(CC1C2N(CCC1)CCCC2)C)c1c(-c2cc(OC)c(OC)cc2)n[nH]c1 | UMB22_19 | 91 | UMB91 | 5000 |
| O═C(OCCc1ccccc1)C1═C(C)NC(═O)NC1c1cc(OC)c(OCCC)cc1 | UMB22_8 | 92 | UMB92 | 2000 |
| O═C(OC1CCCCCCC1)C1═C(C)NC(═O)NC1c1cc(OC)c(OCc2ccccc2)cc1 | UMB22_4 | 93 | UMB93 | 0 |
| O═C(OCC1OCCC1)C1═C(C)NC(═O)NC1c1cc(OC)c(OCCC)cc1 | UMB22_9 | 94 | UMB94 | 2000 |
| O═C(OC(CC(C)C)C)c1c(-c2ccccc2)ncnc1 | UMB22_12 | 95 | UMB95 | 5000 |
| O═C(NCC1OCCC1)c1c(NC(═O)c2ccc(OCCc3ccccc3)cc2)cccc1 | UMB23_8 | 96 | UMB96 | 5000 |
| O═C(N(C)C1CCCCC1)c1c(NC(═O)c2ccc(OCCOC)cc2)cccc1 | UMB23_5 | 97 | UMB97 | 7000 |
| O═C(NC1CCCCCC1)c1c(-c2cc3OCCOc3cc2)[nH]nc1 | UMB22_16 | 98 | UMB98 | 0 |
| O═C(Nc1cc(C(═O)NCC2(N(C)C)CCCCCC2)ccc1)C(C)(C)C | UMB40_3 | 99 | UMB99 | 24500 |
| O═C(NC1CCCCC1)c1c(-c2cc3OCCCOc3cc2)nn(C)c1 | UMB22_17 | 100 | UMB100 | 10500 |
| | | | DMSO | 140000 |
| | | | (0.5%) | |
| | | | UMB18 | 0 |
| | | | UMB18 | 0 |
| | | | (gel) | |
| O═C(NC1CCCC1)c1c(NC(═O)c2ccc(OCC3OCCC3)cc2)cccc1 | UMB23_2 | 101 | UMB101 | 690000 |
| O═C(Nc1c(CN2CCOCC2)cccc1)c1ccc(OCC2OCCC2)cc1 | UMB23_11 | 102 | UMB102 | 240000 |
| O═C(OCC(═O)c1ccccc1)c1c(-c2cc3OCCOc3cc2)[nH]nc1 | UMB22_14 | 103 | UMB103 | 50000 |
| O═C(Nc1cc(NC(═O)C)cc(C(═O)NC2C(C)CCCC2)c1)C | UMB40_9 | 104 | UMB104 | 355000 |
| O═C(Nc1cc(C(═O)NC2C3N(CC2)CCCC3)ccc1)C | UMB40_5 | 105 | UMB105 | 140000 |
| O═C(OCC)C═1C(c2ccccc2)═NC(═O)NC═1C | UMB22_13 | 106 | UMB106 | 90000 |
| O═C(NCC1OCCC1)c1c(NC(═O)c2ccc(OCC(C)C)cc2)cccc1 | UMB23_12 | 107 | UMB107 | 60000 |
| O═C(N1CCCCC1)c1c(-c2cc3OCCOc3cc2)[nH]nc1 | UMB22_20 | 108 | UMB108 | 18000 |
| O═C(Nc1c(C(═O)N2CC(C)CC(C)C2)cccc1)c1cc2OCCOc2cc1 | UMB23_14 | 109 | UMB109 | 130000 |
| O═C(NCC)CN(Cc1sccc1)Cc1sccc1 | UMB28_8 | 110 | UMB110 | 150000 |
| N(CCNCc1sccc1)(C)C | UMB28_7 | 111 | UMB111 | 600000 |
| O═C(OC1CCCCCCC1)C1═C(C)NC(═O)NC1c1cc(OC)c(OCCC)cc1 | UMB22_1 | 112 | UMB112 | 85000 |
| O═C(Nc1c(C(═O)N2C(CCO)CCCC2)cccc1)c1ccc(OC)cc1 | UMB23_9 | 113 | UMB113 | 195000 |
| O═C(OC1CCCCCC1)C1═C(C)NC(═O)NC1c1cc(OCc2ccccc2)ccc1 | UMB22_10 | 114 | UMB114 | 170000 |
| O═C(OC1CCCCCCC1)C1═C(C)NC(═O)NC1c1cc2OCOc2cc1 | UMB22_5 | 115 | UMB115 | 0 |
| O═C(OC1CCCCCC1)C1═C(C)NC(═O)NC1c1cc2OCOc2cc1 | UMB22_7 | 116 | UMB116 | 0 |

| | 50 μM treatment | | | Average |
|---|---|---|---|---|
| Molecule | 80000 | 23000 | | 45333.33333 |
| | 3000 | 0 | 1000 | 1000 |
| O═C(OCC)C1═C(C)NC(═O)NC1c1cc(OC)c(OCCc2ccccc2)cc1 | 0 | | | 16500 |
| O═C(Nc1c(-c2oc(C)c(CN(C/C═C/c3cc(OC)c(O)cc3)C)n2)cccc1)c1cscc1 | 2000 | | | 6000 |
| O═C(NCCC1═CCCCC1)C═1C(═O)C(C(═O)N2CCCCC2)═CN | 5000 | | | 20000 |
| (Cc2c(OC)cccc2)C = 1 | | | | |
| OCC#Cc1cc(CN(Cc2c(C)ccs2)C)sc1 | 16000 | | | 24500 |
| O═C(Nc1cc(C(═O)N2C[C@H]3N(C)C[C@@H] | 0 | | | 16000 |
| (C2)CCC3)ccc1)C | | | | |
| O(C[C@H]1N(Cc2scc(C#CCO)c2)CCC1)C | 4000 | | | 19000 |
| OCC#Cc1cc(CN2CC(C)N(C(C)C)CC2)sc1 | 37000 | | | 113500 |
| OCC#Cc1cc(CN(CCN2CCCCCC2)C)sc1 | 4000 | | | 13500 |
| OCC#Cc1cc(CN(CC2CCOCC2)C)sc1 | 27000 | | | 36500 |
| O(CC(O)CN1CCCCC1)c1c(CN2CC3(OCC2)CCCC3)cccc1 | 0 | | | 0 |
| S(C)c1nc(c(C(═O)O)cn1)-c1cc(OC)c(OC)cc1 | 7000 | | | 13000 |
| O(CCO)c1c(CN2CC(O)(CO)CCC2)cccc1 | 13000 | | | 14500 |
| O═C(NC)CCN(Cc1scc(C#CCO)c1)C | 7000 | | | 58500 |
| OCC#Cc1cc(CN(CCN2CCCCC2)CC)sc1 | 0 | | | 1000 |
| O(CCN1CCC(CN(Cc2c(OCCO)cccc2)CC2OCCC2)CC1)C | 0 | | | 4000 |
| O(CCN1CCC(CN(Cc2c(O)cccc2)CC2OCCC2)CC1)C | 2000 | | | 6500 |
| O(CCN1CCC(CN(Cc2c(OCC)cccc2)CC2OCCC2)CC1)C | 3000 | | | 3000 |
| Fc1c(CN2CCC(CN(Cc3c(OC)cccc3O)CC3OCCC3)CC2)cccc1 | 3000 | | | 1500 |
| O(CCO)c1c(CN(CC(C)C)CC2CCN(Cc3c(OC)cccc3)CC2)cccc1 | 2000 | | | 1500 |
| Fc1c(CN2CCC(CN(CCOC)Cc3c(OCCO)cccc3)CC2)cccc1 | 4000 | | | 3500 |
| | 28000 | 16000 | | 20833.33333 |
| | 0 | 1000 | | 333.3333333 |
| | 0 | | | 0 |
| O═C(Nc1cc(C(═O)NC2CN(C3CCCCCC3)CCC2)ccc1)C | 3000 | | | 2000 |
| O(CCN1CCC(CN(Cc2c(OCC═C)cccc2)CC2OCCC2)CC1)C | 0 | | | 0 |
| O(C)c1c(CN(CC2OCCC2)CC2CCN(C3CCCC3)CC2)ccc(OC)c1 | 0 | | | 500 |
| O(CC)c1c(CN(CC2OCCC2)CC2CCN(C3CCCC3)CC2)cccc1 | 0 | | | 0 |
| O═C(Nc1cc(C(═O)N2CCN(C)CC2)cc(CNC2C3CC4CC2CC(C3)C4)c1)C | 10000 | | | 6000 |
| O═C(N(C(C)C)CCO)C═1C(═O)C(C(═O)NCCC2═CCCCC2)═CN | 0 | | | 0 |
| (Cc2c(OC)cccc2)C = 1 | | | | |
| O═C(N(CCCN(C)C)C)C═1C(═O)C(C(═O)NCCC(C)C)═CN | 0 | | | 500 |
| (Cc2c(OC)cccc2)C = 1 | | | | |
| Fc1c(F)ccc(CNCc2cc(NC(═O)C)cc(C(═O)N3CCN(C)CC3)c2)c1 | 5000 | | | 5500 |
| O═C(NCCC1═CCCCC1)C═1C(═O)C(C(═O)N2CC(O)CCC2)═CN | 2000 | | | 5250 |
| (Cc2c(OC)cccc2)C = 1 | | | | |
| O═C(NC1CCCCCC1)C═1C(═O)C(C(═O)N2C(CC)CCCC2)═CN | 2000 | | | 4500 |
| (Cc2cc(OC)ccc2)C = 1 | | | | |
| O═C(NCCC(C)C)C═1C(═O)C(C(═O)N2CCN(CCOC)CC2)═CN | 0 | | | 1500 |
| (Cc2c(OC)cccc2)C = 1 | | | | |
| O═C(Nc1cc(C(═O)N2CCCC2)cc(CNC2C3CC4CC2CC(C3)C4)c1)c1ncccc1 | 6000 | | | 3000 |
| O═C(Nc1cc(C(═O)N2C(C)CN(C)CCC2)ccc1)CC | 5000 | | | 3500 |
| OCC#Cc1cc(CN(CCN(C)C)Cc2sc(C)cc2)sc1 | 1000 | | | 500 |
| OCC#Cc1cc(CN(CCCO)Cc2sc(C)cc2)sc1 | 25000 | | | 18500 |
| OCC#Cc1cc(CN(CC(C)C)Cc2c(C)cccc2)sc1 | 15500 | | | 10250 |
| N(CCN(C)C)(Cc1c(C)ccs1)Cc1c(C)ccs1 | 0 | | | 0 |
| OCC#Cc1cc(CN(C(CC)C)Cc2cscc2)sc1 | 4000 | | | 3500 |
| | 20500 | | | 13750 |
| | | | | 0 |
| | 0 | | | 0 |
| O═C(OC1CCCCC1)C1═C(C)NC(═O)NC1c1cc(OC)c(OC)cc1 | 15500 | | | 15500 |
| O═C(NC1CCCCCC1)c1c(-c2cc(OC)c(OC)cc2)n[nH]c1 | 1000 | | | 4750 |
| O═C(Nc1c(C(═O)N2CCCCC2)cccc1)c1ccc(OCCCC)cc1 | 33500 | | | 21250 |
| O═C(Nc1c(C(═O)N2CCCCC2)cccc1)c1c(OCCOCC)cccc1 | 2000 | | | 1000 |
| O═C(Nc1c(C(═O)N2CCOCC2)cccc1)c1ccc(OCC2OCCC2)cc1 | 25500 | | | 12750 |
| O═C(Nc1ccc(C(═O)N2CCC(C)CC2)cc1)c1ccc(OCC2OCCC2)cc1 | 19500 | | | 10750 |
| O═C(Nc1c(C(═O)N2CCCCC2)cccc1)c1ccc(OCC2OCCC2)cc1 | 17000 | | | 11000 |
| O═C(Nc1c(C(═O)N2CCN(C)CC2)cccc1)c1ccc(OCC2OCCC2)cc1 | 29500 | | | 17250 |
| N(Cc1sc(C)cc1)(Cc1sc(C)cc1)C | 17500 | | | 9750 |
| O═C(OC1CCCCCCC1)C1═C(C)NC(═O)NC1c1cc(O)ccc1 | 18000 | | | 11500 |
| O═C(NCC1OCCC1)C1CCN(Cc2c(OCC)cccc2)CC1 | 16000 | | | 11500 |
| N(CCN(Cc1oc(C)cc1)Cc1oc(C)cc1)(CC)CC | 0 | | | 0 |
| O═C(N(CC1C2N(CCC1)CCCC2)C)c1c(-c2cc(OC)c(OC)cc2)n[nH]c1 | 19500 | | | 12250 |
| O═C(OCCc1ccccc1)C1═C(C)NC(═O)NC1c1cc(OC)c(OCCC)cc1 | 12000 | | | 7000 |
| O═C(OC1CCCCCCC1)C1═C(C)NC(═O)NC1c1cc(OC)c(OCc2ccccc2)cc1 | 0 | | | 0 |
| O═C(OCC1OCCC1)C1═C(C)NC(═O)NC1c1cc(OC)c(OCCC)cc1 | 3000 | | | 2500 |
| O═C(OC(CC(C)C)C)c1c(-c2ccccc2)ncnc1 | 16000 | | | 10500 |
| O═C(NCC1OCCC1)c1c(NC(═O)c2ccc(OCCc3ccccc3)cc2)cccc1 | 24500 | | | 14750 |
| O═C(N(C)C1CCCCC1)c1c(NC(═O)c2ccc(OCCOC)cc2)cccc1 | 11000 | | | 9000 |
| O═C(NC1CCCCCC1)c1c(-c2cc3OCCOc3cc2)[nH]nc1 | 0 | | | 0 |
| O═C(Nc1cc(C(═O)NCC2(N(C)C)CCCCCC2)ccc1)C(C)(C)C | 9000 | | | 16750 |
| O═C(NC1CCCCC1)c1c(-c2cc3OCCCOc3cc2)nn(C)c1 | 17000 | | | 13750 |
| | 2700000 | | | 1420000 |
| | 0 | | | 0 |
| | 0 | | | 0 |
| O═C(NC1CCCC1)c1c(NC(═O)c2ccc(OCC3OCCC3)cc2)cccc1 | 3300000 | | | 1995000 |
| O═C(Nc1c(CN2CCOCC2)cccc1)c1ccc(OCC2OCCC2)cc1 | 5000000 | | | 2620000 |
| O═C(OCC(═O)c1ccccc1)c1c(-c2cc3OCCOc3cc2)[nH]nc1 | 1600000 | | | 825000 |
| O═C(Nc1cc(NC(═O)C)cc(C(═O)NC2C(C)CCCC2)c1)C | 5000000 | | | 2677500 |
| O═C(Nc1cc(C(═O)NC2C3N(CC2)CCCC3)ccc1)C | 1100000 | | | 620000 |
| O═C(OCC)C═1C(c2ccccc2)═NC(═O)NC═1C | 1100000 | | | 595000 |
| O═C(NCC1OCCC1)c1c(NC(═O)c2ccc(OCC(C)C)cc2)cccc1 | 5000000 | | | 2530000 |
| O═C(N1CCCCC1)c1c(-c2cc3OCCOc3cc2)[nH]nc1 | 150000 | | | 84000 |
| O═C(Nc1c(C(═O)N2CC(C)CC(C)C2)cccc1)c1cc2OCCOc2cc1 | 1600000 | | | 865000 |
| O═C(NCC)CN(Cc1sccc1)Cc1sccc1 | 1300000 | | | 725000 |
| N(CCNCc1sccc1)(C)C | 1800000 | | | 1200000 |
| O═C(OC1CCCCCCC1)C1═C(C)NC(═O)NC1c1cc(OC)c(OCCC)cc1 | 120000 | | | 102500 |
| O═C(Nc1c(C(═O)N2C(CCO)CCCC2)cccc1)c1ccc(OC)cc1 | 5000000 | | | 2597500 |
| O═C(OC1CCCCCC1)C1═C(C)NC(═O)NC1c1cc(OCc2ccccc2)ccc1 | 1900000 | | | 1035000 |
| O═C(OC1CCCCCCC1)C1═C(C)NC(═O)NC1c1cc2OCOc2cc1 | 0 | | | 0 |
| O═C(OC1CCCCCC1)C1═C(C)NC(═O)NC1c1cc2OCOc2cc1 | 0 | | | 0 |

| | | | | Mol | |
|---|---|---|---|---|---|
| Molecule | Supplier | ID | Alt ID | Wt | CB ID |
| O═C(OCC)C1═C(C)NC(═O)NC1c1cc(OC)c(OCCc2ccccc2)cc1 | Hit2Lead | 7679642 | | 410 | 5612793_6 |
| O═C(Nc1c(-c2oc(C)c(CN(C/C═C/c3cc(OC)c(O)cc3)C)n2)cccc1)c1cscc1 | Hit2Lead | 48325627 | | 490 | 17295424_7 |
| O═C(NCCC1═CCCCC1)C═1C(═O)C(C(═O)N2CCCCC2)═CN | Hit2Lead | 93701800 | | 478 | 55345532_2 |
| (Cc2c(OC)cccc2)C = 1 | | | | | |
| OCC#Cc1cc(CN(Cc2c(C)ccs2)C)sc1 | Hit2Lead | 91240494 | | 291 | 10253964_6 |
| O═C(Nc1cc(C(═O)N2C[C@H]3N(C)C[C@@H] | Hit2Lead | 49602830 | | 315 | 70444941_4 |
| (C2)CCC3)ccc1)C | | | | | |
| O(C[C@H]1N(Cc2scc(C#CCO)c2)CCC1)C | Hit2Lead | 10434393 | | 265 | 10253964_14 |
| OCC#Cc1cc(CN2CC(C)N(C(C)C)CC2)sc1 | Hit2Lead | 30933538 | | 292 | 10253964_3 |
| OCC#Cc1cc(CN(CCN2CCCCCC2)C)sc1 | Hit2Lead | 37497358 | | 306 | 10253964_5 |
| OCC#Cc1cc(CN(CC2CCOCC2)C)sc1 | Hit2Lead | 37644792 | | 279 | 10253964_15 |
| O(CC(O)CN1CCCCC1)c1c(CN2CC3(OCC2)CCCC3)cccc1 | Hit2Lcad | 43103865 | | 389 | 78409968_12 |
| S(C)c1nc(c(C(═O)O)cn1)-c1cc(OC)c(OC)cc1 | Hit2Lead | 15578104 | | 306 | 5612793_15 |
| O(CCO)c1c(CN2CC(O)(CO)CCC2)cccc1 | Hit2Lead | 75846711 | | 281 | 78409968_7 |
| O═C(NC)CCN(Cc1scc(C#CCO)c1)C | Hit2Lead | 41503365 | | 266 | 10253964_18 |
| OCC#Cc1cc(CN(CCN2CCCCC2)CC)sc1 | Hit2Lead | 68133153 | | 306 | 10253964_4 |
| O(CCN1CCC(CN(Cc2c(OCCO)cccc2)CC2OCCC2)CC1)C | Hit2Lead | 78409968 | | 407 | 78409968_1 |
| O(CCN1CCC(CN(Cc2c(O)cccc2)CC2OCCC2)CC1)C | Hit2Lead | 49010376 | | 363 | 78409968_5 |
| O(CCN1CCC(CN(Cc2c(OCC)cccc2)CC2OCCC2)CC1)C | Hit2Lead | 35161547 | | 391 | 78409968_2 |
| Fc1c(CN2CCC(CN(Cc3c(OC)cccc3O)CC3OCCC3)CC2)cccc1 | Hit2Lead | 66893200 | | 443 | 78409968_10 |
| O(CCO)c1c(CN(CC(C)C)CC2CCN(Cc3c(OC)cccc3)CC2)cccc1 | Hit2Lead | 97913321 | | 441 | 78409968_9 |
| Fc1c(CN2CCC(CN(CCOC)Cc3c(OCCO)cccc3)CC2)cccc1 | Hit2Lead | 66918704 | | 431 | 78409968_11 |
| O═C(Nc1cc(C(═O)NC2CN(C3CCCCCC3)CCC2)ccc1)C | Hit2Lead | 38740381 | | 357 | 70444941_2 |
| O(CCN1CCC(CN(Cc2c(OCC═C)cccc2)CC2OCCC2)CC1)C | Hit2Lead | 97880594 | | 403 | 78409968_4 |
| O(C)c1c(CN(CC2OCCC2)CC2CCN(C3CCCC3)CC2)ccc(OC)c1 | Hit2Lead | 58156196 | | 417 | 78409968_8 |
| O(CC)c1c(CN(CC2OCCC2)CC2CCN(C3CCCC3)CC2)cccc1 | Hit2Lead | 38521191 | | 401 | 78409968_3 |
| O═C(Nc1cc(C(═O)N2CCN(C)CC2)cc(CNC2C3CC4CC2CC(C3)C4)c1)C | Hit2Lead | 70444941 | | 425 | 70444941_1 |
| O═C(N(C(C)C)CCO)C═1C(═O)C(C(═O)NCCC2═CCCCC2)═CN | Hit2Lead | 55345532 | | 496 | 55345532_1 |
| (Cc2c(OC)cccc2)C = 1 | | | | | |
| O═C(N(CCCN(C)C)C)C═1C(═O)C(C(═O)NCCC(C)C)═CN | Hit2Lead | 23614835 | | 471 | 55345532_5 |
| (Cc2c(OC)cccc2)C = 1 | | | | | |
| Fc1c(F)ccc(CNCc2cc(NC(═O)C)cc(C(═O)N3CCN(C)CC3)c2)c1 | Hit2Lead | 73759021 | | 416 | 70444941_6 |
| O═C(NCCC1═CCCCC1)C═1C(═O)C(C(═O)N2CC(O)CCC2)═CN | Hit2Lead | 53145516 | | 494 | 55345532_3 |
| (Cc2c(OC)cccc2)C = 1 | | | | | |
| O═C(NC1CCCCCC1)C═1C(═O)C(C(═O)N2C(CC)CCCC2)═CN | Hit2Lead | 47976750 | | 494 | 55345532_7 |
| (Cc2cc(OC)ccc2)C = 1 | | | | | |
| O═C(NCCC(C)C)C═1C(═O)C(C(═O)N2CCN(CCOC)CC2)═CN | Hit2Lead | 24668741 | | 499 | 55345532_6 |
| (Cc2c(OC)cccc2)C = 1 | | | | | |
| O═C(Nc1cc(C(═O)N2CCCC2)cc(CNC2C3CC4CC2CC(C3)C4)c1)c1ncccc1 | Hit2Lead | 92881721 | | 459 | 70444941_7 |
| O═C(Nc1cc(C(═O)N2C(C)CN(C)CCC2)ccc1)CC | Hit2Lead | 41610622 | | 303 | 70444941_10 |
| OCC#Cc1cc(CN(CCN(C)C)Cc2sc(C)cc2)sc1 | Hit2Lead | 10253964 | | 349 | 10253964_1 |
| OCC#Cc1cc(CN(CCCO)Cc2sc(C)cc2)sc1 | Hit2Lead | 24200140 | | 335 | 10253964_2 |
| OCC#Cc1cc(CN(CC(C)C)Cc2c(C)cccc2)sc1 | Hit2Lead | 18052219 | | 327 | 10253964_17 |
| N(CCN(C)C)(Cc1c(C)ccs1)Cc1c(C)ccs1 | Hit2Lead | 10355732 | | 309 | 10253964_9 |
| OCC#Cc1cc(CN(C(CC)C)Cc2cscc2)sc1 | Hit2Lead | 26918198 | | 319 | 10253964_13 |
| O═C(OC1CCCCC1)C1═C(C)NC(═O)NC1c1cc(OC)c(OC)cc1 | Vitas-M | STK364138 | | 374.44 | 5612793_3 |
| | Labs | | | | |
| O═C(NC1CCCCCC1)c1c(-c2cc(OC)c(OC)cc2)n[nH]c1 | Vitas-M | STL184833 | | 343.43 | 5612793_18 |
| | Labs | | | | |
| O═C(Nc1c(C(═O)N2CCCCC2)cccc1)c1ccc(OCCCC)cc1 | Vitas-M | STL071122 | | 380.49 | 9131650_3 |
| | Labs | | | | |
| O═C(Nc1c(C(═O)N2CCCCC2)cccc1)c1c(OCCOCC)cccc1 | Vitas-M | STL066877 | | 396.49 | 9131650_13 |
| | Labs | | | | |
| O═C(Nc1c(C(═O)N2CCOCC2)cccc1)c1ccc(OCC2OCCC2)cc1 | Vitas-M | STL068656 | | 410.47 | 9131650_4 |
| | Labs | | | | |
| O═C(Nc1ccc(C(═O)N2CCC(C)CC2)cc1)c1ccc(OCC2OCCC2)cc1 | Vitas-M | STL068664 | | 422.52 | 9131650_7 |
| | Labs | | | | |
| O═C(Nc1c(C(═O)N2CCCCC2)cccc1)c1ccc(OCC2OCCC2)cc1 | Vitas-M | STL068657 | | 408.5 | 9131650_1 |
| | Labs | | | | |
| O═C(Nc1c(C(═O)N2CCN(C)CC2)cccc1)c1ccc(OCC2OCCC2)cc1 | Vitas-M | STL068659 | | 423.51 | 9131650_6 |
| | Labs | | | | |
| N(Cc1sc(C)cc1)(Cc1sc(C)cc1)C | Vitas-M | STL069512 | | 251.42 | 10253964_10 |
| | Labs | | | | |
| O═C(OC1CCCCCCC1)C1═C(C)NC(═O)NC1c1cc(O)ccc1 | Vitas-M | STK062421 | | 358.44 | 5612793_11 |
| | Labs | | | | |
| O═C(NCC1OCCC1)C1CCN(Cc2c(OCC)cccc2)CC1 | Vitas-M | STK017119 | | 346.47 | 78409968_6 |
| | Labs | | | | |
| N(CCN(Cc1oc(C)cc1)Cc1oc(C)cc1)(CC)CC | Vitas-M | STK161925 | | 304.43 | 10253964_12 |
| | Labs | | | | |
| O═C(N(CC1C2N(CCC1)CCCC2)C)c1c(-c2cc(OC)c(OC)cc2)n[nH]c1 | Vitas-M | STL546978 | | 412.53 | 5612793_19 |
| | Labs | | | | |
| O═C(OCCc1ccccc1)C1═C(C)NC(═O)NC1c1cc(OC)c(OCCC)cc1 | Vitas-M | STK083274 | | 424.5 | 5612793_8 |
| | Labs | | | | |
| O═C(OC1CCCCCCC1)C1═C(C)NC(═O)NC1c1cc(OC)c(OCc2ccccc2)cc1 | Vitas-M | STK075753 | | 478.59 | 5612793_4 |
| | Labs | | | | |
| O═C(OCC1OCCC1)C1═C(C)NC(═O)NC1c1cc(OC)c(OCCC)cc1 | Vitas-M | STK054478 | | 404.46 | 5612793_9 |
| | Labs | | | | |
| O═C(OC(CC(C)C)C)c1c(-c2ccccc2)ncnc1 | Vitas-M | STK244118 | | 396.49 | 5612793_12 |
| | Labs | | | | |
| O═C(NCC1OCCC1)c1c(NC(═O)c2ccc(OCCc3ccccc3)cc2)cccc1 | Vitas-M | STK276413 | | 444.53 | 9131650_8 |
| | Labs | | | | |
| O═C(N(C)C1CCCCC1)c1c(NC(═O)c2ccc(OCCOC)cc2)cccc1 | Vitas-M | STK345854 | | 410.51 | 9131650_5 |
| | Labs | | | | |
| O═C(NC1CCCCCC1)c1c(-c2cc3OCCOc3cc2)[nH]nc1 | Enamine | Z224428262 | MolPort- | 341.4 | 5612793_16 |
| | Ltd | | 009- | | |
| | | | 439-585 | | |
| O═C(Nc1cc(C(═O)NCC2(N(C)C)CCCCCC2)ccc1)C(C)(C)C | Enamine | Z253654054 | MolPort- | 373.5 | 70444941_3 |
| | Ltd | | 009- | | |
| | | | 471-445 | | |
| O═C(NC1CCCCC1)c1c(-c2cc3OCCCOc3cc2)nn(C)c1 | Enamine | Z79410098 | MolPort- | 355.4 | 5612793_17 |
| | Ltd | | 009- | | |
| | | | 228-990 | | |
| O═C(NC1CCCC1)c1c(NC(═O)c2ccc(OCC3OCCC3)cc2)cccc1 | Enamine | Z28085941 | MolPort- | 408.5 | 9131650_2 |
| | Ltd | | 009- | | |
| | | | 496-837 | | |
| O═C(Nc1c(CN2CCOCC2)cccc1)c1ccc(OCC2OCCC2)cc1 | UkrOrgSyn- | PB146722878 | MolPort- | 471.6 | 9131650_11 |
| | thesis | | 005- | | |
| | Ltd. Stock | | 781-557 | | |
| O═C(OCC(═O)c1ccccc1)c1c(-c2cc3OCCOc3cc2)[nH]nc1 | Enamine | Z217871108 | MolPort- | 364.4 | 5612793_14 |
| | Ltd | | 005- | | |
| | | | 524-491 | | |
| O═C(Nc1cc(NC(═O)C)cc(C(═O)NC2C(C)CCCC2)c1)C | Enamine | Z27075709 | MolPort- | 331.4 | 70444941_9 |
| | Ltd | | 004- | | |
| | | | 209-658 | | |
| O═C(Nc1cc(C(═O)NC2C3N(CC2)CCCC3)ccc1)C | Enamine | Z767436704 | MolPort- | 301.4 | 70444941_5 |
| | Ltd | | 020- | | |
| | | | 114-021 | | |
| O═C(OCC)C═1C(c2ccccc2)═NC(═O)NC═1C | Enamine | Z1665450493 | MolPort- | 284.4 | 5612793_13 |
| | Ltd | | 028- | | |
| | | | 802-725 | | |
| O═C(NCC1OCCC1)c1c(NC(═O)c2ccc(OCC(C)C)cc2)cccc1 | Enamine | Z739206878 | MolPort- | 396.5 | 9131650_12 |
| | Ltd | | 009- | | |
| | | | 088-584 | | |
| O═C(N1CCCCC1)c1c(-c2cc3OCCOc3cc2)[nH]nc1 | Enamine | Z224448688 | MolPort- | 313.4 | 5612793_20 |
| | Ltd | | 009- | | |
| | | | 527-391 | | |
| O═C(Nc1c(C(═O)N2CC(C)CC(C)C2)cccc1)c1cc2OCCOc2cc1 | Enamine | Z394427336 | MolPort- | 394.5 | 9131650_14 |
| | Ltd | | 009- | | |
| | | | 306-253 | | |
| O═C(NCC)CN(Cc1sccc1)Cc1sccc1 | Enamine | Z162917606 | MolPort- | 294.4 | 10253964_8 |
| | Ltd | | 009- | | |
| | | | 133-665 | | |
| N(CCNCc1sccc1)(C)C | Enamine | Z90512807 | MolPort- | 184.3 | 10253964_7 |
| | Ltd | | 000- | | |
| | | | 938-899 | | |
| O═C(OC1CCCCCCC1)C1═C(C)NC(═O)NC1c1cc(OC)c(OCCC)cc1 | Specs | AG-205/ | MolPort- | 430.5 | 5612793_1 |
| | | 11867180 | 000- | | |
| | | | 907-180 | | |
| O═C(Nc1c(C(═O)N2C(CCO)CCCC2)cccc1)c1ccc(OC)cc1 | Specs | AP-064/ | MolPort- | 382.5 | 9131650_9 |
| | | 41684584 | 002- | | |
| | | | 090-971 | | |
| O═C(OC1CCCCCC1)C1═C(C)NC(═O)NC1c1cc(OCc2ccccc2)ccc1 | Specs | AG-205/ | MolPort- | 434.5 | 5612793_10 |
| | | 11867172 | 000- | | |
| | | | 911-785 | | |
| O═C(OC1CCCCCCC1)C1═C(C)NC(═O)NC1c1cc2OCOc2cc1 | Specs | AG-690/ | MolPort- | 386.4 | 5612793_5 |
| | | 33475017 | 000- | | |
| | | | 906-361 | | |
| O═C(OC1CCCCCC1)C1═C(C)NC(═O)NC1c1cc2OCOc2cc1 | ChemDiv, | 1308- | MolPort- | 372.4 | 5612793_7 |
| | Inc | 0451 | 001- | | |
| | | | 928-269 | | |

| | | | 10 μM treatment | |
|---|---|---|---|---|
| | | UMB ID | DMSO | |
| Molecule | UMB ID | new | (0.1%) | 100000 |
| | | | UMB18 | 27000 |
| O═C(OCC)C1═C(C)NC(═O)NC1c1cc(OC)c(OCCc2ccccc2)cc1 | UMB22_6 | 41 | UMB41 | 31000 |
| O═C(Nc1c(-c2oc(C)c(CN(C/C═C/c3cc(OC)c(O)cc3)C)n2)cccc1)c1cscc1 | UMB5_7 | 42 | UMB42 | 20000 |
| O═C(NCCC1═CCCCC1)C═1C(═O)C(C(═O)N2CCCCC2)═CN | UMB10_2 | 43 | UMB43 | 170000 |
| (Cc2c(OC)cccc2)C = 1 | | | | |
| OCC#Cc1cc(CN(Cc2c(C)ccs2)C)sc1 | UMB28_6 | 44 | UMB44 | 40000 |
| O═C(Nc1cc(C(═O)N2C[C@H]3N(C)C[C@@H] | UMB40_4 | 45 | UMB45 | 24000 |
| (C2)CCC3)ccc1)C | | | | |
| O(C[C@H]1N(Cc2scc(C#CCO)c2)CCC1)C | UMB28_14 | 46 | UMB46 | 39000 |
| OCC#Cc1cc(CN2CC(C)N(C(C)C)CC2)sc1 | UMB28_3 | 47 | UMB47 | 80000 |
| OCC#Cc1cc(CN(CCN2CCCCCC2)C)sc1 | UMB28_5 | 48 | UMB48 | 110000 |
| OCC#Cc1cc(CN(CC2CCOCC2)C)sc1 | UMB28_15 | 49 | UMB49 | 100000 |
| O(CC(O)CN1CCCCC1)c1c(CN2CC3(OCC2)CCCC3)cccc1 | UMB42_12 | 50 | UMB50 | 11000 |
| S(C)c1nc(c(C(═O)O)cn1)-c1cc(OC)c(OC)cc1 | UMB22_15 | 51 | UMB51 | 100000 |
| O(CCO)c1c(CN2CC(O)(CO)CCC2)cccc1 | UMB42_7 | 52 | UMB52 | 38000 |
| O═C(NC)CCN(Cc1scc(C#CCO)c1)C | UMB28_18 | 53 | UMB53 | 50000 |
| OCC#Cc1cc(CN(CCN2CCCCC2)CC)sc1 | UMB28_4 | 54 | UMB54 | 9000 |
| O(CCN1CCC(CN(Cc2c(OCCO)cccc2)CC2OCCC2)CC1)C | UMB42_1 | 55 | UMB55 | 30000 |
| O(CCN1CCC(CN(Cc2c(O)cccc2)CC2OCCC2)CC1)C | UMB42_5 | 56 | UMB56 | 49000 |
| O(CCN1CCC(CN(Cc2c(OCC)cccc2)CC2OCCC2)CC1)C | UMB42_2 | 57 | UMB57 | 40000 |
| Fc1c(CN2CCC(CN(Cc3c(OC)cccc3O)CC3OCCC3)CC2)cccc1 | UMB42_10 | 58 | UMB58 | 28000 |
| O(CCO)c1c(CN(CC(C)C)CC2CCN(Cc3c(OC)cccc3)CC2)cccc1 | UMB42_9 | 59 | UMB59 | 11000 |
| Fc1c(CN2CCC(CN(CCOC)Cc3c(OCCO)cccc3)CC2)cccc1 | UMB42_11 | 60 | UMB60 | 29000 |
| | | | DMSO | 18500 |
| | | | (0.1%) | |
| | | | UMB18 | 1000 |
| | | | UMB18 | 0 |
| | | | (gel) | |
| O═C(Nc1cc(C(═O)NC2CN(C3CCCCCC3)CCC2)ccc1)C | UMB40_2 | 61 | UMB61 | 6500 |
| O(CCN1CCC(CN(Cc2c(OCC═C)cccc2)CC2OCCC2)CC1)C | UMB42_4 | 62 | UMB62 | 4000 |
| O(C)c1c(CN(CC2OCCC2)CC2CCN(C3CCCC3)CC2)ccc(OC)c1 | UMB42_8 | 63 | UMB63 | 14000 |
| O(CC)c1c(CN(CC2OCCC2)CC2CCN(C3CCCC3)CC2)cccc1 | UMB42_3 | 64 | UMB64 | 6500 |
| O═C(Nc1cc(C(═O)N2CCN(C)CC2)cc(CNC2C3CC4CC2CC(C3)C4)c1)C | UMB40_1 | 65 | UMB65 | 43000 |
| O═C(N(C(C)C)CCO)C═1C(═O)C(C(═O)NCCC2═CCCCC2)═CN | UMB10_1 | 66 | UMB66 | 17000 |
| (Cc2c(OC)cccc2)C = 1 | | | | |
| O═C(N(CCCN(C)C)C)C═1C(═O)C(C(═O)NCCC(C)C)═CN | UMB10_5 | 67 | UMB67 | 8500 |
| (Cc2c(OC)cccc2)C = 1 | | | | |
| Fc1c(F)ccc(CNCc2cc(NC(═O)C)cc(C(═O)N3CCN(C)CC3)c2)c1 | UMB40_6 | 68 | UMB68 | 0 |
| O═C(NCCC1═CCCCC1)C═1C(═O)C(C(═O)N2CC(O)CCC2)═CN | UMB10_3 | 69 | UMB69 | 4000 |
| (Cc2c(OC)cccc2)C = 1 | | | | |
| O═C(NC1CCCCCC1)C═1C(═O)C(C(═O)N2C(CC)CCCC2)═CN | UMB10_7 | 70 | UMB70 | 24000 |
| (Cc2cc(OC)ccc2)C = 1 | | | | |
| O═C(NCCC(C)C)C═1C(═O)C(C(═O)N2CCN(CCOC)CC2)═CN | UMB10_6 | 71 | UMB71 | 4000 |
| (Cc2c(OC)cccc2)C = 1 | | | | |
| O═C(Nc1cc(C(═O)N2CCCC2)cc(CNC2C3CC4CC2CC(C3)C4)c1)c1ncccc1 | UMB40_7 | 72 | UMB72 | 16500 |
| O═C(Nc1cc(C(═O)N2C(C)CN(C)CCC2)ccc1)CC | UMB40_10 | 73 | UMB73 | 6000 |
| OCC#Cc1cc(CN(CCN(C)C)Cc2sc(C)cc2)sc1 | UMB28_1 | 74 | UMB74 | 0 |
| OCC#Cc1cc(CN(CCCO)Cc2sc(C)cc2)sc1 | UMB28_2 | 75 | UMB75 | 14000 |
| OCC#Cc1cc(CN(CC(C)C)Cc2c(C)cccc2)sc1 | UMB28_17 | 76 | UMB76 | 22000 |
| N(CCN(C)C)(Cc1c(C)ccs1)Cc1c(C)ccs1 | UMB28_9 | 77 | UMB77 | 0 |
| OCC#Cc1cc(CN(C(CC)C)Cc2cscc2)sc1 | UMB28_13 | 78 | UMB78 | 5000 |
| | | | DMSO | 57000 |
| | | | (0.1%) | |
| | | | UMB18 | 2000 |
| | | | UMB18 | |
| | | | (gel) | |
| O═C(OC1CCCCC1)C1═C(C)NC(═O)NC1c1cc(OC)c(OC)cc1 | UMB22_3 | 79 | UMB79 | 11500 |
| O═C(NC1CCCCCC1)c1c(-c2cc(OC)c(OC)cc2)n[nH]c1 | UMB22_18 | 80 | UMB80 | 47000 |
| O═C(Nc1c(C(═O)N2CCCCC2)cccc1)c1ccc(OCCCC)cc1 | UMB23_3 | 81 | UMB81 | 17500 |
| O═C(Nc1c(C(═O)N2CCCCC2)cccc1)c1c(OCCOCC)cccc1 | UMB23_13 | 82 | UMB82 | 9500 |
| O═C(Nc1c(C(═O)N2CCOCC2)cccc1)c1ccc(OCC2OCCC2)cc1 | UMB23_4 | 83 | UMB83 | 6000 |
| O═C(Nc1ccc(C(═O)N2CCC(C)CC2)cc1)c1ccc(OCC2OCCC2)cc1 | UMB23_7 | 84 | UMB84 | 9500 |
| O═C(Nc1c(C(═O)N2CCCCC2)cccc1)c1ccc(OCC2OCCC2)cc1 | UMB23_1 | 85 | UMB85 | 19500 |
| O═C(Nc1c(C(═O)N2CCN(C)CC2)cccc1)c1ccc(OCC2OCCC2)cc1 | UMB23_6 | 86 | UMB86 | 7500 |
| N(Cc1sc(C)cc1)(Cc1sc(C)cc1)C | UMB28_10 | 87 | UMB87 | 1000 |
| O═C(OC1CCCCCCC1)C1═C(C)NC(═O)NC1c1cc(O)ccc1 | UMB22_11 | 88 | UMB88 | 6000 |
| O═C(NCC1OCCC1)C1CCN(Cc2c(OCC)cccc2)CC1 | UMB42_6 | 89 | UMB89 | 10500 |
| N(CCN(Cc1oc(C)cc1)Cc1oc(C)cc1)(CC)CC | UMB28_12 | 90 | UMB90 | 1000 |
| O═C(N(CC1C2N(CCC1)CCCC2)C)c1c(-c2cc(OC)c(OC)cc2)n[nH]c1 | UMB22_19 | 91 | UMB91 | 8000 |
| O═C(OCCc1ccccc1)C1═C(C)NC(═O)NC1c1cc(OC)c(OCCC)cc1 | UMB22_8 | 92 | UMB92 | 3000 |
| O═C(OC1CCCCCCC1)C1═C(C)NC(═O)NC1c1cc(OC)c(OCc2ccccc2)cc1 | UMB22_4 | 93 | UMB93 | 2000 |
| O═C(OCC1OCCC1)C1═C(C)NC(═O)NC1c1cc(OC)c(OCCC)cc1 | UMB22_9 | 94 | UMB94 | 1000 |
| O═C(OC(CC(C)C)C)c1c(-c2ccccc2)ncnc1 | UMB22_12 | 95 | UMB95 | 8000 |
| O═C(NCC1OCCC1)c1c(NC(═O)c2ccc(OCCc3ccccc3)cc2)cccc1 | UMB23_8 | 96 | UMB96 | 16000 |
| O═C(N(C)C1CCCCC1)c1c(NC(═O)c2ccc(OCCOC)cc2)cccc1 | UMB23_5 | 97 | UMB97 | 20000 |
| O═C(NC1CCCCCC1)c1c(-c2cc3OCCOc3cc2)[nH]nc1 | UMB22_16 | 98 | UMB98 | 0 |
| O═C(Nc1cc(C(═O)NCC2(N(C)C)CCCCCC2)ccc1)C(C)(C)C | UMB40_3 | 99 | UMB99 | 13500 |
| O═C(NC1CCCCC1)c1c(-c2cc3OCCCOc3cc2)nn(C)c1 | UMB22_17 | 100 | UMB100 | 14500 |
| | | | DMSO | 620000 |
| | | | (0.1%) | |
| | | | UMB18 | 10000 |
| | | | UMB18 | 0 |
| | | | (gel) | |
| O═C(NC1CCCC1)c1c(NC(═O)c2ccc(OCC3OCCC3)cc2)cccc1 | UMB23_2 | 101 | UMB101 | 665000 |
| O═C(Nc1c(CN2CCOCC2)cccc1)c1ccc(OCC2OCCC2)cc1 | UMB23_11 | 102 | UMB102 | 800000 |
| O═C(OCC(═O)c1ccccc1)c1c(-c2cc3OCCOc3cc2)[nH]nc1 | UMB22_14 | 103 | UMB103 | 800000 |
| O═C(Nc1cc(NC(═O)C)cc(C(═O)NC2C(C)CCCC2)c1)C | UMB40_9 | 104 | UMB104 | 490000 |
| O═C(Nc1cc(C(═O)NC2C3N(CC2)CCCC3)ccc1)C | UMB40_5 | 105 | UMB105 | 610000 |
| O═C(OCC)C═1C(c2ccccc2)═NC(═O)NC═1C | UMB22_13 | 106 | UMB106 | 505000 |
| O═C(NCC1OCCC1)c1c(NC(═O)c2ccc(OCC(C)C)cc2)cccc1 | UMB23_12 | 107 | UMB107 | 440000 |
| O═C(N1CCCCC1)c1c(-c2cc3OCCOc3cc2)[nH]nc1 | UMB22_20 | 108 | UMB108 | 370000 |
| O═C(Nc1c(C(═O)N2CC(C)CC(C)C2)cccc1)c1cc2OCCOc2cc1 | UMB23_14 | 109 | UMB109 | 800000 |
| O═C(NCC)CN(Cc1sccc1)Cc1sccc1 | UMB28_8 | 110 | UMB110 | 1400000 |
| N(CCNCc1sccc1)(C)C | UMB28_7 | 111 | UMB111 | 800000 |
| O═C(OC1CCCCCCC1)C1═C(C)NC(═O)NC1c1cc(OC)c(OCCC)cc1 | UMB22_1 | 112 | UMB112 | 405000 |
| O═C(Nc1c(C(═O)N2C(CCO)CCCC2)cccc1)c1ccc(OC)cc1 | UMB23_9 | 113 | UMB113 | 3100000 |
| O═C(OC1CCCCCC1)C1═C(C)NC(═O)NC1c1cc(OCc2ccccc2)ccc1 | UMB22_10 | 114 | UMB114 | 1700000 |
| O═C(OC1CCCCCCC1)C1═C(C)NC(═O)NC1c1cc2OCOc2cc1 | UMB22_5 | 115 | UMB115 | 2000 |
| O═C(OC1CCCCCC1)C1═C(C)NC(═O)NC1c1cc2OCOc2cc1 | UMB22_7 | 116 | UMB116 | 2000 |

| | 10 μM treatment | | | Average |
|---|---|---|---|---|
| Molecule | 200000 | 26000 | 30000 | 89000 |
| | 12000 | 1000 | 5000 | 11250 |
| O═C(OCC)C1═C(C)NC(═O)NC1c1cc(OC)c(OCCc2ccccc2)cc1 | 13000 | | | 22000 |
| O═C(Nc1c(-c2oc(C)c(CN(C/C═C/c3cc(OC)c(O)cc3)C)n2)cccc1)c1cscc1 | 3000 | | | 11500 |
| O═C(NCCC1═CCCCC1)C═1C(═O)C(C(═O)N2CCCCC2)═CN | 10000 | | | 90000 |
| (Cc2c(OC)cccc2)C = 1 | | | | |
| OCC#Cc1cc(CN(Cc2c(C)ccs2)C)sc1 | 12000 | | | 26000 |
| O═C(Nc1cc(C(═O)N2C[C@H]3N(C)C[C@@H] | 3000 | | | 13500 |
| (C2)CCC3)ccc1)C | | | | |
| O(C[C@H]1N(Cc2scc(C#CCO)c2)CCC1)C | 7000 | | | 23000 |
| OCC#Cc1cc(CN2CC(C)N(C(C)C)CC2)sc1 | 19000 | | | 49500 |
| OCC#Cc1cc(CN(CCN2CCCCCC2)C)sc1 | 9000 | | | 59500 |
| OCC#Cc1cc(CN(CC2CCOCC2)C)sc1 | 2000 | | | 51000 |
| O(CC(O)CN1CCCCC1)c1c(CN2CC3(OCC2)CCCC3)cccc1 | 1000 | | | 6000 |
| S(C)c1nc(c(C(═O)O)cn1)-c1cc(OC)c(OC)cc1 | 27000 | | | 63500 |
| O(CCO)c1c(CN2CC(O)(CO)CCC2)cccc1 | 16000 | | | 27000 |
| O═C(NC)CCN(Cc1scc(C#CCO)c1)C | 6000 | | | 28000 |
| OCC#Cc1cc(CN(CCN2CCCCC2)CC)sc1 | 1000 | | | 5000 |
| O(CCN1CCC(CN(Cc2c(OCCO)cccc2)CC2OCCC2)CC1)C | 9000 | | | 19500 |
| O(CCN1CCC(CN(Cc2c(O)cccc2)CC2OCCC2)CC1)C | 15000 | | | 32000 |
| O(CCN1CCC(CN(Cc2c(OCC)cccc2)CC2OCCC2)CC1)C | 4000 | | | 22000 |
| Fc1c(CN2CCC(CN(Cc3c(OC)cccc3O)CC3OCCC3)CC2)cccc1 | 3000 | | | 15500 |
| O(CCO)c1c(CN(CC(C)C)CC2CCN(Cc3c(OC)cccc3)CC2)cccc1 | 0 | | | 5500 |
| Fc1c(CN2CCC(CN(CCOC)Cc3c(OCCO)cccc3)CC2)cccc1 | 2000 | | | 15500 |
| | 29500 | 23000 | 9500 | 20125 |
| | 1000 | 2000 | 1000 | 1250 |
| | 0 | | | 0 |
| O═C(Nc1cc(C(═O)NC2CN(C3CCCCCC3)CCC2)ccc1)C | 3000 | | | 4750 |
| O(CCN1CCC(CN(Cc2c(OCC═C)cccc2)CC2OCCC2)CC1)C | 1000 | | | 2500 |
| O(C)c1c(CN(CC2OCCC2)CC2CCN(C3CCCC3)CC2)ccc(OC)c1 | 2000 | | | 8000 |
| O(CC)c1c(CN(CC2OCCC2)CC2CCN(C3CCCC3)CC2)cccc1 | 3000 | | | 4750 |
| O═C(Nc1cc(C(═O)N2CCN(C)CC2)cc(CNC2C3CC4CC2CC(C3)C4)c1)C | 1000 | | | 22000 |
| O═C(N(C(C)C)CCO)C═1C(═O)C(C(═O)NCCC2═CCCCC2)═CN | 13000 | | | 15000 |
| (Cc2c(OC)cccc2)C = 1 | | | | |
| O═C(N(CCCN(C)C)C)C═1C(═O)C(C(═O)NCCC(C)C)═CN | 13500 | | | 11000 |
| (Cc2c(OC)cccc2)C = 1 | | | | |
| Fc1c(F)ccc(CNCc2cc(NC(═O)C)cc(C(═O)N3CCN(C)CC3)c2)c1 | 14000 | | | 7000 |
| O═C(NCCC1═CCCCC1)C═1C(═O)C(C(═O)N2CC(O)CCC2)═CN | 4000 | | | 4000 |
| (Cc2c(OC)cccc2)C = 1 | | | | |
| O═C(NC1CCCCCC1)C═1C(═O)C(C(═O)N2C(CC)CCCC2)═CN | 21500 | | | 22750 |
| (Cc2cc(OC)ccc2)C = 1 | | | | |
| O═C(NCCC(C)C)C═1C(═O)C(C(═O)N2CCN(CCOC)CC2)═CN | 8000 | | | 6000 |
| (Cc2c(OC)cccc2)C = 1 | | | | |
| O═C(Nc1cc(C(═O)N2CCCC2)cc(CNC2C3CC4CC2CC(C3)C4)c1)c1ncccc1 | 2000 | | | 9250 |
| O═C(Nc1cc(C(═O)N2C(C)CN(C)CCC2)ccc1)CC | 16500 | | | 11250 |
| OCC#Cc1cc(CN(CCN(C)C)Cc2sc(C)cc2)sc1 | 1000 | | | 500 |
| OCC#Cc1cc(CN(CCCO)Cc2sc(C)cc2)sc1 | 8000 | | | 11000 |
| OCC#Cc1cc(CN(CC(C)C)Cc2c(C)cccc2)sc1 | 9500 | | | 15750 |
| N(CCN(C)C)(Cc1c(C)ccs1)Cc1c(C)ccs1 | 0 | | | 0 |
| OCC#Cc1cc(CN(C(CC)C)Cc2cscc2)sc1 | 2000 | | | 3500 |
| | 28500 | | | 42750 |
| | | | | 2000 |
| | 0 | | | 0 |
| O═C(OC1CCCCC1)C1═C(C)NC(═O)NC1c1cc(OC)c(OC)cc1 | 10000 | | | 10750 |
| O═C(NC1CCCCCC1)c1c(-c2cc(OC)c(OC)cc2)n[nH]c1 | 34000 | | | 40500 |
| O═C(Nc1c(C(═O)N2CCCCC2)cccc1)c1ccc(OCCCC)cc1 | 28500 | | | 23000 |
| O═C(Nc1c(C(═O)N2CCCCC2)cccc1)c1c(OCCOCC)cccc1 | 4000 | | | 6750 |
| O═C(Nc1c(C(═O)N2CCOCC2)cccc1)c1ccc(OCC2OCCC2)cc1 | 18500 | | | 12250 |
| O═C(Nc1ccc(C(═O)N2CCC(C)CC2)cc1)c1ccc(OCC2OCCC2)cc1 | 25000 | | | 17250 |
| O═C(Nc1c(C(═O)N2CCCCC2)cccc1)c1ccc(OCC2OCCC2)cc1 | 12500 | | | 16000 |
| O═C(Nc1c(C(═O)N2CCN(C)CC2)cccc1)c1ccc(OCC2OCCC2)cc1 | 25000 | | | 16250 |
| N(Cc1sc(C)cc1)(Cc1sc(C)cc1)C | 9000 | | | 5000 |
| O═C(OC1CCCCCCC1)C1═C(C)NC(═O)NC1c1cc(O)ccc1 | 37000 | | | 21500 |
| O═C(NCC1OCCC1)C1CCN(Cc2c(OCC)cccc2)CC1 | 24000 | | | 17250 |
| N(CCN(Cc1oc(C)cc1)Cc1oc(C)cc1)(CC)CC | 1000 | | | 1000 |
| O═C(N(CC1C2N(CCC1)CCCC2)C)c1c(-c2cc(OC)c(OC)cc2)n[nH]c1 | 20500 | | | 14250 |
| O═C(OCCc1ccccc1)C1═C(C)NC(═O)NC1c1cc(OC)c(OCCC)cc1 | 18000 | | | 10500 |
| O═C(OC1CCCCCCC1)C1═C(C)NC(═O)NC1c1cc(OC)c(OCc2ccccc2)cc1 | 29500 | | | 15750 |
| O═C(OCC1OCCC1)C1═C(C)NC(═O)NC1c1cc(OC)c(OCCC)cc1 | 0 | | | 500 |
| O═C(OC(CC(C)C)C)c1c(-c2ccccc2)ncnc1 | 19500 | | | 13750 |
| O═C(NCC1OCCC1)c1c(NC(═O)c2ccc(OCCc3ccccc3)cc2)cccc1 | 24500 | | | 20250 |
| O═C(N(C)C1CCCCC1)c1c(NC(═O)c2ccc(OCCOC)cc2)cccc1 | 20000 | | | 20000 |
| O═C(NC1CCCCCC1)c1c(-c2cc3OCCOc3cc2)[nH]nc1 | 0 | | | 0 |
| O═C(Nc1cc(C(═O)NCC2(N(C)C)CCCCCC2)ccc1)C(C)(C)C | 23000 | | | 18250 |
| O═C(NC1CCCCC1)c1c(-c2cc3OCCCOc3cc2)nn(C)c1 | 3000 | | | 8750 |
| | 5000000 | | | 2810000 |
| | 35000 | | | 18000 |
| | 1000 | | | 500 |
| O═C(NC1CCCC1)c1c(NC(═O)c2ccc(OCC3OCCC3)cc2)cccc1 | | | | 665000 |
| O═C(Nc1c(CN2CCOCC2)cccc1)c1ccc(OCC2OCCC2)cc1 | 3700000 | | | 2250000 |
| O═C(OCC(═O)c1ccccc1)c1c(-c2cc3OCCOc3cc2)[nH]nc1 | 5000000 | | | 2900000 |
| O═C(Nc1cc(NC(═O)C)cc(C(═O)NC2C(C)CCCC2)c1)C | 5000000 | | | 2745000 |
| O═C(Nc1cc(C(═O)NC2C3N(CC2)CCCC3)ccc1)C | 2200000 | | | 1405000 |
| O═C(OCC)C═1C(c2ccccc2)═NC(═O)NC═1C | 3400000 | | | 1952500 |
| O═C(NCC1OCCC1)c1c(NC(═O)c2ccc(OCC(C)C)cc2)cccc1 | 2800000 | | | 1620000 |
| O═C(N1CCCCC1)c1c(-c2cc3OCCOc3cc2)[nH]nc1 | 2300000 | | | 1335000 |
| O═C(Nc1c(C(═O)N2CC(C)CC(C)C2)cccc1)c1cc2OCCOc2cc1 | 4200000 | | | 2500000 |
| O═C(NCC)CN(Cc1sccc1)Cc1sccc1 | 3100000 | | | 2250000 |
| N(CCNCc1sccc1)(C)C | 3800000 | | | 2300000 |
| O═C(OC1CCCCCCC1)C1═C(C)NC(═O)NC1c1cc(OC)c(OCCC)cc1 | 190000 | | | 1152500 |
| O═C(Nc1c(C(═O)N2C(CCO)CCCC2)cccc1)c1ccc(OC)cc1 | 5000000 | | | 4050000 |
| O═C(OC1CCCCCC1)C1═C(C)NC(═O)NC1c1cc(OCc2ccccc2)ccc1 | 5000000 | | | 3350000 |
| O═C(OC1CCCCCCC1)C1═C(C)NC(═O)NC1c1cc2OCOc2cc1 | 5000 | | | 3500 |
| O═C(OC1CCCCCC1)C1═C(C)NC(═O)NC1c1cc2OCOc2cc1 | 6000 | | | 4000 |

Structures for Molecules Used for Experimental Data

| Molecule | Structure | ID |
|---|---|---|
| O═C(OCC)C1═C(C)NC(═O)NC1c1cc(OC)c(OCCc2ccccc2)cc1 | 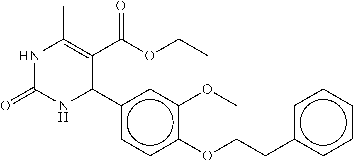 | 7679642 |
| O═C(Nc1c(-c2oc(C)c(CN(C/C═C/c3cc(OC)c(O)cc3)C)n2)cccc1)c1cscc1 | 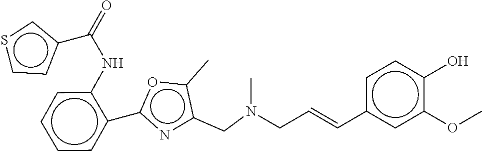 | 48325627 |
| O═C(NCCC1═CCCCC1)C═1C(═O)C(C(═O)N2CCCCC2)═CN(Cc2c(OC)cccc2)C═1 | 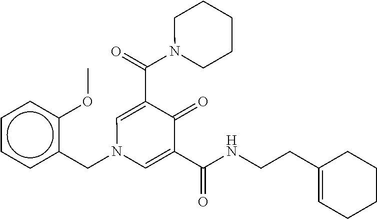 | 93701800 |
| OCC#Cc1cc(CN(Cc2c(C)ccs2)C)sc1 | 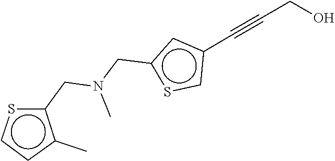 | 91240494 |
| O═C(Nc1cc(C(═O)N2C[C@H]3N(C)C[C@@H](C2)CC3)ccc1)C | 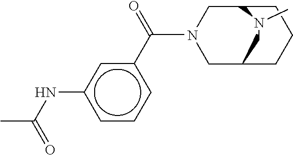 | 49602830 |
| O(C[C@H]1N(Cc2scc(C#CCO)c2)CCC1)C | 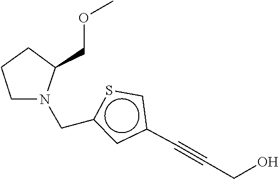 | 10434393 |
| OCC#Cc1cc(CN2CC(C)N(C(C)C)CC2)sc1 | 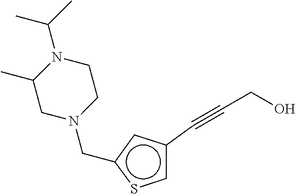 | 30933538 |
| OCC#Cc1cc(CN(CCN2CCCCCC2)C)sc1 | 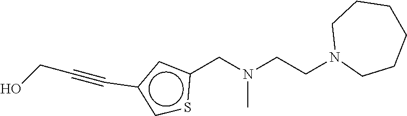 | 379497358 |
| OCC#Cc1cc(CN(CC2CCOCC2)C)sc1 | 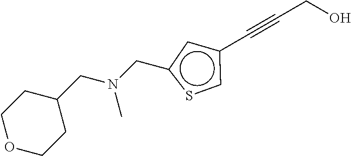 | 37644792 |
| O(CC(O)CN1CCCCC1)c1c(CN2CC3(OCC2)CCCC3)cccc1 | 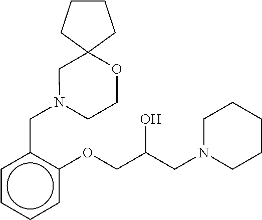 | 43103865 |
| S(C)c1nc(c(C(═O)O)cn1)-c1cc(OC)c(OC)cc1 | 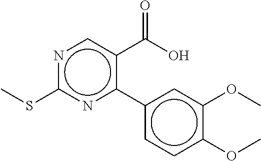 | 15578104 |
| O(CCO)c1c(CN2CC(O)CO)CCC2)cccc1 | 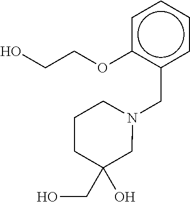 | 75846711 |
| O═C(NC)CCN(Cc1scc(C#CCO)c1)C | 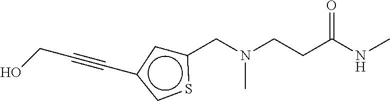 | 41503365 |
| OCC#Cc1cc(CN(CCN2CCCCC2)CC)sc1 | 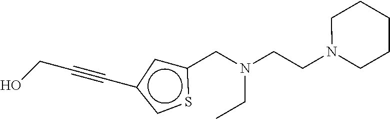 | 68133153 |
| O(CCN1CCC(CN(Cc2c(OCCO)cccc2)CC2OCCC2)CCl)C | 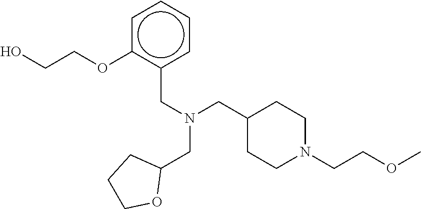 | 78409968 |
| O(CCN1CCC(CN(Cc2c(O)cccc2)CC2OCCC2)CC1)C | 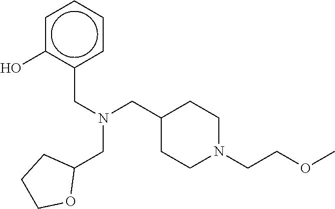 | 49010376 |
| O(CCN1CCC(CN(Cc2c(OCC)cccc2)CC2OCCC2)CC1)C | 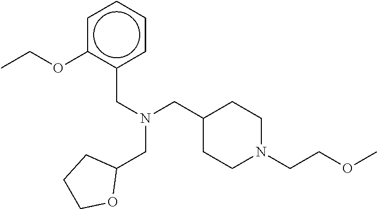 | 35161547 |
| Fc1c(CN2CCC(CN(Cc3c(OC)cccc3O)CC3OCCC3)CC2)cccc1 | 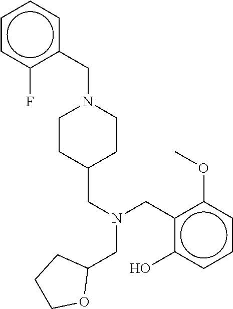 | 66893200 |
| O(CCO)c1c(CN(CC(C)C)CC2CCN(Cc3c(OC)cccc3)CC2)cccc1 | 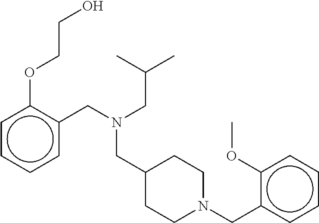 | 97913321 |
| Fc1c(CN2CCC(CN(CCOC)Cc3c(OCCO)cccc3)CC2)cccc1 | 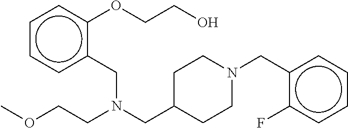 | 66918704 |
| O═C(Nc1cc(C(═)NC2CN(C3CCCCCC3)CC2)ccc1)C | 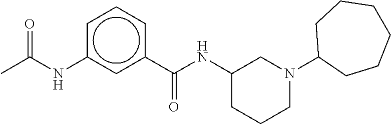 | 38740381 |
| O(CCN1CCC(CN(Cc2c(OCC═C)cccc2)CC2OCCC2)CC1)C | 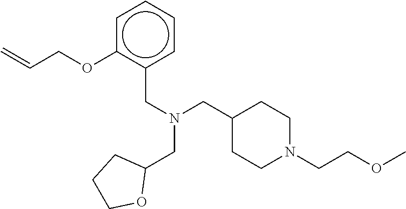 | 97880594 |
| O(C)c1c(CN(CC2OCCC2)CC2CCN(C3CCCC3)CC2)ccc(OC)c1 | 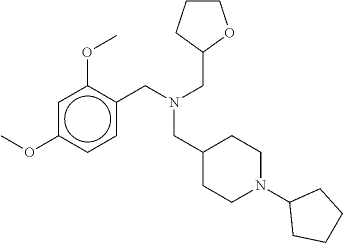 | 58156196 |
| O(CC)c1c(CN(CC2OCCC2)CC2CCN(C3CCCC3)CC2)cccc1 | 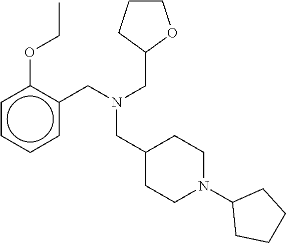 | 38521191 |
| O═C(Nc1cc(C(═O)N2CCN(C)CC2)cc(CNC2C3CC4CC2CC(C3)C4)c1)C | 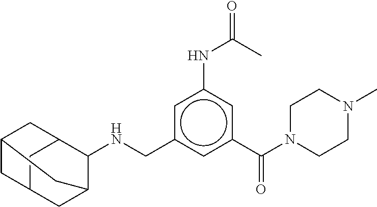 | 70444941 |
| O═C(N(C(C)C)CCO)C═1C(═O)C(C(═O)NCCC2═CCCCC2)═CN(Cc2c(OC)cccc2)C═1 | 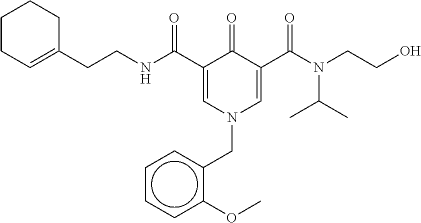 | 55345532 |
| O═C(N(CCCN(C)C)C)C═1C(═O)C(C(═O)NCCC(C)C)═CN(Cc2c(OC)cccc2)C═1 | 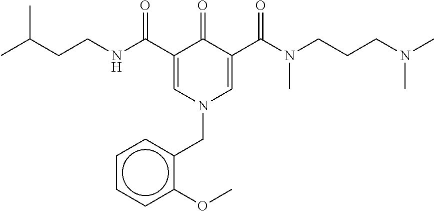 | 23614835 |
| Fc1c(F)ccc(CNCc2cc(NC(═O)C)cc(C(═O)N3CCN(C)CC3)c2)c1 | 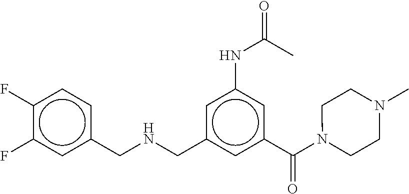 | 73759021 |
| O═C(NCCC1═CCCCC1)C═1C(═O)C(C(═O)N2CC(O)CCC2)═CN(Cc2c(OC)cccc2)C═1 | 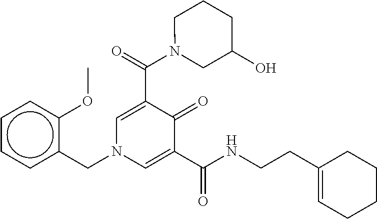 | 53145516 |
| O═C(NC1CCCCCC1)C═1C(═O)C(C(═O)N2C(CC)CCCC2)═CN(Cc2cc(OC)ccc2)C═1 | 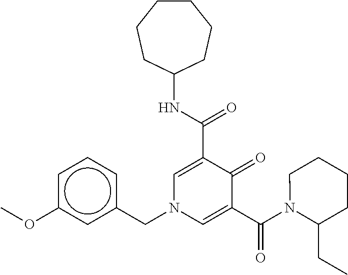 | 47976750 |
| O═C(NCCC(C)C)C═1C(═O)C(C(═O)N2CCN(CCOC)CC2)═CN(Cc2c(OC)cccc2)C═1 | 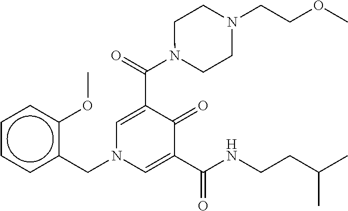 | 24668741 |
| O═C(Nc1cc(C(═O)N2CCCC2)cc(CNC2C3CC4CC2CC(C3)C4)c1)c1ncccc1 | 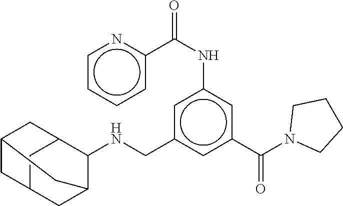 | 92881721 |
| O═C(Nc1cc(C(═O)N2C(C)CN(C)CCC2)ccc1)CC | 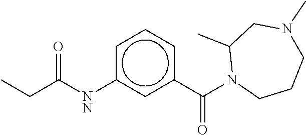 | 41610622 |
| OCC#Cc1cc(CN(CCN(C)C)Cc2sc(C)cc2)sc1 | 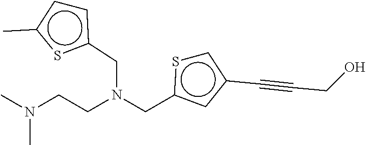 | 10253964 |
| OCC#Cc1cc(CN(CCCO)Cc2sc(C)cc2)sc1 | 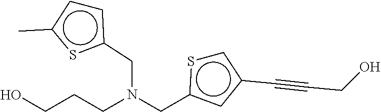 | 24200140 |
| OCC#Cc1cc(CN(CC(C)C)Cc2c(C)cccc2)sc1 | 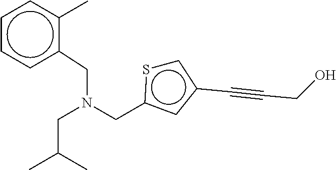 | 18052219 |
| N(CCN(C)C)(Cc1c(C)ccs1)Cc1c(C)ccs1 | 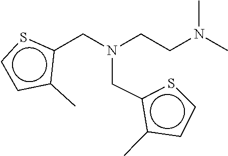 | 10355732 |
| OCC#Cc1cc(CN(C(CC)C)Cc2scc2)sc1 | 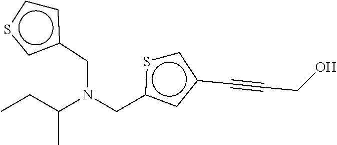 | 26918198 |
| O═C(OC1CCCCC1)C1═C(C)NC(═O)NC1c1cc(OC)c(OC)cc1 | 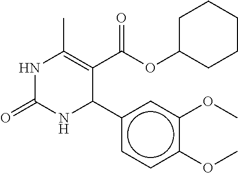 | STK364138 |
| O═C(NC1CCCCCC1)c1c(-c2cc(OC)c(OC)cc2)n[nH]c1 | 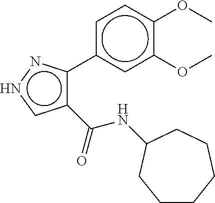 | STL184833 |
| O═C(Nc1c(C(═O)N2CCCCC2)cccc1)c1ccc(OCCCC)cc1 | 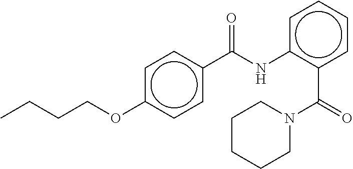 | STL071122 |
| O═C(Nc1c(C(═O)N2CCCCC2)cccc1)c1c(OCCOCC)cccc1 | 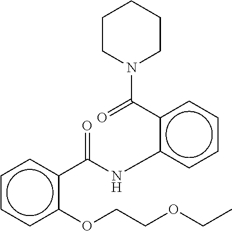 | STL06687 |
| O═C(Nc1c(C(═O)N2CCOCC2)cccc1)c1cccc(OCC2OCCC2)cc1 | 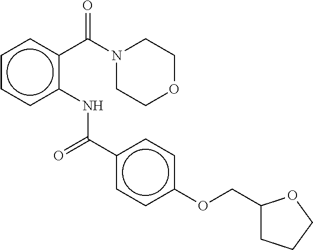 | STL068656 |
| O═C(Nc1ccc(C(═O)N2CCC(C)CC2)cc1)c1ccc(OCC2OCCC2)ccl | 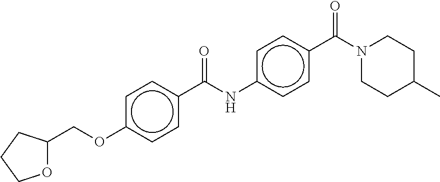 | STL068664 |
| O═C(Nc1c(C(═O)N2CCCCC2)cccc1)c1ccc(OCC2OCCC2)cc1 | 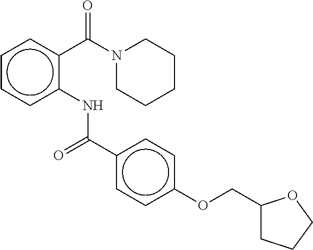 | STL068657 |
| O═C(Nc1c(C(═O)N2CCN(C)CC2)cccc1)c1ccc(OCC2OCCC2)cc1 | 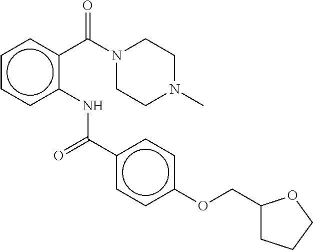 | STL068659 |
| N(Cc1sc(C)cc1)(Cc1sc(C)cc1)C | 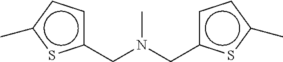 | STL069512 |
| O═C(OC1CCCCCCC1)C1═C(C)NC(═O)NC1c1cc(O)ccc1 | 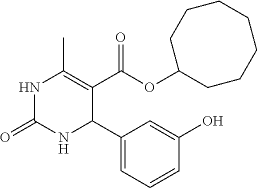 [SMILES: CC1=C(C(=O)OC2CCCCCCC2)C(c2cccc(O)c2)NC(=O)N1] | STK062421 |
| O═C(NCC1OCCC1)C1CCN(Cc2c(OCC)cccc2)CC1 | 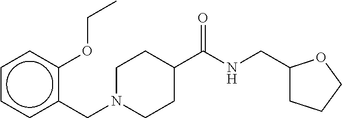 | STK01719 |
| N(CCN(Cc1oc(C)cc1)Cc1oc(C)cc1)(CC)CC | 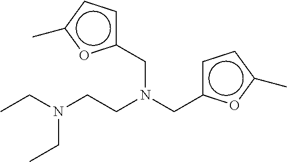 | STK161925 |
| O═C(N(CC1C2N(CCC1)CCCC2)C)clc(-c2cc(OC)c(OC)cc2)n[nH]c1 | 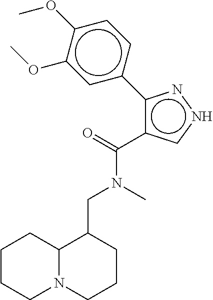 | STL546978 |
| O═C(OCCc1ccccc1)C1═C(C)NC(═O)NC1c1cc(OC)c(OCCC)cc1 | 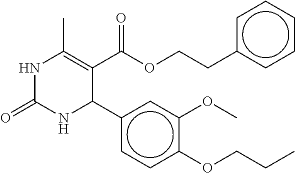 | STK083274 |
| O═C(OC1CCCCCCC1)C1═C(C)NC(═O)NC1c1cc(OC)c(OCc2ccccc2)cc1 | 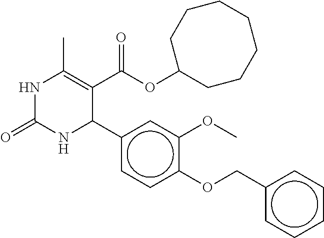 | STK075753 |
| O═C(OCC1OCCC1)C1═C(C)NC(═O)NC1c1cc(OC)c(OCCC)cc1 | 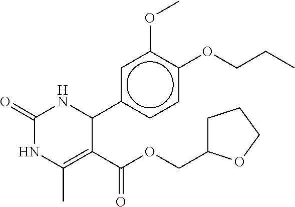 | STK054478 |
| O═C(OC(CC(C)C)C)c1c(-c2ccccc2)ncnc1 | 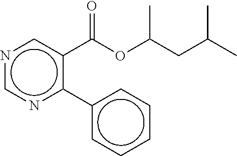 | STK244118 |
| O═C(NCC1OCCC1)c1c(NC(═O)c2ccc(OCCc3ccccc3)cc2)cccc1 | 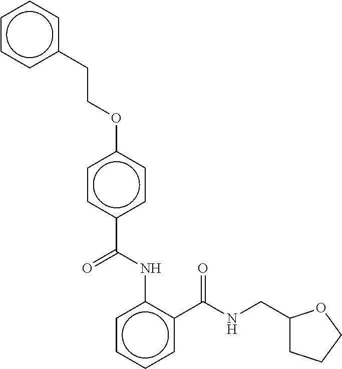 | STK276413 |
| O═C(N(C)C1CCCCC1)c1c(NC(═O)c2ccc(OCCOC(cc2)cccc1 | 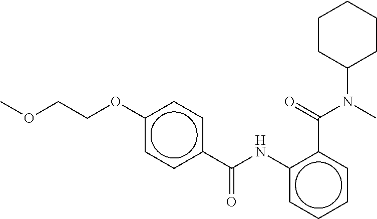 | STK345854 |
| O═C(NC1CCCCCC1)c1c(-c2cc3OCCOc3cc2)[nH]nc1 | 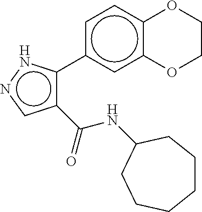 | Z224428262 |
| O═C(Nc1cc(C(═O)NCC2(N(C)C)CCCCCC2)ccc1)C(C)(C)C | 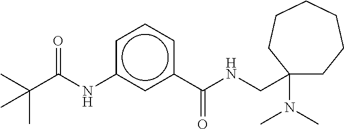 | Z253654054 |
| O═C(NC1CCCCC1)c1c(-c2cc3OCCCOc3cc2)nn(C)c1 | 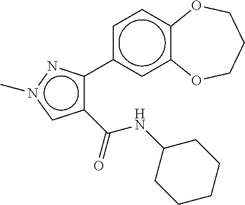 | Z79410098 |
| O═C(NC1CCCC1)c1c(NC(═O)c2ccc(OCC3OCCC3)cc2)cccc1 | 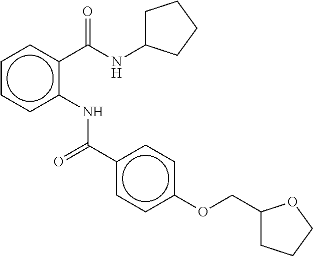 | Z28085941 |
| O═C(Nc1c(CN2CCOCC2)cccc1)c1ccc(OCC2OCCC2)cc1 | 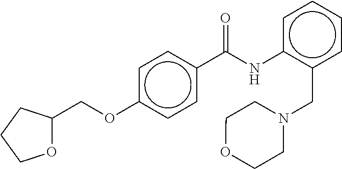 | PB146722878 |
| O═C(OCC(═O)c1cccc1)c1c(-c2cc3OCCOc3cc2)[mH]nc1 | 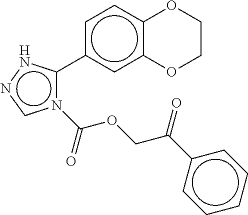 | Z217871108 |
| O═C(Nc1cc(NC(═O)C)cc(C(═O)NC2C(C)CCCC2)c1)C | 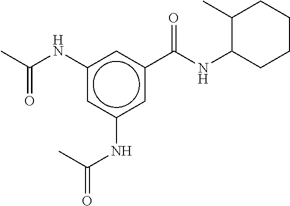 | Z27075709 |
| O═C(Nc1cc(C(═O)NC2C3N(CC2)CCCC3)ccc1)C | 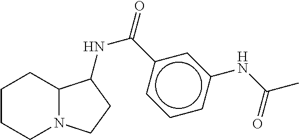 | Z767436704 |
| O═C(OCC)C═1C(c2ccccc2)═NC(═O)NC═1C | 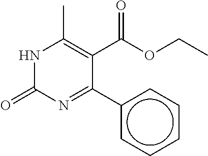 | Z1665450493 |
| O═C(NCC1OCCC1)c1c(NC(═O)c2ccc(OCC(C)C)cc2)cccc1 | 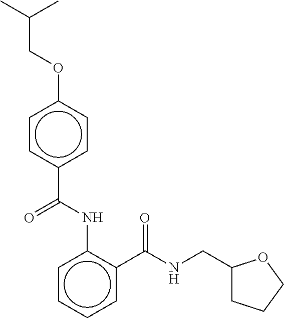 | Z739206878 |
| O═C(N1CCCCC1)c1c(-c2cc3OCCOc3cc2)[nH]nc1 | 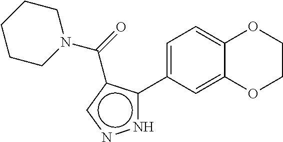 | Z224448688 |
| O═C(Nc1c(C(═O)N2CC(C)CC(C)C2)cccc1)c1cc2OCCOc2cc1 | 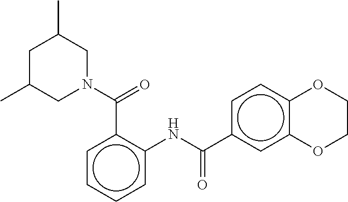 | Z39442736 |
| O═C(NCC)CN(Cc1sccc1)Cc1sccc1 | 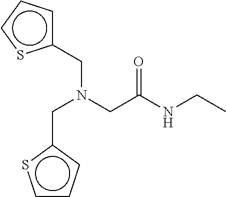 | Z162917606 |
| N(CCNCc1sccc1)(C)C | 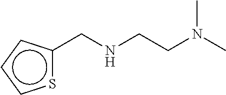 | Z90512807 |
| O═C(OC1CCCCCCC1)C1═C(C)NC(═O)NC1c1cc(OC)c(OCCC)cc1 | 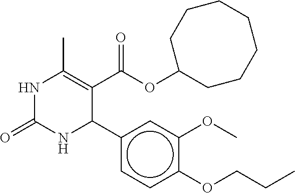 | AG- 205/11867180 |
| O═C(Nc1c(C(═O)N2C(CCO)CCCC2)cccc1)c1ccc(OC)cc1 | 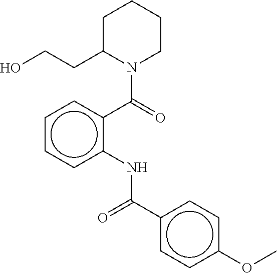 | AP- 064/41684584 |
| O═C(OC1CCCCCC1)C1═C(C)NC(═O)NC1c1cc(OCc2ccccc2)ccc1 | 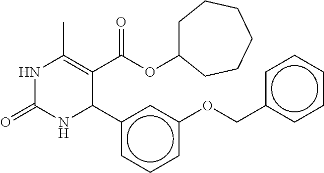 | AG- 205/11867172 |
| O═C(OC1CCCCCCC1)C1═C(O)NC(═O)NC1c1cc2OCOc2cc1 | 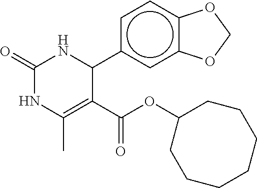 | AG- 690/33475017 |
| O═C(OC1CCCCCC1)C1═C(C)NC(═O)NC1c1cc2OCOc2cc1 | 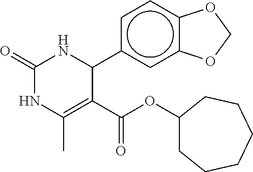 | 1308-0451 |

### Example 6. SKI8 Project Ligand Development

Summary

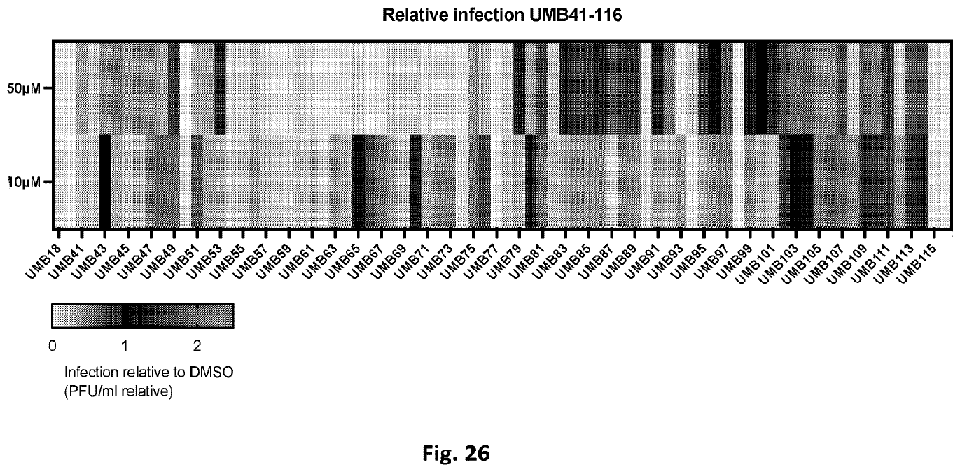

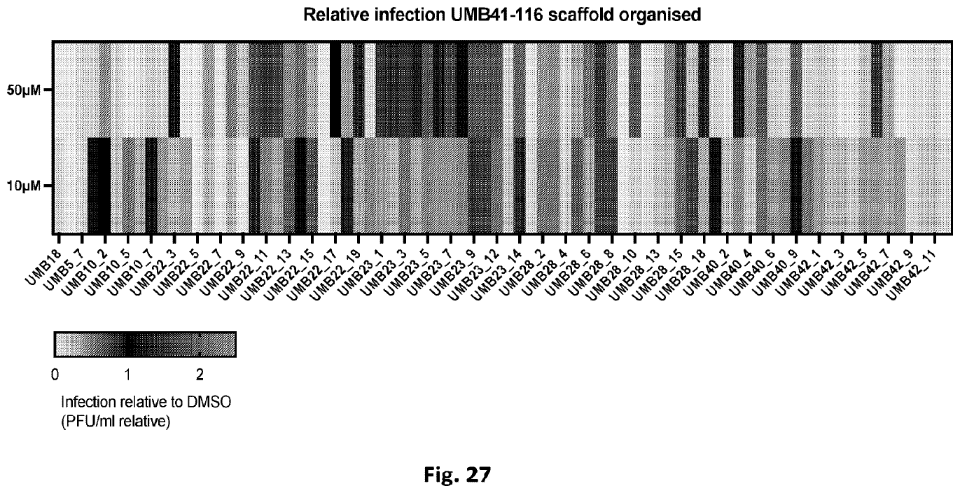

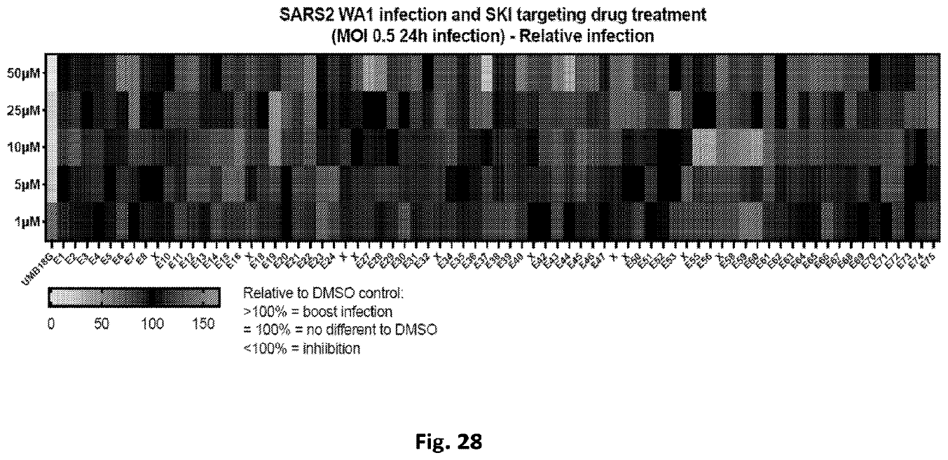

Similarity analysis of previously identified compounds was undertaken on the original along with additional compounds that had weak activity. Compounds numbered 41 to 116 were purchased and subjected to experimental analysis (FIG. 26 and FIG. 27). Based on this data, 4 scaffolds were identified for further analysis and optimization: UMB18, UMB28, UMB22_5 and UMB22_16. An additional round of similarity searching was done on UMB22_5 and UMB22_16 (FIG. 28). This did not yield compounds with significant activity. Subsequent design efforts involved use of SILCS in collaboration with SilcsBio LLC (CADD modeling) and Dalriada (medicinal chemistry). From these efforts a total of 72 compounds were designed, synthesized and tested. SARS2 inhibition and cellular toxicity data are available on these compounds along with the SILCS analysis. In addition, on a subset of these compounds Surface Plasmon Resonance data was obtained, indicating direct interactions with the target protein WDR61.

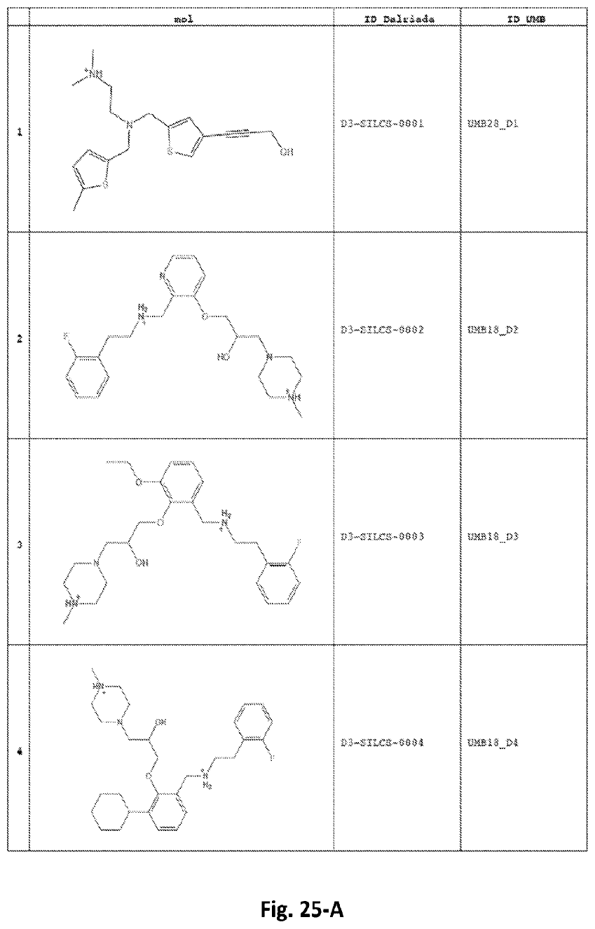

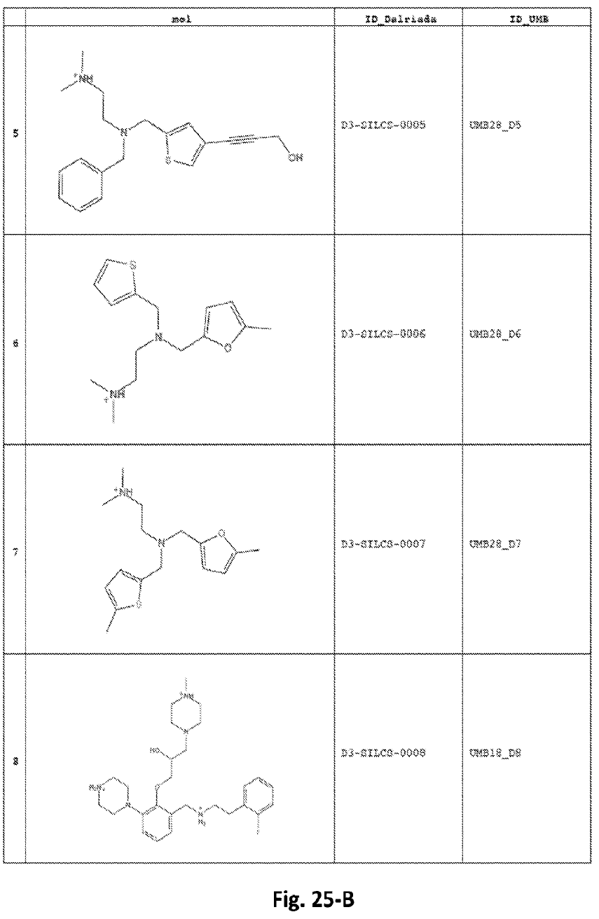

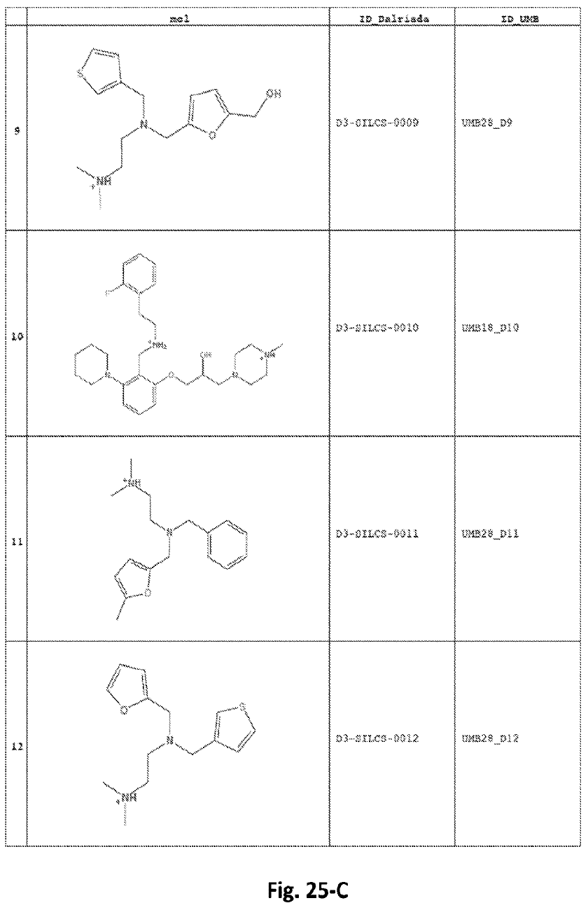

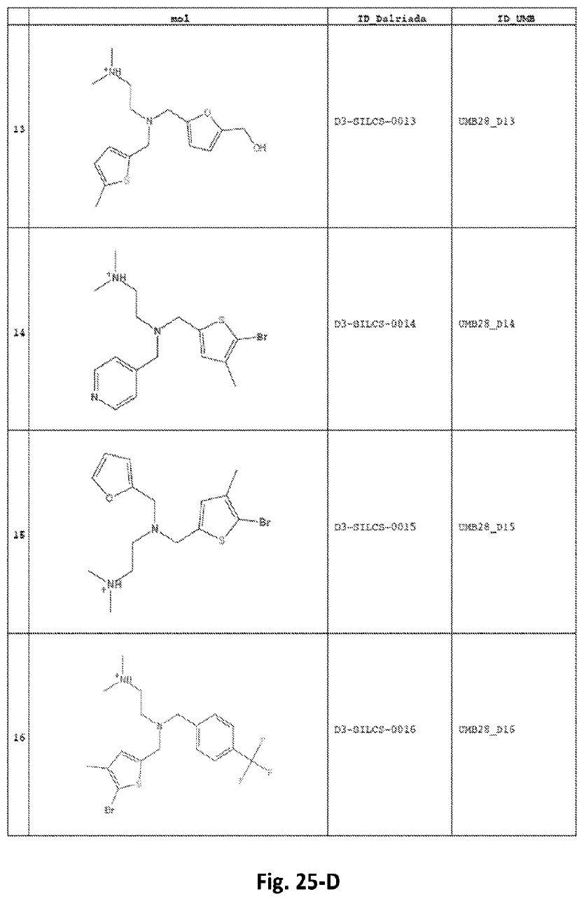

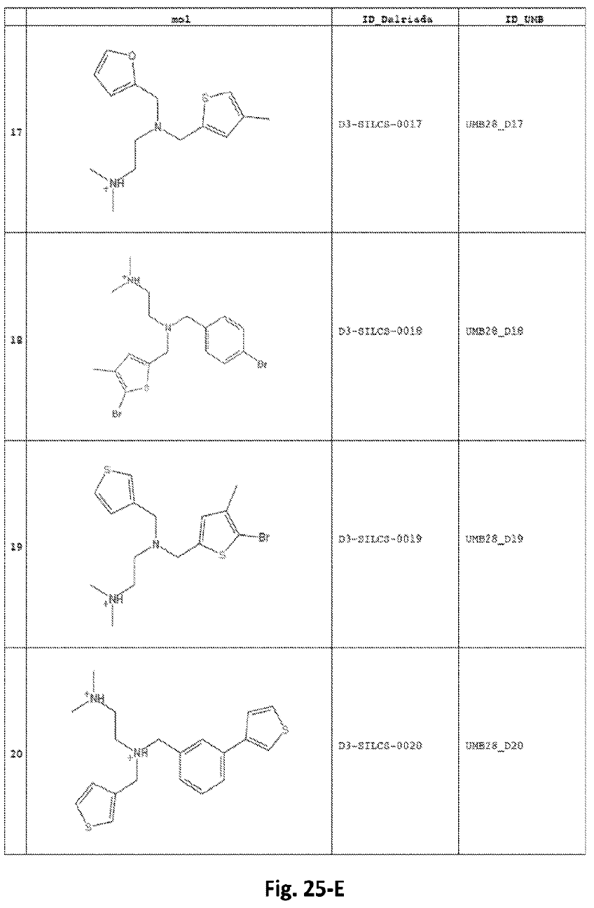

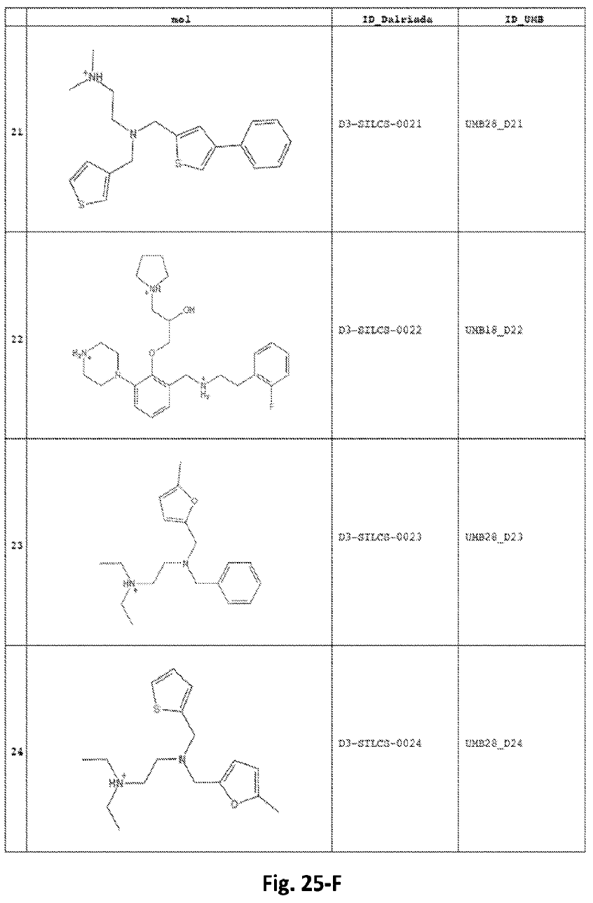

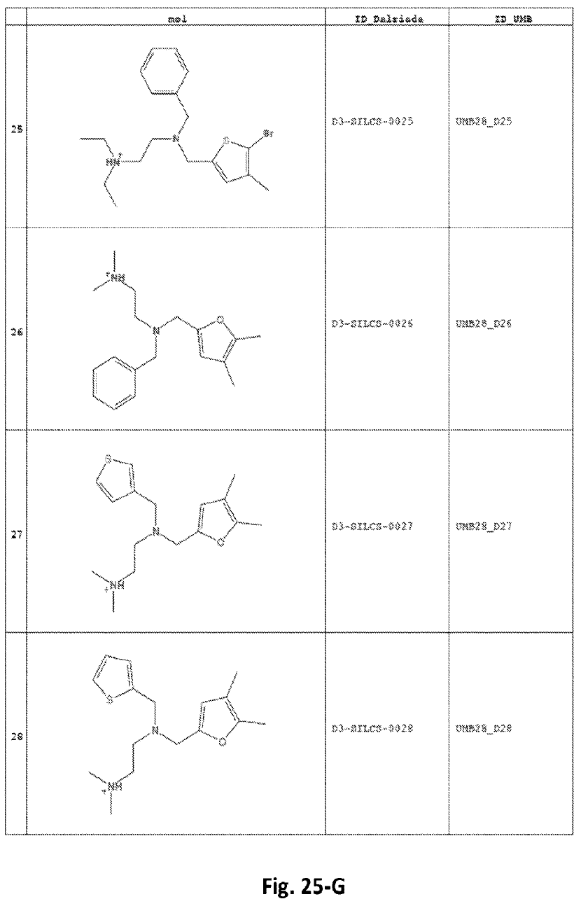

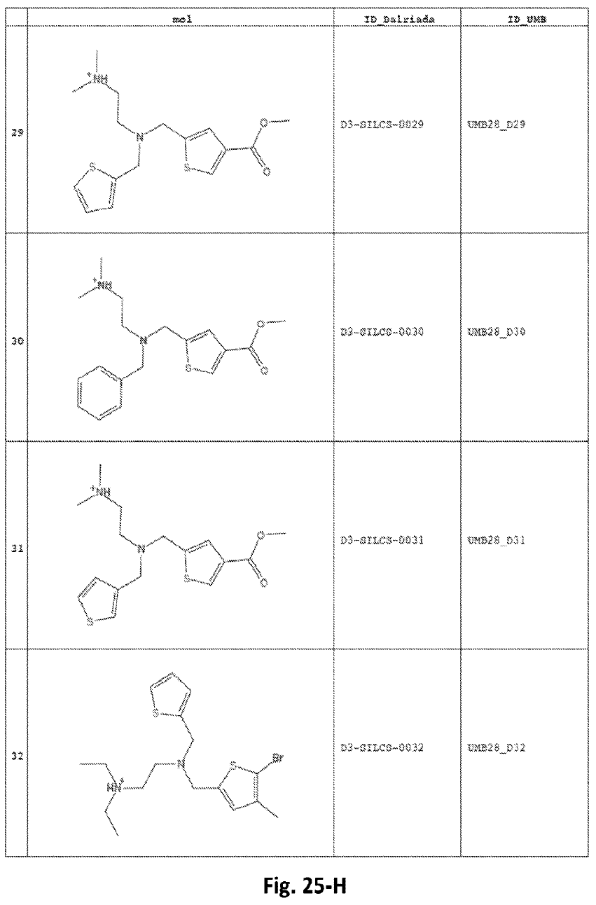

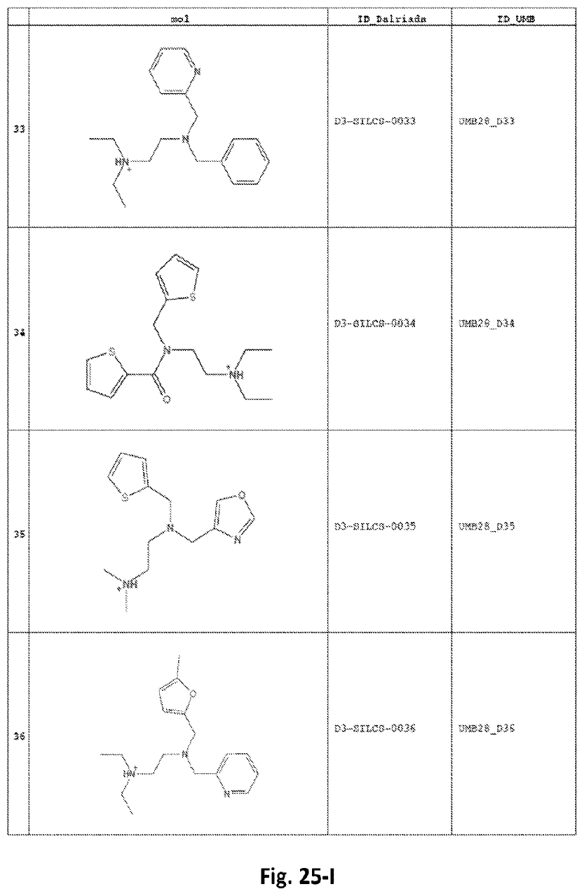

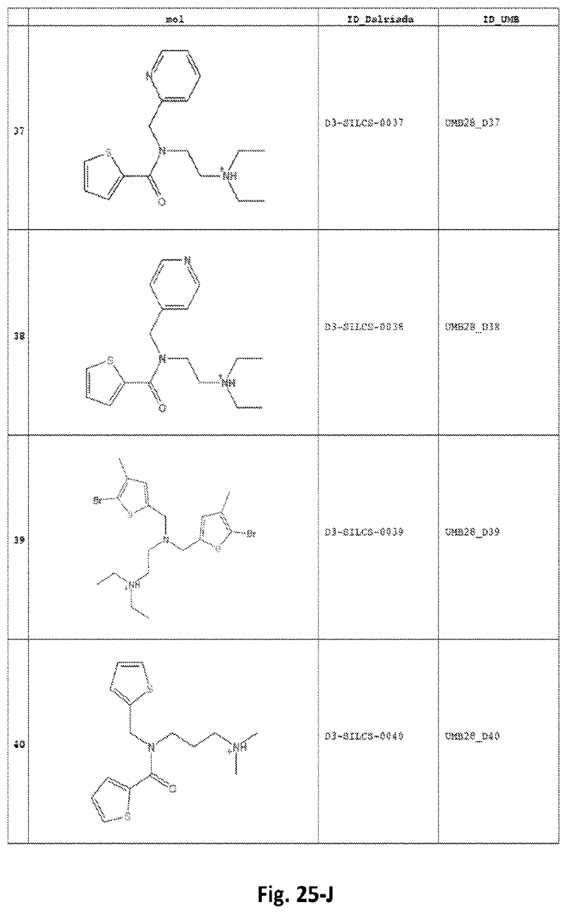

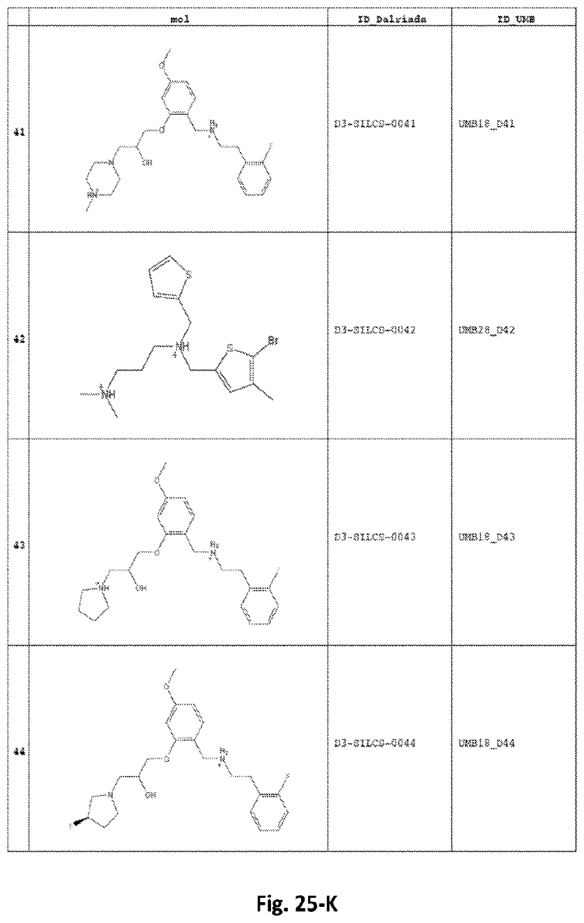

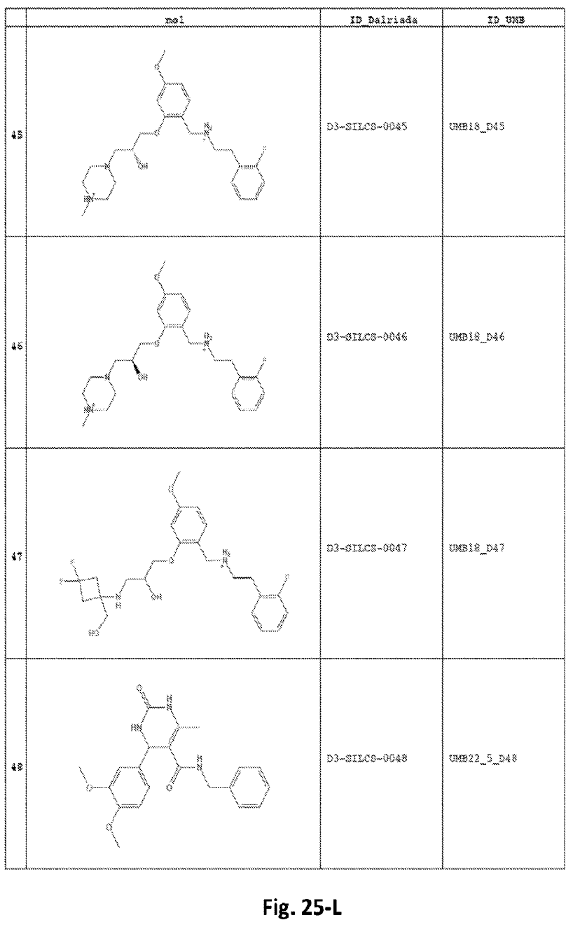

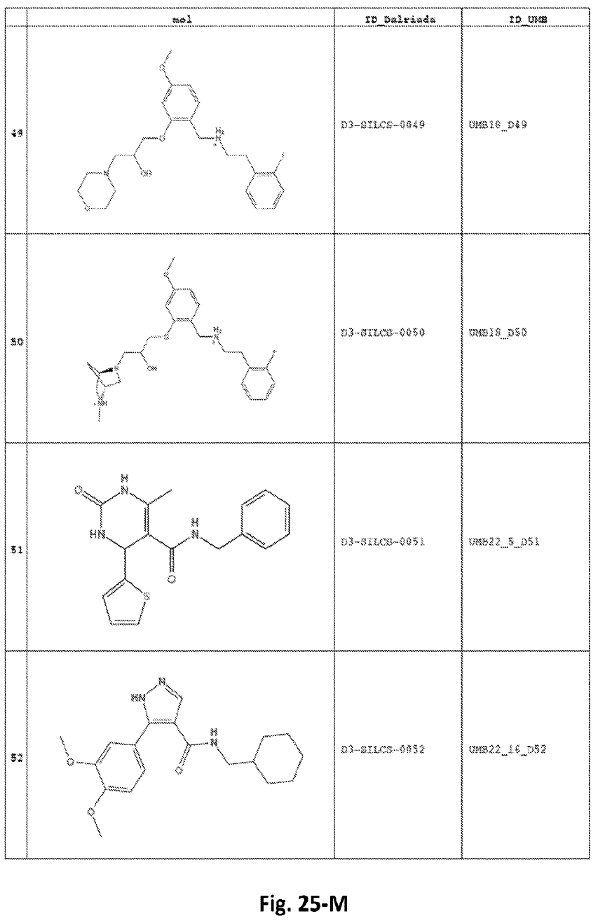

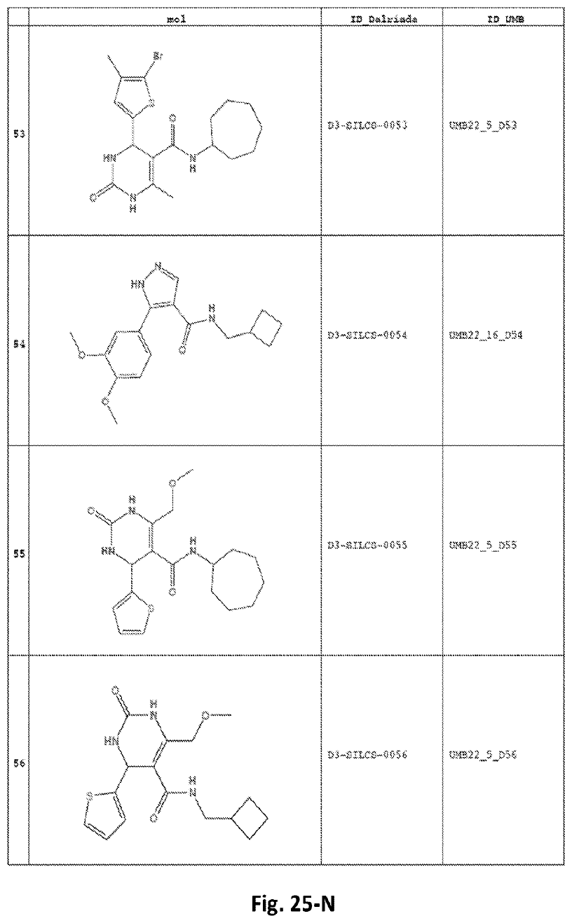

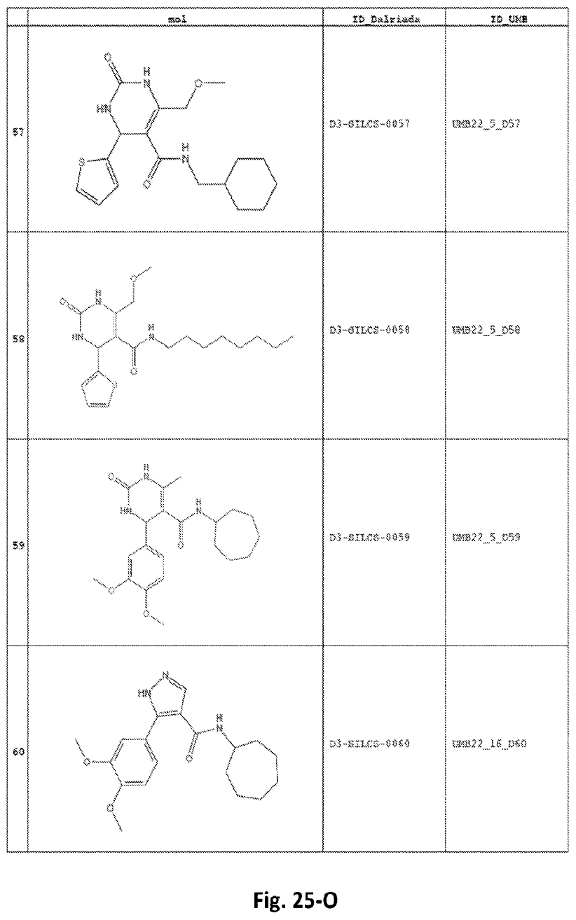

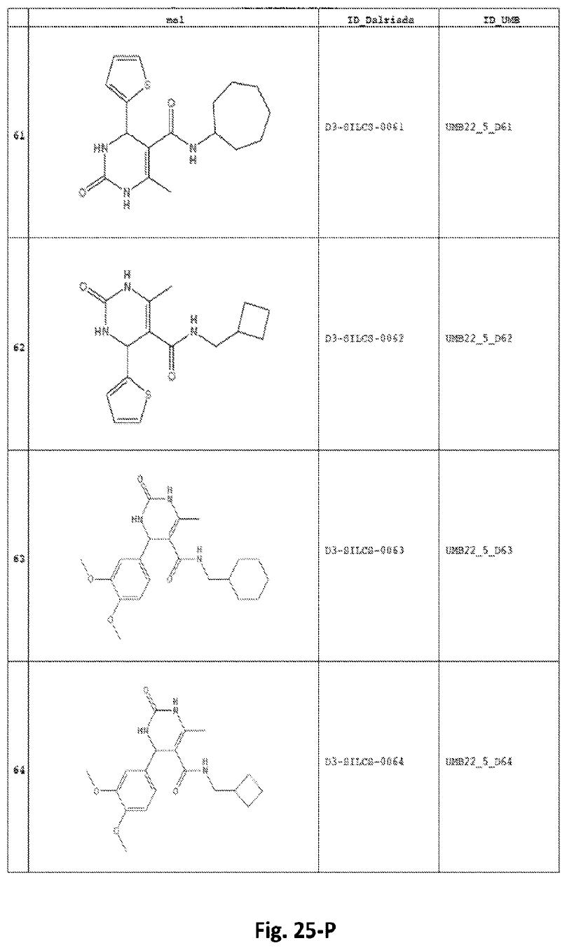

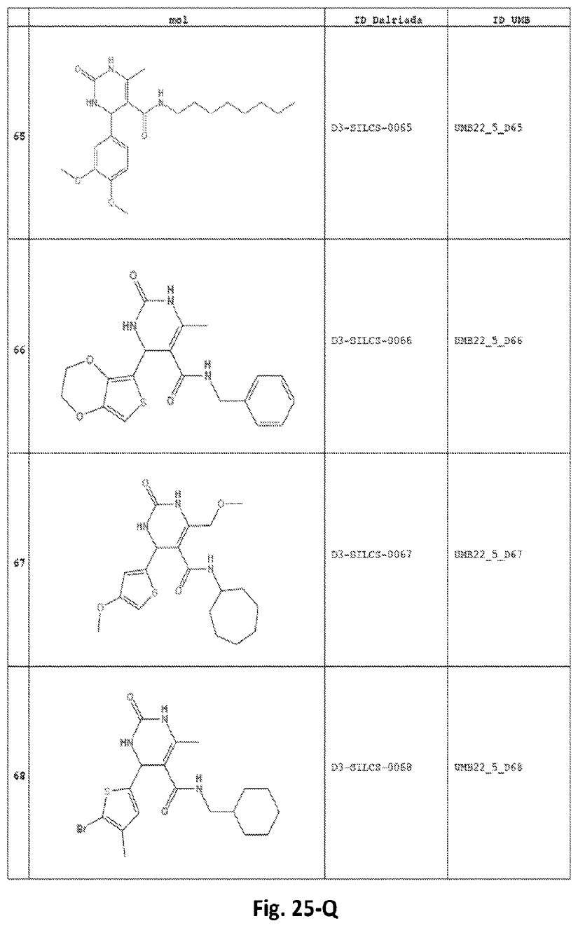

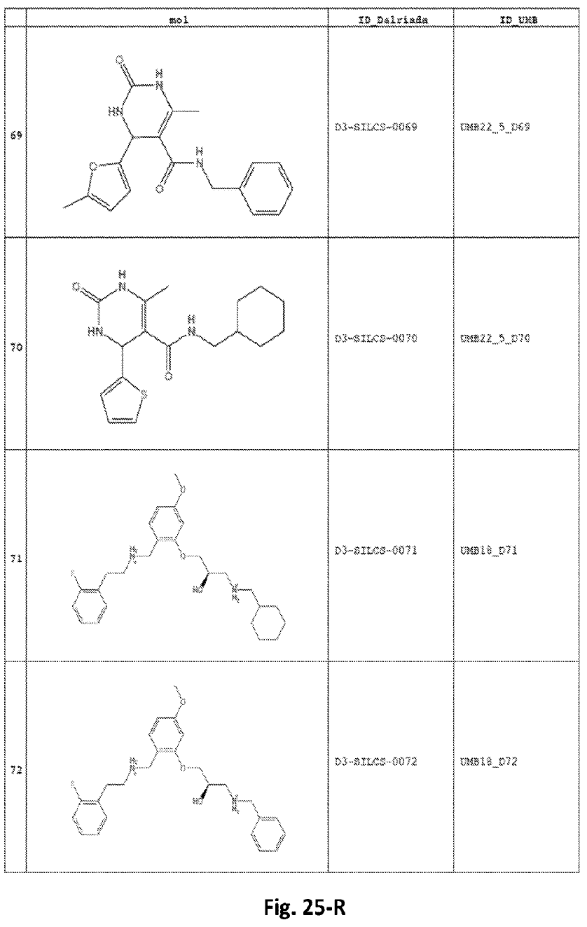

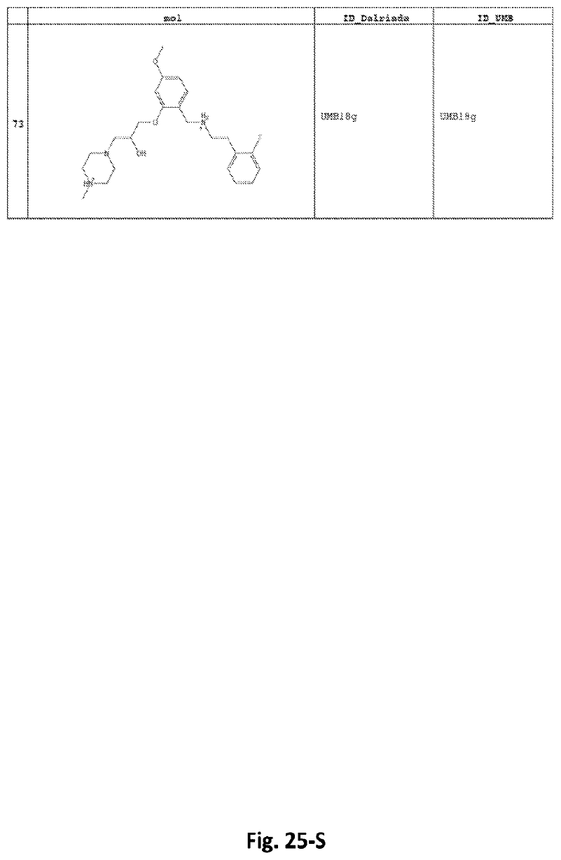

FIGS. 25-A through 25-S depict the structures of SILCS 1-72.

Nomenclature: UMB18_D2: UMB18, UMB22 or UMB28 indicates the scaffold. Nomenclature for biological data is based on the name SILCSx, where x corresponds to the D2 number: UMB18_D2 corresponds to SILCS2.

Methods

SILCS FragMap Generation for Ligand Design

SILCS Simulations were undertaken on the human analog of the Ski8 protein, termed WDR61. The protein structure was obtained from the protein databank and the structure was prepared for the SILCS simulations using the PDB reader in the CHARMM-GUI where an acetyl group was added to residue 5 which is the N terminus of the protein due to the initial 4 residues not being observed in the crystal structure. The protein was then subjected to both the standard SILCS simulation protocol and the SILCS-Halogen (SILCS-X) protocol using version 2020.1 of the SILCS software suite (SilcsBio LLC). The SILCS FragMaps, including conversion to grid free energies (GFE), and exclusion maps were extracted from the SILCS simulation trajectories for use in subsequent modeling and binding affinity predictions. Details of the SILCS simulations and FragMaps and exclusion map calculations are described in Ustach et al. 2019.

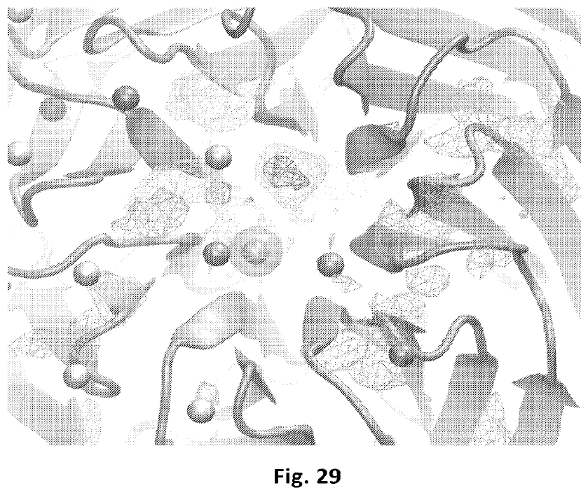

Identification of the ligand binding site on WDR61 applied the SILCS-Hotspots approach as performed in the initial SKI8 ligand discovery study. Multiple hotspots were identified in the region of the protein previously identified as the binding site on SKI8 (with the site deeper in the binding pocket selected to define the center of the binding site in WDR61 for subsequent docking calculations, as shown in FIG. 29.

Ligand docking used the exhaustive SILCS-MC docking protocol. This involves overlaying ligands on the SILCS GFE FragMaps with classified ligand atoms assigned the grid free energy (GFE) value associated with the corresponding FragMap. These atom-based GFE scores are then summed to yield the ligand grid free energy (LGFE) that approximates the binding affinity of the ligand. SILCS-MC docking was performed using the generic atom classification scheme that includes explicit treatment of halogen atoms. All compounds were assigned their dominant protonation state and subjected to energy minimization using the program MOE (Chemical Computing Group). To initiate docking the ligands were subjected to additional energy minimizations using CGenFF and placed in a random location, orientation and conformation in the binding pocket as defined by the pocket center and within a 5 Aradius. The compounds were then subjected to MC translations, rotations and dihedral rotations (maximum of 1 Å, 180° and 180°, respectively) for 10,000 MC steps followed by 40,000 simulated annealing MC steps with a maximum translations, rotations and dihedral rotations of 0.2 Å, 9° and 9°, respectively). The docking run was repeated for a minimum of 50 runs with convergence based on the most favorable LGFE scores being within −0.5 kcal/mol up to a maximum of 250 runs. For MC conformational sampling the LGFE and the CGenFF intramolecular energy (Dielectric=4r, where r is the distance between non-excluded atoms) were used as the Metropolis criteria, while the final binding free energy as based only on the LGFE score. The most favorable LGFE conformer was selected for ranking and analysis including contributions of the atom-based GFE scores that indicate the contribution of classified atoms in the ligand to the overall predicted binding affinity.

Results and Discussion

Similarity search was performed for analogs of compounds showing any activity from an initial set of 40 compounds. 88 compounds were selected based on similarity search of MolPort database against UMB5, 10, 22, 23, 28, 40 (similar to UMB18), 42 (similar to UMB28). IAV data on all compounds is shown in FIGS. 26-28. Following a screening of all compounds 1-116, UMB18, UMB28, UMB22_5 and UMB22_16 scaffolds are identified as leads for further ligand development (FIG. 27). FIG. 28 demonstrates that, based on average over all concentrations tested, only a few compounds show activity with the best being 4- to 5-fold worse than UMB18G as well as worse than the leads UMB22_5 and UMB22_16. Subsequent development of these leads was undertaken in the optimization efforts.

FIG. 29 depicts SILCS-Hotspots overlaid on the structure of WDR61 (cyan cartoon) in the binding region previously targeted on the SKI8 protein. Hotspots are shown as spheres colored by the average LGFE score for the fragments in each hotspots (Red, most favorable, blue, least favorable). The Hotspots indicated by the larger transparent sphere around the smaller sphere was used to define the center of the ligand binding pocket for SILCS-MC docking calculations. Included in the figure are the SILCS Fragmaps in mesh for generic apolar (green), generic hydrogen-bond donor (blue) and acceptor (red), negatively charged (orange) and positively charged (cyan) functional groups at a GFE cutoff of −1.2 kcal/mol or −1.5 kcal/mol in the case of the positive FragMaps.

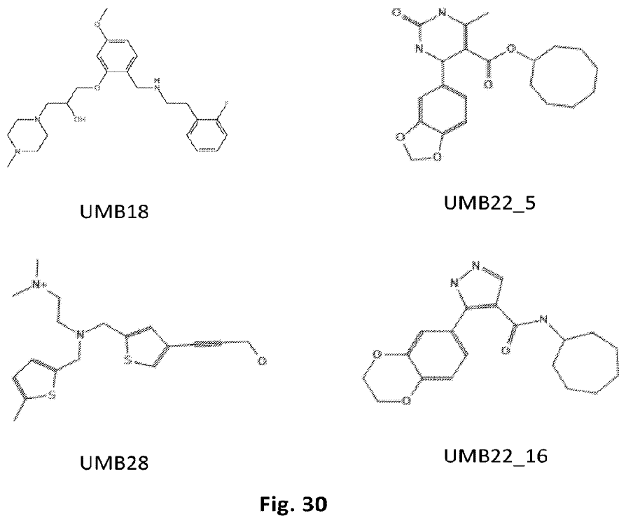

Data from the Weston et al. study and subsequent experiments on chemically similar analogs of compounds identified in the initial study (see FIGS. 26-28) led to the identification of 4 lead scaffolds, UMB18 and UMB28 represented the top 2 leads and UMB22_5 and UMB22_16 represented second tier lead compounds (FIG. 30). These compounds were subjected to SILCS-MC docking from which the binding orientations, overlap of the ligands with the SILCS FragMaps, the LGFE scores and the atom-based GFE scores were obtained. This information along with synthetic and computed bioavailability considerations using the 4DBA metric (Oashi et al. 2011) were used to design analogs of the four scaffolds. These compounds were then subjected to experimental SARS2 antiviral and cellular toxicity assays.

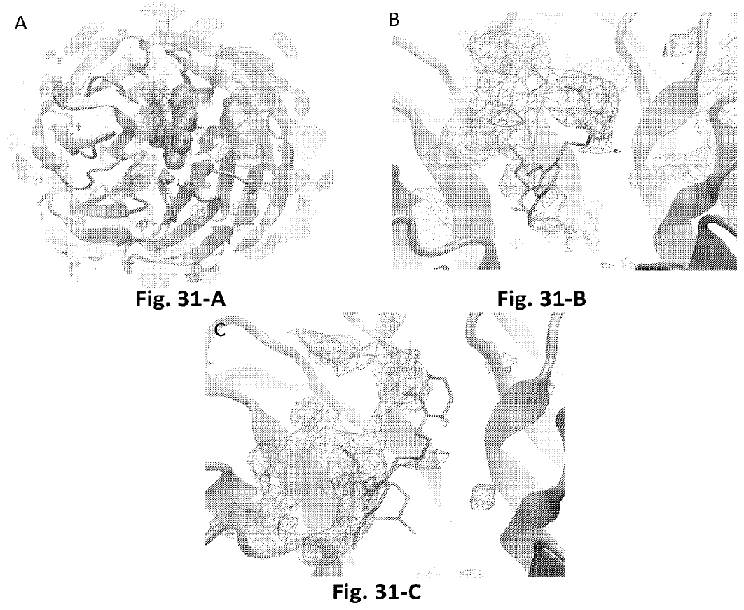

UMB18 as well as the remaining lead compounds are predicted to bind in the central pocket of WDR61 as shown in FIG. 31-A. Details of the binding orientation along with the overlap of the ligands with the SILCS FragMaps are shown on FIG. 31-B for the standard FragMaps and FIG. 31C for the aromatic halogen FragMaps. The overlap of UMB18 with the FragMaps is good, indicating that all regions of the ligand are contributing to binding. Notable are the overlap of the basic amine in the piperazine ring with the positive charge FragMaps (FIG. 31-B) and the fluorophenyl moiety with both generic apolar (FIG. 31-B) and fluorobenzene (FIG. 31-C) FragMaps. Consistent with these observations are the atom-based GFE scores shown in FIG. 32. As may be seen the GFE contributions for all the atoms in the molecule are favorable indicating their favorable contributions to binding. The largest GFE contributions are from the piperazine basic N, with −1.7 kcal/mol, and from the fluorine of the fluorobenzene group, being −1.4 kcal/mol. Accordingly, ligand design focused on maintaining these types of functional groups on the ligands, while attempting to optimize the overlap of these types of moieties with the FragMaps in the ligand binding site. Concerning the central aromatic ring in UMB18, additional apolar FragMaps were located in the region around this group. The presence of these FragMaps indicated that variations of the central ring, including substitution of that ring with additional rings was considered as such modifications may maintain or lead to improved binding as well as yield novel chemical scaffolds for further optimization.

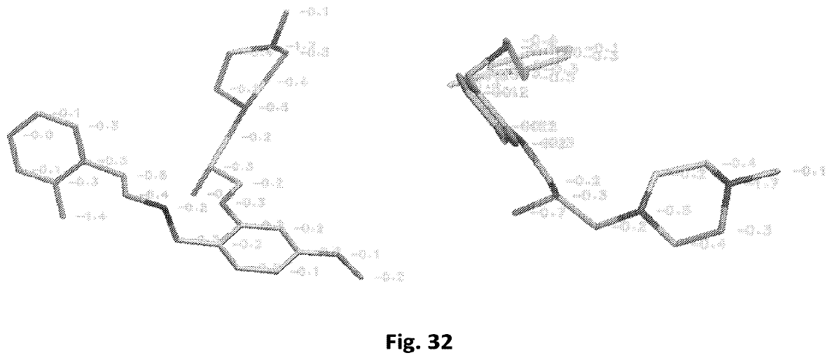

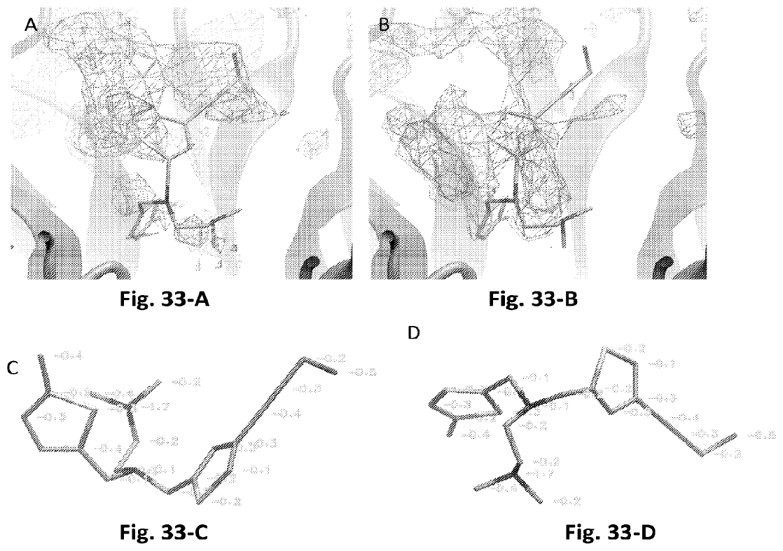

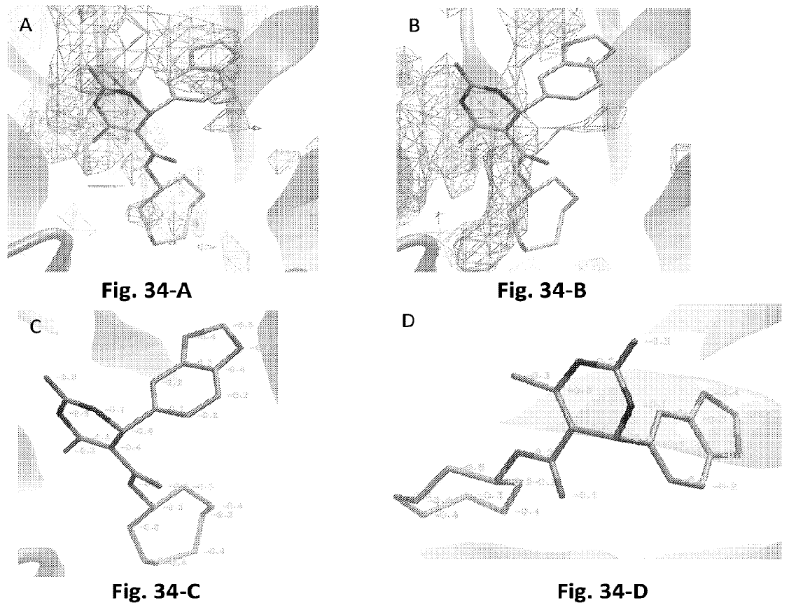

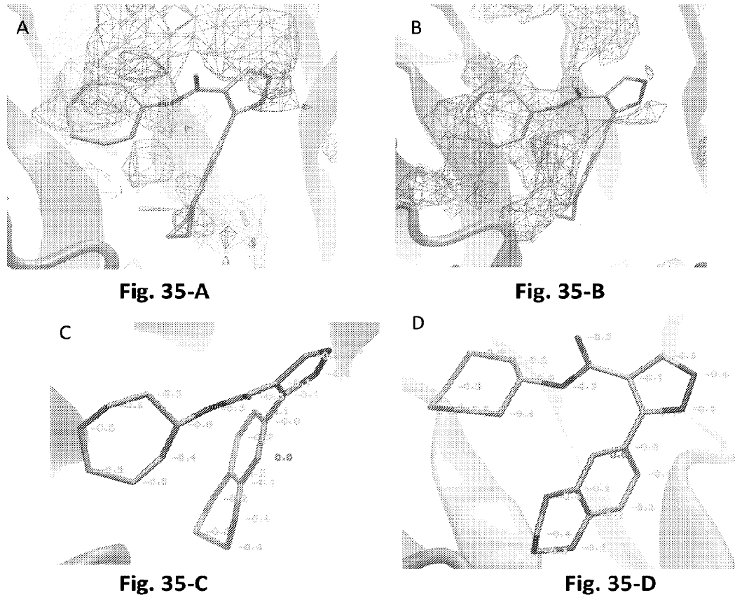

Analysis of the binding orientations, overlap with SILCS FragMaps, and atom-based GFE contributions are shown in FIGS. 33-A to 33-D for UMB28, FIGS. 34-A to 34-D for UMB22_5, and FIGS. 35-A to 35-D for UMB22_16. The overlap of the ligands with the FragMaps and the GFE scores again show most of the atoms to contribute to binding. The basic nitrogen in UMB28 makes a large favorable contribution to binding, −1.7 kcal/mol, as seen with the basic nitrogen in UMB18 above, indicating that it should be maintained during optimization. In addition, the halogen FragMaps overlap with the terminal ring moieties of the compounds suggesting the potential for the addition of halogens to improve activity. Notably, the two UMB22 compounds are neutral making them of interest for further optimization. Also, the orientations of UMB22_5 and UMB22_16 in the binding site are rotated relative to each other, indicating the need systematically identification modifications to clarify binding.

### Example 7. Analog SARS2 Infection Data and Cellular Toxicity Data

Infection: MOI 0.5 for 24 h then fix and stain for N protein. Celigo image for green + or −. Analysis. Infected cells set relative to appropriate DMSO controls. Toxicity based on nuclei count and set relative to DMSO controls.

UMB18G and SILCS 1-3 Plate Setup (Greiner Celigo Plates)

| | 1 | 2 | 3 | 4 | 5 | 6 | 7 | 8 | 9 | 10 | 11 | 12 | Plate 1 |
|---|---|---|---|---|---|---|---|---|---|---|---|---|---|
| A | UMB18G | UMB18G | UMB18G | SILCS1 | SILCS1 | SILCS1 | SILCS2 | SILCS2 | SILCS2 | SILCS3 | SILCS3 | SILCS3 | 50 uM |
| B | | | | | | | | | | | | | 25 uM |
| C | | | | | | | | | | | | | 10 uM |
| D | | | | | | | | | | | | | 5 uM |
| E | | | | | | | | | | | | | 1 uM |
| F | DMSO | | | | | | | | | | | | 0.50% |
| G | DMSO | | | | | | | | | | | | 0.10% |
| H | Uninfect | | | | | | | | | | | | N/A |

Infection Data

Exp 1 (SMW454), Percentage Infection Relative to Appropriate DMSO Control (e.g. Set DMSO as 100%) with Avg Background Subtraction

| | UMB18G | UMB18G | UMB18G | SILCS1 | SILCS1 | SILCS1 | SILCS2 | SILCS2 | SILCS2 | SILCS3 | SILCS3 | SILCS3 |
|---|---|---|---|---|---|---|---|---|---|---|---|---|
| 50 uM | 28.95% | 40.45% | 40.98% | 103.48% | 93.04% | 95.93% | 0.73% | 1.01% | 0.37% | 38.76% | 39.55% | 35.10% |
| 25 uM | 54.89% | 58.50% | 60.94% | 103.38% | 104.01% | 105.61% | −0.21% | 3.73% | 0.29% | 73.36% | 69.10% | 58.07% |
| 10 uM | 84.64% | 105.70% | 105.02% | 107.68% | 118.84% | 98.07% | 41.48% | 34.96% | 28.74% | 98.93% | 87.99% | 82.55% |
| 5 uM | 82.33% | 99.01% | 94.32% | 112.30% | 117.54% | 117.35% | 10.51% | 10.94% | 5.54% | 110.38% | 99.87% | 94.40% |
| 1 uM | 79.68% | 100.00% | 102.30% | 99.35% | 98.10% | 99.72% | 59.76% | 58.66% | 55.59% | 102.18% | 96.08% | 79.31% |

Exp 2 (SMW466), Percentage Infection Relative to Appropriate DMSO Control (e.g. Set DMSO as 1000%) with Avg Background Subtraction

| | UMB18G | UMB18G | UMB18G | SILCS1 | SILCS1 | SILCS1 | SILCS2 | SILCS2 | SILCS2 | SILCS3 | SILCS3 | SILCS3 |
|---|---|---|---|---|---|---|---|---|---|---|---|---|
| 50 uM | −0.97% | −1.80% | 1.96% | 3.78% | 15.08% | 7.39% | 0.67% | 1.28% | 0.54% | −2.52% | −3.00% | 0.03% |
| 25 uM | −0.69% | −1.02% | −1.54% | 28.51% | 36.47% | 30.25% | 8.95% | 10.21% | 7.18% | −2.25% | −2.53% | 0.99% |
| 10 uM | 10.03% | 6.17% | 9.36% | 102.74% | 102.52% | 110.68% | 46.39% | 32.07% | 48.47% | 15.71% | 12.38% | 8.39% |
| 5 uM | 7.98% | 19.27% | 24.86% | 114.43% | 126.13% | 128.85% | 79.60% | 78.23% | 76.50% | 25.85% | 18.37% | 25.06% |
| 1 uM | 48.81% | 100.42% | 112.37% | 148.68% | 144.39% | 143.85% | 119.85% | 132.07% | 119.49% | 97.83% | 85.97% | 85.21% |

Exp 3 (SMW480), Percentage Infection Relative to Appropriate DMSO Control (e.g. Set DMSO as 1000%) with Avg Background Subtraction

| | UMB18G | UMB18G | UMB18G | SILCS1 | SILCS1 | SILCS1 | SILCS2 | SILCS2 | SILCS2 | SILCS3 | SILCS3 | SILCS3 |
|---|---|---|---|---|---|---|---|---|---|---|---|---|
| 50 uM | −1.69% | −2.08% | −2.66% | 27.26% | 20.30% | 23.61% | 3.26% | 6.68% | 5.70% | 9.09% | −2.67% | −1.61% |
| 25 uM | −0.67% | 2.60% | 2.37% | 55.94% | 38.83% | 53.44% | 9.05% | 3.84% | 9.53% | 6.43% | −0.12% | −2.91% |
| 10 uM | 53.71% | 59.47% | 34.88% | 73.36% | 110.69% | 80.10% | 65.71% | 67.81% | 54.75% | 32.26% | 56.01% | 19.98% |
| 5 uM | 19.54% | 24.35% | 30.13% | 127.64% | 114.03% | 119.89% | 75.95% | 53.53% | 59.93% | 27.99% | 23.67% | 10.48% |
| 1 uM | 54.21% | 61.59% | 57.93% | 128.15% | 110.09% | 129.44% | 75.80% | 68.72% | 81.23% | 55.68% | 51.29% | 40.12% |

Average

| | UMB18G | | | SILCS1 | | | SILCS2 | | | SILCS3 | | |
|---|---|---|---|---|---|---|---|---|---|---|---|---|
| | Mean | STD | n | Mean | STD | n | Mean | STD | n | Mean | STD | n |
| 50 uM | 11.46% | 19.35% | 9 | 43.32% | 41.36% | 9 | 2.25% | 2.41% | 9 | 12.53% | 19.35% | 9 |
| 25 uM | 19.49% | 29.04% | 9 | 61.83% | 33.18% | 9 | 5.84% | 4.04% | 9 | 22.24% | 33.80% | 9 |
| 10 uM | 52.11% | 39.99% | 9 | 100.52% | 14.84% | 9 | 46.71% | 14.04% | 9 | 46.02% | 35.93% | 9 |
| 5 uM | 44.64% | 36.19% | 9 | 119.79% | 6.25% | 9 | 50.08% | 32.06% | 9 | 48.45% | 40.35% | 9 |
| 1 uM | 79.70% | 24.56% | 9 | 122.42% | 20.93% | 9 | 85.68% | 29.95% | 9 | 77.07% | 22.53% | 9 |

Toxicity Data

Exp 1 (SMW454), Percentage Infection Relative to Appropriate DMSO Control (e.g. Set DMSO as 1000%) with Avg Background Subtraction

| | UMB18G | UMB18G | UMB18G | SILCS1 | SILCS1 | SILCS1 | SILCS2 | SILCS2 | SILCS2 | SILCS3 | SILCS3 | SILCS3 |
|---|---|---|---|---|---|---|---|---|---|---|---|---|
| 50 uM | 95.5% | 101.6% | 97.7% | 83.8% | 87.1% | 84.9% | 101.0% | 100.7% | 97.3% | 93.7% | 93.9% | 95.7% |
| 25 uM | 98.3% | 107.4% | 105.6% | 94.7% | 94.9% | 95.9% | 74.9% | 75.7% | 66.4% | 102.5% | 101.9% | 95.9% |
| 10 uM | 94.4% | 107.2% | 106.3% | 107.4% | 106.9% | 105.6% | 121.0% | 124.9% | 121.5% | 112.5% | 109.3% | 98.7% |
| 5 uM | 92.6% | 104.0% | 109.1% | 102.9% | 104.5% | 102.2% | 125.8% | 126.0% | 124.9% | 105.8% | 104.1% | 93.5% |
| 1 uM | 94.8% | 106.7% | 107.9% | 105.2% | 108.8% | 106.3% | 115.9% | 118.7% | 118.0% | 108.7% | 108.1% | 97.2% |

Exp 2 (SMW466), Percentage Infection Relative to Appropriate DMSO Control (e.g. Set DMSO as 1000%) with Avg Background Subtraction

| | UMB18G | UMB18G | UMB18G | SILCS1 | SILCS1 | SILCS1 | SILCS2 | SILCS2 | SILCS2 | SILCS3 | SILCS3 | SILCS3 |
|---|---|---|---|---|---|---|---|---|---|---|---|---|
| 50 uM | 35.4% | 68.6% | 89.2% | 88.5% | 77.1% | 78.5% | 106.6% | 79.5% | 90.4% | 81.8% | 76.6% | 53.5% |
| 25 uM | 68.6% | 126.9% | 132.7% | 130.6% | 124.7% | 133.0% | 142.9% | 140.7% | 125.1% | 131.0% | 122.3% | 75.1% |
| 10 uM | 78.2% | 135.1% | 144.2% | 107.3% | 102.2% | 104.0% | 129.5% | 133.5% | 129.4% | 139.8% | 146.3% | 105.1% |
| 5 uM | 80.8% | 132.0% | 142.0% | 107.1% | 94.1% | 106.7% | 120.5% | 119.7% | 121.4% | 143.6% | 141.9% | 102.7% |
| 1 uM | 77.1% | 105.3% | 105.6% | 93.5% | 94.5% | 96.6% | 103.5% | 102.5% | 106.6% | 110.2% | 122.4% | 99.6% |

Exp 3 (SMW480), Percentage Infection Relative to Appropriate DMSO Control (e.g. Set DMSO as 1000%) with Avg Background Subtraction

| | UMB18G | UMB18G | UMB18G | SILCS1 | SILCS1 | SILCS1 | SILCS2 | SILCS2 | SILCS2 | SILCS3 | SILCS3 | SILCS3 |
|---|---|---|---|---|---|---|---|---|---|---|---|---|
| 50 uM | 60.8% | 69.5% | 85.1% | 72.1% | 98.8% | 86.4% | 120.3% | 125.4% | 128.4% | 117.7% | 89.8% | 80.0% |
| 25 uM | 70.7% | 138.9% | 163.1% | 120.8% | 130.3% | 121.5% | 168.2% | 165.8% | 166.8% | 157.9% | 155.8% | 114.5% |
| 10 uM | 89.6% | 123.8% | 150.9% | 124.6% | 98.4% | 120.2% | 131.9% | 128.0% | 135.9% | 156.3% | 129.5% | 122.8% |
| 5 uM | 113.4% | 154.3% | 157.5% | 88.1% | 95.7% | 94.5% | 120.7% | 133.1% | 133.9% | 154.5% | 147.8% | 136.1% |
| 1 uM | 110.1% | 127.5% | 137.1% | 89.7% | 102.3% | 92.4% | 124.8% | 128.6% | 118.0% | 131.1% | 136.5% | 118.6% |

Average

| | UMB18G | | | SILCS1 | | | SILCS2 | | | SILCS3 | | |
|---|---|---|---|---|---|---|---|---|---|---|---|---|
| | Mean | STD | n | Mean | STD | n | Mean | STD | n | Mean | STD | n |
| 50 uM | 78.15% | 21.50% | 9 | 84.14% | 7.70% | 9 | 105.51% | 16.41% | 9 | 86.97% | 17.41% | 9 |
| 25 uM | 112.46% | 31.28% | 9 | 116.28% | 16.36% | 9 | 125.16% | 42.16% | 9 | 117.43% | 27.51% | 9 |
| 10 uM | 114.41% | 25.45% | 9 | 108.52% | 8.45% | 9 | 128.40% | 5.15% | 9 | 124.47% | 19.87% | 9 |
| 5 uM | 120.64% | 27.18% | 9 | 99.54% | 6.63% | 9 | 125.09% | 5.31% | 9 | 125.56% | 23.55% | 9 |
| 1 uM | 108.03% | 17.25% | 9 | 98.82% | 6.94% | 9 | 115.19% | 9.17% | 9 | 114.70% | 13.49% | 9 |

SILCS 4-7 Plate Setup (Greiner Celigo Plates)

| | 1 | 2 | 3 | 4 | 5 | 6 | 7 | 8 | 9 | 10 | 11 | 12 | Plate 2 |
|---|---|---|---|---|---|---|---|---|---|---|---|---|---|
| A | SILCS4 | SILCS4 | SILCS4 | SILCS5 | SILCS5 | SILCS5 | SILCS6 | SILCS6 | SILCS6 | SIL CS7 | SILCS7 | SILCS7 | 50 uM |
| B | | | | | | | | | | | | | 25 uM |
| C | | | | | | | | | | | | | 10 uM |
| D | | | | | | | | | | | | | 5 uM |
| E | | | | | | | | | | | | | 1 uM |
| F | DMSO | | | | | | | | | | | | 0.50% |
| G | DMSO | | | | | | | | | | | | 0.10% |
| H | Uninfect | | | | | | | | | | | | N/A |

Infection Data

Exp 1 (SMW454), Percentage Infection Relative to Appropriate DMSO Control (e.g. Set DMSO as 1000%) with Avg Background Subtraction

| | SILCS4 | SILCS4 | SILCS4 | SILCS5 | SILCS5 | SILCS5 | SILCS6 | SILCS6 | SILCS6 | SILCS7 | SILCS7 | SILCS7 |
|---|---|---|---|---|---|---|---|---|---|---|---|---|
| 50 uM | 1.78% | 4.05% | 5.38% | 99.43% | 98.03% | 105.18% | 64.09% | 59.84% | 71.42% | 30.26% | 28.66% | 29.62% |
| 25 uM | 7.53% | 9.36% | 6.29% | 101.61% | 86.58% | 84.86% | 85.42% | 90.94% | 91.57% | 55.66% | 51.00% | 52.63% |
| 10 uM | 0.31% | 1.23% | 1.40% | 107.17% | 100.08% | 106.58% | 119.50% | 97.81% | 119.68% | 100.29% | 93.67% | 81.37% |
| 5 uM | 41.46% | 35.03% | 29.67% | 120.29% | 104.68% | 72.54% | 117.29% | 106.47% | 108.56% | 109.44% | 103.86% | 95.56% |
| 1 uM | 75.22% | 94.87% | 93.18% | 118.07% | 104.73% | 116.82% | 121.30% | 114.60% | 105.92% | 117.21% | 101.49% | 86.01% |

Exp 2 (SMW466), Percentage Infection Relative to Appropriate DMSO Control (e.g. Set DMSO as 1000%) with Avg Background Subtraction

| | SILCS4 | SILCS4 | SILCS4 | SILCS5 | SILCS5 | SILCS5 | SILCS6 | SILCS6 | SILCS6 | SILCS7 | SILCS7 | SILCS7 |
|---|---|---|---|---|---|---|---|---|---|---|---|---|
| 50 uM | NaN | NaN | NaN | 8.11% | 13.10% | 6.71% | 26.76% | 16.78% | 39.12% | 15.27% | 10.01% | 41.20% |
| 25 uM | NaN | NaN | NaN | 37.53% | 50.58% | 37.18% | 59.24% | 75.43% | 85.65% | 25.13% | 12.79% | 12.59% |
| 10 uM | 1.95% | −0.35% | 1.22% | 122.53% | 131.50% | 102.34% | 141.78% | 132.35% | 72.38% | 72.63% | 48.59% | 28.00% |
| 5 uM | 10.04% | 20.92% | 14.96% | 112.69% | 142.31% | 85.76% | 106.84% | 100.88% | 92.01% | 118.70% | 71.95% | 54.80% |
| 1 uM | 77.30% | 92.74% | 93.22% | 146.66% | 109.55% | 128.21% | 87.69% | 82.40% | 130.25% | 120.54% | 113.42% | 103.47% |

Exp 3 (SMW480), Percentage Infection Relative to Appropriate DMSO Control (e.g. Set DMSO as 1000%) with Avg Background Subtraction

| | SILCS4 | SILCS4 | SILCS4 | SILCS5 | SILCS5 | SILCS5 | SILCS6 | SILCS6 | SILCS6 | SILCS7 | SILCS7 | SILCS7 |
|---|---|---|---|---|---|---|---|---|---|---|---|---|
| 50 uM | 12.74% | 12.96% | 18.11% | 119.00% | 119.00% | 112.15% | 2.07% | 1.65% | 1.29% | 51.47% | 43.21% | 30.10% |
| 25 uM | 27.97% | 30.69% | 30.39% | 115.50% | 101.91% | 115.75% | 1.31% | 1.94% | 3.21% | 65.89% | 49.71% | 52.28% |
| 10 uM | 45.89% | 58.06% | 14.41% | 62.23% | 78.25% | 76.57% | 63.68% | 16.71% | 29.68% | 26.92% | 38.92% | 28.98% |
| 5 uM | 65.26% | 83.05% | 60.10% | 100.67% | 104.29% | 100.35% | 6.41% | 2.68% | 2.48% | 82.34% | 97.08% | 101.56% |
| 1 uM | 80.50% | 89.48% | 45.87% | 100.33% | 97.57% | 85.79% | 31.72% | 15.60% | 26.77% | 70.06% | 59.76% | 73.40% |

Average

| | SILCS4 | | | SILCS5 | | | SILCS6 | | | SILCS7 | | |
|---|---|---|---|---|---|---|---|---|---|---|---|---|
| | Mean | STD | n | Mean | STD | n | Mean | STD | n | Mean | STD | n |
| 50 uM | 9.17% | 6.36% | 9 | 75.63% | 50.32% | 9 | 31.45% | 28.34% | 9 | 31.09% | 13.08% | 9 |
| 25 uM | 18.71% | 12.10% | 9 | 81.28% | 31.70% | 9 | 54.97% | 40.79% | 9 | 41.96% | 19.75% | 9 |
| 10 uM | 13.79% | 22.31% | 9 | 98.58% | 22.45% | 9 | 88.17% | 45.10% | 9 | 57.71% | 29.54% | 9 |
| 5 uM | 40.06% | 24.79% | 9 | 104.84% | 19.84% | 9 | 71.51% | 51.19% | 9 | 92.81% | 19.88% | 9 |
| 1 uM | 82.49% | 15.66% | 9 | 111.97% | 18.08% | 9 | 79.58% | 43.98% | 9 | 93.93% | 22.36% | 9 |

Toxicity Data

Exp 1 (SMW454), Percentage Infection Relative to Appropriate DMSO Control (e.g. Set DMSO as 1000%) with Avg Background Subtraction

| | SILCS4 | SILCS4 | SILCS4 | SILCS5 | SILCS5 | SILCS5 | SILCS6 | SILCS6 | SILCS6 | SILCS7 | SILCS7 | SILCS7 |
|---|---|---|---|---|---|---|---|---|---|---|---|---|
| 50 uM | 6.0% | 6.8% | 5.2% | 83.7% | 83.6% | 81.3% | 88.0% | 87.4% | 83.4% | 91.4% | 92.5% | 88.1% |
| 25 uM | 10.0% | 10.3% | 10.1% | 97.3% | 98.6% | 95.6% | 95.7% | 97.9% | 97.2% | 100.4% | 105.0% | 94.5% |
| 10 uM | 107.4% | 120.3% | 122.0% | 103.9% | 104.5% | 100.7% | 99.2% | 104.5% | 97.6% | 100.9% | 106.3% | 90.3% |
| 5 uM | 99.1% | 113.1% | 119.4% | 100.0% | 103.1% | 104.5% | 99.3% | 103.7% | 101.5% | 102.2% | 102.9% | 89.0% |
| 1 uM | 93.8% | 104.8% | 105.3% | 102.7% | 101.6% | 101.1% | 99.7% | 106.1% | 102.4% | 98.8% | 103.0% | 90.4% |

Exp 2 (SMW466), Percentage Infection Relative to Appropriate DMSO Control (e.g. Set DMSO as 1000%) with Avg Background Subtraction

| | SILCS4 | SILCS4 | SILCS4 | SILCS5 | SILCS5 | SILCS5 | SILCS6 | SILCS6 | SILCS6 | SILCS7 | SILCS7 | SILCS7 |
|---|---|---|---|---|---|---|---|---|---|---|---|---|
| 50 uM | 0.0% | 0.0% | 0.0% | 47.0% | 67.4% | 63.3% | 63.7% | 63.4% | 66.1% | 63.4% | 55.1% | 33.8% |
| 25 uM | 0.1% | 0.0% | 0.0% | 111.8% | 111.4% | 111.9% | 110.3% | 98.9% | 107.6% | 111.8% | 98.0% | 71.7% |
| 10 uM | 76.2% | 122.0% | 120.7% | 108.4% | 112.0% | 116.9% | 94.3% | 117.7% | 112.8% | 114.8% | 117.9% | 92.0% |
| 5 uM | 88.5% | 134.2% | 112.4% | 118.9% | 109.4% | 120.2% | 118.1% | 122.1% | 119.7% | 109.7% | 104.5% | 89.4% |
| 1 uM | 87.2% | 116.8% | 103.4% | 108.6% | 118.9% | 114.4% | 124.1% | 115.9% | 104.8% | 111.7% | 103.8% | 88.9% |

Exp 3 (SMW480), Percentage Infection Relative to Appropriate DMSO Control (e.g. Set DMSO as 1000%) with Avg Background Subtraction

| | SILCS4 | SILCS4 | SILCS4 | SILCS5 | SILCS5 | SILCS5 | SILCS6 | SILCS6 | SILCS6 | SILCS7 | SILCS7 | SILCS7 |
|---|---|---|---|---|---|---|---|---|---|---|---|---|
| 50 uM | 0.5% | 0.7% | 0.4% | 89.3% | 70.5% | 94.1% | 90.9% | 89.3% | 70.8% | 85.6% | 77.1% | 76.6% |
| 25 uM | 1.7% | 1.5% | 1.4% | 105.5% | 106.5% | 109.0% | 105.9% | 112.1% | 106.9% | 138.8% | 118.3% | 92.9% |
| 10 uM | 102.1% | 156.6% | 171.2% | 81.5% | 98.7% | 98.4% | 100.5% | 109.0% | 102.5% | 126.6% | 113.9% | 96.4% |
| 5 uM | 117.7% | 171.4% | 159.3% | 105.0% | 111.0% | 107.0% | 115.4% | 125.2% | 124.7% | 125.4% | 111.7% | 98.0% |
| 1 uM | 129.2% | 152.6% | 152.9% | 134.4% | 112.0% | 130.0% | 131.8% | 128.0% | 148.1% | 148.0% | 143.4% | 109.5% |

Average

| | SILCS4 | | | SILCS5 | | | SILCS6 | | | SILCS7 | | |
|---|---|---|---|---|---|---|---|---|---|---|---|---|
| | Mean | STD | n | Mean | STD | n | Mean | STD | n | Mean | STD | n |
| 50 uM | 2.18% | 2.90% | 9 | 75.59% | 14.82% | 9 | 78.10% | 11.86% | 9 | 73.73% | 19.61% | 9 |
| 25 uM | 3.91% | 4.71% | 9 | 105.30% | 6.53% | 9 | 103.61% | 6.19% | 9 | 103.49% | 18.65% | 9 |
| 10 uM | 122.04% | 28.12% | 9 | 102.76% | 10.09% | 9 | 104.23% | 7.59% | 9 | 106.58% | 12.56% | 9 |
| 5 uM | 123.91% | 26.90% | 9 | 108.79% | 6.92% | 9 | 114.40% | 10.20% | 9 | 103.66% | 11.33% | 9 |
| 1 uM | 116.22% | 23.97% | 9 | 113.75% | 12.11% | 9 | 117.87% | 16.33% | 9 | 110.82% | 21.22% | 9 |

SILCS 8-11 Plate Setup (Greiner Celigo Plates)

| | 1 | 2 | 3 | 4 | 5 | 6 | 7 | 8 | 9 | 10 | 11 | 12 | Plate 3 |
|---|---|---|---|---|---|---|---|---|---|---|---|---|---|
| A | SILCS8 | SILCS8 | SILCS8 | SILCS9 | SILCS9 | SILCS9 | SILCS10 | SILCS10 | SILCS10 | SILCS11 | SILCS11 | SILCS11 | 50 uM |
| B | | | | | | | | | | | | | 25 uM |
| C | | | | | | | | | | | | | 10 uM |
| D | | | | | | | | | | | | | 5 uM |
| E | | | | | | | | | | | | | 1 uM |
| F | DMSO | | | | | | | | | | | | 0.50% |
| G | DMSO | | | | | | | | | | | | 0.10% |
| H | Uninfect | | | | | | | | | | | | N/A |

Infection Data

Exp 1 (SMW454), Percentage Infection Relative to Appropriate DMSO Control (e.g. Set DMSO as 1000%) with Avg Background Subtraction

| | SILCS8 | SILCS8 | SILCS8 | SILCS9 | SILCS9 | SILCS9 | SILCS10 | SILCS10 | SILCS10 | SILCS11 | SILCS11 | SILCS11 |
|---|---|---|---|---|---|---|---|---|---|---|---|---|
| 50 uM | 6.94% | 0.29% | 1.14% | 37.04% | 35.36% | 27.11% | 13.95% | 10.70% | 8.82% | 1.77% | −1.21% | −1.14% |
| 25 uM | 3.90% | 2.54% | 4.06% | 65.27% | 68.54% | 70.06% | 30.35% | 25.20% | 10.04% | 7.81% | 1.22% | 1.37% |
| 10 uM | 29.85% | 29.73% | 18.98% | 114.87% | 99.89% | 93.84% | 51.56% | 53.74% | 59.11% | 24.55% | 29.13% | 19.97% |
| 5 uM | 47.89% | 55.36% | 47.76% | 111.45% | 100.42% | 106.27% | 75.16% | 58.30% | 59.92% | 46.20% | 46.44% | 40.61% |
| 1 uM | 81.05% | 90.21% | 96.89% | 117.17% | 102.02% | 127.17% | 93.22% | 90.46% | 93.41% | 81.09% | 69.28% | 64.76% |

Exp 2 (SMW466), Percentage Infection Relative to Appropriate DMSO Control (e.g. Set DMSO as 1000%) with Avg Background Subtraction

| | SILCS8 | SILCS8 | SILCS8 | SILCS9 | SILCS9 | SILCS9 | SILCS10 | SILCS10 | SILCS10 | SILCS11 | SILCS11 | SILCS11 |
|---|---|---|---|---|---|---|---|---|---|---|---|---|
| 50 uM | 22.51% | 12.92% | 11.13% | 69.35% | 253.51% | 89.82% | 0.88% | 8.12% | 18.84% | 27.08% | 20.27% | 16.47% |
| 25 uM | 30.23% | 25.86% | 33.22% | 137.05% | 187.92% | 140.10% | 100.45% | 82.69% | 59.57% | 70.22% | 49.24% | 21.32% |
| 10 uM | 76.22% | 83.74% | 125.84% | 183.60% | 143.74% | 234.68% | 33.34% | 37.62% | 34.35% | 127.25% | 120.97% | 76.77% |
| 5 uM | 90.06% | 122.88% | 109.63% | 175.91% | 148.50% | 137.95% | 75.47% | 61.37% | 60.31% | 112.35% | 123.89% | 56.94% |
| 1 uM | 94.60% | 126.80% | 88.18% | 107.67% | 125.65% | 155.98% | 52.44% | 26.67% | 49.75% | 114.56% | 97.31% | 54.00% |

Exp 3 (SMW480), Percentage Infection Relative to Appropriate DMSO Control (e.g. Set DMSO as 1000%) with Avg Background Subtraction

| | SILCS8 | SILCS8 | SILCS8 | SILCS9 | SILCS9 | SILCS9 | SILCS10 | SILCS10 | SILCS10 | SILCS11 | SILCS11 | SILCS11 |
|---|---|---|---|---|---|---|---|---|---|---|---|---|
| 50 uM | 10.14% | 10.37% | 0.00% | 119.56% | 119.57% | 112.52% | −0.84% | −1.28% | −1.65% | 50.03% | 41.52% | 28.02% |
| 25 uM | 25.83% | 0.00% | 0.00% | 115.96% | 101.97% | 116.22% | −1.63% | −0.97% | 0.33% | 64.88% | 48.22% | 50.86% |
| 10 uM | 44.30% | 56.83% | 11.89% | 61.12% | 77.61% | 75.88% | 62.62% | 14.26% | 27.61% | 24.77% | 37.12% | 26.89% |
| 5 uM | 64.24% | 82.55% | 58.93% | 100.69% | 104.42% | 100.36% | 3.66% | −0.18% | −0.39% | 81.82% | 96.99% | 101.60% |
| 1 uM | 79.93% | 89.17% | 44.28% | 100.34% | 97.50% | 85.37% | 29.71% | 13.12% | 24.61% | 69.18% | 58.57% | 72.62% |

Average

| | SILCS8 | | | SILCS9 | | | SILCS10 | | | SILCS11 | | |
|---|---|---|---|---|---|---|---|---|---|---|---|---|
| | Mean | STD | n | Mean | STD | n | Mean | STD | n | Mean | STD | n |
| 50 uM | 8.38% | 7.29% | 9 | 95.98% | 69.68% | 9 | 6.39% | 7.46% | 9 | 20.31% | 18.44% | 9 |
| 25 uM | 13.96% | 14.31% | 9 | 111.45% | 40.53% | 9 | 34.00% | 38.29% | 9 | 35.01% | 27.30% | 9 |
| 10 uM | 53.04% | 36.75% | 9 | 120.58% | 57.10% | 9 | 41.58% | 16.10% | 9 | 54.16% | 43.14% | 9 |
| 5 uM | 75.48% | 27.41% | 9 | 120.66% | 27.16% | 9 | 43.73% | 32.67% | 9 | 78.54% | 31.75% | 9 |
| 1 uM | 87.90% | 21.39% | 9 | 113.21% | 21.03% | 9 | 52.60% | 32.20% | 9 | 75.71% | 19.31% | 9 |

Toxicity Data

Exp 1 (SMW454), Percentage Infection Relative to Appropriate DMSO Control (e.g. Set DMSO as 1000%) with Avg Background Subtraction

| | SILCS8 | SILCS8 | SILCS8 | SILCS9 | SILCS9 | SILCS9 | SILCS10 | SILCS10 | SILCS10 | SILCS11 | SILCS11 | SILCS11 |
|---|---|---|---|---|---|---|---|---|---|---|---|---|
| 50 uM | 101.9% | 105.0% | 108.1% | 96.3% | 98.8% | 97.1% | 106.3% | 104.3% | 105.2% | 105.9% | 104.0% | 100.5% |
| 25 uM | 110.5% | 120.6% | 123.5% | 107.1% | 104.3% | 103.9% | 116.2% | 116.2% | 119.3% | 120.8% | 120.1% | 106.2% |
| 10 uM | 109.0% | 120.6% | 127.6% | 109.1% | 108.1% | 106.5% | 121.9% | 116.2% | 118.2% | 132.7% | 124.1% | 108.9% |
| 5 uM | 103.8% | 112.3% | 118.6% | 104.3% | 108.1% | 102.8% | 112.2% | 115.0% | 114.9% | 120.3% | 117.9% | 103.9% |
| 1 uM | 100.6% | 104.0% | 108.8% | 102.3% | 106.6% | 101.3% | 106.9% | 108.4% | 105.4% | 114.1% | 111.2% | 97.9% |

Exp 2 (SMW466), Percentage Infection Relative to Appropriate DMSO Control (e.g. Set DMSO as 1000%) with Avg Background Subtraction

| | SILCS8 | SILCS8 | SILCS8 | SILCS9 | SILCS9 | SILCS9 | SILCS10 | SILCS10 | SILCS10 | SILCS11 | SILCS11 | SILCS11 |
|---|---|---|---|---|---|---|---|---|---|---|---|---|
| 50 uM | 43.7% | 65.6% | 67.9% | 61.9% | 3.8% | 63.8% | 42.1% | 72.2% | 56.0% | 63.7% | 60.9% | 41.5% |
| 25 uM | 65.0% | 102.6% | 110.3% | 97.3% | 91.8% | 94.9% | 0.1% | 0.2% | 0.3% | 102.1% | 96.3% | 67.0% |
| 10 uM | 78.4% | 105.1% | 112.6% | 95.2% | 102.9% | 84.9% | 110.8% | 124.8% | 118.7% | 104.4% | 102.6% | 79.6% |
| 5 uM | 80.1% | 109.7% | 113.1% | 100.6% | 109.3% | 89.5% | 107.1% | 108.2% | 116.3% | 108.3% | 104.7% | 79.8% |
| 1 uM | 82.0% | 102.2% | 115.5% | 113.0% | 110.8% | 80.0% | 112.5% | 119.2% | 120.6% | 108.4% | 103.5% | 83.4% |

Exp 3 (SMW480), Percentage Infection Relative to Appropriate DMSO Control (e.g. Set DMSO as 1000%) with Avg Background Subtraction

| | SILCS8 | SILCS8 | SILCS8 | SILCS9 | SILCS9 | SILCS9 | SILCS10 | SILCS10 | SILCS10 | SILCS11 | SILCS11 | SILCS11 |
|---|---|---|---|---|---|---|---|---|---|---|---|---|
| 50 uM | 63.5% | 67.0% | 63.2% | 39.2% | 40.8% | 44.4% | 67.5% | 102.6% | 100.5% | 92.4% | 82.7% | 71.9% |
| 25 uM | 65.5% | 111.5% | 139.7% | 70.1% | 90.1% | 70.4% | 46.5% | 25.8% | 29.4% | 118.7% | 129.2% | 92.9% |
| 10 uM | 115.4% | 139.7% | 181.2% | 139.0% | 125.6% | 129.3% | 130.1% | 169.7% | 168.7% | 155.3% | 146.8% | 130.4% |
| 5 uM | 102.0% | 120.9% | 144.1% | 106.6% | 104.5% | 106.4% | 197.5% | 197.2% | 202.4% | 123.1% | 108.3% | 89.0% |
| 1 uM | 99.7% | 113.0% | 147.7% | 105.4% | 108.5% | 120.5% | 163.1% | 179.3% | 172.2% | 134.0% | 134.4% | 112.1% |

Average

| | SILCS8 | | | SILCS9 | | | SILCS10 | | | SILCS11 | | |
|---|---|---|---|---|---|---|---|---|---|---|---|---|
| | Mean | STD | n | Mean | STD | n | Mean | STD | n | Mean | STD | n |
| 50 uM | 76.22% | 22.80% | 9 | 60.67% | 32.42% | 9 | 84.08% | 24.81% | 9 | 80.38% | 22.37% | 9 |
| 25 uM | 105.46% | 25.15% | 9 | 92.21% | 13.72% | 9 | 50.46% | 52.46% | 9 | 105.91% | 19.09% | 9 |
| 10 uM | 121.05% | 28.12% | 9 | 111.19% | 17.19% | 9 | 131.00% | 22.32% | 9 | 120.55% | 23.84% | 9 |
| 5 uM | 111.63% | 17.12% | 9 | 103.56% | 5.91% | 9 | 141.21% | 43.52% | 9 | 106.15% | 14.30% | 9 |
| 1 uM | 108.17% | 17.70% | 9 | 105.37% | 11.14% | 9 | 131.94% | 30.39% | 9 | 111.01% | 16.14% | 9 |

SILCS 12-13 and Untreated Plate Setup (Wherein UT Indicates Untreated, Greiner Celigo Plates)

| | 1 | 2 | 3 | 4 | 5 | 6 | 7 | 8 | 9 | 10 | 11 | 12 | Plate 4 |
|---|---|---|---|---|---|---|---|---|---|---|---|---|---|
| A | SILCS12 | SILCS12 | SILCS12 | SILCS13 | SILCS13 | SILCS13 | UT | UT | UT | UT | UT | UT | 50 uM |
| B | | | | | | | | | | | | | 25 uM |
| C | | | | | | | | | | | | | 10 uM |
| D | | | | | | | | | | | | | 5 uM |
| E | | | | | | | | | | | | | 1 uM |
| F | DMSO | | | | | | | | | | | | 0.50% |
| G | DMSO | | | | | | | | | | | | 0.10% |
| H | Uninfect | | | | | | | | | | | | N/A |

Infection Data

Exp 1 (SMW454), Percentage Infection Relative to Appropriate DMSO Control (e.g. Set DMSO as 1000%) with Avg Background Subtraction

| | SILCS12 | SILCS12 | SILCS12 | SILCS13 | SILCS13 | SILCS13 | UT | UT | UT | UT | UT | UT |
|---|---|---|---|---|---|---|---|---|---|---|---|---|
| 50 | 71.33% | 77.54% | 78.82% | 119.43% | 105.93% | 3.73% | 129.32% | 114.68% | 129.27% | 374.26% | 191.64% | 105.34% |
| uM | | | | | | | | | | | | |
| 25 | 146.63% | 130.77% | 107.50% | 110.77% | 84.96% | 110.16% | 108.25% | 111.43% | 230.65% | 115.90% | 118.82% | 130.14% |
| uM | | | | | | | | | | | | |
| 10 | 118.13% | 105.44% | 132.64% | 93.49% | 94.85% | 117.66% | 72.75% | 64.00% | 127.37% | 92.22% | 98.74% | 99.91% |
| uM | | | | | | | | | | | | |
| 5 | 166.01% | 110.80% | 130.31% | 94.82% | 87.57% | 103.74% | 81.28% | 89.08% | 79.17% | 79.91% | 79.91% | 76.87% |
| uM | | | | | | | | | | | | |
| 1 | 130.66% | 219.91% | 120.32% | 99.33% | 94.08% | 104.59% | 122.13% | 117.79% | 100.66% | 85.09% | 111.02% | 114.12% |
| uM | | | | | | | | | | | | |

Exp 2 (SMW466), Percentage Infection Relative to Appropriate DMSO Control (e.g. Set DMSO as 1000%) with Avg Background Subtraction

| | SILCS12 | SILCS12 | SILCS12 | SILCS13 | SILCS13 | SILCS13 | UT | UT | UT | UT | UT | UT |
|---|---|---|---|---|---|---|---|---|---|---|---|---|
| 50 | 106.93% | 63.16% | 51.38% | 89.22% | 98.62% | 79.14% | 147.09% | 181.92% | 204.82% | 154.12% | 149.98% | 172.32% |
| uM | | | | | | | | | | | | |
| 25 | 127.15% | 129.27% | 188.78% | 232.43% | 190.48% | 194.70% | 292.34% | 393.94% | 281.20% | 231.69% | 178.04% | 112.83% |
| uM | | | | | | | | | | | | |
| 10 | 79.79% | 119.41% | 128.53% | 146.42% | 173.51% | 123.77% | 128.89% | 141.81% | 92.42% | 95.12% | 109.15% | 55.97% |
| uM | | | | | | | | | | | | |
| 5 | 75.86% | 109.63% | 75.26% | 101.09% | 94.30% | 84.67% | 219.23% | 158.54% | 125.92% | 98.18% | 78.51% | 60.41% |
| uM | | | | | | | | | | | | |
| 1 | 115.89% | 104.49% | 72.23% | 105.44% | 58.84% | 84.14% | 137.50% | 129.24% | 154.30% | 115.26% | 127.06% | 56.39% |
| uM | | | | | | | | | | | | |

Exp 3 (SMW480), Percentage Infection Relative to Appropriate DMSO Control (e.g. Set DMSO as 1000%) with a Background Subtraction

| | SILCS12 | SILCS12 | SILCS12 | SILCS13 | SILCS13 | SILCS13 | UT | UT | UT | UT | UT | UT |
|---|---|---|---|---|---|---|---|---|---|---|---|---|
| 50 | 85.05% | 77.01% | 78.70% | 93.99% | 93.46% | 87.24% | 108.21% | 118.63% | 118.15% | 113.54% | 81.90% | 82.02% |
| uM | | | | | | | | | | | | |
| 25 | 129.73% | 138.95% | 117.31% | 122.63% | 116.94% | 116.45% | 135.51% | 142.18% | 123.12% | 124.69% | 108.80% | 101.50% |
| uM | | | | | | | | | | | | |
| 10 | 120.31% | 99.46% | 89.00% | 121.26% | 103.45% | 113.61% | 86.09% | 92.41% | 91.05% | 105.21% | 103.80% | 92.29% |
| uM | | | | | | | | | | | | |
| 5 | 104.83% | 118.10% | 90.23% | 127.22% | 94.60% | 92.81% | 83.92% | 90.30% | 85.85% | 99.50% | 114.31% | 93.20% |
| uM | | | | | | | | | | | | |
| 1 | 100.22% | 108.59% | 111.59% | 119.98% | 98.99% | 86.87% | 89.48% | 79.29% | 105.90% | 99.33% | 118.58% | 93.10% |
| uM | | | | | | | | | | | | |

Average

| | SILCS12 | | | SILCS13 | | |
|---|---|---|---|---|---|---|
| | Mean | STD | n | Mean | STD | n |
| 50 uM | 76.66% | 15.19% | 9 | 85.64% | 32.82% | 9 |
| 25 uM | 135.12% | 23.05% | 9 | 142.17% | 50.25% | 9 |
| 10 uM | 110.30% | 18.03% | 9 | 120.89% | 25.56% | 9 |
| 5 uM | 109.01% | 28.28% | 9 | 97.87% | 12.47% | 9 |
| 1 uM | 120.43% | 40.66% | 9 | 94.70% | 17.14% | 9 |

Toxicity Data

Exp 1 (SMW454), Percentage Infection Relative to Appropriate DMSO Control (e.g. Set DMSO as 100%) with Avg Background Subtraction

| | SILCS12 | SILCS12 | SILCS12 | SILCS13 | SILCS13 | SILCS13 | UT | UT | UT | UT | UT | UT |
|---|---|---|---|---|---|---|---|---|---|---|---|---|
| 50 uM | 102.6% | 95.4% | 100.8% | 98.4% | 98.6% | 107.6% | 87.7% | 87.9% | 86.6% | 92.8% | 90.7% | 87.9% |
| 25 uM | 104.3% | 104.3% | 113.9% | 105.6% | 109.3% | 107.3% | 96.1% | 94.4% | 97.8% | 100.7% | 97.7% | 90.2% |
| 10 uM | 88.5% | 97.4% | 104.5% | 104.8% | 102.5% | 98.4% | 97.5% | 102.8% | 97.5% | 99.4% | 100.6% | 92.8% |
| 5 uM | 88.1% | 100.0% | 101.7% | 106.4% | 100.2% | 95.1% | 96.5% | 104.6% | 96.9% | 101.4% | 100.6% | 96.8% |
| 1 uM | 88.5% | 96.8% | 100.3% | 96.9% | 95.4% | 97.7% | 94.9% | 98.9% | 100.0% | 105.6% | 95.4% | 90.8% |

Exp 2 (SMW466), Percentage Infection Relative to Appropriate DMSO Control (e.g. Set DMSO as 1000%) with Avg Background Subtraction

| | SILCS12 | SILCS12 | SILCS12 | SILCS13 | SILCS13 | SILCS13 | UT | UT | UT | UT | UT | UT |
|---|---|---|---|---|---|---|---|---|---|---|---|---|
| 50 uM | 45.4% | 63.4% | 64.6% | 67.2% | 62.1% | 72.8% | 57.5% | 66.5% | 67.7% | 63.0% | 59.8% | 34.4% |
| 25 uM | 68.6% | 94.4% | 90.0% | 94.5% | 94.0% | 99.0% | 92.4% | 84.5% | 93.3% | 90.7% | 92.9% | 60.7% |
| 10 uM | 83.2% | 106.6% | 106.4% | 94.9% | 107.6% | 109.2% | 95.8% | 113.5% | 114.3% | 115.7% | 107.0% | 84.2% |
| 5 uM | 96.3% | 115.2% | 122.1% | 114.5% | 116.2% | 117.2% | 97.6% | 103.2% | 107.4% | 109.8% | 113.1% | 90.3% |
| 1 uM | 93.5% | 120.4% | 118.4% | 109.7% | 124.2% | 115.2% | 107.2% | 110.0% | 105.6% | 114.6% | 99.7% | 86.6% |

Exp 3 (SMW480), Percentage Infection Relative to Appropriate DMSO Control (e.g. Set DMSO as 1000%) with Avg Background Subtraction

| | SILCS12 | SILCS12 | SILCS12 | SILCS13 | SILCS13 | SILCS13 | UT | UT | UT | UT | UT | UT |
|---|---|---|---|---|---|---|---|---|---|---|---|---|
| 50 uM | 36.1% | 45.0% | 49.2% | 69.3% | 66.5% | 65.1% | 71.3% | 86.7% | 73.2% | 75.9% | 64.3% | 51.2% |
| 25 uM | 65.8% | 108.2% | 108.6% | 109.3% | 108.4% | 111.3% | 127.2% | 121.7% | 113.4% | 118.1% | 101.3% | 69.4% |
| 10 uM | 87.2% | 99.1% | 92.0% | 115.0% | 104.7% | 110.2% | 92.7% | 96.7% | 92.8% | 108.0% | 102.2% | 79.0% |
| 5 uM | 87.2% | 119.0% | 94.6% | 124.9% | 93.9% | 96.2% | 89.0% | 93.7% | 91.5% | 100.3% | 112.8% | 84.9% |
| 1 uM | 92.4% | 113.2% | 108.8% | 117.7% | 103.8% | 90.9% | 96.0% | 84.4% | 110.0% | 102.1% | 113.9% | 84.9% |

Average

| | SILCS12 | | | SILCS13 | | |
|---|---|---|---|---|---|---|
| | Mean | STD | n | Mean | STD | n |
| 50 uM | 66.97% | 26.12% | 9 | 78.61% | 17.63% | 9 |
| 25 uM | 95.34% | 17.55% | 9 | 104.32% | 6.68% | 9 |
| 10 uM | 96.10% | 8.79% | 9 | 105.25% | 6.16% | 9 |
| 5 uM | 102.70% | 13.08% | 9 | 107.19% | 11.39% | 9 |
| 1 uM | 103.58% | 11.90% | 9 | 105.73% | 11.52% | 9 |

UMB18G and SILCS 14-16 Plate Setup, Greiner Celigo Plates

| | 1 | 2 | 3 | 4 | 5 | 6 |
|---|---|---|---|---|---|---|
| A | UMB18G | UMB18G | UMB18G | SILCS14 | SILCS14 | SILCS14 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 1 |
|---|---|---|---|---|---|---|---|
| A | SILCS15 | SILCS15 | SILCS15 | SILCS16 | SILCS16 | SILCS16 | 50 uM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW475), Percentage Infection Relative to Appropriate DMSO Control (e.g. Set DMSO as 1000%) with Avg Background Subtraction

| | UMB18G | UMB18G | UMB18G | SILCS14 | SILCS14 | SILCS14 | SILCS15 | SILCS15 | SILCS15 | SILCS16 | SILCS16 | SILCS16 |
|---|---|---|---|---|---|---|---|---|---|---|---|---|
| 50 | −0.48% | −1.48% | 0.34% | 2.09% | 3.52% | 1.22% | 23.93% | 15.87% | 20.00% | −1.80% | −1.80% | −1.80% |
| uM | | | | | | | | | | | | |
| 25 | 0.07% | −0.35% | −1.40% | 11.84% | 13.81% | 11.60% | 30.31% | 27.68% | 26.54% | −1.80% | −0.64% | −1.40% |
| uM | | | | | | | | | | | | |
| 10 | 7.16% | 14.38% | 4.59% | 92.53% | 120.55% | 101.62% | 79.51% | 121.98% | 93.64% | 84.49% | 69.44% | 29.80% |
| uM | | | | | | | | | | | | |
| 5 | 11.41% | 21.71% | 5.92% | 112.88% | 98.55% | 115.50% | 131.70% | 141.65% | 132.54% | 104.63% | 118.17% | 80.41% |
| uM | | | | | | | | | | | | |
| 1 | 59.52% | 81.22% | 48.39% | 112.00% | 92.09% | 122.87% | 117.62% | 124.56% | 121.12% | 120.84% | 121.98% | 121.54% |
| uM | | | | | | | | | | | | |

Exp 2 (SMW477), Percentage Infection Relative to Appropriate DMSO Control (e.g. Set DMSO as 100%) with Avg Background Subtraction

| | UMB18G | UMB18G | UMB18G | SILCS14 | SILCS14 | SILCS14 | SILCS15 | SILCS15 | SILCS15 | SILCS16 | SILCS16 | SILCS16 |
|---|---|---|---|---|---|---|---|---|---|---|---|---|
| 50 | 1.36% | 0.07% | 1.60% | 1.91% | −0.25% | −0.24% | 18.29% | 18.14% | 19.12% | 4.62% | 3.74% | 1.88% |
| uM | | | | | | | | | | | | |
| 25 | 1.11% | 0.67% | 1.76% | 19.70% | 19.93% | 22.53% | 35.02% | 27.68% | 29.29% | 0.28% | −0.25% | 0.94% |
| uM | | | | | | | | | | | | |
| 10 | 10.76% | 16.40% | 10.07% | 84.89% | 81.65% | 69.99% | 75.81% | 72.41% | 82.10% | 92.84% | 88.64% | 30.91% |
| uM | | | | | | | | | | | | |
| 5 | 9.13% | 26.77% | 20.94% | 105.78% | 98.21% | 86.55% | 102.31% | 90.53% | 98.90% | 53.88% | 91.95% | 70.12% |
| uM | | | | | | | | | | | | |
| 1 | 30.54% | 44.61% | 49.97% | 91.97% | 93.96% | 99.35% | 103.78% | 103.22% | 100.05% | 105.51% | 118.94% | 105.01% |
| uM | | | | | | | | | | | | |

Exp 3 (SMW478), Percentage Infection Relative to Appropriate DMSO Control (e.g. Set DMSO as 1000%) with Avg Background Subtraction

| | UMB18G | UMB18G | UMB18G | SILCS14 | SILCS14 | SILCS14 | SILCS15 | SILCS15 | SILCS15 | SILCS16 | SILCS16 | SILCS16 |
|---|---|---|---|---|---|---|---|---|---|---|---|---|
| 50 | 0.69% | 2.33% | 1.45% | 3.06% | 0.62% | 3.25% | 20.62% | 25.98% | 33.79% | 9.62% | 2.45% | 0.60% |
| uM | | | | | | | | | | | | |
| 25 | 3.99% | 4.14% | 2.78% | 9.11% | 11.10% | 12.21% | 35.78% | 39.52% | 32.45% | 13.70% | 19.61% | 7.38% |
| uM | | | | | | | | | | | | |
| 10 | 46.05% | 27.62% | 30.93% | 92.27% | 101.81% | 99.27% | 111.15% | 106.59% | 85.32% | 91.68% | 79.30% | 103.54% |
| uM | | | | | | | | | | | | |
| 5 | 28.65% | 43.74% | 34.06% | 109.15% | 113.38% | 106.22% | 98.36% | 110.89% | 93.32% | 96.27% | 87.70% | 87.03% |
| uM | | | | | | | | | | | | |
| 1 | 92.92% | 83.96% | 93.97% | 112.26% | 106.71% | 118.52% | 107.90% | 99.19% | 97.03% | 112.96% | 101.80% | 99.42% |
| uM | | | | | | | | | | | | |

Average

| | UMB18G | | | SILCS14 | | | SILCS15 | | | SILCS16 | |
|---|---|---|---|---|---|---|---|---|---|---|---|
| | Mean | STD | n | Mean | STD | n | Mean | STD | n | Mean | STD |
| 50 uM | 0.65% | 1.18% | 9 | 1.69% | 1.45% | 9 | 21.75% | 5.46% | 9 | 1.95% | 3.77% |
| 25 uM | 1.42% | 1.92% | 9 | 14.65% | 4.78% | 9 | 31.59% | 4.42% | 9 | 4.14% | 7.74% |
| 10 uM | 18.66% | 13.56% | 9 | 93.84% | 14.44% | 9 | 92.05% | 17.41% | 9 | 74.52% | 26.74% |
| 5 uM | 22.48% | 12.35% | 9 | 105.13% | 9.28% | 9 | 111.13% | 19.19% | 9 | 87.79% | 18.75% |
| 1 uM | 65.01% | 23.39% | 9 | 105.53% | 11.70% | 9 | 108.27% | 10.25% | 9 | 112.00% | 9.16% |

Toxicity Data

Exp 1 (SMW475), Percentage Infection Relative to Appropriate DMSO Control (e.g. Set DMSO as 1000%) with Avg Background Subtraction

| | UMB18G | UMB18G | UMB18G | SILCS14 | SILCS14 | SILCS14 | SILCS15 | SILCS15 | SILCS15 | SILCS16 | SILCS16 | SILCS16 |
|---|---|---|---|---|---|---|---|---|---|---|---|---|
| 50 | 30.3% | 41.5% | 40.5% | 46.1% | 39.9% | 33.0% | 29.7% | 30.1% | 28.3% | 2.1% | 2.8% | 2.5% |
| uM | | | | | | | | | | | | |
| 25 | 57.0% | 119.7% | 117.5% | 129.2% | 124.6% | 104.8% | 71.3% | 66.3% | 75.0% | 13.4% | 74.6% | 33.0% |
| uM | | | | | | | | | | | | |
| 10 | 106.9% | 138.3% | 145.5% | 117.4% | 101.9% | 106.1% | 113.4% | 98.0% | 106.9% | 111.3% | 108.9% | 71.3% |
| uM | | | | | | | | | | | | |
| 5 | 102.4% | 139.5% | 144.7% | 105.6% | 107.7% | 109.1% | 103.7% | 93.4% | 98.1% | 104.1% | 108.3% | 78.5% |
| uM | | | | | | | | | | | | |
| 1 | 99.1% | 116.8% | 134.0% | 113.3% | 109.8% | 105.4% | 104.6% | 106.6% | 103.4% | 99.6% | 105.0% | 78.2% |
| uM | | | | | | | | | | | | |

Exp 2 (SMW477), Percentage Infection Relative to Appropriate DMSO Control (e.g. Set DMSO as 1000%) with Avg Background Subtraction

| | UMB18G | UMB18G | UMB18G | SILCS14 | SILCS14 | SILCS14 | SILCS15 | SILCS15 | SILCS15 | SILCS16 | SILCS16 | SILCS16 |
|---|---|---|---|---|---|---|---|---|---|---|---|---|
| 50 | 49.9% | 79.1% | 138.0% | 5.6% | 4.9% | 4.9% | 100.1% | 102.3% | 96.2% | 100.0% | 102.3% | 79.8% |
| uM | | | | | | | | | | | | |
| 25 | 72.5% | 121.3% | 172.1% | 157.4% | 157.1% | 157.0% | 146.0% | 147.3% | 142.5% | 105.5% | 110.1% | 88.8% |
| uM | | | | | | | | | | | | |
| 10 | 145.0% | 201.7% | 219.5% | 116.1% | 126.8% | 135.8% | 122.9% | 127.5% | 118.4% | 113.5% | 115.3% | 160.2% |
| uM | | | | | | | | | | | | |
| 5 | 145.2% | 192.4% | 204.5% | 99.7% | 104.3% | 115.7% | 98.8% | 110.6% | 105.3% | 155.4% | 108.6% | 114.2% |
| uM | | | | | | | | | | | | |
| 1 | 148.6% | 175.9% | 166.7% | 116.0% | 111.6% | 109.4% | 102.1% | 104.5% | 108.6% | 99.9% | 88.7% | 86.9% |
| uM | | | | | | | | | | | | |

Exp 3 (SMW478), Percentage Infection Relative to Appropriate DMSO Control (e.g. Set DMSO as 1000%) with Avg Background Subtraction

| | UMB18G | UMB18G | UMB18G | SILCS14 | SILCS14 | SILCS14 | SILCS15 | SILCS15 | SILCS15 | SILCS16 | SILCS16 | SILCS16 |
|---|---|---|---|---|---|---|---|---|---|---|---|---|
| 50 | 38.2% | 36.8% | 73.7% | 4.4% | 5.5% | 6.8% | 69.9% | 76.8% | 53.3% | 65.2% | 92.6% | 59.3% |
| uM | | | | | | | | | | | | |
| 25 | 64.7% | 113.2% | 156.4% | 127.0% | 137.1% | 163.6% | 150.9% | 83.0% | 159.5% | 155.0% | 152.7% | 95.7% |
| uM | | | | | | | | | | | | |
| 10 | 79.8% | 147.9% | 153.9% | 94.9% | 78.5% | 97.3% | 81.9% | 80.3% | 120.4% | 107.5% | 122.5% | 85.5% |
| uM | | | | | | | | | | | | |
| 5 | 114.8% | 163.0% | 157.7% | 95.8% | 93.4% | 104.0% | 111.1% | 99.3% | 115.6% | 119.4% | 124.0% | 109.1% |
| uM | | | | | | | | | | | | |
| 1 | 70.4% | 106.8% | 110.0% | 95.0% | 98.8% | 90.1% | 100.6% | 110.6% | 112.4% | 101.0% | 109.8% | 83.7% |
| uM | | | | | | | | | | | | |

Average

| | UMB18G | | | SILCS14 | | | SILCS15 | | | SILCS16 | |
|---|---|---|---|---|---|---|---|---|---|---|---|
| | Mean | STD | n | Mean | STD | n | Mean | STD | n | Mean | STD |
| 50 uM | 58.67% | 34.16% | 9 | 16.80% | 17.47% | 9 | 65.19% | 31.05% | 9 | 56.28% | 42.84% |
| 25 uM | 110.49% | 39.62% | 9 | 139.75% | 20.04% | 9 | 115.74% | 40.20% | 9 | 92.09% | 47.63% |
| 10 uM | 148.72% | 42.52% | 9 | 108.29% | 17.59% | 9 | 107.75% | 17.47% | 9 | 110.67% | 24.56% |
| 5 uM | 151.57% | 32.86% | 9 | 103.93% | 6.87% | 9 | 104.00% | 7.32% | 9 | 113.52% | 20.25% |
| 1 uM | 125.37% | 33.99% | 9 | 105.49% | 8.92% | 9 | 105.96% | 3.95% | 9 | 94.77% | 10.70% |

SILCS 17-20 Plate Setup, Greiner Celigo Plates

| | 1 | 2 | 3 | 4 | 5 | 6 |
|---|---|---|---|---|---|---|
| A | SILCS17 | SILCS17 | SILCS17 | SILCS18 | SILCS18 | SILCS18 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 2 |
|---|---|---|---|---|---|---|---|
| A | SILCS19 | SILCS19 | SILCS19 | SILCS20 | SILCS20 | SILCS20 | 50 uM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW475), Percentage Infection Relative to Appropriate DMSO Control (e.g. Set DMSO as 1000%) with Avg Background Subtraction

| | SILCS17 | SILCS17 | SILCS17 | SILCS18 | SILCS18 | SILCS18 | SILCS19 | SILCS19 | SILCS19 | SILCS20 | SILCS20 | SILCS20 |
|---|---|---|---|---|---|---|---|---|---|---|---|---|
| 50 | 15.74% | 49.90% | 30.92% | 8.18% | 18.26% | −0.47% | 0.98% | 6.82% | 3.76% | −0.47% | 0.47% | 1.44% |
| uM | | | | | | | | | | | | |
| 25 | 41.54% | 68.32% | 68.68% | 1.62% | −0.47% | 3.59% | 19.30% | 19.93% | 21.08% | 13.25% | 8.47% | 6.46% |
| uM | | | | | | | | | | | | |
| 10 | 75.56% | 86.80% | 108.19% | 26.68% | 81.65% | 51.38% | 65.05% | 80.15% | 68.60% | 45.14% | 27.86% | 26.38% |
| uM | | | | | | | | | | | | |
| 5 | 86.61% | 84.44% | 77.45% | 65.62% | 57.21% | 60.22% | 79.20% | 104.07% | 98.78% | 71.77% | 48.90% | 69.38% |
| uM | | | | | | | | | | | | |
| 1 | 78.55% | 85.49% | 89.26% | 88.24% | 91.40% | 99.09% | 108.88% | 96.64% | 120.36% | 104.96% | 108.84% | 98.09% |
| uM | | | | | | | | | | | | |

Exp 2 (SMW477), Percentage Infection Relative to Appropriate DMSO Control (e.g. Set DMSO as 1000%) with Avg Background Subtraction

| | SILCS17 | SILCS17 | SILCS17 | SILCS18 | SILCS18 | SILCS18 | SILCS19 | SILCS19 | SILCS19 | SILCS20 | SILCS20 | SILCS20 |
|---|---|---|---|---|---|---|---|---|---|---|---|---|
| 50 | 11.74% | 25.42% | 30.96% | −0.97% | −1.03% | 0.05% | −1.03% | −1.64% | −0.70% | −0.57% | −0.19% | −1.43% |
| uM | | | | | | | | | | | | |
| 25 | 38.35% | 67.19% | 82.12% | −0.92% | −1.43% | −1.52% | 32.49% | 36.71% | 35.36% | 9.75% | 7.72% | 7.01% |
| uM | | | | | | | | | | | | |
| 10 | 130.54% | 160.59% | 172.84% | 109.44% | 62.81% | 37.01% | 160.35% | 105.70% | 149.57% | 76.01% | 46.79% | 30.46% |
| uM | | | | | | | | | | | | |
| 5 | 142.87% | 172.05% | 157.06% | 162.61% | 95.92% | 92.01% | 186.48% | 158.05% | 169.99% | 133.79% | 116.24% | 114.33% |
| uM | | | | | | | | | | | | |
| 1 | 101.46% | 156.03% | 155.63% | 167.15% | 158.27% | 150.30% | 158.54% | 153.60% | 151.26% | 151.25% | 151.34% | 136.14% |
| uM | | | | | | | | | | | | |

Exp 3 (SMW478), Percentage Infection Relative to Appropriate DMSO Control (e.g. Set DMSO as 100%) with Avg Background Subtraction

| | SILCS17 | SILCS17 | SILCS17 | SILCS18 | SILCS18 | SILCS18 | SILCS19 | SILCS19 | SILCS19 | SILCS20 | SILCS20 | SILCS20 |
|---|---|---|---|---|---|---|---|---|---|---|---|---|
| 50 | 20.44% | 62.60% | 81.13% | 78.38% | 59.07% | 78.40% | 9.83% | 5.42% | 8.96% | 125.56% | 159.57% | 149.88% |
| uM | | | | | | | | | | | | |
| 25 | 68.44% | 109.27% | 278.92% | 1.81% | −0.13% | −0.15% | 81.21% | 37.46% | 59.56% | 6.76% | 9.52% | 8.68% |
| uM | | | | | | | | | | | | |
| 10 | 90.38% | 97.98% | 103.28% | 81.65% | 75.08% | 69.78% | 99.40% | 69.65% | 98.62% | 30.07% | 13.27% | 56.68% |
| uM | | | | | | | | | | | | |
| 5 | 97.85% | 101.86% | 101.30% | 91.92% | 68.10% | 79.87% | 101.17% | 84.71% | 92.10% | 62.84% | 53.65% | 64.98% |
| uM | | | | | | | | | | | | |
| 1 | 90.81% | 93.25% | 105.34% | 85.49% | 91.60% | 87.40% | 72.63% | 88.40% | 96.76% | 92.92% | 94.78% | 98.52% |
| uM | | | | | | | | | | | | |

Average

| | SILCS17 | | | SILCS18 | | | SILCS19 | | | SILCS20 | |
|---|---|---|---|---|---|---|---|---|---|---|---|
| | Mean | STD | n | Mean | STD | n | Mean | STD | n | Mean | STD |
| 50 uM | 36.54% | 23.29% | 9 | 26.65% | 34.98% | 9 | 3.60% | 4.41% | 9 | 48.15% | 73.17% |
| 25 uM | 91.43% | 73.34% | 9 | 0.27% | 1.72% | 9 | 38.12% | 20.41% | 9 | 8.63% | 2.09% |
| 10 uM | 114.02% | 33.70% | 9 | 66.17% | 25.21% | 9 | 99.68% | 34.74% | 9 | 39.18% | 18.97% |
| 5 uM | 113.50% | 34.61% | 9 | 85.94% | 32.22% | 9 | 119.39% | 40.48% | 9 | 81.76% | 31.06% |
| 1 uM | 106.20% | 29.23% | 9 | 113.22% | 34.49% | 9 | 116.34% | 31.47% | 9 | 115.20% | 24.17% |

Toxicity Data

Exp 1 (SMW475), Percentage Infection Relative to Appropriate DMSO Control (e.g. Set DMSO as 100%) with Avg Background Subtraction

| | SILCS17 | SILCS17 | SILCS17 | SILCS18 | SILCS18 | SILCS18 | SILCS19 | SILCS19 | SILCS19 | SILCS20 | SILCS20 | SILCS20 |
|---|---|---|---|---|---|---|---|---|---|---|---|---|
| 50 | 52.4% | 60.8% | 53.4% | 2.5% | 2.6% | 2.5% | 53.5% | 54.0% | 45.4% | 3.7% | 2.9% | 2.9% |
| uM | | | | | | | | | | | | |
| 25 | 91.0% | 107.8% | 107.3% | 50.3% | 50.9% | 40.6% | 104.1% | 99.4% | 95.9% | 95.0% | 81.0% | 57.6% |
| uM | | | | | | | | | | | | |
| 10 | 104.1% | 115.8% | 104.9% | 131.4% | 113.7% | 119.9% | 120.8% | 110.1% | 115.0% | 131.7% | 138.1% | 89.4% |
| uM | | | | | | | | | | | | |
| 5 | 98.3% | 112.7% | 113.0% | 123.2% | 122.7% | 128.8% | 111.6% | 100.0% | 104.9% | 111.8% | 122.3% | 95.9% |
| uM | | | | | | | | | | | | |
| 1 | 102.6% | 110.5% | 108.1% | 106.2% | 102.1% | 106.8% | 104.1% | 103.5% | 98.7% | 100.9% | 96.9% | 88.2% |
| uM | | | | | | | | | | | | |

Exp 2 (SMW477), Percentage Infection Relative to Appropriate DMSO Control (e.g. Set DMSO as 100%) with Avg Background Subtraction

| | SILCS17 | SILCS17 | SILCS17 | SILCS18 | SILCS18 | SILCS18 | SILCS19 | SILCS19 | SILCS19 | SILCS20 | SILCS20 | SILCS20 |
|---|---|---|---|---|---|---|---|---|---|---|---|---|
| 50 | 54.9% | 77.9% | 82.8% | 3.1% | 3.3% | 4.3% | 69.3% | 69.7% | 63.9% | 38.5% | 5.0% | 12.4% |
| uM | | | | | | | | | | | | |
| 25 | 92.7% | 108.6% | 107.2% | 29.0% | 31.1% | 7.7% | 131.4% | 127.8% | 125.0% | 128.4% | 126.1% | 106.4% |
| uM | | | | | | | | | | | | |
| 10 | 68.4% | 75.4% | 67.0% | 92.5% | 114.4% | 122.9% | 66.3% | 92.7% | 75.6% | 108.3% | 119.1% | 112.4% |
| uM | | | | | | | | | | | | |
| 5 | 65.9% | 70.9% | 75.3% | 72.4% | 97.4% | 100.4% | 61.6% | 70.9% | 69.7% | 84.2% | 90.4% | 81.7% |
| uM | | | | | | | | | | | | |
| 1 | 87.5% | 78.3% | 79.0% | 72.4% | 78.5% | 79.1% | 75.9% | 75.7% | 80.4% | 79.2% | 79.4% | 77.6% |
| uM | | | | | | | | | | | | |

Exp 3 (SMW478), Percentage Infection Relative to Appropriate DMSO Control (e.g. Set DMSO as 1000%) with Avg Background Subtraction

| | SILCS17 | SILCS17 | SILCS17 | SILCS18 | SILCS18 | SILCS18 | SILCS19 | SILCS19 | SILCS19 | SILCS20 | SILCS20 | SILCS20 |
|---|---|---|---|---|---|---|---|---|---|---|---|---|
| 50 | 32.7% | 33.2% | 33.5% | 1.1% | 0.9% | 1.8% | 43.6% | 73.1% | 48.2% | 0.5% | 0.2% | 0.2% |
| uM | | | | | | | | | | | | |
| 25 | 24.0% | 40.6% | 0.0% | 74.6% | 85.8% | 72.1% | 7.0% | 80.4% | 36.6% | 65.0% | 28.2% | 14.9% |
| uM | | | | | | | | | | | | |
| 10 | 68.1% | 100.9% | 95.1% | 109.0% | 125.2% | 147.8% | 112.5% | 150.3% | 111.3% | 211.7% | 210.8% | 128.4% |
| uM | | | | | | | | | | | | |
| 5 | 58.7% | 99.9% | 107.9% | 112.8% | 152.9% | 142.0% | 110.2% | 134.0% | 125.5% | 171.3% | 163.3% | 130.7% |
| uM | | | | | | | | | | | | |
| 1 | 91.6% | 122.8% | 86.2% | 126.5% | 117.2% | 137.4% | 141.1% | 140.5% | 126.5% | 134.3% | 130.7% | 84.3% |
| uM | | | | | | | | | | | | |

Average

| | | SILCS17 | | | SILCS18 | | | SILCS19 | | | SILCS20 | |
|---|---|---|---|---|---|---|---|---|---|---|---|---|
| | | Mean | STD | n | Mean | STD | n | Mean | STD | n | Mean | STD |
| 50 | uM | 53.52% | 18.56% | 9 | 2.46% | 1.08% | 9 | 57.85% | 11.30% | 9 | 7.37% | 12.26% |
| 25 | uM | 75.47% | 42.23% | 9 | 49.15% | 25.12% | 9 | 89.74% | 42.61% | 9 | 78.06% | 40.32% |
| 10 | uM | 88.87% | 19.07% | 9 | 119.65% | 15.34% | 9 | 106.06% | 25.05% | 9 | 138.88% | 43.45% |
| 5 | m uM | 89.17% | 21.41% | 9 | 116.95% | 24.47% | 9 | 98.71% | 25.71% | 9 | 116.85% | 33.18% |
| 1 | uM | 96.29% | 15.44% | 9 | 102.92% | 22.56% | 9 | 105.15% | 25.93% | 9 | 96.85% | 21.74% |

SILCS 21-24 Plate Setup, Greiner Celigo Plates

| | 1 | 2 | 3 | 4 | 5 | 6 |
|---|---|---|---|---|---|---|
| A | SILCS21 | SILCS21 | SILCS21 | SILCS22 | SILCS22 | SILCS22 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 3 |
|---|---|---|---|---|---|---|---|
| A | SILCS23 | SILCS23 | SILCS23 | SILCS24 | SILCS24 | SILCS24 | 50 uM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW475), Percentage Infection Relative to Appropriate DMSO Control (e.g. Set DMSO as 100%) with Avg Background Subtraction

| | SILCS21 | SILCS21 | SILCS21 | SILCS22 | SILCS22 | SILCS22 | SILCS23 | SILCS23 | SILCS23 | SILCS24 | SILCS24 | SILCS24 |
|---|---|---|---|---|---|---|---|---|---|---|---|---|
| 50 | 24.78% | 23.95% | 1.03% | 0.02% | 3.92% | 4.65% | 11.05% | 12.93% | 12.05% | 20.92% | 16.99% | 16.36% |
| uM | | | | | | | | | | | | |
| 25 | 3.01% | 1.01% | 0.28% | 2.49% | 6.38% | 4.75% | 23.73% | 16.03% | 21.54% | 36.92% | 31.50% | 14.81% |
| uM | | | | | | | | | | | | |
| 10 | 10.06% | 15.00% | 23.04% | 15.62% | 28.80% | 29.55% | 57.63% | 66.75% | 71.82% | 93.85% | 91.06% | 50.31% |
| uM | | | | | | | | | | | | |
| 5 | 36.07% | 38.44% | 40.11% | 32.75% | 46.98% | 35.47% | 76.20% | 84.17% | 85.48% | 90.16% | 99.17% | 50.26% |
| uM | | | | | | | | | | | | |
| 1 | 68.59% | 72.89% | 90.21% | 68.46% | 64.64% | 77.37% | 84.61% | 99.83% | 96.53% | 124.65% | 102.70% | 66.42% |
| uM | | | | | | | | | | | | |

Exp 2 (SMW477), Percentage Infection Relative to Appropriate DMSO Control (e.g. Set DMSO as 1000%) with Avg Background Subtraction

| | SILCS21 | SILCS21 | SILCS21 | SILCS22 | SILCS22 | SILCS22 | SILCS23 | SILCS23 | SILCS23 | SILCS24 | SILCS24 | SILCS24 |
|---|---|---|---|---|---|---|---|---|---|---|---|---|
| 50 | 0.68% | 1.71% | −0.90% | 3.65% | 0.99% | 1.90% | 22.07% | 19.29% | 19.84% | 18.59% | 11.16% | 9.24% |
| uM | | | | | | | | | | | | |
| 25 | 2.28% | 2.27% | 4.22% | 5.13% | 4.50% | 4.04% | 42.76% | 36.42% | 27.04% | 35.03% | 22.44% | 7.58% |
| uM | | | | | | | | | | | | |
| 10 | 11.15% | 28.56% | 68.83% | 68.54% | 46.53% | 47.36% | 81.53% | 45.87% | 61.92% | 54.07% | 57.64% | 38.68% |
| uM | | | | | | | | | | | | |
| 5 | 72.98% | 102.95% | 79.59% | 93.66% | 92.49% | 88.62% | 77.18% | 66.56% | 59.07% | 78.38% | 98.62% | 72.17% |
| uM | | | | | | | | | | | | |
| 1 | 74.32% | 88.48% | 89.84% | 76.88% | 75.65% | 76.95% | 76.88% | 82.87% | 95.55% | 88.56% | 86.90% | 93.54% |
| uM | | | | | | | | | | | | |

Exp 3 (SMW478), Percentage Infection Relative to Appropriate DMSO Control (e.g. Set DMSO as 1000%) with Avg Background Subtraction

| | SILCS21 | SILCS21 | SILCS21 | SILCS22 | SILCS22 | SILCS22 | SILCS23 | SILCS23 | SILCS23 | SILCS24 | SILCS24 | SILCS24 |
|---|---|---|---|---|---|---|---|---|---|---|---|---|
| 50 | 17.92% | 10.72% | 13.59% | 1.60% | 3.82% | 3.54% | 42.28% | 47.99% | 44.36% | 37.54% | 29.46% | 13.09% |
| uM | | | | | | | | | | | | |
| 25 | 19.44% | 14.86% | 8.13% | 8.14% | 10.91% | 10.99% | 59.17% | 74.77% | 81.13% | 74.88% | 62.40% | 29.78% |
| uM | | | | | | | | | | | | |
| 10 | 17.12% | 37.03% | 26.97% | 92.75% | 69.87% | 79.13% | 84.08% | 95.62% | 82.31% | 97.16% | 81.93% | 73.95% |
| uM | | | | | | | | | | | | |
| 5 | 49.98% | 72.69% | 79.52% | 69.69% | 88.68% | 84.47% | 80.66% | 70.90% | 56.23% | 51.31% | 74.26% | 29.57% |
| uM | | | | | | | | | | | | |
| 1 | 103.05% | 98.14% | 114.44% | 62.06% | 83.81% | 65.64% | 74.83% | 73.82% | 64.40% | 60.27% | −2.73% | −3.55% |
| uM | | | | | | | | | | | | |

Average

| | SILCS21 | | | SILCS22 | | | SILCS23 | | | SILCS24 | |
|---|---|---|---|---|---|---|---|---|---|---|---|
| | Mean | STD | n | Mean | STD | n | Mean | STD | n | Mean | STD |
| 50 uM | 10.39% | 10.26% | 9 | 2.68% | 1.58% | 9 | 25.76% | 14.89% | 9 | 19.26% | 9.08% |
| 25 uM | 6.17% | 6.72% | 9 | 6.37% | 3.03% | 9 | 42.51% | 23.93% | 9 | 35.04% | 21.51% |
| 10 uM | 26.42% | 18.18% | 9 | 53.13% | 25.97% | 9 | 71.95% | 15.49% | 9 | 70.96% | 21.43% |
| 5 uM | 63.59% | 23.34% | 9 | 70.31% | 25.20% | 9 | 72.94% | 10.54% | 9 | 71.54% | 23.78% |
| 1 uM | 88.88% | 15.09% | 9 | 72.38% | 7.37% | 9 | 83.26% | 12.04% | 9 | 68.53% | 44.76% |

Toxicity Data

Exp 1 (SMW475), Percentage Infection Relative to Appropriate DMSO Control (e.g. Set DMSO as 1000%) with Avg Background Subtraction

| | SILCS21 | SILCS21 | SILCS21 | SILCS22 | SILCS22 | SILCS22 | SILCS23 |
|---|---|---|---|---|---|---|---|
| 50 uM | 1.1% | 3.3% | 2.6% | 41.8% | 77.5% | 60.9% | 90.9% |
| 25 uM | 54.6% | 75.4% | 81.0% | 106.1% | 127.1% | 120.6% | 127.9% |
| 10 uM | 89.5% | 126.1% | 131.8% | 140.4% | 135.1% | 129.4% | 118.2% |
| 5 uM | 98.6% | 135.7% | 134.1% | 136.9% | 138.4% | 132.4% | 119.4% |
| 1 uM | 105.6% | 113.2% | 110.4% | 123.4% | 115.6% | 114.9% | 109.5% |
| | | | SILCS23 | SILCS23 | SILCS24 | SILCS24 | SILCS24 |
| | | 50 uM | 82.1% | 81.8% | 73.3% | 60.2% | 40.3% |
| | | 25 uM | 123.9% | 121.7% | 121.1% | 114.5% | 70.5% |
| | | 10 uM | 121.8% | 118.2% | 109.2% | 106.2% | 82.0% |
| | | 5 uM | 110.1% | 112.1% | 112.1% | 107.3% | 85.1% |
| | | 1 uM | 101.2% | 105.8% | 98.9% | 102.5% | 78.9% |

Exp 2 (SMW477), Percentage Infection Relative to Appropriate DMSO Control (e.g. Set DMSO as 1000%) with Avg Background Subtraction

| | SILCS21 | SILCS21 | SILCS21 | SILCS22 | SILCS22 | SILCS22 | SILCS23 |
|---|---|---|---|---|---|---|---|
| 50 uM | 4.8% | 5.8% | 3.9% | 83.6% | 88.9% | 81.2% | 129.7% |
| 25 uM | 69.1% | 102.4% | 123.6% | 157.8% | 155.6% | 155.4% | 124.2% |
| 10 uM | 154.6% | 175.9% | 131.5% | 135.1% | 157.2% | 157.1% | 124.6% |
| 5 uM | 111.0% | 105.9% | 127.8% | 115.1% | 115.4% | 114.0% | 121.6% |
| 1 uM | 109.5% | 116.7% | 112.5% | 125.8% | 129.1% | 123.3% | 130.7% |
| | | | SILCS23 | SILCS23 | SILCS24 | SILCS24 | SILCS24 |
| | | 50 uM | 128.1% | 120.5% | 108.6% | 102.6% | 78.2% |
| | | 25 uM | 115.1% | 88.0% | 96.5% | 89.1% | 46.2% |
| | | 10 uM | 153.0% | 141.3% | 145.1% | 143.1% | 147.9% |
| | | 5 uM | 136.4% | 146.2% | 127.1% | 108.1% | 112.3% |
| | | 1 uM | 121.3% | 106.3% | 119.8% | 117.8% | 94.4% |

Exp 3 (SMW478), Percentage Infection Relative to Appropriate DMSO Control (e.g. Set DMSO as 1000%) with Avg Background Subtraction

| | SILCS21 | SILCS21 | SILCS21 | SILCS22 | SILCS22 | SILCS22 | SILCS23 |
|---|---|---|---|---|---|---|---|
| 50 uM | 38.1% | 40.8% | 58.0% | 69.3% | 63.4% | 52.5% | 64.7% |
| 25 uM | 26.2% | 70.4% | 131.4% | 132.3% | 116.6% | 115.1% | 121.7% |
| 10 uM | 97.3% | 158.5% | 161.9% | 114.0% | 134.9% | 131.5% | 122.5% |
| 5 uM | 110.7% | 119.3% | 122.1% | 140.3% | 114.5% | 123.8% | 123.2% |
| 1 uM | 80.5% | 101.2% | 89.8% | 141.2% | 115.4% | 149.0% | 143.7% |
| | | | SILCS23 | SILCS23 | SILCS24 | SILCS24 | SILCS24 |
| | | 50 uM | 54.0% | 84.1% | 86.2% | 64.3% | 74.6% |
| | | 25 uM | 80.2% | 110.6% | 113.9% | 112.6% | 101.6% |
| | | 10 uM | 104.9% | 126.0% | 113.5% | 132.5% | 103.3% |
| | | 5 uM | 136.3% | 157.5% | 167.1% | 135.5% | 161.4% |
| | | 1 uM | 141.2% | 153.5% | 159.1% | 239.8% | 186.0% |

Average

| | SILCS21 | | | SILCS22 | | | SILCS23 | | | SILCS24 | |
|---|---|---|---|---|---|---|---|---|---|---|---|
| | Mean | STD | n | Mean | STD | n | Mean | STD | n | Mean | STD |
| 50 uM | 17.58% | 21.76% | 9 | 68.81% | 15.56% | 9 | 92.88% | 27.36% | 9 | 76.48% | 21.05% |
| 25 uM | 81.56% | 33.15% | 9 | 131.85% | 19.73% | 9 | 112.58% | 17.07% | 9 | 96.21% | 24.43% |
| 10 uM | 136.35% | 29.39% | 9 | 137.18% | 13.47% | 9 | 125.61% | 13.97% | 9 | 120.30% | 22.83% |
| 5 uM | 118.34% | 12.81% | 9 | 125.64% | 11.35% | 9 | 129.20% | 15.96% | 9 | 124.00% | 26.75% |
| 1 uM | 104.37% | 12.02% | 9 | 126.41% | 11.86% | 9 | 123.69% | 19.30% | 9 | 133.02% | 52.23% |

SILCS 25-28 Plate Setup, Greiner Celigo Plates

| | 1 | 2 | 3 | 4 | 5 | 6 | 7 |
|---|---|---|---|---|---|---|---|
| A | SILCS25 | SILCS25 | SILCS25 | SILCS26 | SILCS26 | SILCS26 | SILCS27 |
| B | | | | | | | |
| C | | | | | | | |
| D | | | | | | | |
| E | | | | | | | |
| F | DMSO | | | | | | |
| G | DMSO | | | | | | |
| H | Uninfect | | | | | | |
| | | 8 | 9 | 10 | 11 | 12 | Plate 4 |
| | A | SILCS27 | SILCS27 | SILCS28 | SILCS28 | SILCS28 | 50 uM |
| | B | | | | | | 25 uM |
| | C | | | | | | 10 uM |
| | D | | | | | | 5 uM |
| | E | | | | | | 1 uM |
| | F | | | | | | 0.50% |
| | G | | | | | | 0.10% |
| | H | | | | | | N/A |

Infection Data

Exp 1 (SMW475), Percentage Infection Relative to Appropriate DMSO Control (e.g. Set DMSO as 100%) with Avg Background Subtraction

| | SILCS25 | SILCS25 | SILCS25 | SILCS26 | SILCS26 | SILCS26 | SILCS27 |
|---|---|---|---|---|---|---|---|
| 50 uM | 0.58% | 10.54% | −0.25% | 13.43% | 10.58% | 11.39% | 13.28% |
| 25 uM | 20.10% | 17.15% | 22.52% | 32.94% | 41.18% | 36.66% | 58.04% |
| 10 uM | 32.49% | 38.59% | 41.39% | 81.62% | 92.60% | 103.50% | 122.43% |
| 5 uM | 41.49% | 44.11% | 56.02% | 73.53% | 79.08% | 98.16% | 106.69% |
| 1 uM | 76.11% | 63.77% | 82.42% | 82.69% | 84.63% | 91.55% | 95.32% |
| | | | SILCS27 | SILCS27 | SILCS28 | SILCS28 | SILCS28 |
| | | 50 uM | 15.03% | 16.97% | 15.48% | 17.28% | 6.19% |
| | | 25 uM | 63.59% | 56.84% | 50.56% | 52.12% | 24.53% |
| | | 10 uM | 137.00% | 134.73% | 115.38% | 121.28% | 72.10% |
| | | 5 uM | 113.52% | 118.94% | 99.56% | 112.68% | 101.18% |
| | | 1 uM | 107.33% | 128.47% | 109.97% | 110.09% | 93.33% |

Exp 2 (SMW477), Percentage Infection Relative to Appropriate DMSO Control (e.g. Set DMSO as 1000%) with Avg Background Subtraction

| | SILCS25 | SILCS25 | SILCS25 | SILCS26 | SILCS26 | SILCS26 | SILCS27 |
|---|---|---|---|---|---|---|---|
| 50 uM | 6.25% | −1.44% | 3.55% | 25.13% | 26.03% | 27.02% | 21.80% |
| 25 uM | 22.26% | 32.75% | 41.71% | 61.78% | 45.22% | 63.04% | 73.05% |
| 10 uM | 24.58% | 1.57% | 1.66% | 83.72% | 87.01% | 0.23% | 89.00% |
| 5 uM | 14.80% | 46.57% | 49.99% | 94.71% | 83.96% | 93.46% | 94.77% |
| 1 uM | 58.66% | 84.02% | 102.25% | 96.35% | 106.86% | 103.16% | 110.54% |
| | | | SILCS27 | SILCS27 | SILCS28 | SILCS28 | SILCS28 |
| | | 50 uM | 26.32% | 19.89% | 20.84% | 16.29% | 18.57% |
| | | 25 uM | 62.89% | 48.77% | 43.28% | 44.60% | 39.31% |
| | | 10 uM | 76.80% | 77.09% | 81.88% | 83.65% | 95.63% |
| | | 5 uM | 92.70% | 91.91% | 80.94% | 101.92% | 113.29% |
| | | 1 uM | 100.41% | 105.41% | 99.59% | 104.43% | 122.03% |

Exp 3 (SMW478), Percentage Infection Relative to Appropriate DMSO Control (e.g. Set DMSO as 1000%) with Avg Background Subtraction

| | SILCS25 | SILCS25 | SILCS25 | SILCS26 | SILCS26 | SILCS26 | SILCS27 |
|---|---|---|---|---|---|---|---|
| 50 uM | 6.88% | −1.19% | −1.09% | 28.61% | 29.92% | 14.89% | 26.23% |
| 25 uM | 32.71% | 55.47% | 67.66% | 79.03% | 71.83% | 64.72% | 86.74% |
| 10 uM | 99.71% | 91.75% | 106.45% | 73.78% | 69.60% | 68.94% | 63.55% |
| 5 uM | 95.72% | 88.47% | 77.81% | 89.39% | 93.72% | 82.15% | 64.83% |
| 1 uM | 97.82% | 84.39% | 100.08% | 81.75% | 100.71% | 99.49% | 90.68% |
| | | | SILCS27 | SILCS27 | SILCS28 | SILCS28 | SILCS28 |
| | | 50 uM | 18.61% | 22.87% | 3.55% | 3.70% | 2.61% |
| | | 25 uM | 80.23% | 76.86% | 36.60% | 40.12% | 18.42% |
| | | 10 uM | 89.71% | 51.99% | 26.96% | 41.66% | −1.43% |
| | | 5 uM | 79.11% | 61.06% | 41.71% | 49.62% | 20.36% |
| | | 1 uM | 87.75% | 89.34% | 80.30% | 82.86% | 62.40% |

Average

| | SILCS25 | | | SILCS26 | | | SILCS27 | | | SILCS28 | |
|---|---|---|---|---|---|---|---|---|---|---|---|
| | Mean | STD | n | Mean | STD | n | Mean | STD | n | Mean | STD |
| 50 uM | 2.65% | 4.36% | 9 | 20.78% | 8.00% | 9 | 20.11% | 4.62% | 9 | 11.61% | 7.42% |
| 25 uM | 34.70% | 17.32% | 9 | 55.15% | 16.49% | 9 | 67.45% | 12.46% | 9 | 38.84% | 11.15% |
| 10 uM | 48.69% | 40.66% | 9 | 73.44% | 29.63% | 9 | 93.59% | 30.89% | 9 | 70.79% | 40.97% |
| 5 uM | 57.22% | 25.66% | 9 | 87.57% | 8.29% | 9 | 91.50% | 20.22% | 9 | 80.14% | 34.34% |
| 1 uM | 83.28% | 15.39% | 9 | 94.13% | 9.36% | 9 | 101.69% | 13.00% | 9 | 96.11% | 18.41% |

Toxicity Data

Exp 1 (SMW475), Percentage Infection Relative to Appropriate DMSO Control (e.g. Set DMSO as 1000%) with Avg Background Subtraction

| | SILCS25 | SILCS25 | SILCS25 | SILCS26 | SILCS26 | SILCS26 | SILCS27 |
|---|---|---|---|---|---|---|---|
| 50 uM | 3.3% | 2.4% | 6.3% | 43.3% | 50.6% | 56.8% | 66.6% |
| 25 uM | 37.4% | 74.2% | 88.9% | 103.4% | 102.4% | 111.2% | 103.8% |
| 10 uM | 87.4% | 111.2% | 121.9% | 108.0% | 104.3% | 100.2% | 93.7% |
| 5 uM | 96.6% | 130.6% | 128.6% | 120.1% | 117.3% | 104.4% | 99.0% |
| 1 uM | 97.1% | 109.0% | 104.5% | 98.5% | 102.4% | 105.0% | 102.0% |
| | | | SILCS27 | SILCS27 | SILCS28 | SILCS28 | SILCS28 |
| | | 50 uM | 65.1% | 76.3% | 76.5% | 70.0% | 54.0% |
| | | 25 uM | 98.9% | 107.4% | 105.0% | 99.7% | 78.8% |
| | | 10 uM | 86.9% | 89.2% | 94.3% | 96.4% | 88.7% |
| | | 5 uM | 100.2% | 95.6% | 105.5% | 101.2% | 90.2% |
| | | 1 uM | 95.0% | 91.0% | 100.0% | 96.1% | 86.9% |

Exp 2 (SMW477), Percentage Infection Relative to Appropriate DMSO Control (e.g. Set DMSO as 1000%) with Avg Background Subtraction

| | SILCS25 | SILCS25 | SILCS25 | SILCS26 | SILCS26 | SILCS26 | SILCS27 |
|---|---|---|---|---|---|---|---|
| 50 uM | 2.0% | 11.2% | 16.0% | 104.6% | 92.8% | 95.0% | 108.8% |
| 25 uM | 86.9% | 110.5% | 115.2% | 112.1% | 117.2% | 112.4% | 107.7% |
| 10 uM | 136.9% | 210.1% | 203.0% | 129.9% | 123.4% | 208.6% | 120.7% |
| 5 uM | 155.4% | 159.7% | 158.9% | 120.5% | 127.0% | 112.6% | 111.1% |
| 1 uM | 122.9% | 124.2% | 110.7% | 115.5% | 103.1% | 108.5% | 103.9% |
| | | | SILCS27 | SILCS27 | SILCS28 | SILCS28 | SILCS28 |
| | | 50 uM | 88.1% | 91.5% | 81.5% | 70.9% | 49.7% |
| | | 25 uM | 116.1% | 119.9% | 125.4% | 113.3% | 87.6% |
| | | 10 uM | 131.2% | 134.0% | 125.4% | 122.6% | 89.9% |
| | | 5 uM | 117.5% | 116.5% | 130.8% | 106.0% | 87.3% |
| | | 1 uM | 110.3% | 108.0% | 114.3% | 108.2% | 81.8% |

Exp 3 (SMW478), Percentage Infection Relative to Appropriate DMSO Control (e.g. Set DMSO as 1000%) with Avg Background Subtraction

| | SILCS25 | SILCS25 | SILCS25 | SILCS26 | SILCS26 | SILCS26 | SILCS27 |
|---|---|---|---|---|---|---|---|
| 50 uM | 54.8% | 54.3% | 47.1% | 67.4% | 63.0% | 82.7% | 83.3% |
| 25 uM | 51.7% | 52.4% | 99.4% | 70.0% | 93.8% | 92.3% | 72.0% |
| 10 uM | 49.7% | 98.8% | 86.8% | 106.7% | 126.7% | 135.8% | 134.5% |
| 5 uM | 60.3% | 105.0% | 132.4% | 116.9% | 98.2% | 121.1% | 151.6% |
| 1 uM | 81.9% | 123.9% | 101.8% | 120.1% | 106.6% | 110.0% | 120.8% |
| | | | SILCS27 | SILCS27 | SILCS28 | SILCS28 | SILCS28 |
| | | 50 uM | 102.7% | 94.6% | 91.4% | 55.7% | 16.1% |
| | | 25 uM | 109.0% | 94.3% | 109.6% | 46.7% | 40.1% |
| | | 10 uM | 121.6% | 169.7% | 201.2% | 167.0% | 176.6% |
| | | 5 uM | 134.3% | 160.2% | 176.2% | 162.3% | 173.2% |
| | | 1 uM | 126.4% | 124.3% | 136.9% | 131.4% | 130.5% |

Average

| | SILCS25 | | | SILCS26 | | | SILCS27 | | | SILCS28 | |
|---|---|---|---|---|---|---|---|---|---|---|---|
| | Mean | STD | n | Mean | STD | n | Mean | STD | n | Mean | STD |
| 50 uM | 21.94% | 23.14% | 9 | 72.93% | 21.65% | 9 | 86.33% | 15.09% | 9 | 62.87% | 22.22% |
| 25 uM | 79.63% | 27.57% | 9 | 101.64% | 14.65% | 9 | 103.23% | 14.09% | 9 | 89.57% | 29.57% |
| 10 uM | 122.86% | 53.45% | 9 | 127.07% | 33.10% | 9 | 120.18% | 26.80% | 9 | 129.12% | 42.43% |
| 5 uM | 125.28% | 33.12% | 9 | 115.35% | 8.97% | 9 | 120.66% | 23.29% | 9 | 125.86% | 35.89% |
| 1 uM | 108.44% | 14.12% | 9 | 107.75% | 6.73% | 9 | 109.07% | 12.63% | 9 | 109.57% | 20.14% |

SILCS 29-32-Plate Setup, Greiner Celigo Plates

| | 1 | 2 | 3 | 4 | 5 | 6 | 7 |
|---|---|---|---|---|---|---|---|
| A | SILCS29 | SILCS29 | SILCS29 | SILCS30 | SILCS30 | SILCS30 | SILCS31 |
| B | | | | | | | |
| C | | | | | | | |
| D | | | | | | | |
| E | | | | | | | |
| F | DMSO | | | | | | |
| G | DMSO | | | | | | |
| H | Uninfect | | | | | | |
| | | 8 | 9 | 10 | 11 | 12 | Plate 4 |
| | A | SILCS31 | SILCS31 | SILCS32 | SILCS32 | SILCS32 | 50 uM |
| | B | | | | | | 25 uM |
| | C | | | | | | 10 uM |
| | D | | | | | | 5 uM |
| | E | | | | | | 1 uM |
| | F | | | | | | 0.50% |
| | G | | | | | | 0.10% |
| | H | | | | | | N/A |

Infection Data

Exp 1 (SMW475), Percentage Infection Relative to Appropriate DMSO Control (e.g. Set DMSO as 1000%) with Avg Background Subtraction

| | SILCS29 | SILCS29 | SILCS29 | SILCS30 | SILCS30 | SILCS30 | SILCS31 | SILCS31 | SILCS31 | SILCS32 | SILCS32 | SILCS32 |
|---|---|---|---|---|---|---|---|---|---|---|---|---|
| 50 | 13.24% | 10.68% | 10.82% | 32.13% | 25.36% | 23.71% | 27.95% | 23.70% | 21.94% | −0.21% | 3.87% | −0.14% |
| uM | | | | | | | | | | | | |
| 25 | 13.47% | 21.51% | 26.64% | 63.92% | 51.24% | 55.41% | 64.20% | 68.29% | 61.79% | 33.08% | 28.83% | 23.83% |
| uM | | | | | | | | | | | | |
| 10 | 57.43% | 82.75% | 79.22% | 111.40% | 92.91% | 108.37% | 122.44% | 140.70% | 131.42% | 85.84% | 57.04% | 33.27% |
| uM | | | | | | | | | | | | |
| 5 | 1.25% | −0.49% | 2.25% | 73.51% | 0.79% | 92.35% | 96.46% | 105.10% | 110.06% | 48.21% | 38.83% | 27.06% |
| uM | | | | | | | | | | | | |
| 1 | 54.22% | −0.38% | 93.47% | 105.27% | 104.72% | 85.58% | 89.46% | 114.99% | 104.36% | 112.58% | 93.90% | 79.63% |
| uM | | | | | | | | | | | | |

Exp 2 (SMW477), Percentage Infection Relative to Appropriate DMSO Control (e.g. Set DMSO as 100%) with Avg Background Subtraction

| | SILCS29 | SILCS29 | SILCS29 | SILCS30 | SILCS30 | SILCS30 | SILCS31 | SILCS31 | SILCS31 | SILCS32 | SILCS32 | SILCS32 |
|---|---|---|---|---|---|---|---|---|---|---|---|---|
| 50 | 2.61% | 6.87% | 8.07% | 17.82% | 25.00% | 26.06% | 19.41% | 19.72% | 23.27% | 1.33% | −1.47% | 8.36% |
| uM | | | | | | | | | | | | |
| 25 | 11.30% | 29.76% | 35.99% | 71.18% | 69.34% | 78.15% | 73.95% | 82.92% | 76.95% | 30.47% | 28.29% | 32.96% |
| uM | | | | | | | | | | | | |
| 10 | 78.23% | 124.37% | 115.30% | 121.60% | 100.99% | 139.28% | 115.31% | 105.13% | 102.55% | 41.52% | 51.82% | 43.68% |
| uM | | | | | | | | | | | | |
| 5 | 84.54% | 117.80% | 116.09% | 117.23% | 99.87% | 114.31% | 126.44% | 106.51% | 105.32% | 45.28% | 44.69% | 40.75% |
| uM | | | | | | | | | | | | |
| 1 | 96.79% | 118.82% | 112.77% | 113.51% | 96.27% | 114.57% | 99.50% | 100.31% | 97.32% | 91.49% | 71.31% | 55.56% |
| uM | | | | | | | | | | | | |

Exp 3 (SMW478), Percentage Infection Relative to Appropriate DMSO Control (e.g. Set DMSO as 100%) with Avg Background Subtraction

| | SILCS29 | SILCS29 | SILCS29 | SILCS30 | SILCS30 | SILCS30 | SILCS31 | SILCS31 | SILCS31 | SILCS32 | SILCS32 | SILCS32 |
|---|---|---|---|---|---|---|---|---|---|---|---|---|
| 50 | 6.88% | −1.19% | −1.09% | 28.61% | 29.92% | 14.89% | 26.23% | 18.61% | 22.87% | 3.55% | 3.70% | 2.61% |
| uM | | | | | | | | | | | | |
| 25 | 32.71% | 55.47% | 67.66% | 79.03% | 71.83% | 64.72% | 86.74% | 80.23% | 76.86% | 36.60% | 40.12% | 18.42% |
| uM | | | | | | | | | | | | |
| 10 | 99.71% | 91.75% | 106.45% | 73.78% | 69.60% | 68.94% | 63.55% | 89.71% | 51.99% | 26.96% | 41.66% | −1.43% |
| uM | | | | | | | | | | | | |
| 5 | 95.72% | 88.47% | 77.81% | 89.39% | 93.72% | 82.15% | 64.83% | 79.11% | 61.06% | 41.71% | 49.62% | 20.36% |
| uM | | | | | | | | | | | | |
| 1 | 97.82% | 84.39% | 100.08% | 81.75% | 100.71% | 99.49% | 90.68% | 87.75% | 89.34% | 80.30% | 82.86% | 62.40% |
| uM | | | | | | | | | | | | |

Average

| | SILCS29 | | | SILCS30 | | | SILCS31 | | | SILCS32 | |
|---|---|---|---|---|---|---|---|---|---|---|---|
| | Mean | STD | n | Mean | STD | n | Mean | STD | n | Mean | STD |
| 50 uM | 6.32% | 5.19% | 9 | 24.84% | 5.53% | 9 | 22.63% | 3.13% | 9 | 2.40% | 2.96% |
| 25 uM | 32.72% | 18.54% | 9 | 67.20% | 9.44% | 9 | 74.66% | 8.45% | 9 | 30.29% | 6.52% |
| 10 uM | 92.80% | 20.87% | 9 | 98.54% | 24.54% | 9 | 102.53% | 29.78% | 9 | 42.26% | 23.55% |
| 5 uM | 64.83% | 49.66% | 9 | 84.81% | 34.46% | 9 | 94.99% | 22.00% | 9 | 39.61% | 9.79% |
| 1 uM | 84.22% | 36.66% | 9 | 100.21% | 11.18% | 9 | 97.08% | 8.92% | 9 | 81.11% | 17.20% |

Toxicity Data

Exp 1 (SMW475), Percentage Infection Relative to Appropriate DMSO Control (e.g. Set DMSO as 100%) with Avg Background Subtraction

| | SILCS29 | SILCS29 | SILCS29 | SILCS30 | SILCS30 | SILCS30 | SILCS31 | SILCS31 | SILCS31 | SILCS32 | SILCS32 | SILCS32 |
|---|---|---|---|---|---|---|---|---|---|---|---|---|
| 50 | 37.8% | 61.7% | 87.0% | 75.8% | 84.5% | 91.9% | 100.7% | 96.9% | 99.2% | 58.6% | 50.8% | 26.2% |
| uM | | | | | | | | | | | | |
| 25 | 60.0% | 124.5% | 126.3% | 114.8% | 118.5% | 113.1% | 110.8% | 105.1% | 109.5% | 105.4% | 106.2% | 77.6% |
| uM | | | | | | | | | | | | |
| 10 | 89.0% | 115.5% | 107.6% | 92.4% | 102.3% | 100.7% | 97.1% | 83.8% | 93.3% | 108.0% | 112.2% | 98.7% |
| uM | | | | | | | | | | | | |
| 5 | 100.0% | 154.3% | 156.3% | 121.3% | 145.0% | 114.2% | 108.3% | 102.7% | 99.9% | 127.5% | 124.0% | 108.8% |
| uM | | | | | | | | | | | | |
| 1 | 99.6% | 154.9% | 111.9% | 106.4% | 108.6% | 113.8% | 112.6% | 107.1% | 108.6% | 102.9% | 109.5% | 87.8% |
| uM | | | | | | | | | | | | |

Exp 2 (SMW477), Percentage Infection Relative to Appropriate DMSO Control (e.g. Set DMSO as 1000%) with Avg Background Subtraction

| | SILCS29 | SILCS29 | SILCS29 | SILCS30 | SILCS30 | SILCS30 | SILCS31 | SILCS31 | SILCS31 | SILCS32 | SILCS32 | SILCS32 |
|---|---|---|---|---|---|---|---|---|---|---|---|---|
| 50 | 58.7% | 102.3% | 106.6% | 116.2% | 91.9% | 105.8% | 101.8% | 100.2% | 88.6% | 14.2% | 7.3% | 6.8% |
| uM | | | | | | | | | | | | |
| 25 | 113.3% | 143.7% | 139.1% | 117.7% | 112.6% | 106.7% | 109.0% | 105.0% | 108.4% | 116.7% | 115.0% | 74.7% |
| uM | | | | | | | | | | | | |
| 10 | 95.9% | 84.6% | 91.0% | 85.3% | 98.4% | 75.1% | 89.8% | 95.5% | 100.1% | 135.8% | 127.4% | 94.7% |
| uM | | | | | | | | | | | | |
| 5 | 98.5% | 92.1% | 98.3% | 92.7% | 97.0% | 89.3% | 83.5% | 96.7% | 95.7% | 131.8% | 126.5% | 116.2% |
| uM | | | | | | | | | | | | |
| 1 | 83.9% | 90.0% | 89.3% | 100.2% | 112.4% | 96.0% | 107.2% | 105.7% | 108.2% | 107.2% | 105.3% | 113.7% |
| uM | | | | | | | | | | | | |

Exp 3 (SMW478), Percentage Infection Relative to Appropriate DMSO Control (e.g. Set DMSO as 1000%) with Avg Background Subtraction

| | SILCS29 | SILCS29 | SILCS29 | SILCS30 | SILCS30 | SILCS30 | SILCS31 | SILCS31 | SILCS31 | SILCS32 | SILCS32 | SILCS32 |
|---|---|---|---|---|---|---|---|---|---|---|---|---|
| 50 | 54.8% | 54.3% | 47.1% | 67.4% | 63.0% | 82.7% | 83.3% | 102.7% | 94.6% | 91.4% | 55.7% | 16.1% |
| uM | | | | | | | | | | | | |
| 25 | 51.7% | 52.4% | 99.4% | 70.0% | 93.8% | 92.3% | 72.0% | 109.0% | 94.3% | 109.6% | 46.7% | 40.1% |
| uM | | | | | | | | | | | | |
| 10 | 49.7% | 98.8% | 86.8% | 106.7% | 126.7% | 135.8% | 134.5% | 121.6% | 169.7% | 201.2% | 167.0% | 176.6% |
| uM | | | | | | | | | | | | |
| 5 | 60.3% | 105.0% | 132.4% | 116.9% | 98.2% | 121.1% | 151.6% | 134.3% | 160.2% | 176.2% | 162.3% | 173.2% |
| uM | | | | | | | | | | | | |
| 1 | 81.9% | 123.9% | 101.8% | 120.1% | 106.6% | 110.0% | 120.8% | 126.4% | 124.3% | 136.9% | 131.4% | 130.5% |
| uM | | | | | | | | | | | | |

Average

| | SILCS29 | | | SILCS30 | | | SILCS31 | | | SILCS32 | |
|---|---|---|---|---|---|---|---|---|---|---|---|
| | Mean | STD | n | Mean | STD | n | Mean | STD | n | Mean | STD |
| 50 uM | 67.81% | 24.64% | 9 | 86.57% | 17.17% | 9 | 96.45% | 6.57% | 9 | 36.36% | 29.20% |
| 25 uM | 101.15% | 37.24% | 9 | 104.38% | 16.11% | 9 | 102.57% | 12.49% | 9 | 88.00% | 29.46% |
| 10 uM | 90.99% | 18.46% | 9 | 102.59% | 19.00% | 9 | 109.50% | 27.72% | 9 | 135.74% | 37.71% |
| 5 uM | 110.81% | 31.21% | 9 | 110.62% | 17.93% | 9 | 114.77% | 27.10% | 9 | 138.50% | 25.24% |
| 1 uM | 104.12% | 23.39% | 9 | 108.24% | 7.20% | 9 | 113.44% | 8.14% | 9 | 113.92% | 16.01% |

SILCS 33-36 Plate Setup, Greiner Celigo Plates

| | 1 | 2 | 3 | 4 | 5 | 6 |
|---|---|---|---|---|---|---|
| A | SILCS33 | SILCS33 | SILCS33 | SILCS34 | SILCS34 | SILCS34 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 4 |
|---|---|---|---|---|---|---|---|
| A | SILCS35 | SILCS35 | SILCS35 | SILCS36 | SILCS36 | SILCS36 | 50 uM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW475), Percentage Infection Relative to Appropriate DMSO Control (e.g. Set DMSO as 100%) with Avg Background Subtraction

| | SILCS33 | SILCS33 | SILCS33 | SILCS34 | SILCS34 | SILCS34 | SILCS35 | SILCS35 | SILCS35 | SILCS36 | SILCS36 | SILCS36 |
|---|---|---|---|---|---|---|---|---|---|---|---|---|
| 50 | 52.39% | 69.09% | 65.56% | 98.27% | 86.62% | 67.73% | 83.26% | 106.28% | 78.51% | 13.30% | 10.90% | 30.52% |
| uM | | | | | | | | | | | | |
| 25 | 52.02% | 4.81% | 0.00% | 73.54% | 67.42% | 88.43% | 79.86% | 56.98% | 87.77% | 42.48% | 40.16% | 33.52% |
| uM | | | | | | | | | | | | |
| 10 | 79.81% | 90.19% | 84.97% | 120.42% | 77.92% | 112.44% | 105.47% | 116.87% | 111.43% | 100.05% | 88.33% | 67.06% |
| uM | | | | | | | | | | | | |
| 5 | 69.33% | 82.60% | 84.98% | 83.89% | 91.52% | 85.89% | 93.18% | 101.74% | 91.54% | 103.78% | 96.97% | 70.88% |
| uM | | | | | | | | | | | | |
| 1 | 53.62% | 101.44% | 86.72% | 84.46% | 79.06% | 78.77% | 90.30% | 94.24% | 95.59% | 112.32% | 102.95% | 77.60% |
| uM | | | | | | | | | | | | |

Exp 2 (SMW477), Percentage Infection Relative to Appropriate DMSO Control (e.g. Set DMSO as 100%) with Avg Background Subtraction

| | SILCS33 | SILCS33 | SILCS33 | SILCS34 | SILCS34 | SILCS34 | SILCS35 | SILCS35 | SILCS35 | SILCS36 | SILCS36 | SILCS36 |
|---|---|---|---|---|---|---|---|---|---|---|---|---|
| 50 | 31.41% | 36.50% | 41.52% | 62.30% | 66.46% | 58.63% | 109.53% | 98.67% | 105.03% | 35.38% | 21.21% | 7.69% |
| uM | | | | | | | | | | | | |
| 25 | 73.62% | 97.76% | 95.99% | 109.38% | 111.61% | 103.44% | 125.42% | 107.58% | 104.40% | 70.58% | 67.21% | 40.18% |
| uM | | | | | | | | | | | | |
| 10 | 114.36% | 136.38% | 130.63% | 142.94% | 144.38% | 155.80% | 155.83% | 113.76% | 119.79% | 111.26% | 112.57% | 100.88% |
| uM | | | | | | | | | | | | |
| 5 | 128.80% | 118.55% | 121.74% | 137.43% | 146.89% | 126.80% | 138.25% | 128.36% | 147.80% | 140.43% | 143.68% | 122.53% |
| uM | | | | | | | | | | | | |
| 1 | 120.88% | 113.91% | 111.55% | 117.81% | 129.29% | 121.32% | 131.21% | 119.10% | 117.97% | 136.49% | 133.25% | 131.77% |
| uM | | | | | | | | | | | | |

Exp 3 (SMW478), Percentage Infection Relative to Appropriate DMSO Control (e.g. Set DMSO as 100%) with Avg Background Subtraction

| | SILCS33 | SILCS33 | SILCS33 | SILCS34 | SILCS34 | SILCS34 | SILCS35 | SILCS35 | SILCS35 | SILCS36 | SILCS36 | SILCS36 |
|---|---|---|---|---|---|---|---|---|---|---|---|---|
| 50 | 71.82% | 89.49% | 84.99% | 145.01% | 171.02% | 138.34% | 140.10% | 158.92% | 157.74% | 62.41% | 47.18% | 17.75% |
| uM | | | | | | | | | | | | |
| 25 | 126.24% | 109.99% | 117.46% | 164.75% | 165.91% | 139.33% | 126.08% | 121.76% | 139.01% | 99.36% | 76.23% | 47.27% |
| uM | | | | | | | | | | | | |
| 10 | 159.81% | 126.46% | 162.16% | 127.49% | 93.45% | 116.79% | 151.59% | 95.94% | 77.50% | 99.80% | 128.16% | 117.51% |
| uM | | | | | | | | | | | | |
| 5 | 121.27% | 137.96% | 122.71% | 108.72% | 96.17% | 137.98% | 134.19% | 78.79% | 94.59% | 103.27% | 118.27% | 106.75% |
| uM | | | | | | | | | | | | |
| 1 | 106.36% | −29.50% | −23.99% | 120.13% | 101.31% | 117.84% | 105.76% | 80.57% | 90.16% | 116.69% | 121.41% | 101.10% |
| uM | | | | | | | | | | | | |

Average

| | SILCS33 | | | SILCS34 | | | SILCS35 | | | SILCS36 | |
|---|---|---|---|---|---|---|---|---|---|---|---|
| | Mean | STD | n | Mean | STD | n | Mean | STD | n | Mean | STD |
| 50 uM | 60.31% | 20.96% | 9 | 99.38% | 41.86% | 9 | 115.34% | 29.99% | 9 | 27.37% | 18.31% |
| 25 uM | 75.32% | 47.03% | 9 | 113.76% | 36.25% | 9 | 105.43% | 26.31% | 9 | 57.44% | 22.01% |
| 10 uM | 120.53% | 30.71% | 9 | 121.29% | 24.97% | 9 | 116.46% | 24.72% | 9 | 102.85% | 17.80% |
| 5 uM | 109.77% | 24.15% | 9 | 112.81% | 24.76% | 9 | 112.05% | 25.04% | 9 | 111.84% | 22.50% |
| 1 uM | 71.22% | 58.97% | 9 | 105.55% | 20.03% | 9 | 102.77% | 16.76% | 9 | 114.84% | 18.92% |

Toxicity Data

Exp 1 (SMW475), Percentage Infection Relative to Appropriate DMSO Control (e.g. Set DMSO as 100%) with Avg Background Subtraction

| | SILCS33 | SILCS33 | SILCS33 | SILCS34 | SILCS34 | SILCS34 | SILCS35 | SILCS35 | SILCS35 | SILCS36 | SILCS36 | SILCS36 |
|---|---|---|---|---|---|---|---|---|---|---|---|---|
| 50 | 70.2% | 73.2% | 76.5% | 89.3% | 79.2% | 68.3% | 73.0% | 72.5% | 63.4% | 54.0% | 68.3% | 44.0% |
| uM | | | | | | | | | | | | |
| 25 | 78.0% | 127.6% | 150.1% | 116.7% | 129.0% | 105.0% | 107.9% | 126.0% | 108.3% | 118.7% | 122.3% | 69.9% |
| uM | | | | | | | | | | | | |
| 10 | 98.7% | 102.4% | 110.1% | 95.5% | 113.4% | 95.7% | 100.2% | 100.7% | 99.3% | 108.1% | 100.9% | 83.1% |
| uM | | | | | | | | | | | | |
| 5 | 108.2% | 111.1% | 108.0% | 111.3% | 114.1% | 115.7% | 104.7% | 105.2% | 108.0% | 95.7% | 101.1% | 78.9% |
| uM | | | | | | | | | | | | |
| 1 | 122.2% | 99.5% | 109.4% | 111.3% | 110.2% | 117.4% | 108.1% | 106.1% | 111.5% | 94.6% | 95.5% | 78.6% |
| uM | | | | | | | | | | | | |

Exp 2 (SMW477), Percentage Infection Relative to Appropriate DMSO Control (e.g. Set DMSO as 100%) with Avg Background Subtraction

| | SILCS33 | SILCS33 | SILCS33 | SILCS34 | SILCS34 | SILCS34 | SILCS35 | SILCS35 | SILCS35 | SILCS36 | SILCS36 | SILCS36 |
|---|---|---|---|---|---|---|---|---|---|---|---|---|
| 50 | 61.9% | 53.5% | 75.3% | 83.0% | 92.7% | 94.1% | 74.1% | 77.4% | 63.9% | 97.0% | 94.2% | 86.8% |
| uM | | | | | | | | | | | | |
| 25 | 64.1% | 48.9% | 89.4% | 87.6% | 90.8% | 95.6% | 84.4% | 91.4% | 95.9% | 117.5% | 112.5% | 98.3% |
| uM | | | | | | | | | | | | |
| 10 | 59.6% | 71.7% | 81.9% | 74.5% | 72.3% | 67.3% | 72.6% | 93.8% | 92.3% | 93.9% | 93.4% | 77.7% |
| uM | | | | | | | | | | | | |
| 5 | 66.5% | 87.6% | 88.4% | 77.9% | 73.3% | 86.3% | 84.0% | 86.1% | 75.0% | 79.2% | 74.5% | 67.8% |
| uM | | | | | | | | | | | | |
| 1 | 81.8% | 91.0% | 92.4% | 86.8% | 85.0% | 85.6% | 78.7% | 82.9% | 87.9% | 81.9% | 81.7% | 69.4% |
| uM | | | | | | | | | | | | |

Exp 3 (SMW478), Percentage Infection Relative to Appropriate DMSO Control (e.g. Set DMSO as 100%) with Avg Background Subtraction

| | SILCS33 | SILCS33 | SILCS33 | SILCS34 | SILCS34 | SILCS34 | SILCS35 | SILCS35 | SILCS35 | SILCS36 | SILCS36 | SILCS36 |
|---|---|---|---|---|---|---|---|---|---|---|---|---|
| 50 | 37.6% | 38.1% | 46.5% | 40.3% | 26.4% | 37.4% | 47.1% | 39.3% | 33.9% | 52.4% | 38.9% | 33.4% |
| uM | | | | | | | | | | | | |
| 25 | 53.2% | 80.7% | 80.4% | 68.3% | 46.8% | 65.2% | 90.9% | 69.3% | 65.7% | 86.5% | 53.5% | 47.4% |
| uM | | | | | | | | | | | | |
| 10 | 53.8% | 96.3% | 47.1% | 91.1% | 110.1% | 98.8% | 12.3% | 119.6% | 117.2% | 121.0% | 70.1% | 45.3% |
| uM | | | | | | | | | | | | |
| 5 | 83.2% | 92.6% | 102.1% | 108.6% | 115.4% | 94.0% | 98.2% | 128.0% | 116.6% | 114.1% | 93.4% | 59.7% |
| uM | | | | | | | | | | | | |
| 1 | 98.3% | 191.4% | 182.5% | 101.5% | 119.9% | 104.4% | 112.9% | 133.1% | 126.7% | 110.8% | 103.0% | 72.9% |
| uM | | | | | | | | | | | | |

Average

| | SILCS33 | | | SILCS34 | | | SILCS35 | | | SILCS36 | |
|---|---|---|---|---|---|---|---|---|---|---|---|
| | Mean | STD | n | Mean | STD | n | Mean | STD | n | Mean | STD |
| 50 uM | 59.20% | 15.78% | 9 | 67.85% | 26.30% | 9 | 60.50% | 16.31% | 9 | 63.21% | 24.33% |
| 25 uM | 85.80% | 33.37% | 9 | 89.42% | 26.09% | 9 | 93.31% | 19.16% | 9 | 91.83% | 29.02% |
| 10 uM | 80.17% | 23.13% | 9 | 91.00% | 16.40% | 9 | 89.77% | 32.17% | 9 | 88.15% | 22.37% |
| 5 uM | 94.18% | 14.58% | 9 | 99.62% | 17.01% | 9 | 100.64% | 16.76% | 9 | 84.91% | 17.36% |
| 1 uM | 118.72% | 40.40% | 9 | 102.47% | 13.71% | 9 | 105.31% | 18.86% | 9 | 87.60% | 14.05% |

SILCS 37-42 Plate Setup, Nunc Celigo Plates

| | 1 | 2 | 3 | 4 | 5 | 6 |
|---|---|---|---|---|---|---|
| A | SILCS37 | SILCS37 | SILCS38 | SILCS38 | SICLS39 | SICLS39 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 1 |
|---|---|---|---|---|---|---|---|
| A | SILCS40 | SILCS40 | SILCS41 | SILCS41 | SILCS42 | SILCS42 | 50 uM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW491), Percentage Infection Relative to Appropriate DMSO Control (e.g. Set DMSO as 100%) with Avg Background Subtraction

| | SILCS37 | SILCS37 | SILCS38 | SILCS38 | SICLS39 | SICLS39 | SILCS40 | SILCS40 | SILCS41 | SILCS41 | SILCS42 | SILCS42 |
|---|---|---|---|---|---|---|---|---|---|---|---|---|
| 50 | 136.02% | 127.57% | 96.25% | 89.03% | −5.92% | 15.97% | 46.53% | 56.51% | 3.84% | 5.27% | 2.10% | 3.15% |
| uM | | | | | | | | | | | | |
| 25 | 117.51% | 105.36% | 110.71% | 103.81% | 1.98% | 2.03% | 122.15% | 145.90% | 4.77% | 2.26% | 47.94% | 34.15% |
| uM | | | | | | | | | | | | |
| 10 | 109.85% | 92.25% | 91.03% | 87.80% | 26.66% | 13.52% | 125.55% | 160.68% | 1.51% | 2.55% | 132.40% | 13.35% |
| uM | | | | | | | | | | | | |
| 5 | 95.46% | 97.68% | 101.51% | 104.07% | 61.21% | 63.14% | 118.41% | 120.39% | 20.73% | 20.40% | 26.18% | 31.73% |
| uM | | | | | | | | | | | | |
| 1 | 88.98% | 80.18% | 101.61% | 80.30% | 94.45% | 94.76% | 161.72% | 144.95% | 78.53% | 92.14% | 93.43% | 88.84% |
| uM | | | | | | | | | | | | |

Exp 2 (SMW492), Percentage Infection Relative to Appropriate DMSO Control (e.g. Set DMSO as 1000%) with Avg Background Subtraction

| | SILCS37 | SILCS37 | SILCS38 | SILCS38 | SICLS39 | SICLS39 | SILCS40 | SILCS40 | SILCS41 | SILCS41 | SILCS42 | SILCS42 |
|---|---|---|---|---|---|---|---|---|---|---|---|---|
| 50 | 88.75% | 46.65% | 90.22% | 70.45% | 5.19% | 10.37% | 61.71% | 43.46% | 6.25% | 8.27% | 88.75% | 86.52% |
| uM | | | | | | | | | | | | |
| 25 | 121.75% | 63.91% | 123.54% | 56.46% | 5.42% | 6.99% | 185.38% | 112.33% | 3.10% | 1.58% | 50.85% | 76.10% |
| uM | | | | | | | | | | | | |
| 10 | 73.60% | 28.54% | 63.76% | 49.65% | 21.95% | 19.13% | 101.31% | 73.96% | 2.35% | 1.08% | 59.18% | 61.61% |
| uM | | | | | | | | | | | | |
| 5 | 71.36% | 74.06% | 72.80% | 60.72% | 40.93% | 42.02% | 88.04% | 68.60% | 12.91% | 15.34% | 42.63% | 60.22% |
| uM | | | | | | | | | | | | |
| 1 | 80.82% | 84.84% | 74.34% | 48.74% | 55.17% | 68.37% | 86.70% | 77.51% | 10.73% | 11.86% | 50.77% | 51.19% |
| uM | | | | | | | | | | | | |

Average (SILCS 37-39)

| | SILCS37 | | | SILCS38 | | | SICLS39 | | |
|---|---|---|---|---|---|---|---|---|---|
| | Mean | STD | n | Mean | STD | n | Mean | STD | n |
| 50 uM | 99.75% | 40.95% | 4 | 86.49% | 11.15% | 4 | 6.40% | 9.32% | 4 |
| 25 uM | 102.13% | 26.41% | 4 | 98.63% | 29.28% | 4 | 4.11% | 2.50% | 4 |
| 10 uM | 76.06% | 34.97% | 4 | 73.06% | 19.79% | 4 | 20.32% | 5.49% | 4 |
| 5 uM | 84.64% | 13.85% | 4 | 84.78% | 21.40% | 4 | 51.83% | 11.99% | 4 |
| 1 uM | 83.71% | 4.07% | 4 | 76.25% | 21.76% | 4 | 78.19% | 19.71% | 4 |

Average (SILCS 40-42)

| | SILCS40 | | | SILCS41 | | | SILCS42 | | |
|---|---|---|---|---|---|---|---|---|---|
| | Mean | STD | n | Mean | STD | n | Mean | STD | n |
| 50 uM | 52.05% | 8.51% | 4 | 5.91% | 1.86% | 4 | 45.13% | 49.09% | 4 |
| 25 uM | 141.44% | 32.51% | 4 | 2.93% | 1.38% | 4 | 52.26% | 17.48% | 4 |
| 10 uM | 115.37% | 36.83% | 4 | 1.87% | 0.69% | 4 | 66.63% | 49.14% | 4 |
| 5 uM | 98.86% | 25.02% | 4 | 17.35% | 3.85% | 4 | 40.19% | 15.00% | 4 |
| 1 uM | 117.72% | 41.86% | 4 | 48.32% | 43.11% | 4 | 71.06% | 23.26% | 4 |

Toxicity Data

Exp 1 (SMW491), Percentage Infection Relative to Appropriate DMSO Control (e.g. Set DMSO as 1000%) with Avg Background Subtraction

| | SILCS37 | SILCS37 | SILCS38 | SILCS38 | SICLS39 | SICLS39 | SILCS40 | SILCS40 | SILCS41 | SILCS41 | SILCS42 | SILCS42 |
|---|---|---|---|---|---|---|---|---|---|---|---|---|
| 50 | 91% | 109% | 125% | 112% | 1% | 0% | 136% | 147% | 105% | 116% | 1% | 1% |
| uM | | | | | | | | | | | | |
| 25 | 99% | 112% | 119% | 110% | 145% | 138% | 86% | 80% | 115% | 117% | 0% | 1% |
| uM | | | | | | | | | | | | |
| 10 | 99% | 122% | 99% | 114% | 164% | 152% | 83% | 78% | 122% | 111% | 0% | 0% |
| uM | | | | | | | | | | | | |
| 5 | 116% | 104% | 121% | 98% | 112% | 111% | 78% | 100% | 138% | 144% | 140% | 135% |
| uM | | | | | | | | | | | | |
| 1 | 101% | 124% | 113% | 105% | 96% | 114% | 66% | 73% | 96% | 101% | 111% | 107% |
| uM | | | | | | | | | | | | |

Exp 2 (SMW492), Percentage Infection Relative to Appropriate DMSO Control (e.g. Set DMSO as 1000%) with Avg background Subtraction

| | SILCS37 | SILCS37 | SILCS38 | SILCS38 | SICLS39 | SICLS39 | SILCS40 | SILCS40 | SILCS41 | SILCS41 | SILCS42 | SILCS42 |
|---|---|---|---|---|---|---|---|---|---|---|---|---|
| 50 | 0% | 101% | 88% | 96% | 93% | 91% | 89% | 99% | 90% | 95% | 0% | 0% |
| uM | | | | | | | | | | | | |
| 25 | 96% | 110% | 92% | 109% | 120% | 109% | 71% | 98% | 121% | 120% | 17% | 1% |
| uM | | | | | | | | | | | | |
| 10 | 112% | 132% | 117% | 131% | 139% | 144% | 106% | 104% | 127% | 135% | 0% | 0% |
| uM | | | | | | | | | | | | |
| 5 | 108% | 113% | 109% | 112% | 111% | 127% | 97% | 110% | 136% | 141% | 126% | 114% |
| uM | | | | | | | | | | | | |
| 1 | 104% | 107% | 108% | 122% | 112% | 109% | 95% | 112% | 140% | 136% | 123% | 117% |
| uM | | | | | | | | | | | | |

Average (SILCS 37-39)

| | SILCS37 | | | SILCS38 | | | SICLS39 | | |
|---|---|---|---|---|---|---|---|---|---|
| | Mean | STD | n | Mean | STD | n | Mean | STD | n |
| 50 uM | 75.29% | 50.53% | 4 | 105.21% | 16.22% | 4 | 46.35% | 52.65% | 4 |
| 25 uM | 104.07% | 7.94% | 4 | 107.71% | 11.14% | 4 | 128.15% | 16.75% | 4 |
| 10 uM | 116.31% | 14.26% | 4 | 115.05% | 12.92% | 4 | 149.83% | 11.11% | 4 |
| 5 uM | 110.16% | 5.39% | 4 | 110.00% | 9.14% | 4 | 114.94% | 7.88% | 4 |
| 1 uM | 108.95% | 10.35% | 4 | 111.85% | 7.73% | 4 | 107.69% | 8.28% | 4 |

Average (SILCS 40-42)

| | SILCS40 | | | SILCS41 | | | SILCS42 | | |
|---|---|---|---|---|---|---|---|---|---|
| | Mean | STD | n | Mean | STD | n | Mean | STD | n |
| 50 uM | 117.52% | 28.23% | 4 | 101.49% | 11.51% | 4 | 0.62% | 0.55% | 4 |
| 25 uM | 83.93% | 11.52% | 4 | 118.34% | 2.68% | 4 | 4.52% | 8.07% | 4 |
| 10 uM | 92.49% | 14.36% | 4 | 124.03% | 9.87% | 4 | 0.21% | 0.09% | 4 |
| 5 uM | 96.33% | 13.53% | 4 | 139.55% | 3.76% | 4 | 128.78% | 11.76% | 4 |
| 1 uM | 86.49% | 21.13% | 4 | 118.33% | 22.71% | 4 | 114.38% | 7.28% | 4 |

SILCS 43-48 Plate Setup, Nunc Celigo Plates

| | 1 | 2 | 3 | 4 | 5 | 6 |
|---|---|---|---|---|---|---|
| A | SILCS43 | SILCS43 | SILCS44 | SILCS44 | SILCS45 | SILCS45 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 2 |
|---|---|---|---|---|---|---|---|
| A | SILCS46 | SILCS46 | SILCS47 | SILCS47 | SILCS48 | SILCS48 | 50 uM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW491), Percentage Infection Relative to Appropriate DMSO Control (e.g. Set DMSO as 1000%) with Avg Background Subtraction

| | SILCS43 | SILCS43 | SILCS44 | SILCS44 | SILCS45 | SILCS45 | SILCS46 | SILCS46 | SILCS47 | SILCS47 | SILCS48 | SILCS48 |
|---|---|---|---|---|---|---|---|---|---|---|---|---|
| 50 | −10.07% | −10.07% | 1.28% | 3.26% | 1.17% | −2.53% | 2.55% | −4.15% | −2.14% | −2.13% | 159.63% | 145.37% |
| uM | | | | | | | | | | | | |
| 25 | 1.98% | −0.83% | 1.08% | −0.14% | −0.41% | −2.68% | −3.35% | 0.11% | 72.26% | 73.05% | 102.87% | 119.89% |
| uM | | | | | | | | | | | | |
| 10 | 0.28% | −0.66% | 4.70% | 2.01% | 0.12% | −3.09% | −2.65% | −0.77% | 58.60% | 58.62% | 96.64% | 110.53% |
| uM | | | | | | | | | | | | |
| 5 | 5.93% | 4.01% | 84.11% | 74.83% | −1.31% | −0.48% | −0.70% | 0.86% | 92.36% | 69.21% | 70.35% | 83.94% |
| uM | | | | | | | | | | | | |
| 1 | 11.14% | 9.36% | 90.57% | 106.30% | 102.92% | 112.49% | 73.61% | 132.86% | 92.39% | 87.95% | 87.03% | 82.70% |
| uM | | | | | | | | | | | | |

Exp 2 (SMW492), Percentage Infection Relative to Appropriate DMSO Control (e.g. Set DMSO as 1000%) with Avg Background Subtraction

| | SILCS43 | SILCS43 | SILCS44 | SILCS44 | SILCS45 | SILCS45 | SILCS46 | SILCS46 | SILCS47 | SILCS47 | SILCS48 | SILCS48 |
|---|---|---|---|---|---|---|---|---|---|---|---|---|
| 50 | −16.61% | −16.61% | −2.01% | 0.90% | 7.94% | 7.68% | 3.90% | 1.08% | 15.36% | 31.15% | 177.82% | 157.32% |
| uM | | | | | | | | | | | | |
| 25 | 18.29% | 5.04% | −2.55% | 2.35% | 0.00% | −2.03% | 0.98% | 2.39% | 33.79% | 62.95% | 97.57% | 81.03% |
| uM | | | | | | | | | | | | |
| 10 | 1.81% | 1.99% | 11.68% | 9.67% | 7.69% | 10.80% | 47.59% | 42.39% | 71.20% | 79.16% | 49.42% | 83.79% |
| uM | | | | | | | | | | | | |
| 5 | 13.08% | 2.98% | 5.29% | 6.25% | −2.43% | 0.22% | 0.99% | 1.92% | 74.13% | 90.49% | 84.51% | 61.96% |
| uM | | | | | | | | | | | | |
| 1 | 18.42% | 33.91% | 122.34% | 89.19% | 3.72% | −0.43% | 49.01% | 32.97% | 101.71% | 94.46% | 59.24% | 57.80% |
| uM | | | | | | | | | | | | |

Average (SILCS 43-45)

| | SILCS43 | | | SILCS44 | | | SILCS45 | | |
|---|---|---|---|---|---|---|---|---|---|
| | Mean | STD | n | Mean | STD | n | Mean | STD | n |
| 50 uM | −13.34% | 3.78% | 4 | 0.86% | 2.18% | 4 | 3.57% | 5.13% | 4 |
| 25 uM | 6.12% | 8.46% | 4 | 0.18% | 2.09% | 4 | −1.28% | 1.28% | 4 |
| 10 uM | 0.85% | 1.27% | 4 | 7.02% | 4.44% | 4 | 3.88% | 6.46% | 4 |
| 5 uM | 6.50% | 4.56% | 4 | 42.62% | 42.72% | 4 | −1.00% | 1.14% | 4 |
| 1 uM | 18.21% | 11.18% | 4 | 102.10% | 15.56% | 4 | 54.68% | 61.38% | 4 |

Average (SILCS 46-48)

| | SILCS46 | | | SILCS47 | | | SILCS48 | | |
|---|---|---|---|---|---|---|---|---|---|
| | Mean | STD | n | Mean | STD | n | Mean | STD | n |
| 50 uM | 0.84% | 3.52% | 4 | 10.56% | 16.01% | 4 | 160.04% | 13.41% | 4 |
| 25 uM | 0.03% | 2.44% | 4 | 60.51% | 18.39% | 4 | 100.34% | 16.01% | 4 |
| 10 uM | 21.64% | 27.05% | 4 | 66.90% | 10.10% | 4 | 85.09% | 26.17% | 4 |
| 5 uM | 0.77% | 1.09% | 4 | 81.55% | 11.61% | 4 | 75.19% | 10.98% | 4 |
| 1 uM | 72.12% | 43.81% | 4 | 94.13% | 5.74% | 4 | 71.69% | 15.32% | 4 |

Toxicity Data

Exp 1 (SMW491), Percentage Infection Relative to Appropriate DMSO Control (e.g. Set DMSO as 1000%) with Avg Background Subtraction

| | SILCS43 | SILCS43 | SILCS44 | SILCS44 | SILCS45 | SILCS45 | SILCS46 | SILCS46 | SILCS47 | SILCS47 | SILCS48 | SILCS48 |
|---|---|---|---|---|---|---|---|---|---|---|---|---|
| 50 | 0% | 0% | 61% | 117% | 129% | 121% | 84% | 108% | 67% | 19% | 79% | 77% |
| uM | | | | | | | | | | | | |
| 25 | 121% | 136% | 124% | 133% | 95% | 123% | 118% | 101% | 132% | 112% | 95% | 88% |
| uM | | | | | | | | | | | | |
| 10 | 118% | 121% | 171% | 140% | 111% | 127% | 126% | 116% | 109% | 147% | 86% | 88% |
| uM | | | | | | | | | | | | |
| 5 | 130% | 166% | 94% | 105% | 121% | 114% | 124% | 137% | 98% | 108% | 108% | 106% |
| uM | | | | | | | | | | | | |
| 1 | 151% | 166% | 103% | 87% | 94% | 101% | 87% | 86% | 101% | 109% | 112% | 102% |
| uM | | | | | | | | | | | | |

Exp 2 (SMW492), Percentage Infection Relative to Appropriate DMSO Control (e.g. Set DMSO as 1000%) with Avg Background Subtraction

| | SILCS43 | SILCS43 | SILCS44 | SILCS44 | SILCS45 | SILCS45 | SILCS46 | SILCS46 | SILCS47 | SILCS47 | SILCS48 | SILCS48 |
|---|---|---|---|---|---|---|---|---|---|---|---|---|
| 50 | 0% | 0% | 123% | 124% | 126% | 126% | 118% | 121% | 88% | 59% | 81% | 75% |
| uM | | | | | | | | | | | | |
| 25 | 143% | 145% | 130% | 147% | 135% | 127% | 117% | 135% | 128% | 123% | 109% | 116% |
| uM | | | | | | | | | | | | |
| 10 | 145% | 146% | 141% | 139% | 137% | 151% | 134% | 130% | 125% | 125% | 135% | 121% |
| uM | | | | | | | | | | | | |
| 5 | 155% | 156% | 145% | 132% | 133% | 126% | 120% | 143% | 127% | 118% | 115% | 135% |
| uM | | | | | | | | | | | | |
| 1 | 156% | 145% | 104% | 107% | 139% | 133% | 100% | 140% | 100% | 116% | 130% | 132% |
| uM | | | | | | | | | | | | |

Average (SILCS 43-45)

| | SILCS43 | | | SILCS44 | | | SILCS45 | | |
|---|---|---|---|---|---|---|---|---|---|
| | Mean | STD | n | Mean | STD | n | Mean | STD | n |
| 50 uM | 0.01% | 0.01% | 4 | 106.37% | 30.32% | 4 | 125.46% | 3.28% | 4 |
| 25 uM | 136.27% | 10.51% | 4 | 133.30% | 9.69% | 4 | 119.79% | 17.60% | 4 |
| 10 uM | 132.39% | 14.99% | 4 | 147.85% | 15.63% | 4 | 131.34% | 16.53% | 4 |
| 5 uM | 151.78% | 15.00% | 4 | 118.91% | 23.30% | 4 | 123.47% | 7.81% | 4 |
| 1 uM | 154.73% | 9.02% | 4 | 99.99% | 8.98% | 4 | 116.96% | 22.56% | 4 |

Average (SILCS 46-48)

| | SILCS46 | | | SILCS47 | | | SILCS48 | | |
|---|---|---|---|---|---|---|---|---|---|
| | Mean | STD | n | Mean | STD | n | Mean | STD | n |
| 50 uM | 107.72% | 16.74% | 4 | 58.34% | 29.06% | 4 | 77.81% | 2.65% | 4 |
| 25 uM | 117.77% | 13.94% | 4 | 123.82% | 8.58% | 4 | 102.04% | 13.01% | 4 |
| 10 uM | 126.44% | 7.62% | 4 | 126.65% | 15.27% | 4 | 107.43% | 24.49% | 4 |
| 5 uM | 131.17% | 10.74% | 4 | 112.86% | 12.45% | 4 | 115.84% | 13.25% | 4 |
| 1 uM | 103.33% | 25.48% | 4 | 106.63% | 7.46% | 4 | 119.19% | 14.75% | 4 |

UMB18G Plate Setup, Nunc Celigo Plates

| | | | | | | | | | | | | |
|---|---|---|---|---|---|---|---|---|---|---|---|---|
| 1 | 2 | 3 | 4 | 5 | 6 | 7 | 8 | 9 | 10 | 11 | 12 | Plate 3 |

| | | | | | | | | | |
|---|---|---|---|---|---|---|---|---|---|
| A | UMB18G | UMB18G | UMB18G | UMB18G | UMB18G | UMB18G | UMB18G | UMB18G | 50 uM |
| B | | | | | | | | | 25 uM |
| C | | | | | | | | | 10 uM |
| D | | | | | | | | | 5 uM |
| E | | | | | | | | | 1 uM |
| F | DMSO | | | | | | | | 0.50% |
| G | DMSO | | | | | | | | 0.10% |
| H | Uninfect | | | | | | | | N/A |

Infection Data

Exp 1 (SMW491), Percentage Infection Relative to Appropriate DMSO Control (e.g. Set DMSO as 1000%) with Avg Background Subtraction

| | UMB18G | UMB18G | UMB18G | UMB18G | UMB18G | UMB18G | UMB18G | UMB18G |
|---|---|---|---|---|---|---|---|---|
| 50 uM | 7.21% | 9.69% | 16.26% | −2.23% | 8.91% | 3.89% | 2.69% | −0.89% |
| 25 uM | 15.58% | 3.24% | 4.43% | −1.50% | 12.74% | 10.04% | 9.27% | 10.93% |
| 10 uM | 5.19% | 1.05% | 0.23% | 2.20% | 5.21% | 4.17% | 3.28% | 4.40% |
| 5 uM | 92.29% | 78.91% | 1.36% | 0.22% | 51.36% | 22.54% | 5.97% | 1.33% |
| 1 uM | 51.30% | 56.07% | 25.47% | 32.20% | 36.10% | 42.43% | 29.78% | 22.86% |

Exp 2 (SMW492), Percentage Infection Relative to Appropriate DMSO Control (e.g. Set DMSO as 100%) with Avg Background Subtraction

| | UMB18G | UMB18G | UMB18G | UMB18G | UMB18G | UMB18G | UMB18G | UMB18G |
|---|---|---|---|---|---|---|---|---|
| 50 uM | 6.55% | 8.71% | 5.40% | 10.13% | 20.86% | 6.58% | 1.42% | 0.12% |
| 25 uM | 11.33% | 14.66% | 5.92% | 8.59% | 7.41% | 15.43% | 6.51% | 2.71% |
| 10 uM | 15.18% | 12.71% | 12.92% | 8.48% | 10.42% | 9.51% | 5.72% | 6.91% |
| 5 uM | 23.99% | 22.11% | 86.30% | 83.38% | 20.17% | 18.09% | 70.77% | 84.71% |
| 1 uM | 87.68% | 68.32% | 72.99% | 69.05% | 75.23% | 75.22% | 50.23% | 54.62% |

Average

| | UMB18G | | | UMB18G | | | UMB18G | | | UMB18G | | |
|---|---|---|---|---|---|---|---|---|---|---|---|---|
| | Mean | STD | n | Mean | STD | n | Mean | STD | n | Mean | STD | n |
| 50 uM | 8.04% | 1.43% | 4 | 7.39% | 7.80% | 4 | 10.06% | 7.49% | 4 | 0.83% | 1.56% | 4 |
| 25 uM | 11.20% | 5.61% | 4 | 4.36% | 4.27% | 4 | 11.41% | 3.45% | 4 | 7.36% | 3.60% | 4 |
| 10 uM | 8.53% | 6.55% | 4 | 5.96% | 5.82% | 4 | 7.33% | 3.10% | 4 | 5.08% | 1.58% | 4 |
| 5 uM | 54.32% | 36.53% | 4 | 42.82% | 48.55% | 4 | 28.04% | 15.66% | 4 | 40.70% | 43.19% | 4 |
| 1 uM | 65.84% | 16.23% | 4 | 49.93% | 24.56% | 4 | 57.24% | 20.92% | 4 | 39.37% | 15.44% | 4 |

Toxicity Data

Exp 1 (SMW491), Percentage Infection Relative to Appropriate DMSO Control (e.g. Set DMSO as 100%) with Avg Background N Subtraction

| | UMB18G | UMB18G | UMB18G | UMB18G | UMB18G | UMB18G | UMB18G | UMB18G |
|---|---|---|---|---|---|---|---|---|
| 50 uM | 38% | 120% | 112% | 95% | 101% | 100% | 75% | 105% |
| 25 uM | 144% | 112% | 123% | 135% | 104% | 124% | 129% | 120% |
| 10 uM | 160% | 151% | 181% | 167% | 134% | 134% | 176% | 152% |
| 5 uM | 121% | 135% | 200% | 133% | 112% | 135% | 149% | 159% |
| 1 uM | 140% | 139% | 158% | 79% | 130% | 131% | 125% | 143% |

Exp 2 (SMW492), Percentage Infection Relative to Appropriate DMSO Control (e.g. Set DMSO as 1000%) with Avg Background Subtraction

| | UMB18G | UMB18G | UMB18G | UMB18G | UMB18G | UMB18G | UMB18G | UMB18G |
|---|---|---|---|---|---|---|---|---|
| 50 uM | 65% | 108% | 112% | 109% | 105% | 96% | 97% | 103% |
| 25 uM | 118% | 120% | 120% | 122% | 127% | 104% | 105% | 116% |
| 10 uM | 131% | 130% | 134% | 136% | 130% | 133% | 118% | 111% |
| 5 uM | 131% | 132% | 104% | 105% | 127% | 113% | 95% | 99% |
| 1 uM | 102% | 115% | 109% | 130% | 93% | 117% | 112% | 114% |

Average

| | UMB18G | | | UMB18G | | | UMB18G | | | UMB18G | | |
|---|---|---|---|---|---|---|---|---|---|---|---|---|
| | Mean | STD | n | Mean | STD | n | Mean | STD | n | Mean | STD | n |
| 50 uM | 82.73% | 38.21% | 4 | 106.99% | 8.34% | 4 | 100.66% | 3.46% | 4 | 94.86% | 13.89% | 4 |
| 25 uM | 123.37% | 13.86% | 4 | 125.35% | 6.63% | 4 | 114.63% | 12.43% | 4 | 117.50% | 9.78% | 4 |
| 10 uM | 143.16% | 14.71% | 4 | 154.42% | 23.45% | 4 | 132.79% | 2.03% | 4 | 139.46% | 30.27% | 4 |
| 5 uM | 130.01% | 6.32% | 4 | 135.50% | 45.08% | 4 | 121.59% | 11.19% | 4 | 125.34% | 33.22% | 4 |
| 1 uM | 123.95% | 18.52% | 4 | 118.99% | 33.29% | 4 | 117.65% | 17.55% | 4 | 123.59% | 14.33% | 4 |

SILCS 49-54 Plate Setup, Nunc Celigo Plates

| | 1 | 2 | 3 | 4 | 5 | 6 |
|---|---|---|---|---|---|---|
| A | SILCS49 | SILCS49 | SILCS50 | SILCS50 | SICLS51 | SICLS51 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 1 |
|---|---|---|---|---|---|---|---|
| A | SICLS52 | SICLS52 | SICLS53 | SICLS53 | SICLS54 | SICLS54 | 50 uM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW506), Percentage Infection Relative to Appropriate DMSO Control (e.g. Set DMSO as 100%) with Avg Background Subtraction

| | SILCS49 | SILCS49 | SILCS50 | SILCS50 | SICLS51 | SICLS51 | SICLS52 | SICLS52 | SICLS53 | SICLS53 | SICLS54 | SICLS54 |
|---|---|---|---|---|---|---|---|---|---|---|---|---|
| 50 | 9.17% | 7.35% | −3.31% | −2.97% | 91.33% | 101.02% | 124.01% | 110.56% | 112.23% | 123.43% | 104.55% | 95.27% |
| uM | | | | | | | | | | | | |
| 25 | 27.25% | 33.31% | 0.28% | −1.38% | 115.11% | 73.79% | 124.78% | 116.32% | 103.57% | 117.02% | 117.47% | 78.17% |
| uM | | | | | | | | | | | | |
| 10 | 38.04% | 34.18% | 0.81% | 1.10% | 83.06% | 110.56% | 98.74% | 87.15% | 103.68% | 100.49% | 84.80% | 72.26% |
| uM | | | | | | | | | | | | |
| 5 | 55.30% | 50.40% | 8.13% | 7.79% | 79.30% | 114.34% | 89.53% | 88.10% | 98.86% | 128.84% | 78.81% | 92.94% |
| uM | | | | | | | | | | | | |
| 1 | 101.13% | 94.70% | 112.76% | 90.67% | 107.82% | 97.24% | 113.17% | 90.52% | 111.09% | 124.83% | 102.36% | 121.99% |
| uM | | | | | | | | | | | | |

Exp 2 (SMW507), Percentage Infection Relative to Appropriate DMSO Control (e.g. Set DMSO as 100%) with Avg Background Subtraction

| | SILCS49 | SILCS49 | SILCS50 | SILCS50 | SICLS51 | SICLS51 | SICLS52 | SICLS52 | SICLS53 | SICLS53 | SICLS54 | SICLS54 |
|---|---|---|---|---|---|---|---|---|---|---|---|---|
| 50 | 19.58% | 18.05% | 3.84% | 3.65% | 106.85% | 122.63% | 116.64% | 140.70% | 147.14% | 134.78% | 127.43% | 125.06% |
| uM | | | | | | | | | | | | |
| 25 | 29.98% | 17.91% | 7.51% | 1.52% | 85.61% | 108.10% | 136.99% | 105.83% | 152.39% | 136.24% | 120.70% | 104.05% |
| uM | | | | | | | | | | | | |
| 10 | 23.33% | 20.87% | 7.60% | 8.55% | 79.31% | 97.68% | 114.17% | 117.66% | 118.96% | 132.33% | 105.38% | 94.77% |
| uM | | | | | | | | | | | | |
| 5 | 52.91% | 60.38% | 11.62% | 15.41% | 92.28% | 116.72% | 142.42% | 150.99% | 109.50% | 156.88% | 85.51% | 114.28% |
| uM | | | | | | | | | | | | |
| 1 | 67.31% | 97.84% | 51.03% | 60.73% | 102.77% | 92.24% | 91.91% | 73.53% | 113.17% | 102.75% | 73.20% | 100.02% |
| uM | | | | | | | | | | | | |

Average (SILCS 49-51)

| | SILCS49 | | | SILCS50 | | | SICLS51 | | |
|---|---|---|---|---|---|---|---|---|---|
| | Mean | STD | n | Mean | STD | n | Mean | STD | n |
| 50 uM | 13.54% | 6.17% | 4 | 0.30% | 3.98% | 4 | 105.46% | 13.12% | 4 |
| 25 uM | 27.11% | 6.62% | 4 | 1.99% | 3.87% | 4 | 95.65% | 19.26% | 4 |
| 10 uM | 29.10% | 8.30% | 4 | 4.51% | 4.13% | 4 | 92.65% | 14.33% | 4 |
| 5 uM | 54.75% | 4.25% | 4 | 10.74% | 3.56% | 4 | 100.66% | 18.00% | 4 |
| 1 uM | 90.24% | 15.51% | 4 | 78.80% | 28.24% | 4 | 100.02% | 6.75% | 4 |

Average (SILCS 52-54)

| | SICLS52 | | | SICLS53 | | | SICLS54 | | |
|---|---|---|---|---|---|---|---|---|---|
| | Mean | STD | n | Mean | STD | n | Mean | STD | n |
| 50 uM | 122.98% | 13.03% | 4 | 129.39% | 14.99% | 4 | 113.08% | 15.70% | 4 |
| 25 uM | 120.98% | 13.19% | 4 | 127.31% | 21.43% | 4 | 105.10% | 19.34% | 4 |
| 10 uM | 104.43% | 14.15% | 4 | 113.86% | 14.72% | 4 | 89.30% | 14.13% | 4 |
| 5 uM | 117.76% | 33.61% | 4 | 123.52% | 25.46% | 4 | 92.88% | 15.39% | 4 |
| 1 uM | 92.28% | 16.24% | 4 | 112.96% | 9.11% | 4 | 99.39% | 20.05% | 4 |

Toxicity Data

Exp 1 (SMW506), Percentage Infection Relative to Appropriate DMSO Control (e.g. Set DMSO as 1000%) with Avg Background Subtraction

| | SILCS49 | SILCS49 | SILCS50 | SILCS50 | SICLS51 | SICLS51 | SICLS52 | SICLS52 | SICLS53 | SICLS53 | SICLS54 | SICLS54 |
|---|---|---|---|---|---|---|---|---|---|---|---|---|
| 50 | 97% | 101% | 101% | 93% | 85% | 82% | 80% | 83% | 87% | 88% | 90% | 88% |
| uM | | | | | | | | | | | | |
| 25 | 109% | 115% | 113% | 112% | 97% | 90% | 89% | 95% | 101% | 97% | 100% | 95% |
| uM | | | | | | | | | | | | |
| 10 | 111% | 115% | 119% | 120% | 103% | 95% | 93% | 103% | 99% | 99% | 103% | 92% |
| uM | | | | | | | | | | | | |
| 5 | 103% | 110% | 128% | 122% | 103% | 97% | 96% | 103% | 101% | 96% | 103% | 98% |
| uM | | | | | | | | | | | | |
| 1 | 91% | 100% | 97% | 102% | 99% | 97% | 97% | 103% | 103% | 95% | 100% | 89% |
| uM | | | | | | | | | | | | |

Exp 2 (SMW507), Percentage Infection Relative to Appropriate DMSO Control (e.g. Set DMSO as 1000%) with Avg Background Subtraction

| | SILCS49 | SILCS49 | SILCS50 | SILCS50 | SICLS51 | SICLS51 | SICLS52 | SICLS52 | SICLS53 | SICLS53 | SICLS54 | SICLS54 |
|---|---|---|---|---|---|---|---|---|---|---|---|---|
| 50 | 91% | 107% | 104% | 105% | 90% | 85% | 82% | 80% | 79% | 85% | 88% | 88% |
| uM | | | | | | | | | | | | |
| 25 | 112% | 125% | 116% | 124% | 111% | 98% | 99% | 97% | 91% | 99% | 103% | 100% |
| uM | | | | | | | | | | | | |
| 10 | 124% | 133% | 135% | 138% | 113% | 106% | 100% | 96% | 103% | 111% | 104% | 103% |
| uM | | | | | | | | | | | | |
| 5 | 115% | 126% | 136% | 132% | 113% | 104% | 96% | 97% | 105% | 102% | 106% | 100% |
| uM | | | | | | | | | | | | |
| 1 | 111% | 116% | 130% | 129% | 104% | 102% | 104% | 109% | 102% | 113% | 110% | 104% |
| uM | | | | | | | | | | | | |

Average (SILCS 49-51)

| | SILCS49 | | | SILCS50 | | | SICLS51 | | |
|---|---|---|---|---|---|---|---|---|---|
| | Mean | STD | n | Mean | STD | n | Mean | STD | n |
| 50 uM | 98.89% | 6.93% | 4 | 100.61% | 5.24% | 4 | 85.53% | 3.43% | 4 |
| 25 uM | 115.31% | 6.74% | 4 | 116.14% | 5.22% | 4 | 98.78% | 8.80% | 4 |
| 10 uM | 120.89% | 9.91% | 4 | 128.03% | 9.84% | 4 | 104.12% | 7.59% | 4 |
| 5 uM | 113.44% | 9.69% | 4 | 129.23% | 5.92% | 4 | 104.19% | 6.76% | 4 |
| 1 uM | 104.43% | 11.21% | 4 | 114.44% | 17.14% | 4 | 100.38% | 3.33% | 4 |

Average (SILCS 52-54)

| | SICLS52 | | | SICLS53 | | | SICLS54 | | |
|---|---|---|---|---|---|---|---|---|---|
| | Mean | STD | n | Mean | STD | n | Mean | STD | n |
| 50 uM | 81.04% | 1.36% | 4 | 84.68% | 3.90% | 4 | 88.29% | 1.20% | 4 |
| 25 uM | 94.91% | 4.24% | 4 | 97.25% | 4.15% | 4 | 99.71% | 3.24% | 4 |
| 10 uM | 97.92% | 4.52% | 4 | 103.08% | 5.47% | 4 | 100.51% | 5.90% | 4 |
| 5 uM | 97.63% | 3.36% | 4 | 101.22% | 3.62% | 4 | 101.65% | 3.30% | 4 |
| 1 uM | 103.30% | 4.65% | 4 | 103.19% | 7.67% | 4 | 100.58% | 8.93% | 4 |

SILCS 55-60 Plate Setup, Nunc Celigo Plates

| | 1 | 2 | 3 | 4 | 5 | 6 |
|---|---|---|---|---|---|---|
| A | SICLS55 | SICLS55 | SICLS56 | SICLS56 | SICLS57 | SICLS57 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 2 |
|---|---|---|---|---|---|---|---|
| A | SICLS58 | SICLS58 | SICLS59 | SICLS59 | SICLS60 | SICLS60 | 50 μM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW491), Percentage Infection Relative to Appropriate DMSO Control (e.g. Set DMSO as 1000%) with Avg Background Subtraction

| | SICLS55 | SICLS55 | SICLS56 | SICLS56 | SICLS57 | SICLS57 | SICLS58 | SICLS58 | SICLS59 | SICLS59 | SICLS60 | SICLS60 |
|---|---|---|---|---|---|---|---|---|---|---|---|---|
| 50 | 68.88% | 84.52% | 74.83% | 107.20% | 94.49% | 111.14% | 106.18% | 113.23% | 107.59% | 114.78% | 120.98% | 129.76% |
| uM | | | | | | | | | | | | |
| 25 | 88.82% | 82.14% | 80.12% | 111.94% | 88.92% | 107.25% | 108.96% | 129.63% | 89.36% | 110.94% | 76.90% | 85.86% |
| uM | | | | | | | | | | | | |
| 10 | 62.02% | 59.23% | 51.81% | 91.39% | 78.25% | 77.86% | 88.71% | 95.89% | 71.95% | 86.54% | 80.03% | 73.77% |
| uM | | | | | | | | | | | | |
| 5 | 62.01% | 85.43% | 60.80% | 55.53% | 79.43% | 67.80% | 83.84% | 71.68% | 53.07% | 84.53% | 73.27% | 70.96% |
| uM | | | | | | | | | | | | |
| 1 | 62.62% | 68.27% | 59.44% | 63.31% | 62.79% | 71.70% | 66.78% | 64.64% | 39.98% | 59.33% | 61.78% | 74.39% |
| uM | | | | | | | | | | | | |

Exp 2 (SMW492), Percentage Infection Relative to Appropriate DMSO Control (e.g. Set DMSO as 1000%) with Avg Background Subtraction

| | SICLS55 | SICLS55 | SICLS56 | SICLS56 | SICLS57 | SICLS57 | SICLS58 | SICLS58 | SICLS59 | SICLS59 | SICLS60 | SICLS60 |
|---|---|---|---|---|---|---|---|---|---|---|---|---|
| 50 | 159.48% | 157.07% | 158.54% | 143.59% | 172.07% | 140.05% | 210.18% | 210.79% | 156.85% | 134.25% | 102.12% | 137.23% |
| uM | | | | | | | | | | | | |
| 25 | 95.14% | 90.14% | 89.20% | 109.64% | 90.23% | 137.50% | 170.43% | 133.54% | 115.70% | 118.35% | 114.30% | 117.10% |
| uM | | | | | | | | | | | | |
| 10 | 94.76% | 68.34% | 81.46% | 58.38% | 87.33% | 73.50% | 131.95% | 104.23% | 84.59% | 89.40% | 64.76% | 75.58% |
| uM | | | | | | | | | | | | |
| 5 | 80.11% | 81.65% | 60.26% | 46.79% | 67.31% | 57.99% | 86.19% | 73.65% | 55.93% | 73.69% | 70.65% | 79.71% |
| uM | | | | | | | | | | | | |
| 1 | 83.36% | 102.06% | 78.25% | 72.63% | 82.13% | 81.80% | 75.50% | 67.58% | 81.95% | 101.06% | 83.16% | 98.20% |
| uM | | | | | | | | | | | | |

Average (SILCS 55-57)

| | SICLS55 | | | SICLS56 | | | SICLS57 | | |
|---|---|---|---|---|---|---|---|---|---|
| | Mean | STD | n | Mean | STD | n | Mean | STD | n |
| 50 uM | 117.49% | 47.54% | 4 | 121.04% | 37.60% | 4 | 129.44% | 34.09% | 4 |
| 25 uM | 89.06% | 5.36% | 4 | 97.73% | 15.56% | 4 | 105.98% | 22.61% | 4 |
| 10 uM | 71.09% | 16.23% | 4 | 70.76% | 18.73% | 4 | 79.23% | 5.81% | 4 |
| 5 uM | 77.30% | 10.44% | 4 | 55.85% | 6.48% | 4 | 68.13% | 8.78% | 4 |
| 1 uM | 79.08% | 17.64% | 4 | 68.41% | 8.59% | 4 | 74.61% | 9.25% | 4 |

Average (SILCS 58-60)

| | SICLS58 | | | SICLS59 | | | SICLS60 | | |
|---|---|---|---|---|---|---|---|---|---|
| | Mean | STD | n | Mean | STD | n | Mean | STD | n |
| 50 uM | 160.09% | 58.26% | 4 | 128.37% | 22.08% | 4 | 122.52% | 15.13% | 4 |
| 25 uM | 135.64% | 25.58% | 4 | 108.59% | 13.18% | 4 | 98.54% | 20.18% | 4 |
| 10 uM | 105.19% | 18.93% | 4 | 83.12% | 7.71% | 4 | 73.53% | 6.41% | 4 |
| 5 uM | 78.84% | 7.24% | 4 | 66.80% | 14.93% | 4 | 73.65% | 4.21% | 4 |
| 1 uM | 68.63% | 4.74% | 4 | 70.58% | 26.59% | 4 | 79.38% | 15.31% | 4 |

Toxicity Data

Exp 1 (SMW491), Percentage Infection Relative to Appropriate DMSO Control (e.g. Set DMSO as 1000%) with Avg Background Subtraction

| | SICLS55 | SICLS55 | SICLS56 | SICLS56 | SICLS57 | SICLS57 | SICLS58 | SICLS58 | SICLS59 | SICLS59 | SICLS60 | SICLS60 |
|---|---|---|---|---|---|---|---|---|---|---|---|---|
| 50 | 91% | 95% | 100% | 93% | 96% | 91% | 86% | 90% | 91% | 88% | 87% | 85% |
| uM | | | | | | | | | | | | |
| 25 | 97% | 103% | 102% | 97% | 102% | 96% | 96% | 98% | 101% | 100% | 103% | 98% |
| uM | | | | | | | | | | | | |
| 10 | 109% | 119% | 121% | 109% | 112% | 104% | 105% | 105% | 111% | 116% | 112% | 103% |
| uM | | | | | | | | | | | | |
| 5 | 103% | 112% | 116% | 115% | 103% | 104% | 102% | 116% | 115% | 115% | 111% | 104% |
| uM | | | | | | | | | | | | |
| 1 | 112% | 126% | 124% | 122% | 113% | 113% | 111% | 116% | 122% | 121% | 119% | 111% |
| uM | | | | | | | | | | | | |

Exp 2 (SMW492), Percentage Infection Relative to Appropriate DMSO Control (e.g. Set DMSO as 100%) with Avg Background Subtraction

| | SICLS55 | SICLS55 | SICLS56 | SICLS56 | SICLS57 | SICLS57 | SICLS58 | SICLS58 | SICLS59 | SICLS59 | SICLS60 | SICLS60 |
|---|---|---|---|---|---|---|---|---|---|---|---|---|
| 50 | 81% | 84% | 83% | 82% | 77% | 80% | 73% | 72% | 80% | 88% | 87% | 81% |
| uM | | | | | | | | | | | | |
| 25 | 99% | 114% | 108% | 111% | 109% | 94% | 88% | 95% | 97% | 102% | 95% | 91% |
| uM | | | | | | | | | | | | |
| 10 | 112% | 135% | 127% | 122% | 118% | 114% | 97% | 106% | 120% | 118% | 117% | 106% |
| uM | | | | | | | | | | | | |
| 5 | 108% | 122% | 122% | 124% | 113% | 114% | 108% | 113% | 126% | 123% | 113% | 113% |
| uM | | | | | | | | | | | | |
| 1 | 110% | 114% | 121% | 114% | 119% | 111% | 106% | 118% | 103% | 115% | 111% | 105% |
| uM | | | | | | | | | | | | |

Average (SILCS 55-57)

| | SICLS55 | | | SICLS56 | | | SICLS57 | | |
|---|---|---|---|---|---|---|---|---|---|
| | Mean | STD | n | Mean | STD | n | Mean | STD | n |
| 50 uM | 87.84% | 6.57% | 4 | 89.59% | 8.68% | 4 | 85.77% | 8.90% | 4 |
| 25 uM | 103.46% | 7.39% | 4 | 104.41% | 6.13% | 4 | 100.22% | 6.79% | 4 |
| 10 uM | 119.11% | 11.73% | 4 | 119.65% | 7.49% | 4 | 111.79% | 5.82% | 4 |
| 5 uM | 111.39% | 8.22% | 4 | 119.22% | 4.50% | 4 | 108.63% | 5.75% | 4 |
| 1 uM | 115.35% | 7.23% | 4 | 120.34% | 4.54% | 4 | 114.12% | 3.28% | 4 |

Average (SILCS 58-60)

| | SICLS58 | | | SICLS59 | | | SICLS60 | | |
|---|---|---|---|---|---|---|---|---|---|
| | Mean | STD | n | Mean | STD | n | Mean | STD | n |
| 50 uM | 80.27% | 9.02% | 4 | 86.44% | 4.80% | 4 | 85.19% | 3.21% | 4 |
| 25 uM | 94.22% | 4.34% | 4 | 100.03% | 2.15% | 4 | 96.61% | 4.94% | 4 |
| 10 uM | 103.42% | 4.26% | 4 | 116.30% | 3.84% | 4 | 109.31% | 5.97% | 4 |
| 5 uM | 109.77% | 6.16% | 4 | 119.78% | 5.95% | 4 | 110.17% | 4.31% | 4 |
| 1 uM | 112.69% | 5.79% | 4 | 115.37% | 8.47% | 4 | 111.59% | 5.90% | 4 |

SILCS 61-66 Plate Setup, Nunc Celigo Plates

| | 1 | 2 | 3 | 4 | 5 | 6 |
|---|---|---|---|---|---|---|
| A | SICLS61 | SICLS61 | SICLS62 | SICLS62 | SICLS63 | SICLS63 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 3 |
|---|---|---|---|---|---|---|---|
| A | SICLS64 | SICLS64 | SICLS65 | SICLS65 | SICLS66 | SICLS66 | 50 μM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW506), Percentage Infection Relative to Appropriate DMSO Control (e.g. Set DMSO as 100%) with Avg Background Subtraction

| | SICLS61 | SICLS61 | SICLS62 | SICLS62 | SICLS63 | SICLS63 | SICLS64 | SICLS64 | SICLS65 | SICLS65 | SICLS66 | SICLS66 |
|---|---|---|---|---|---|---|---|---|---|---|---|---|
| 50 | −4.48% | −4.80% | −4.48% | 98.49% | 144.51% | 104.64% | 164.92% | 136.46% | 133.11% | 121.27% | 166.70% | 96.11% |
| uM | | | | | | | | | | | | |
| 25 | 51.60% | 90.77% | 83.54% | 119.74% | 110.41% | 124.07% | 141.09% | 135.48% | 138.60% | 119.76% | 104.41% | 99.66% |
| uM | | | | | | | | | | | | |
| 10 | 73.81% | 87.85% | 84.98% | 89.35% | 84.81% | 93.19% | 108.20% | 93.82% | 86.07% | 102.23% | 95.95% | 98.64% |
| uM | | | | | | | | | | | | |
| 5 | 41.43% | 63.29% | 45.05% | 53.24% | 56.72% | 52.03% | 59.52% | 71.93% | 76.32% | 72.80% | 72.63% | 86.93% |
| uM | | | | | | | | | | | | |
| 1 | 73.61% | 82.18% | 84.10% | 102.05% | 82.08% | 74.71% | 85.66% | 73.68% | 94.61% | 81.44% | 104.82% | 102.32% |
| uM | | | | | | | | | | | | |

Exp 2 (SMW492), Percentage Infection Relative to Appropriate DMSO Control (e.g. Set DMSO as 1000%) with Avg Background Subtraction

| | SICLS61 | SICLS61 | SICLS62 | SICLS62 | SICLS63 | SICLS63 | SICLS64 | SICLS64 | SICLS65 | SICLS65 | SICLS66 | SICLS66 |
|---|---|---|---|---|---|---|---|---|---|---|---|---|
| 50 | 11.69% | 10.06% | −0.82% | 2.34% | 113.19% | 123.56% | 167.52% | 165.88% | 151.04% | 179.08% | 104.11% | 145.81% |
| uM | | | | | | | | | | | | |
| 25 | 22.17% | 9.12% | 1.62% | 0.75% | 104.92% | 132.74% | 138.76% | 107.38% | 103.96% | 170.02% | 99.75% | 145.81% |
| uM | | | | | | | | | | | | |
| 10 | 33.45% | 32.58% | 5.57% | 5.50% | 99.35% | 91.41% | 128.37% | 114.29% | 127.42% | 119.00% | 97.92% | 136.11% |
| uM | | | | | | | | | | | | |
| 5 | 38.94% | 52.11% | 9.05% | 14.15% | 77.06% | −1.67% | 0.64% | 2.00% | −1.04% | 83.42% | −1.77% | 86.51% |
| uM | | | | | | | | | | | | |
| 1 | 57.97% | 58.62% | 50.01% | 36.38% | 66.32% | 100.57% | 78.71% | 80.27% | 73.37% | 99.17% | 111.34% | 96.12% |
| uM | | | | | | | | | | | | |

Average (SICLS 61-63)

| | SICLS61 | | | SICLS62 | | | SICLS63 | | |
|---|---|---|---|---|---|---|---|---|---|
| | Mean | STD | n | Mean | STD | n | Mean | STD | n |
| 50 uM | 3.12% | 8.98% | 4 | 23.88% | 49.82% | 4 | 121.48% | 17.20% | 4 |
| 25 uM | 43.41% | 36.23% | 4 | 51.41% | 59.85% | 4 | 118.04% | 12.68% | 4 |
| 10 uM | 56.92% | 28.20% | 4 | 46.35% | 47.16% | 4 | 92.19% | 5.98% | 4 |
| 5 uM | 48.94% | 11.14% | 4 | 30.37% | 22.03% | 4 | 46.03% | 33.61% | 4 |
| 1 uM | 68.10% | 11.85% | 4 | 68.14% | 30.23% | 4 | 80.92% | 14.60% | 4 |

Average (SICLS 64-66)

| | SICLS64 | | | SICLS65 | | | SICLS66 | | |
|---|---|---|---|---|---|---|---|---|---|
| | Mean | STD | n | Mean | STD | n | Mean | STD | n |
| 50 uM | 158.69% | 14.86% | 4 | 146.13% | 25.15% | 4 | 128.18% | 33.68% | 4 |
| 25 uM | 130.68% | 15.70% | 4 | 133.09% | 28.41% | 4 | 112.41% | 22.37% | 4 |
| 10 uM | 111.17% | 14.32% | 4 | 108.68% | 18.36% | 4 | 107.16% | 19.34% | 4 |
| 5 uM | 33.52% | 37.53% | 4 | 57.87% | 39.53% | 4 | 61.08% | 42.42% | 4 |
| 1 uM | 79.58% | 4.93% | 4 | 87.15% | 11.87% | 4 | 103.65% | 6.30% | 4 |

Toxicity Data

Exp 1 (SMW506), Percentage Infection Relative to Appropriate DMSO Control (e.g. Set DMSO as 100%) with Avg Background Subtraction

| | SICLS61 | SICLS61 | SICLS62 | SICLS62 | SICLS63 | SICLS63 | SICLS64 | SICLS64 | SICLS65 | SICLS65 | SICLS66 | SICLS66 |
|---|---|---|---|---|---|---|---|---|---|---|---|---|
| 50 | 108% | 113% | 110% | 90% | 77% | 81% | 78% | 82% | 80% | 81% | 89% | 89% |
| uM | | | | | | | | | | | | |
| 25 | 106% | 105% | 103% | 95% | 96% | 88% | 90% | 85% | 89% | 93% | 98% | 92% |
| uM | | | | | | | | | | | | |
| 10 | 97% | 106% | 107% | 103% | 105% | 102% | 103% | 100% | 101% | 102% | 102% | 93% |
| uM | | | | | | | | | | | | |
| 5 | 110% | 112% | 115% | 117% | 112% | 110% | 109% | 114% | 109% | 105% | 103% | 95% |
| uM | | | | | | | | | | | | |
| 1 | 100% | 107% | 107% | 103% | 105% | 104% | 101% | 107% | 103% | 102% | 100% | 97% |
| uM | | | | | | | | | | | | |

Exp 2 (SMW507), Percentage Infection Relative to Appropriate DMSO Control (e.g. Set DMSO as 100%) with Avg Background Subtraction

| | SICLS61 | SICLS61 | SICLS62 | SICLS62 | SICLS63 | SICLS63 | SICLS64 | SICLS64 | SICLS65 | SICLS65 | SICLS66 | SICLS66 |
|---|---|---|---|---|---|---|---|---|---|---|---|---|
| 50 | 93% | 105% | 99% | 93% | 83% | 74% | 70% | 72% | 68% | 74% | 80% | 81% |
| uM | | | | | | | | | | | | |
| 25 | 105% | 122% | 117% | 111% | 94% | 87% | 77% | 87% | 89% | 87% | 94% | 79% |
| uM | | | | | | | | | | | | |
| 10 | 106% | 123% | 126% | 129% | 104% | 97% | 84% | 84% | 90% | 100% | 97% | 83% |
| uM | | | | | | | | | | | | |
| 5 | 104% | 117% | 132% | 131% | 106% | 130% | 126% | 128% | 137% | 112% | 136% | 102% |
| uM | | | | | | | | | | | | |
| 1 | 102% | 117% | 120% | 120% | 113% | 106% | 103% | 102% | 109% | 104% | 102% | 97% |
| uM | | | | | | | | | | | | |

Average (SICLS 61-63)

| | SICLS61 | | | SICLS62 | | | SICLS63 | | |
|---|---|---|---|---|---|---|---|---|---|
| | Mean | STD | n | Mean | STD | n | Mean | STD | n |
| 50 uM | 104.46% | 8.56% | 4 | 98.08% | 8.94% | 4 | 78.57% | 3.97% | 4 |
| 25 uM | 109.52% | 8.34% | 4 | 106.51% | 9.54% | 4 | 91.46% | 4.33% | 4 |
| 10 uM | 107.86% | 10.83% | 4 | 116.29% | 13.02% | 4 | 101.91% | 3.73% | 4 |
| 5 uM | 110.71% | 5.29% | 4 | 123.92% | 8.93% | 4 | 114.55% | 10.50% | 4 |
| 1 uM | 106.46% | 7.93% | 4 | 112.47% | 9.22% | 4 | 106.90% | 3.94% | 4 |

Average (SICLS 64-66)

| | SICLS64 | | | SICLS65 | | | SICLS66 | | |
|---|---|---|---|---|---|---|---|---|---|
| | Mean | STD | n | Mean | STD | n | Mean | STD | n |
| 50 uM | 75.66% | 5.38% | 4 | 75.91% | 6.01% | 4 | 84.65% | 4.78% | 4 |
| 25 uM | 84.89% | 5.57% | 4 | 89.35% | 2.61% | 4 | 90.79% | 8.26% | 4 |
| 10 uM | 92.78% | 10.24% | 4 | 98.27% | 5.75% | 4 | 93.98% | 8.09% | 4 |
| 5 uM | 119.20% | 9.02% | 4 | 115.73% | 14.33% | 4 | 109.01% | 18.33% | 4 |
| 1 uM | 103.13% | 2.53% | 4 | 104.64% | 2.84% | 4 | 98.81% | 2.40% | 4 |

SILCS 67-72 Plate Setup, Nunc Celigo Plates

| | 1 | 2 | 3 | 4 | 5 | 6 |
|---|---|---|---|---|---|---|
| A | SICLS67 | SICLS67 | SICLS68 | SICLS68 | SICLS69 | SICLS69 |
| B | | | | | | |
| C | | | | | | |
| D | | | | | | |
| E | | | | | | |
| F | DMSO | | | | | |
| G | DMSO | | | | | |
| H | Uninfect | | | | | |

| | 7 | 8 | 9 | 10 | 11 | 12 | Plate 3 |
|---|---|---|---|---|---|---|---|
| A | SICLS70 | SICLS70 | SICLS71 | SICLS71 | SICLS72 | SICLS72 | 50 μM |
| B | | | | | | | 25 uM |
| C | | | | | | | 10 uM |
| D | | | | | | | 5 uM |
| E | | | | | | | 1 uM |
| F | | | | | | | 0.50% |
| G | | | | | | | 0.10% |
| H | | | | | | | N/A |

Infection Data

Exp 1 (SMW506), Percentage Infection Relative to Appropriate DMSO Control (e.g. Set DMSO as 1000%) with Avg Background Subtraction

| | SICLS67 | SICLS67 | SICLS68 | SICLS68 | SICLS69 | SICLS69 | SICLS70 | SICLS70 | SICLS71 | SICLS71 | SICLS72 | SICLS72 |
|---|---|---|---|---|---|---|---|---|---|---|---|---|
| 50 | −1.60% | −0.98% | −1.44% | 100.79% | 105.31% | 121.51% | 111.83% | 152.85% | −1.77% | 41.57% | 14.10% | 14.56% |
| uM | | | | | | | | | | | | |
| 25 | 102.18% | 80.90% | 71.52% | 132.39% | 78.77% | 116.07% | 109.91% | 124.39% | 7.78% | 8.47% | 0.49% | 1.19% |
| uM | | | | | | | | | | | | |
| 10 | 83.44% | 69.09% | 80.49% | 90.65% | 60.14% | 83.28% | 79.96% | 77.02% | 0.11% | 1.06% | 0.89% | 1.38% |
| uM | | | | | | | | | | | | |
| 5 | 89.80% | 75.44% | 84.71% | 82.83% | 78.81% | 80.38% | 66.65% | 54.35% | 3.00% | 2.82% | 3.97% | 2.35% |
| uM | | | | | | | | | | | | |
| 1 | 83.30% | 67.42% | 65.59% | 73.94% | 46.90% | 53.41% | 50.53% | 62.80% | 44.86% | 32.37% | 71.72% | 70.60% |
| uM | | | | | | | | | | | | |

Exp 2 (SMW492), Percentage Infection Relative to Appropriate DMSO Control (e.g. Set DMSO as 1000%) with Avg Background Subtraction

| | SICLS67 | SICLS67 | SICLS68 | SICLS68 | SICLS69 | SICLS69 | SICLS70 | SICLS70 | SICLS71 | SICLS71 | SICLS72 | SICLS72 |
|---|---|---|---|---|---|---|---|---|---|---|---|---|
| 50 | −1.66% | 120.99% | 88.17% | 129.30% | 173.42% | 192.48% | 158.83% | 168.05% | 169.02% | 167.89% | 162.29% | 155.40% |
| uM | | | | | | | | | | | | |
| 25 | 145.96% | 116.44% | 110.95% | 127.91% | 144.62% | 164.80% | 148.87% | 200.49% | 127.56% | 151.50% | 137.50% | 132.65% |
| uM | | | | | | | | | | | | |
| 10 | 104.02% | 65.08% | 97.13% | 65.35% | 72.57% | 85.97% | 105.01% | 94.06% | 72.78% | 87.07% | 120.39% | 123.40% |
| uM | | | | | | | | | | | | |
| 5 | 72.46% | 51.38% | 61.16% | 56.79% | 62.20% | 60.81% | 72.37% | 51.32% | 58.63% | 68.31% | 83.70% | 83.51% |
| uM | | | | | | | | | | | | |
| 1 | 75.58% | 62.77% | 71.68% | 57.90% | 70.61% | 52.16% | −0.72% | −0.51% | 0.48% | 72.06% | 61.87% | 84.23% |
| uM | | | | | | | | | | | | |

Average (SICLS 67-69)

| | SICLS67 | | | SICLS68 | | | SICLS69 | | |
|---|---|---|---|---|---|---|---|---|---|
| | Mean | STD | n | Mean | STD | n | Mean | STD | n |
| 50 uM | 29.19% | 61.20% | 4 | 79.20% | 56.45% | 4 | 148.18% | 41.43% | 4 |
| 25 uM | 111.37% | 27.30% | 4 | 110.69% | 27.70% | 4 | 126.06% | 37.33% | 4 |
| 10 uM | 80.41% | 17.60% | 4 | 83.40% | 13.85% | 4 | 75.49% | 11.76% | 4 |
| 5 uM | 72.27% | 15.85% | 4 | 71.37% | 14.45% | 4 | 70.55% | 10.48% | 4 |
| 1 uM | 72.27% | 9.06% | 4 | 67.28% | 7.18% | 4 | 55.77% | 10.29% | 4 |

Average (SICLS 70-72)

| | SICLS70 | | | SICLS71 | | | SICLS72 | | |
|---|---|---|---|---|---|---|---|---|---|
| | Mean | STD | n | Mean | STD | n | Mean | STD | n |
| 50 uM | 147.89% | 24.84% | 4 | 94.18% | 87.58% | 4 | 86.59% | 83.48% | 4 |
| 25 uM | 145.91% | 39.78% | 4 | 73.83% | 76.49% | 4 | 67.96% | 77.53% | 4 |
| 10 uM | 89.01% | 13.00% | 4 | 40.26% | 46.18% | 4 | 61.51% | 69.73% | 4 |
| 5 uM | 61.17% | 9.98% | 4 | 33.19% | 35.19% | 4 | 43.38% | 46.45% | 4 |
| 1 uM | 28.02% | 33.45% | 4 | 37.44% | 29.70% | 4 | 72.10% | 9.20% | 4 |

Toxicity Data

Exp 1 (SMW506), Percentage Infection Relative to Appropriate DMSO Control (e.g. Set DMSO as 1000%) with Avg Background Subtraction

| | SICLS67 | SICLS67 | SICLS68 | SICLS68 | SICLS69 | SICLS69 | SICLS70 | SICLS70 | SICLS71 | SICLS71 | SICLS72 | SICLS72 |
|---|---|---|---|---|---|---|---|---|---|---|---|---|
| 50 | 105% | 108% | 111% | 91% | 91% | 88% | 89% | 85% | 0% | 0% | 1% | 0% |
| uM | | | | | | | | | | | | |
| 25 | 97% | 107% | 112% | 103% | 103% | 97% | 102% | 104% | 1% | 1% | 89% | 47% |
| uM | | | | | | | | | | | | |
| 10 | 107% | 113% | 109% | 111% | 109% | 108% | 111% | 106% | 38% | 51% | 139% | 124% |
| uM | | | | | | | | | | | | |
| 5 | 104% | 113% | 110% | 106% | 106% | 107% | 105% | 112% | 135% | 130% | 131% | 126% |
| uM | | | | | | | | | | | | |
| 1 | 109% | 119% | 118% | 112% | 113% | 115% | 111% | 117% | 117% | 128% | 114% | 112% |
| uM | | | | | | | | | | | | |

Exp 2 (SMW507), Percentage Infection Relative to Appropriate DMSO Control (e.g. Set DMSO as 100%) with Avg Background Subtraction

| | SICLS67 | SICLS67 | SICLS68 | SICLS68 | SICLS69 | SICLS69 | SICLS70 | SICLS70 | SICLS71 | SICLS71 | SICLS72 | SICLS72 |
|---|---|---|---|---|---|---|---|---|---|---|---|---|
| 50 | 99% | 88% | 89% | 76% | 73% | 66% | 71% | 73% | 75% | 77% | 71% | 74% |
| uM | | | | | | | | | | | | |
| 25 | 85% | 100% | 95% | 91% | 92% | 82% | 89% | 80% | 90% | 94% | 83% | 86% |
| uM | | | | | | | | | | | | |
| 10 | 95% | 122% | 106% | 115% | 112% | 104% | 100% | 100% | 110% | 105% | 100% | 91% |
| uM | | | | | | | | | | | | |
| 5 | 104% | 124% | 121% | 122% | 125% | 118% | 112% | 116% | 118% | 116% | 107% | 108% |
| uM | | | | | | | | | | | | |
| 1 | 100% | 122% | 119% | 120% | 115% | 116% | 141% | 151% | 152% | 124% | 118% | 109% |
| uM | | | | | | | | | | | | |

Average (SICLS 67-69)

| | SICLS67 | | | SICLS68 | | | SICLS69 | | |
|---|---|---|---|---|---|---|---|---|---|
| | Mean | STD | n | Mean | STD | n | Mean | STD | n |
| 50 uM | 100.17% | 8.70% | 4 | 91.82% | 14.40% | 4 | 79.50% | 11.72% | 4 |
| 25 uM | 97.31% | 9.06% | 4 | 100.14% | 9.11% | 4 | 93.65% | 8.67% | 4 |
| 10 uM | 109.33% | 11.20% | 4 | 110.48% | 3.99% | 4 | 108.32% | 3.15% | 4 |
| 5 uM | 111.48% | 9.57% | 4 | 114.63% | 7.99% | 4 | 113.80% | 9.00% | 4 |
| 1 uM | 112.56% | 9.92% | 4 | 117.26% | 3.39% | 4 | 114.56% | 1.40% | 4 |

Average (SICLS 70-72)

| | SICLS70 | | | SICLS71 | | | SICLS72 | | |
|---|---|---|---|---|---|---|---|---|---|
| | Mean | STD | n | Mean | STD | n | Mean | STD | n |
| 50 uM | 79.59% | 8.62% | 4 | 38.04% | 43.60% | 4 | 36.41% | 41.41% | 4 |
| 25 uM | 93.90% | 11.24% | 4 | 46.48% | 53.00% | 4 | 76.24% | 19.46% | 4 |
| 10 uM | 104.30% | 5.17% | 4 | 75.71% | 36.75% | 4 | 113.46% | 21.65% | 4 |
| 5 uM | 111.32% | 4.53% | 4 | 124.82% | 8.97% | 4 | 118.22% | 12.24% | 4 |
| 1 uM | 130.04% | 19.10% | 4 | 130.44% | 15.29% | 4 | 113.33% | 3.65% | 4 |

UMB_18G and Untreated Plate Setup, (UT Indicates Untreated, Nunc Celigo Plates)

| | 1 | 2 | 3 | 4 | 5 | 6 | 7 | 8 | 9 | 10 | 11 | 12 | Plate 3 |
|---|---|---|---|---|---|---|---|---|---|---|---|---|---|
| A | UMB18G | UMB18G | UMB18G | UMB18G | UT | UT | UT | UT | UT | UT | UT | UT | 50 μM |
| B | | | | | | | | | | | | | 25 uM |
| C | | | | | | | | | | | | | 10 uM |
| D | | | | | | | | | | | | | 5 uM |
| E | | | | | | | | | | | | | 1 uM |
| F | DMSO | | | | | | | | | | | | 0.50% |
| G | DMSO | | | | | | | | | | | | 0.10% |
| H | Uninfect | | | | | | | | | | | | N/A |

Infection Data

Exp 1 (SMW506), Percentage Infection Relative to Appropriate DMSO Control (e.g. Set DMSO as 1000%) with Avg Background Subtraction

| | UMB18G | UMB18G | UMB18G | UMB18G | UT | UT | UT | UT | UT | UT | UT | UT |
|---|---|---|---|---|---|---|---|---|---|---|---|---|
| 50 | 4.22% | 4.54% | 5.07% | 2.41% | 147.77% | 181.52% | 162.24% | 160.30% | 156.06% | 170.64% | 185.61% | 170.87% |
| uM | | | | | | | | | | | | |
| 25 | 4.49% | 2.30% | 4.05% | 3.63% | 157.97% | 134.56% | 167.95% | 128.62% | 188.88% | 181.52% | 161.72% | 173.87% |
| uM | | | | | | | | | | | | |
| 10 | 6.99% | 10.55% | 9.19% | 3.42% | 92.75% | 102.86% | 82.94% | 63.25% | 52.71% | 74.68% | 71.75% | 103.31% |
| uM | | | | | | | | | | | | |
| 5 | 53.61% | 41.20% | 33.67% | 11.96% | 77.82% | 90.29% | 80.27% | 72.77% | 64.98% | 97.09% | 94.05% | 105.24% |
| uM | | | | | | | | | | | | |
| 1 | 77.75% | 93.71% | 74.09% | 43.33% | 82.05% | 81.66% | 80.16% | 63.63% | 90.71% | 85.56% | 97.54% | 125.01% |
| uM | | | | | | | | | | | | |

Exp 2 (SMW492), Percentage Infection Relative to Appropriate DMSO Control (e.g. Set DMSO as 1000%) with Avg Background Subtraction

| | UMB18G | UMB18G | UMB18G | UMB18G | UT | UT | UT | UT | UT | UT | UT | UT |
|---|---|---|---|---|---|---|---|---|---|---|---|---|
| 50 | 1.27% | 0.52% | 1.57% | 1.49% | 127.75% | 129.45% | 146.05% | 106.26% | 115.52% | 160.21% | 102.93% | 131.88% |
| uM | | | | | | | | | | | | |
| 25 | 4.70% | 2.08% | 5.51% | 0.65% | 85.08% | 99.96% | 101.00% | 119.76% | 92.22% | 123.82% | 99.91% | 102.83% |
| uM | | | | | | | | | | | | |
| 10 | 3.43% | 3.30% | 3.52% | 1.74% | 67.66% | 60.92% | 53.54% | 51.85% | 60.96% | 55.51% | 73.61% | 82.99% |
| uM | | | | | | | | | | | | |
| 5 | 5.38% | 4.98% | 4.24% | 4.87% | 54.62% | 51.40% | 54.67% | 46.19% | 49.15% | 54.66% | 59.06% | 112.16% |
| uM | | | | | | | | | | | | |
| 1 | 34.50% | 31.39% | 22.69% | 24.33% | 64.70% | 48.00% | 44.86% | 49.35% | 60.51% | 51.08% | 65.57% | 63.94% |
| uM | | | | | | | | | | | | |

Average (UMB18G)

| | UMB18G | | | UMB18G | | |
|---|---|---|---|---|---|---|
| | Mean | STD | n | Mean | STD | n |
| 50 uM | 2.64% | 2.04% | 4 | 2.63% | 1.67% | 4 |
| 25 uM | 3.40% | 1.40% | 4 | 3.46% | 2.04% | 4 |
| 10 uM | 6.07% | 3.44% | 4 | 4.47% | 3.25% | 4 |
| 5 uM | 26.29% | 24.90% | 4 | 13.69% | 13.77% | 4 |
| 1 uM | 59.34% | 31.19% | 4 | 41.11% | 23.90% | 4 |

Average (Untreated)

| | UT | | | UT | | | UT | | | UT | | |
|---|---|---|---|---|---|---|---|---|---|---|---|---|
| | Mean | STD | n | Mean | STD | n | Mean | STD | n | Mean | STD | n |
| 50 uM | 146.62% | 24.97% | 4 | 143.71% | 25.99% | 4 | 150.61% | 24.18% | 4 | 147.82% | 37.54% | 4 |
| 25 uM | 119.39% | 33.03% | 4 | 129.33% | 28.20% | 4 | 146.61% | 46.49% | 4 | 134.59% | 38.69% | 4 |
| 10 uM | 81.05% | 19.98% | 4 | 62.89% | 14.27% | 4 | 60.96% | 9.77% | 4 | 82.91% | 14.46% | 4 |
| 5 uM | 68.53% | 18.68% | 4 | 63.47% | 15.76% | 4 | 66.47% | 21.44% | 4 | 92.63% | 23.59% | 4 |
| 1 uM | 69.10% | 16.23% | 4 | 59.50% | 15.93% | 4 | 71.97% | 19.18% | 4 | 88.01% | 29.11% | 4 |

Toxicity Data

Exp 1 (SMW506), Percentage Infection Relative to Appropriate DMSO Control (e.g. Set DMSO as 100%) with Avg Background Subtraction

| | UMB18G | UMB18G | UMB18G | UMB18G | UT | UT | UT | UT | UT | UT | UT | UT |
|---|---|---|---|---|---|---|---|---|---|---|---|---|
| 50 uM | 66% | 109% | 113% | 103% | 87% | 86% | 88% | 83% | 85% | 83% | 81% | 79% |
| 25 uM | 111% | 119% | 119% | 121% | 95% | 90% | 93% | 95% | 94% | 93% | 90% | 84% |
| 10 uM | 114% | 126% | 122% | 114% | 104% | 101% | 109% | 112% | 114% | 106% | 104% | 96% |
| 5 uM | 107% | 111% | 113% | 116% | 102% | 95% | 94% | 110% | 104% | 104% | 100% | 93% |
| 1 uM | 105% | 111% | 113% | 112% | 103% | 101% | 96% | 109% | 97% | 105% | 100% | 90% |

Exp 2 (SMW507), Percentage Infection Relative to Appropriate DMSO Control (e.g. Set DMSO as 100%) with Avg Background Subtraction

| | UMB18G | UMB18G | UMB18G | UMB18G | UT | UT | UT | UT | UT | UT | UT | UT |
|---|---|---|---|---|---|---|---|---|---|---|---|---|
| 50 uM | 91% | 107% | 108% | 78% | 88% | 88% | 83% | 90% | 90% | 87% | 96% | 84% |
| 25 uM | 118% | 125% | 119% | 122% | 110% | 98% | 96% | 105% | 105% | 100% | 103% | 98% |
| 10 uM | 148% | 160% | 148% | 140% | 128% | 128% | 121% | 131% | 133% | 136% | 119% | 118% |
| 5 uM | 144% | 156% | 158% | 163% | 131% | 119% | 118% | 133% | 136% | 133% | 122% | 97% |
| 1 uM | 135% | 135% | 160% | 142% | 123% | 126% | 119% | 130% | 112% | 132% | 115% | 120% |

Average (UMB18G)

| | UMB18G | | | UMB18G | | |
|---|---|---|---|---|---|---|
| | Mean | STD | n | Mean | STD | n |
| 50 uM | 93.28% | 19.97% | 4 | 100.37% | 15.66% | 4 |
| 25 uM | 118.22% | 5.44% | 4 | 120.31% | 1.64% | 4 |
| 10 uM | 137.10% | 20.73% | 4 | 131.04% | 15.34% | 4 |
| 5 uM | 129.65% | 24.03% | 4 | 137.65% | 26.67% | 4 |
| 1 uM | 121.38% | 15.78% | 4 | 131.60% | 23.58% | 4 |

Average (Untreated)

| | UT | | | UT | | | UT | | | UT | | |
|---|---|---|---|---|---|---|---|---|---|---|---|---|
| | Mean | STD | n | Mean | STD | n | Mean | STD | n | Mean | STD | n |
| 50 uM | 87.24% | 0.77% | 4 | 85.69% | 3.62% | 4 | 86.46% | 2.91% | 4 | 85.07% | 7.54% | 4 |
| 25 uM | 98.25% | 8.25% | 4 | 97.43% | 5.16% | 4 | 98.04% | 5.61% | 4 | 93.85% | 8.22% | 4 |
| 10 uM | 115.13% | 14.74% | 4 | 118.24% | 9.64% | 4 | 122.07% | 14.57% | 4 | 109.34% | 11.18% | 4 |
| 5 uM | 111.94% | 16.34% | 4 | 113.76% | 16.37% | 4 | 119.47% | 17.62% | 4 | 102.83% | 12.93% | 4 |
| 1 uM | 113.52% | 13.22% | 4 | 113.48% | 14.55% | 4 | 111.51% | 15.23% | 4 | 106.20% | 13.77% | 4 |

SILCS Correlation Analysis of Synthesized and Tested Compounds 1-72 (See Compound Structures in FIGS. 25-A-25-S)

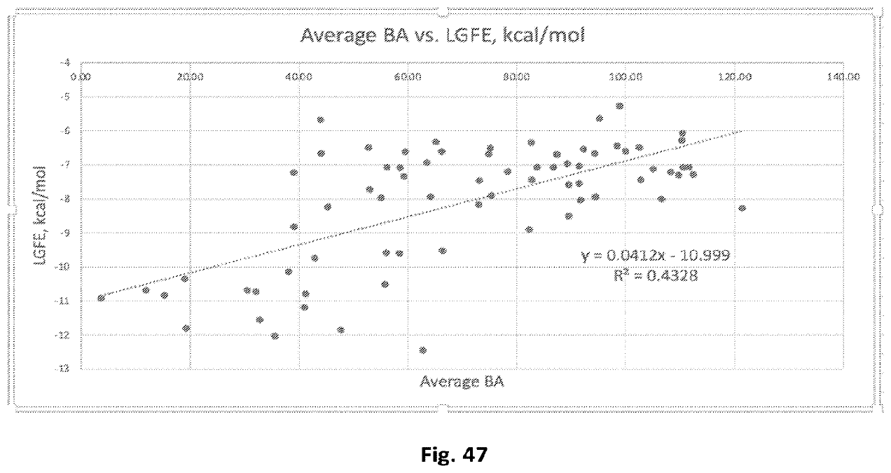

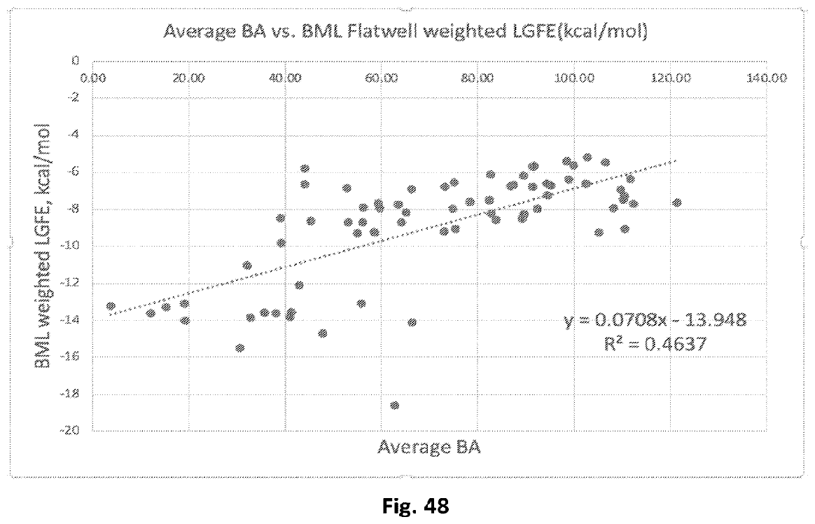

FIGS. 47 and 48 present correlation analysis for the 72 compounds based on SILCS analysis and the experimental SARS2 antiviral data.

SILCS-MC docking for all the 72 synthesized and tested analogs was undertaken from which the LGFE and LE scores were obtained. Correlation analysis was performed between the SILCS LGFE scores and the experimental SARS2 data. For the correlation C analysis, the average SARS2 relative infection to DMSO in the tables below were taken and the average value over the 5 tested concentrations (1 to 50 uM) calculated and used for the correlation analysis. Smaller average values indicate improved biological activity (as 100=DMSO control).

SARS2 and SILCS1-72 Averages (Infection Data)

| Screening data (Relative infection to DMSO controls) | | | | | | | For correlation analysis | | | | | | |
|---|---|---|---|---|---|---|---|---|---|---|---|---|---|
| | Average | Average | Average | Average | Average | | LGFE | LE | logP | | | | |
| Compound | 50 μM | 25 μM | 10 μM | 5 μM | 1 μM | Average | (kcal/mol) | (kcal/mol) | (o/w) | Weight | a_acc | a_don | TPSA |
| UMB18g | 5.35 | 6.86 | 9.95 | 31.21 | 59.58 | 22.59 | −10.775 | −0.348 | 2.49 | 431.55 | 6 | 2 | 57.20 |
| SILCS1 | 43.32 | 61.83 | 100.52 | 119.79 | 122.42 | 89.58 | −8.507 | −0.37 | 3.12 | 348.54 | 3 | 1 | 26.71 |
| SILCS2 | 2.25 | 5.84 | 46.71 | 50.08 | 85.68 | 38.11 | −10.152 | −0.35 | 1.23 | 402.51 | 6 | 2 | 60.86 |
| SILCS3 | 12.53 | 22.24 | 46.02 | 48.45 | 77.07 | 41.26 | −10.801 | −0.338 | 2.54 | 445.58 | 6 | 2 | 57.20 |
| SILCS4 | 9.17 | 18.71 | 13.79 | 40.06 | 82.49 | 32.84 | −11.547 | −0.33 | 4.47 | 483.67 | 5 | 2 | 47.97 |
| SILCS5 | 75.63 | 81.28 | 98.58 | 104.84 | 111.97 | 94.46 | −7.946 | −0.345 | 3.41 | 328.48 | 3 | 1 | 26.71 |
| SILCS6 | 31.45 | 54.97 | 88.17 | 71.51 | 79.58 | 65.14 | −6.328 | −0.333 | 2.04 | 278.42 | 2 | 0 | 19.62 |
| SILCS7 | 31.09 | 41.96 | 57.71 | 92.81 | 93.93 | 63.50 | −6.927 | −0.346 | 2.22 | 292.45 | 2 | 0 | 19.62 |
| SILCS8 | 8.38 | 13.96 | 53.04 | 75.48 | 87.90 | 47.75 | −11.85 | −0.339 | 1.67 | 485.65 | 6 | 3 | 63.24 |
| SILCS9 | 95.98 | 111.45 | 120.58 | 120.66 | 113.21 | 112.38 | −7.284 | −0.364 | 1.34 | 294.42 | 3 | 1 | 39.85 |
| SILCS10 | 6.39 | 34.00 | 41.58 | 43.73 | 52.60 | 35.66 | −12.037 | −0.344 | 3.38 | 484.66 | 5 | 2 | 51.21 |
| SILCS11 | 20.31 | 35.01 | 54.16 | 78.54 | 75.71 | 52.75 | −6.487 | −0.324 | 2.51 | 272.39 | 2 | 0 | 19.62 |
| SILCS12 | 76.66 | 135.12 | 110.30 | 109.01 | 120.43 | 110.30 | −6.281 | −0.349 | 1.99 | 264.39 | 2 | 0 | 19.62 |
| SILCS13 | 85.64 | 142.17 | 120.89 | 97.87 | 94.70 | 108.25 | −7.218 | −0.344 | 1.40 | 308.45 | 3 | 1 | 39.85 |
| SILCS14 | 1.69 | 14.65 | 93.84 | 105.13 | 105.53 | 64.17 | −7.939 | −0.378 | 3.26 | 368.34 | 3 | 0 | 19.37 |
| SILCS15 | 21.75 | 31.59 | 92.05 | 111.13 | 108.27 | 72.96 | −8.166 | −0.408 | 3.21 | 357.32 | 2 | 0 | 19.62 |
| SILCS16 | 1.95 | 4.14 | 74.52 | 87.79 | 112.00 | 56.08 | −9.586 | −0.383 | 5.43 | 435.35 | 2 | 0 | 6.48 |
| SILCS17 | 36.54 | 91.43 | 114.02 | 113.50 | 106.20 | 92.34 | −6.549 | −0.345 | 2.20 | 278.42 | 2 | 0 | 19.62 |
| SILCS18 | 26.65 | 0.27 | 66.17 | 85.94 | 113.22 | 58.45 | −9.6 | −0.436 | 5.29 | 446.25 | 2 | 0 | 6.48 |
| SILCS19 | 3.60 | 38.12 | 99.68 | 119.39 | 116.34 | 75.43 | −7.907 | −0.395 | 4.14 | 373.38 | 2 | 0 | 6.48 |
| SILCS20 | 48.15 | 8.63 | 39.18 | 81.76 | 115.20 | 58.58 | −7.097 | −0.296 | 4.92 | 356.56 | 2 | 0 | 6.48 |
| SILCS21 | 10.39 | 6.17 | 26.42 | 63.59 | 88.88 | 39.09 | −7.226 | −0.301 | 4.80 | 356.56 | 2 | 0 | 6.48 |
| SILCS22 | 2.68 | 6.37 | 53.13 | 70.31 | 72.38 | 40.97 | −11.19 | −0.339 | 2.67 | 456.61 | 5 | 3 | 60.00 |
| SILCS23 | 25.76 | 42.51 | 71.95 | 72.94 | 83.26 | 59.28 | −7.337 | −0.334 | 3.19 | 300.45 | 2 | 0 | 19.62 |
| SILCS24 | 19.26 | 35.04 | 70.96 | 71.54 | 68.53 | 53.07 | −7.731 | −0.368 | 2.72 | 306.47 | 2 | 0 | 19.62 |
| SILCS25 | 2.65 | 34.70 | 48.69 | 57.22 | 83.28 | 45.31 | −8.237 | −0.358 | 5.18 | 395.41 | 2 | 0 | 6.48 |
| SILCS26 | 20.78 | 55.15 | 73.44 | 87.57 | 94.13 | 66.21 | −6.622 | −0.315 | 2.85 | 286.42 | 2 | 0 | 19.62 |
| SILCS27 | 20.11 | 67.45 | 93.59 | 91.50 | 101.69 | 74.87 | −6.684 | −0.334 | 2.49 | 292.45 | 2 | 0 | 19.62 |
| SILCS28 | 11.61 | 38.84 | 70.79 | 80.14 | 96.11 | 59.50 | −6.619 | −0.331 | 2.38 | 292.45 | 2 | 0 | 19.62 |
| SILCS29 | 6.32 | 32.72 | 92.80 | 64.83 | 84.22 | 56.18 | −7.064 | −0.321 | 2.66 | 338.50 | 3 | 0 | 32.78 |
| SILCS30 | 24.84 | 67.20 | 98.54 | 84.81 | 100.21 | 75.12 | −6.512 | −0.283 | 3.13 | 332.47 | 3 | 0 | 32.78 |
| SILCS31 | 22.63 | 74.66 | 102.53 | 94.99 | 97.08 | 78.38 | −7.203 | −0.327 | 2.78 | 338.50 | 3 | 0 | 32.78 |
| SILCS32 | 2.40 | 30.29 | 42.26 | 39.61 | 81.11 | 39.13 | −8.811 | −0.401 | 4.70 | 401.44 | 2 | 0 | 6.48 |
| SILCS33 | 60.31 | 75.32 | 120.53 | 109.77 | 71.22 | 87.43 | −6.699 | −0.305 | 3.03 | 297.45 | 3 | 0 | 19.37 |
| SILCS34 | 99.38 | 113.76 | 121.29 | 112.81 | 105.55 | 110.56 | −7.069 | −0.337 | 2.81 | 322.50 | 2 | 0 | 23.55 |
| SILCS35 | 115.34 | 105.43 | 116.46 | 112.05 | 102.77 | 110.41 | −6.062 | −0.337 | 0.75 | 265.38 | 3 | 0 | 32.51 |
| SILCS36 | 27.37 | 57.44 | 102.85 | 111.84 | 114.84 | 82.87 | −7.436 | −0.338 | 1.92 | 301.43 | 3 | 0 | 32.51 |
| SILCS37 | 99.75 | 102.13 | 76.06 | 84.64 | 83.71 | 89.26 | −6.963 | −0.317 | 2.01 | 317.46 | 3 | 0 | 36.44 |
| SILCS38 | 86.49 | 98.63 | 73.06 | 84.78 | 76.25 | 83.84 | −7.071 | −0.321 | 2.05 | 317.46 | 3 | 0 | 36.44 |
| SILCS39 | 6.40 | 4.11 | 20.32 | 51.83 | 78.19 | 32.17 | −10.721 | −0.447 | 6.05 | 494.36 | 2 | 0 | 6.48 |
| SILCS40 | 52.05 | 141.44 | 115.37 | 98.86 | 117.72 | 105.09 | −7.131 | −0.357 | 2.57 | 308.47 | 2 | 0 | 23.55 |
| SILCS41 | 5.91 | 2.93 | 1.87 | 17.35 | 48.32 | 15.28 | −10.826 | −0.349 | 2.49 | 431.55 | 6 | 2 | 57.20 |
| SILCS42 | 45.13 | 52.26 | 66.63 | 40.19 | 71.06 | 55.05 | −7.97 | −0.38 | 4.47 | 387.41 | 2 | 0 | 6.48 |
| SILCS43 | −13.34 | 6.12 | 0.85 | 6.50 | 18.21 | 3.67 | −10.913 | −0.376 | 3.50 | 402.51 | 5 | 2 | 53.96 |
| SILCS44 | 0.86 | 0.18 | 7.02 | 42.62 | 102.10 | 30.56 | −10.688 | −0.356 | 3.29 | 420.50 | 5 | 2 | 53.96 |
| SILCS45 | 3.57 | −1.28 | 3.88 | −1.00 | 54.68 | 11.97 | −10.686 | −0.345 | 2.49 | 431.55 | 6 | 2 | 57.20 |
| SILCS46 | 0.84 | 0.03 | 21.64 | 0.77 | 72.12 | 19.08 | −10.354 | −0.334 | 2.49 | 431.55 | 6 | 2 | 57.20 |
| SILCS47 | 10.56 | 60.51 | 66.90 | 81.55 | 94.13 | 62.73 | −12.456 | −0.377 | 3.74 | 468.52 | 6 | 4 | 82.98 |
| SILCS48 | 160.04 | 100.34 | 85.09 | 75.19 | 71.69 | 98.47 | −6.44 | −0.23 | 2.23 | 381.43 | 4 | 3 | 88.69 |
| SILCS49 | 13.54 | 27.11 | 29.10 | 54.75 | 90.24 | 42.95 | −9.743 | −0.325 | 2.53 | 418.51 | 6 | 2 | 63.19 |
| SILCS50 | 0.30 | 1.99 | 4.51 | 10.74 | 78.80 | 19.27 | −11.798 | −0.369 | 2.82 | 443.56 | 6 | 2 | 57.20 |
| SILCS51 | 105.46 | 95.65 | 92.65 | 100.66 | 100.02 | 98.89 | −5.278 | −0.229 | 2.06 | 327.41 | 2 | 3 | 70.23 |
| SILCS52 | 122.98 | 120.98 | 104.43 | 117.76 | 92.28 | 111.69 | −7.068 | −0.283 | 3.40 | 343.43 | 5 | 3 | 76.24 |
| SILCS53 | 129.39 | 127.31 | 113.86 | 123.52 | 112.96 | 121.41 | −8.272 | −0.331 | 4.03 | 426.38 | 2 | 3 | 70.23 |
| SILCS54 | 113.08 | 105.10 | 89.30 | 92.88 | 99.39 | 99.95 | −6.601 | −0.287 | 2.51 | 315.37 | 5 | 3 | 76.24 |
| SILCS55 | 117.49 | 89.06 | 71.09 | 77.30 | 79.08 | 86.80 | −7.064 | −0.283 | 2.48 | 363.48 | 3 | 3 | 79.46 |
| SILCS56 | 121.04 | 97.73 | 70.76 | 55.85 | 68.41 | 82.76 | −6.342 | −0.276 | 1.32 | 335.43 | 3 | 3 | 79.46 |
| SILCS57 | 129.44 | 105.98 | 79.23 | 68.13 | 74.61 | 91.48 | −7.041 | −0.282 | 2.21 | 363.48 | 3 | 3 | 79.46 |
| SILCS58 | 160.09 | 135.64 | 105.19 | 78.84 | 68.63 | 109.68 | −7.306 | −0.281 | 3.23 | 379.52 | 3 | 3 | 79.46 |
| SILCS59 | 128.37 | 108.59 | 83.12 | 66.80 | 70.58 | 91.49 | −7.553 | −0.27 | 2.86 | 387.48 | 4 | 3 | 88.69 |
| SILCS60 | 122.52 | 98.54 | 73.53 | 73.65 | 79.38 | 89.52 | −7.582 | −0.303 | 3.67 | 343.43 | 5 | 3 | 76.24 |
| SILCS61 | 3.12 | 43.41 | 56.92 | 48.94 | 68.10 | 44.10 | −6.667 | −0.29 | 2.69 | 333.46 | 2 | 3 | 70.23 |
| SILCS62 | 23.88 | 51.41 | 46.35 | 30.37 | 68.14 | 44.03 | −5.677 | −0.27 | 1.53 | 305.40 | 2 | 3 | 70.23 |
| SILCS63 | 121.48 | 118.04 | 92.19 | 46.03 | 80.92 | 91.73 | −8.047 | −0.287 | 2.58 | 387.48 | 4 | 3 | 88.69 |
| SILCS64 | 158.69 | 130.68 | 111.17 | 33.52 | 79.58 | 102.73 | −7.438 | −0.286 | 1.70 | 359.43 | 4 | 3 | 88.69 |
| SILCS65 | 146.13 | 133.09 | 108.68 | 57.87 | 87.15 | 106.58 | −8.007 | −0.276 | 3.60 | 403.52 | 4 | 3 | 88.69 |
| SILCS66 | 128.18 | 112.41 | 107.16 | 61.08 | 103.65 | 102.50 | −6.493 | −0.24 | 1.61 | 385.44 | 4 | 3 | 88.69 |
| SILCS67 | 29.19 | 111.37 | 80.41 | 72.27 | 72.27 | 73.10 | −7.468 | −0.277 | 2.48 | 393.51 | 4 | 3 | 88.69 |
| SILCS68 | 79.20 | 110.69 | 83.40 | 71.37 | 67.28 | 82.39 | −8.912 | −0.356 | 3.76 | 426.38 | 2 | 3 | 70.23 |
| SILCS69 | 148.18 | 126.06 | 75.49 | 70.55 | 55.77 | 95.21 | −5.643 | −0.235 | 1.42 | 325.37 | 2 | 3 | 83.37 |
| SILCS70 | 147.89 | 145.91 | 89.01 | 61.17 | 28.02 | 94.40 | −6.671 | −0.29 | 2.42 | 333.46 | 2 | 3 | 70.23 |
| SILCS71 | 94.18 | 73.83 | 40.26 | 33.19 | 37.44 | 55.78 | −10.523 | −0.329 | 4.84 | 444.59 | 5 | 3 | 62.75 |
| SILCS72 | 86.59 | 67.96 | 61.51 | 43.38 | 72.10 | 66.31 | −9.527 | −0.298 | 4.49 | 438.54 | 5 | 3 | 62.75 |
| | | | | | R | vs | 0.66 | 0.41 | −0.31 | −0.51 | −0.37 | 0.09 | 0.21 |
| | | | | | | Average | | | | | | | |
| | | | | | R2 | vs | 0.43 | 0.17 | 0.10 | 0.26 | 0.14 | 0.01 | 0.05 |
| | | | | | | Average | | | | | | | |

SARS2 and SILCS1-72 Averages (Percentage Inhibition)

| | Average | Average | Average | Average | Average |
|---|---|---|---|---|---|
| Compound | 50 μM | 25 μM | 10 μM | 5 μM | 1 μM |
| UMB18g | 94.65 | 93.14 | 90.05 | 68.79 | 40.42 |
| SILCS1 | 56.68 | 38.17 | −0.52 | −19.79 | −22.42 |
| SILCS2 | 97.75 | 94.16 | 53.29 | 49.92 | 14.32 |
| SILCS3 | 87.47 | 77.76 | 53.98 | 51.55 | 22.93 |
| SILCS4 | 90.83 | 81.29 | 86.21 | 59.94 | 17.51 |
| SILCS5 | 24.37 | 18.72 | 1.42 | −4.84 | −11.97 |
| SILCS6 | 68.55 | 45.03 | 11.83 | 28.49 | 20.42 |
| SILCS7 | 68.91 | 58.04 | 42.29 | 7.19 | 6.07 |
| SILCS8 | 91.62 | 86.04 | 46.96 | 24.52 | 12.10 |
| SILCS9 | 4.02 | −11.45 | −20.58 | −20.66 | −13.21 |
| SILCS10 | 93.61 | 66.00 | 58.42 | 56.27 | 47.40 |
| SILCS11 | 79.69 | 64.99 | 45.84 | 21.46 | 24.29 |
| SILCS12 | 23.34 | −35.12 | −10.30 | −9.01 | −20.43 |
| SILCS13 | 14.36 | −42.17 | −20.89 | 2.13 | 5.30 |
| SILCS14 | 98.31 | 85.35 | 6.16 | −5.13 | −5.53 |
| SILCS15 | 78.25 | 68.41 | 7.95 | −11.13 | −8.27 |
| SILCS16 | 98.05 | 95.86 | 25.48 | 12.21 | −12.00 |
| SILCS17 | 63.46 | 8.57 | −14.02 | −13.50 | −6.20 |
| SILCS18 | 73.35 | 99.73 | 33.83 | 14.06 | −13.22 |
| SILCS19 | 96.40 | 61.88 | 0.32 | −19.39 | −16.34 |
| SILCS20 | 51.85 | 91.37 | 60.82 | 18.24 | −15.20 |
| SILCS21 | 89.61 | 93.83 | 73.58 | 36.41 | 11.12 |
| SILCS22 | 97.32 | 93.63 | 46.87 | 29.69 | 27.62 |
| SILCS23 | 74.24 | 57.49 | 28.05 | 27.06 | 16.74 |
| SILCS24 | 80.74 | 64.96 | 29.04 | 28.46 | 31.47 |
| SILCS25 | 97.35 | 65.30 | 51.31 | 42.78 | 16.72 |
| SILCS26 | 79.22 | 44.85 | 26.56 | 12.43 | 5.87 |
| SILCS27 | 79.89 | 32.55 | 6.41 | 8.50 | −1.69 |
| SILCS28 | 88.39 | 61.16 | 29.21 | 19.86 | 3.89 |
| SILCS29 | 93.68 | 67.28 | 7.20 | 35.17 | 15.78 |
| SILCS30 | 75.16 | 32.80 | 1.46 | 15.19 | −0.21 |
| SILCS31 | 77.37 | 25.34 | −2.53 | 5.01 | 2.92 |
| SILCS32 | 97.60 | 69.71 | 57.74 | 60.39 | 18.89 |
| SILCS33 | 39.69 | 24.68 | −20.53 | −9.77 | 28.78 |
| SILCS34 | 0.62 | −13.76 | −21.29 | −12.81 | −5.55 |
| SILCS35 | −15.34 | −5.43 | −16.46 | −12.05 | −2.77 |
| SILCS36 | 72.63 | 42.56 | −2.85 | −11.84 | −14.84 |
| SILCS37 | 0.25 | −2.13 | 23.94 | 15.36 | 16.29 |
| SILCS38 | 13.51 | 1.37 | 26.94 | 15.22 | 23.75 |
| SILCS39 | 93.60 | 95.89 | 79.68 | 48.17 | 21.81 |
| SILCS40 | 47.95 | −41.44 | −15.37 | 1.14 | −17.72 |
| SILCS41 | 94.09 | 97.07 | 98.13 | 82.65 | 51.68 |
| SILCS42 | 54.87 | 47.74 | 33.37 | 59.81 | 28.94 |
| SILCS43 | 113.34 | 93.88 | 99.15 | 93.50 | 81.79 |
| SILCS44 | 99.14 | 99.82 | 92.98 | 57.38 | −2.10 |
| SILCS45 | 96.43 | 101.28 | 96.12 | 101.00 | 45.32 |
| SILCS46 | 99.16 | 99.97 | 78.36 | 99.23 | 27.88 |
| SILCS47 | 89.44 | 39.49 | 33.10 | 18.45 | 5.87 |
| SILCS48 | −60.04 | −0.34 | 14.91 | 24.81 | 28.31 |
| SILCS49 | 86.46 | 72.89 | 70.90 | 45.25 | 9.76 |
| SILCS50 | 99.70 | 98.01 | 95.49 | 89.26 | 21.20 |
| SILCS51 | −5.46 | 4.35 | 7.35 | −0.66 | −0.02 |
| SILCS52 | −22.98 | −20.98 | −4.43 | −17.76 | 7.72 |
| SILCS53 | −29.39 | −27.31 | −13.86 | −23.52 | −12.96 |
| SILCS54 | −13.08 | −5.10 | 10.70 | 7.12 | 0.61 |
| SILCS55 | −17.49 | 10.94 | 28.91 | 22.70 | 20.92 |
| SILCS56 | −21.04 | 2.27 | 29.24 | 44.15 | 31.59 |
| SILCS57 | −29.44 | −5.98 | 20.77 | 31.87 | 25.39 |
| SILCS58 | −60.09 | −35.64 | −5.19 | 21.16 | 31.37 |
| SILCS59 | −28.37 | −8.59 | 16.88 | 33.20 | 29.42 |
| SILCS60 | −22.52 | 1.46 | 26.47 | 26.35 | 20.62 |
| SILCS61 | 96.88 | 56.59 | 43.08 | 51.06 | 31.90 |
| SILCS62 | 76.12 | 48.59 | 53.65 | 69.63 | 31.86 |
| SILCS63 | −21.48 | −18.04 | 7.81 | 53.97 | 19.08 |
| SILCS64 | −58.69 | −30.68 | −11.17 | 66.48 | 20.42 |
| SILCS65 | −46.13 | −33.09 | −8.68 | 42.13 | 12.85 |
| SILCS66 | −28.18 | −12.41 | −7.16 | 38.92 | −3.65 |
| SILCS67 | 70.81 | −11.37 | 19.59 | 27.73 | 27.73 |
| SILCS68 | 20.80 | −10.69 | 16.60 | 28.63 | 32.72 |
| SILCS69 | −48.18 | −26.06 | 24.51 | 29.45 | 44.23 |
| SILCS70 | −47.89 | −45.91 | 10.99 | 38.83 | 71.98 |
| SILCS71 | 5.82 | 26.17 | 59.74 | 66.81 | 62.56 |
| SILCS72 | 13.41 | 32.04 | 38.49 | 56.62 | 27.90 |

SARS2 and SILCS1-72 Averages (Toxicity Data)

| Screening data (Relative cell count to DMSO controls) | | | | | | | For correlation analysis | | | | | | |
|---|---|---|---|---|---|---|---|---|---|---|---|---|---|
| | Average | Average | Average | Average | Average | | LGFE | LE | Log P | | | | |
| Compound | 50 μM | 25 μM | 10 μM | 5 μM | 1 μM | Average | (kcal/mol) | (kcal/mol) | (o/w) | Weight | a_acc | a_don | TPSA |
| UMB18g | 85.72 | 116.94 | 139.68 | 134.20 | 123.37 | 119.98 | −10.775 | −0.348 | 2.49 | 431.55 | 6 | 2 | 57.20 |
| SILCS1 | 84.14 | 116.28 | 108.52 | 99.54 | 98.82 | 101.46 | −8.507 | −0.37 | 3.12 | 348.54 | 3 | 1 | 26.71 |
| SILCS2 | 105.51 | 125.16 | 128.4 | 125.09 | 115.19 | 119.87 | −10.152 | 0.35 | 1.23 | 402.51 | 6 | 2 | 60.86 |
| SILCS3 | 86.97 | 117.43 | 124.47 | 125.56 | 114.7 | 113.83 | −10.801 | −0.338 | 2.54 | 445.58 | 6 | 2 | 57.20 |
| SILCS4 | 2.18 | 3.91 | 122.04 | 123.91 | 116.22 | 73.65 | −11.547 | −0.33 | 4.47 | 483.67 | 5 | 2 | 47.97 |
| SILCS5 | 75.59 | 105.3 | 102.76 | 108.79 | 113.75 | 101.24 | −7.946 | −0.345 | 3.41 | 328.48 | 3 | 1 | 26.71 |
| SILCS6 | 78.1 | 103.61 | 104.23 | 114.4 | 117.87 | 103.64 | −6.328 | −0.333 | 2.04 | 278.42 | 2 | 0 | 19.62 |
| SILCS7 | 73.73 | 103.49 | 106.58 | 103.66 | 110.82 | 99.66 | −6.927 | −0.346 | 2.22 | 292.45 | 2 | 0 | 19.62 |
| SILCS8 | 76.22 | 105.46 | 121.05 | 111.63 | 108.17 | 104.51 | −11.85 | −0.339 | 1.67 | 485.65 | 6 | 3 | 63.24 |
| SILCS9 | 60.67 | 92.21 | 111.19 | 103.56 | 105.37 | 94.60 | −7.284 | −0.364 | 1.34 | 294.42 | 3 | 1 | 39.85 |
| SILCS10 | 84.08 | 50.46 | 131 | 141.21 | 131.94 | 107.74 | −12.037 | −0.344 | 3.38 | 484.66 | 5 | 2 | 51.21 |
| SILCS11 | 80.38 | 105.91 | 120.55 | 106.15 | 111.01 | 104.80 | −6.487 | −0.324 | 2.51 | 272.39 | 2 | 0 | 19.62 |
| SILCS12 | 66.97 | 95.34 | 96.1 | 102.7 | 103.58 | 92.94 | −6.281 | −0.349 | 1.99 | 264.39 | 2 | 0 | 19.62 |
| SILCS13 | 78.61 | 104.32 | 105.25 | 107.19 | 105.73 | 100.22 | −7.218 | −0.344 | 1.40 | 308.45 | 3 | 1 | 39.85 |
| SILCS14 | 16.8 | 139.75 | 108.29 | 103.93 | 105.49 | 94.85 | −7.939 | −0.378 | 3.26 | 368.34 | 3 | 0 | 19.37 |
| SILCS15 | 65.19 | 115.74 | 107.75 | 104 | 105.96 | 99.73 | −8.166 | −0.408 | 3.21 | 357.32 | 2 | 0 | 19.62 |
| SILCS16 | 56.28 | 92.09 | 110.67 | 113.52 | 94.77 | 93.47 | −9.586 | −0.383 | 5.43 | 435.35 | 2 | 0 | 6.48 |
| SILCS17 | 53.52 | 75.47 | 88.87 | 89.17 | 96.29 | 80.66 | −6.549 | −0.345 | 2.20 | 278.42 | 2 | 0 | 19.62 |
| SILCS18 | 2.46 | 49.15 | 119.65 | 116.95 | 102.92 | 78.23 | −9.6 | −0.436 | 5.29 | 446.25 | 2 | 0 | 6.48 |
| SILCS19 | 57.85 | 89.74 | 106.06 | 98.71 | 105.15 | 91.50 | −7.907 | −0.395 | 4.14 | 373.38 | 2 | 0 | 6.48 |
| SILCS20 | 7.37 | 78.06 | 138.88 | 116.85 | 96.85 | 87.60 | −7.097 | −0.296 | 4.92 | 356.56 | 2 | 0 | 6.48 |
| SILCS21 | 17.58 | 81.56 | 136.35 | 118.34 | 104.37 | 91.64 | −7.226 | −0.301 | 4.80 | 356.56 | 2 | 0 | 6.48 |
| SILCS22 | 68.81 | 131.85 | 137.18 | 125.64 | 126.41 | 117.98 | −11.19 | −0.339 | 2.67 | 456.61 | 5 | 3 | 60.00 |
| SILCS23 | 92.88 | 112.58 | 125.61 | 129.2 | 123.69 | 116.79 | −7.337 | −0.334 | 3.19 | 300.45 | 2 | 0 | 19.62 |
| SILCS24 | 76.48 | 96.21 | 120.3 | 124 | 133.02 | 110.00 | −7.731 | −0.368 | 2.72 | 306.47 | 2 | 0 | 19.62 |
| SILCS25 | 21.94 | 79.63 | 122.86 | 125.28 | 108.44 | 91.63 | −8.237 | −0.358 | 5.18 | 395.41 | 2 | 0 | 6.48 |
| SILCS26 | 72.93 | 101.64 | 127.07 | 115.35 | 107.75 | 104.95 | −6.622 | −0.315 | 2.85 | 286.42 | 2 | 0 | 19.62 |
| SILCS27 | 86.33 | 103.23 | 120.18 | 120.66 | 109.07 | 107.89 | −6.684 | −0.334 | 2.49 | 292.45 | 2 | 0 | 19.62 |
| SILCS28 | 62.87 | 89.57 | 129.12 | 125.86 | 109.57 | 103.40 | −6.619 | −0.331 | 2.38 | 292.45 | 2 | 0 | 19.62 |
| SILCS29 | 67.81 | 101.15 | 90.99 | 110.81 | 104.12 | 94.98 | −7.064 | −0.321 | 2.66 | 338.50 | 3 | 0 | 32.78 |
| SILCS30 | 86.57 | 104.38 | 102.59 | 110.62 | 108.24 | 102.48 | −6.512 | −0.283 | 3.13 | 332.47 | 3 | 0 | 32.78 |
| SILCS31 | 96.45 | 102.57 | 109.5 | 114.77 | 113.44 | 107.35 | −7.203 | −0.327 | 2.78 | 338.50 | 3 | 0 | 32.78 |
| SILCS32 | 36.36 | 88 | 135.74 | 138.5 | 113.92 | 102.50 | −8.811 | −0.401 | 4.70 | 401.44 | 2 | 0 | 6.48 |
| SILCS33 | 59.2 | 85.8 | 80.17 | 94.18 | 118.72 | 87.61 | −6.699 | −0.305 | 3.03 | 297.45 | 3 | 0 | 19.37 |
| SILCS34 | 67.85 | 89.42 | 91 | 99.62 | 102.47 | 90.07 | −7.069 | −0.337 | 2.81 | 322.50 | 2 | 0 | 23.55 |
| SILCS35 | 60.5 | 93.31 | 89.77 | 100.64 | 105.31 | 89.91 | −6.062 | −0.337 | 0.75 | 265.38 | 3 | 0 | 32.51 |
| SILCS36 | 63.21 | 91.83 | 88.15 | 84.91 | 87.6 | 83.14 | −7.436 | −0.338 | 1.92 | 301.43 | 3 | 0 | 32.51 |
| SILCS37 | 75.29 | 104.07 | 116.31 | 110.16 | 108.95 | 102.96 | −6.963 | −0.317 | 2.01 | 317.46 | 3 | 0 | 36.44 |
| SILCS38 | 105.21 | 107.71 | 115.05 | 110 | 111.85 | 109.96 | −7.071 | −0.321 | 2.05 | 317.46 | 3 | 0 | 36.44 |
| SILCS39 | 46.35 | 128.15 | 149.83 | 114.94 | 107.69 | 109.39 | −10.721 | −0.447 | 6.05 | 494.36 | 2 | 0 | 6.48 |
| SILCS40 | 117.52 | 83.93 | 92.49 | 96.33 | 86.49 | 95.35 | −7.131 | −0.357 | 2.57 | 308.47 | 2 | 0 | 23.55 |
| SILCS41 | 101.49 | 118.34 | 124.03 | 139.55 | 118.33 | 120.35 | −10.826 | −0.349 | 2.49 | 431.55 | 6 | 2 | 57.20 |
| SILCS42 | 0.62 | 4.52 | 0.21 | 128.78 | 114.38 | 49.70 | −7.97 | −0.38 | 4.47 | 387.41 | 2 | 0 | 6.48 |
| SILCS43 | 0.01 | 136.27 | 132.39 | 151.78 | 154.73 | 115.04 | −10.913 | −0.376 | 3.50 | 402.51 | 5 | 2 | 53.96 |
| SILCS44 | 106.37 | 133.3 | 147.85 | 118.91 | 99.99 | 121.28 | −10.688 | −0.356 | 3.29 | 420.50 | 5 | 2 | 53.96 |
| SILCS45 | 125.46 | 119.79 | 131.34 | 123.47 | 116.96 | 123.40 | −10.686 | −0.345 | 2.49 | 431.55 | 6 | 2 | 57.20 |
| SILCS46 | 107.72 | 117.77 | 126.44 | 131.17 | 103.33 | 117.29 | −10.354 | −0.334 | 2.49 | 431.55 | 6 | 2 | 57.20 |
| SILCS47 | 58.34 | 123.82 | 126.65 | 112.86 | 106.63 | 105.66 | −12.456 | −0.377 | 3.74 | 468.52 | 6 | 4 | 82.98 |
| SILCS48 | 77.81 | 102.04 | 107.43 | 115.84 | 119.19 | 104.46 | −6.44 | −0.23 | 2.23 | 381.43 | 4 | 3 | 88.69 |
| SILCS49 | 98.89 | 115.31 | 120.89 | 113.44 | 104.43 | 110.59 | −9.743 | −0.325 | 2.53 | 418.51 | 6 | 2 | 63.19 |
| SILCS50 | 100.61 | 116.14 | 128.03 | 129.23 | 114.44 | 117.69 | −11.798 | −0.369 | 2.82 | 443.56 | 6 | 2 | 57.20 |
| SILCS51 | 85.53 | 98.78 | 104.12 | 104.19 | 100.38 | 98.60 | −5.278 | −0.229 | 2.06 | 327.41 | 2 | 3 | 70.23 |
| SILCS52 | 81.04 | 94.91 | 97.92 | 97.63 | 103.3 | 94.96 | −7.068 | −0.283 | 3.40 | 343.43 | 5 | 3 | 76.24 |
| SILCS53 | 84.68 | 97.25 | 103.08 | 101.22 | 103.19 | 97.88 | −8.272 | −0.331 | 4.03 | 426.38 | 2 | 3 | 70.23 |
| SILCS54 | 88.29 | 99.71 | 100.51 | 101.65 | 100.58 | 98.15 | −6.601 | −0.287 | 2.51 | 315.37 | 5 | 3 | 76.24 |
| SILCS55 | 87.84 | 103.46 | 119.11 | 111.39 | 115.35 | 107.43 | −7.064 | −0.283 | 2.48 | 363.48 | 3 | 3 | 79.46 |
| SILCS56 | 89.59 | 104.41 | 119.65 | 119.22 | 120.34 | 110.64 | −6.342 | −0.276 | 1.32 | 335.43 | 3 | 3 | 79.46 |
| SILCS57 | 85.77 | 100.22 | 111.79 | 108.63 | 114.12 | 104.11 | −7.041 | −0.282 | 2.21 | 363.48 | 3 | 3 | 79.46 |
| SILCS58 | 80.27 | 94.22 | 103.42 | 109.77 | 112.69 | 100.07 | −7.306 | −0.281 | 3.23 | 379.52 | 3 | 3 | 79.46 |
| SILCS59 | 86.44 | 100.03 | 116.3 | 119.78 | 115.37 | 107.58 | −7.553 | −0.27 | 2.86 | 387.48 | 4 | 3 | 88.69 |
| SILCS60 | 85.19 | 96.61 | 109.31 | 110.17 | 111.59 | 102.57 | −7.582 | −0.303 | 3.67 | 343.43 | 5 | 3 | 76.24 |
| SILCS61 | 104.46 | 109.52 | 107.86 | 110.71 | 106.46 | 107.80 | −6.667 | −0.29 | 2.69 | 333.46 | 2 | 3 | 70.23 |
| SILCS62 | 98.08 | 106.51 | 116.29 | 123.92 | 112.47 | 111.45 | −5.677 | −0.27 | 1.53 | 305.40 | 2 | 3 | 70.23 |
| SILCS63 | 78.57 | 91.46 | 101.91 | 114.55 | 106.9 | 98.68 | −8.047 | −0.287 | 2.58 | 387.48 | 4 | 3 | 88.69 |
| SILCS64 | 75.66 | 84.89 | 92.78 | 119.2 | 103.13 | 95.13 | −7.438 | −0.286 | 1.70 | 359.43 | 4 | 3 | 88.69 |
| SILCS65 | 75.91 | 89.35 | 98.27 | 115.73 | 104.64 | 96.78 | −8.007 | −0.276 | 3.60 | 403.52 | 4 | 3 | 88.69 |
| SILCS66 | 84.65 | 90.79 | 93.98 | 109.01 | 98.81 | 95.45 | −6.493 | −0.24 | 1.61 | 385.44 | 4 | 3 | 88.69 |
| SILCS67 | 100.17 | 97.31 | 109.33 | 111.48 | 112.56 | 106.17 | −7.468 | −0.277 | 2.48 | 393.51 | 4 | 3 | 88.69 |
| SILCS68 | 91.82 | 100.14 | 110.48 | 114.63 | 117.26 | 106.87 | −8.912 | −0.356 | 3.76 | 426.38 | 2 | 3 | 70.23 |
| SILCS69 | 79.5 | 93.65 | 108.32 | 113.8 | 114.56 | 101.97 | −5.643 | −0.235 | 1.42 | 325.37 | 2 | 3 | 83.37 |
| SILCS70 | 79.59 | 93.9 | 104.3 | 111.32 | 130.04 | 103.83 | −6.671 | −0.29 | 2.42 | 333.46 | 2 | 3 | 70.23 |
| SILCS71 | 38.04 | 46.48 | 75.71 | 124.82 | 130.44 | 83.10 | −10.523 | −0.329 | 4.84 | 444.59 | 5 | 3 | 62.75 |
| SILCS72 | 36.41 | 76.24 | 113.46 | 118.22 | 113.33 | 91.53 | −9.527 | −0.298 | 4.49 | 438.54 | 5 | 3 | 62.75 |
| | | | | | R | vs | −0.24 | 0.07 | −0.33 | 0.13 | 0.37 | 0.29 | 0.33 |
| | | | | | | Average | | | | | | | |
| | | | | | R2 | vs | 0.06 | 0.01 | 0.11 | 0.02 | 0.14 | 0.08 | 0.11 |
| | | | | | | Average | | | | | | | |

Standard Deviation for the Above Tables Containing SARS2 and SILCS1-72 Averages (Infection Data, Percent Inhibition, and Toxicity Data)

| | STDEV | STDEV | STDEV | STDEV | STDEV |
|---|---|---|---|---|---|
| Compound | 50 μM | 25 μM | 10 μM | 5 μM | 1 μM |
| UMB18g | 2.95 | 3.15 | 6.61 | 21.09 | 22.01 |
| SILCS1 | 41.36 | 33.18 | 14.84 | 6.25 | 20.93 |
| SILCS2 | 2.41 | 4.04 | 14.04 | 32.06 | 29.95 |
| SILCS3 | 19.35 | 33.8 | 35.93 | 40.35 | 22.53 |
| SILCS4 | 6.36 | 12.1 | 22.31 | 24.79 | 15.66 |
| SILCS5 | 50.32 | 31.7 | 22.45 | 19.84 | 18.08 |
| SILCS6 | 28.34 | 40.79 | 45.1 | 51.19 | 43.98 |
| SILCS7 | 13.08 | 19.75 | 29.54 | 19.88 | 22.36 |
| SILCS8 | 7.29 | 14.31 | 36.75 | 27.41 | 21.39 |
| SILCS9 | 69.68 | 40.53 | 57.1 | 27.16 | 21.03 |
| SILCS10 | 7.46 | 38.29 | 16.1 | 32.67 | 32.2 |
| SILCS11 | 18.44 | 27.3 | 43.14 | 31.75 | 19.31 |
| SILCS12 | 76.66 | 135.12 | 110.3 | 109.01 | 120.43 |
| SILCS13 | 32.82 | 50.25 | 25.56 | 12.47 | 17.14 |
| SILCS14 | 1.69 | 14.65 | 93.84 | 105.13 | 105.53 |
| SILCS15 | 5.46 | 4.42 | 17.41 | 19.19 | 10.25 |
| SILCS16 | 3.77 | 7.74 | 26.74 | 18.75 | 9.16 |
| SILCS17 | 36.54 | 91.43 | 114.02 | 113.5 | 106.2 |
| SILCS18 | 34.98 | 1.72 | 25.21 | 32.22 | 34.49 |
| SILCS19 | 4.41 | 20.41 | 34.74 | 40.48 | 31.47 |
| SILCS20 | 73.17 | 2.09 | 18.97 | 31.06 | 24.17 |
| SILCS21 | 10.26 | 6.72 | 18.18 | 23.34 | 15.09 |
| SILCS22 | 1.58 | 3.03 | 25.97 | 25.2 | 7.37 |
| SILCS23 | 14.89 | 23.93 | 15.49 | 10.54 | 12.04 |
| SILCS24 | 9.08 | 21.51 | 21.43 | 23.78 | 44.76 |
| SILCS25 | 4.36 | 17.32 | 40.66 | 25.66 | 15.39 |
| SILCS26 | 8 | 16.49 | 29.63 | 8.29 | 9.36 |
| SILCS27 | 4.62 | 12.46 | 30.89 | 20.22 | 13 |
| SILCS28 | 7.42 | 11.15 | 40.97 | 34.34 | 18.41 |
| SILCS29 | 5.19 | 18.54 | 20.87 | 49.66 | 36.66 |
| SILCS30 | 5.53 | 9.44 | 24.54 | 34.46 | 11.18 |
| SILCS31 | 3.13 | 8.45 | 29.78 | 22 | 8.92 |
| SILCS32 | 2.96 | 6.52 | 23.55 | 9.79 | 17.2 |
| SILCS33 | 20.96 | 47.03 | 30.71 | 24.15 | 58.97 |
| SILCS34 | 41.86 | 36.25 | 24.97 | 24.76 | 20.03 |
| SILCS35 | 29.99 | 26.31 | 24.72 | 25.04 | 16.76 |
| SILCS36 | 18.31 | 22.01 | 17.8 | 22.5 | 18.92 |
| SILCS37 | 40.95 | 26.41 | 34.97 | 13.85 | 4.07 |
| SILCS38 | 11.15 | 29.28 | 19.79 | 21.4 | 21.76 |
| SILCS39 | 9.32 | 2.5 | 5.49 | 11.99 | 19.71 |
| SILCS40 | 8.51 | 32.51 | 36.83 | 25.02 | 41.86 |
| SILCS41 | 1.86 | 1.38 | 0.69 | 3.85 | 43.11 |
| SILCS42 | 49.09 | 17.48 | 49.14 | 15 | 23.26 |
| SILCS43 | 3.78 | 8.46 | 1.27 | 4.56 | 11.18 |
| SILCS44 | 2.18 | 2.09 | 4.44 | 42.72 | 15.56 |
| SILCS45 | 5.13 | 1.28 | 6.46 | 1.14 | 61.38 |
| SILCS46 | 3.52 | 2.44 | 27.05 | 1.09 | 43.81 |
| SILCS47 | 16.01 | 18.39 | 10.1 | 11.61 | 5.74 |
| SILCS48 | 13.41 | 16.01 | 26.17 | 10.98 | 15.32 |
| SILCS49 | 6.17 | 6.62 | 8.3 | 4.25 | 15.51 |
| SILCS50 | 3.98 | 3.87 | 4.13 | 3.56 | 28.24 |
| SILCS51 | 13.12 | 19.26 | 14.33 | 18 | 6.75 |
| SILCS52 | 13.03 | 13.19 | 14.15 | 33.61 | 16.24 |
| SILCS53 | 129.39 | 127.31 | 113.86 | 123.52 | 112.96 |
| SILCS54 | 113.08 | 105.1 | 89.3 | 92.88 | 99.39 |
| SILCS55 | 47.54 | 5.36 | 16.23 | 10.44 | 17.64 |
| SILCS56 | 37.6 | 15.56 | 18.73 | 6.48 | 8.59 |
| SILCS57 | 34.09 | 22.61 | 5.81 | 8.78 | 9.25 |
| SILCS58 | 58.26 | 25.58 | 18.93 | 7.24 | 4.74 |
| SILCS59 | 22.08 | 13.18 | 7.71 | 14.93 | 26.59 |
| SILCS60 | 15.13 | 20.18 | 6.41 | 4.21 | 15.31 |
| SILCS61 | 8.98 | 36.23 | 28.2 | 11.14 | 11.85 |
| SILCS62 | 49.82 | 59.85 | 47.16 | 22.03 | 30.23 |
| SILCS63 | 17.2 | 12.68 | 5.98 | 33.61 | 14.6 |
| SILCS64 | 14.86 | 15.7 | 14.32 | 37.53 | 4.93 |
| SILCS65 | 25.15 | 28.41 | 18.36 | 39.53 | 11.87 |
| SILCS66 | 33.68 | 22.37 | 19.34 | 42.42 | 6.3 |
| SILCS67 | 61.2 | 27.3 | 17.6 | 15.85 | 9.06 |
| SILCS68 | 56.45 | 27.7 | 13.85 | 14.45 | 7.18 |
| SILCS69 | 41.43 | 37.33 | 11.76 | 10.48 | 10.29 |
| SILCS70 | 24.84 | 39.78 | 13 | 9.98 | 33.45 |
| SILCS71 | 87.58 | 76.49 | 46.18 | 35.19 | 29.7 |
| SILCS72 | 83.48 | 77.53 | 69.73 | 46.45 | 9.2 |

Number of wells for the above tables containing SARS2 and SILCS 1-72 averages (infection data, percent inhibition, and toxicity data). n=number of wells total (n=9; triplicate well/triplicate experiment, n=4; duplicate well/duplicate experiment)

| Compound | n 50 μM | n 25 μM | n 10 μM | n 5 μM | n 1 μM |
|---|---|---|---|---|---|
| UMB18g | 34 | 34 | 34 | 34 | 34 |
| SILCS1 | 9 | 9 | 9 | 9 | 9 |
| SILCS2 | 9 | 9 | 9 | 9 | 9 |
| SILCS3 | 9 | 9 | 9 | 9 | 9 |
| SILCS4 | 9 | 9 | 9 | 9 | 9 |
| SILCS5 | 9 | 9 | 9 | 9 | 9 |
| SILCS6 | 9 | 9 | 9 | 9 | 9 |
| SILCS7 | 9 | 9 | 9 | 9 | 9 |
| SILCS8 | 9 | 9 | 9 | 9 | 9 |
| SILCS9 | 9 | 9 | 9 | 9 | 9 |
| SILCS10 | 9 | 9 | 9 | 9 | 9 |
| SILCS11 | 9 | 9 | 9 | 9 | 9 |
| SILCS12 | 9 | 9 | 9 | 9 | 9 |
| SILCS13 | 9 | 9 | 9 | 9 | 9 |
| SILCS14 | 9 | 9 | 9 | 9 | 9 |
| SILCS15 | 9 | 9 | 9 | 9 | 9 |
| SILCS16 | 9 | 9 | 9 | 9 | 9 |
| SILCS17 | 9 | 9 | 9 | 9 | 9 |
| SILCS18 | 9 | 9 | 9 | 9 | 9 |
| SILCS19 | 9 | 9 | 9 | 9 | 9 |
| SILCS20 | 9 | 9 | 9 | 9 | 9 |
| SILCS21 | 9 | 9 | 9 | 9 | 9 |
| SILCS22 | 9 | 9 | 9 | 9 | 9 |
| SILCS23 | 9 | 9 | 9 | 9 | 9 |
| SILCS24 | 9 | 9 | 9 | 9 | 9 |
| SILCS25 | 9 | 9 | 9 | 9 | 9 |
| SILCS26 | 9 | 9 | 9 | 9 | 9 |
| SILCS27 | 9 | 9 | 9 | 9 | 9 |
| SILCS28 | 9 | 9 | 9 | 9 | 9 |
| SILCS29 | 9 | 9 | 9 | 9 | 9 |
| SILCS30 | 9 | 9 | 9 | 9 | 9 |
| SILCS31 | 9 | 9 | 9 | 9 | 9 |
| SILCS32 | 9 | 9 | 9 | 9 | 9 |
| SILCS33 | 9 | 9 | 9 | 9 | 9 |
| SILCS34 | 9 | 9 | 9 | 9 | 9 |
| SILCS35 | 9 | 9 | 9 | 9 | 9 |
| SILCS36 | 9 | 9 | 9 | 9 | 9 |
| SILCS37 | 4 | 4 | 4 | 4 | 4 |
| SILCS38 | 4 | 4 | 4 | 4 | 4 |
| SILCS39 | 4 | 4 | 4 | 4 | 4 |
| SILCS40 | 4 | 4 | 4 | 4 | 4 |
| SILCS41 | 4 | 4 | 4 | 4 | 4 |
| SILCS42 | 4 | 4 | 4 | 4 | 4 |
| SILCS43 | 4 | 4 | 4 | 4 | 4 |
| SILCS44 | 4 | 4 | 4 | 4 | 4 |
| SILCS45 | 4 | 4 | 4 | 4 | 4 |
| SILCS46 | 4 | 4 | 4 | 4 | 4 |
| SILCS47 | 4 | 4 | 4 | 4 | 4 |
| SILCS48 | 4 | 4 | 4 | 4 | 4 |
| SILCS49 | 4 | 4 | 4 | 4 | 4 |
| SILCS50 | 4 | 4 | 4 | 4 | 4 |
| SILCS51 | 4 | 4 | 4 | 4 | 4 |
| SILCS52 | 4 | 4 | 4 | 4 | 4 |
| SILCS53 | 4 | 4 | 4 | 4 | 4 |
| SILCS54 | 4 | 4 | 4 | 4 | 4 |
| SILCS55 | 4 | 4 | 4 | 4 | 4 |
| SILCS56 | 4 | 4 | 4 | 4 | 4 |
| SILCS57 | 4 | 4 | 4 | 4 | 4 |
| SILCS58 | 4 | 4 | 4 | 4 | 4 |
| SILCS59 | 4 | 4 | 4 | 4 | 4 |
| SILCS60 | 4 | 4 | 4 | 4 | 4 |
| SILCS61 | 4 | 4 | 4 | 4 | 4 |
| SILCS62 | 4 | 4 | 4 | 4 | 4 |
| SILCS63 | 4 | 4 | 4 | 4 | 4 |
| SILCS64 | 4 | 4 | 4 | 4 | 4 |
| SILCS65 | 4 | 4 | 4 | 4 | 4 |
| SILCS66 | 4 | 4 | 4 | 4 | 4 |
| SILCS67 | 4 | 4 | 4 | 4 | 4 |
| SILCS68 | 4 | 4 | 4 | 4 | 4 |
| SILCS69 | 4 | 4 | 4 | 4 | 4 |
| SILCS70 | 4 | 4 | 4 | 4 | 4 |
| SILCS71 | 4 | 4 | 4 | 4 | 4 |
| SILCS72 | 4 | 4 | 4 | 4 | 4 |

Correlation analysis in FIG. 47 shows the overall correlation of all the compounds to be reasonable, with R²=0.43. Subsequently, the SILCS Bayesian Markov Chain Monte Carlo Machine Learning (BML) protocol (Ustach et al., 2019) was applied to determine if the predictability of the model could be improved. As shown in FIG. 48, small improvements in the predictability of the model were achieved, with the R² improving to 0.46. Interestingly, the most obvious outlier (SILCS47, BML weighed LGFE=−18.6) has a basic substitutent with two F atoms, in places of the piperazine, suggesting that the BML approach reweighted the contribution of the fluorobenzene FragMap based on the fluorophenyl group in all UMB18 analogs in a fashion the leads to the compound with the addition F atoms having its LGFE predicted to be too favorable. Removal of the outlier lead to improvement in the R² values to 0.53.

Selected physical properties of the compounds were calculated using the program MOE. These physical properties were then combined with the SILCS LGFE scores to yield improved predictive models. Improvements in the R2 values from 0.43 to 0.50 with TPSA as a physical property. This approach should be tried with the BML approach.

2) Correlation analysis of the individual scaffolds was performed, as summarized below. Further correlation analysis including use of physical properties and BML optimization needs to be systematically tested.
1) Overall predictive model is strong (all scaffolds, FIGS. 47 and 48)
2) UMB18 scaffold: weak predictive model for west wing modifications
3) UMB28 scaffold: strong predictive model
4) UMB22_5: poor correlation, some correlation with physical properties.

Results and Discussion of Infection and Toxicity Data

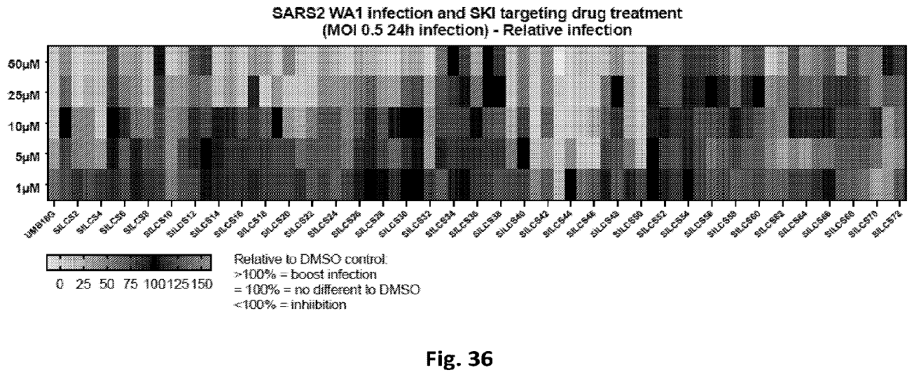

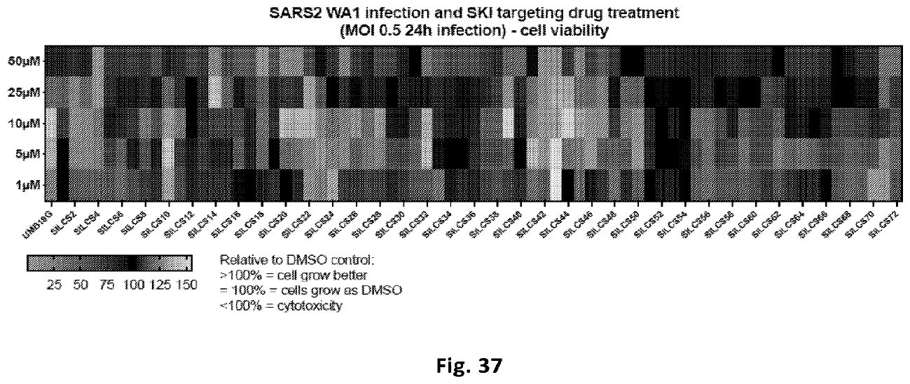

Based on the considerations discussed above for the four lead compounds, 72 analogs were designed and subjected to experimental validation. This development process included several iterations of design, synthesis, and experimental testing with emphasis on UMB18 and UMB28 as discussed above. Information from the experimental data from these iterations was incorporated into subsequent rounds of ligand design. The experimental data for all 72 analogs (SILCS1 is UMB28 and SILCS41 is UMB18) are shown in FIG. 36 for the SARS2 antiviral data and FIG. 37 for the cellular toxicity data (methods from Weston et al., 2020). In both figures the heat maps are adjusted so that bright green indicates maximal SARS2 antiviral data and minimal cellular toxicity. The initial iteration, compounds 1-13, focused on modifications of the central ring of UMB18 to generate additional scaffolds, and variations of the 5-membered rings in UMB28 while maintaining the basic nitrogen. A number of these compounds has strong activity, including SILCS10, which has a piperidine added to the central phenyl ring of UMB18. In addition, SILCS2, 3, 4 and 8 had various modifications of the central indicating that variations in this region of the molecule are feasible and could represent novel IP (SILCS22 that also has an additional ring on the central phenyl, assayed during iteration 2, also showed activity approaching that of UMB18). SILCS11 includes a phenyl ring versus a thiofuran in UMB28. This yielded improved activity over SILCS1 (which is UMB28) indicating that variations of the rings of UMB28 could yield improved activity. However, SILCS9 that added a methyl alcohol to a furan showed a significant loss of activity indicating the importance of nonpolar substitutions.

The second iteration, compounds SILCS14-36, focused on UMB28 analogs. A number of these compounds, 21, 23-29 and 32 had strong activities. These included the inclusion of halogens on the thiofuran rings and taking the basic amine from a dimethyl to a diethyl species. Additional of an ester on a thiofuran in 30 and 31 lead to a decrease in activity as did more polar moieties such as pyridine and oxazole groups, further indicating the importance of nonpolar ring systems.

Compounds SILCS37-48 in iteration 3 continued to test more nonpolar and halogen substitutions on the UMB28 analog terminal rings. Of these SILCS42 with two thiofuran rings one of which contained a Br group showed good activity, while an analog in which the central tertiary N was changed to an amide, SILCS40, was inactive. With UMB18, the role of chirality associated with the hydroxyl group on the sidechain terminated with the basic piperazine was tested by making the R and S stereoisomers (SILCS45, 46), resynthesis of the associated racemate (SILCS41) and basic variants of the piperazine were tested. The remake of UMB18G, SILCS41, has better activity than the original compound suggesting possible impurities. Notably, differential activity was observed in the individual stereoisomers, with SILCS45 showing improved activity over SILCS41 while SILCS46 had lower activity. This indicates that the final candidates should account for the chirality at this site of the molecules. In addition, SILCS43, with a pyrrolidine versus the piperazine in UMB18 had the highest activity to date, being approximately 3-4 more potent with an apparent IC50 in the high nanomolar range, despite being a racemic mixture.

SILCS48 and the final iteration, SILCS49-72, included a number of UMB22_5 and UMB22_16 analogs as well as additional variations of the piperazine of UMB18. The majority of the UMB22 related compounds typically had limited activity, with two of the compounds having activity within 2-fold of UMB18, SILCS61 and 62. Both of these compounds contain a thiofuran, which has also shown to facilitate the activity of UMB28 analogs and suggests that Br substitution may lead to additional improvements consistent with the FragMaps. Concerning the new UMB18 analogs, three of the compounds have 2- to 3-fold lower activity than UMB18, though SILCS50, with a diazo-biccyloheptane moiety versus the piperidine has activity similar to UMB18.

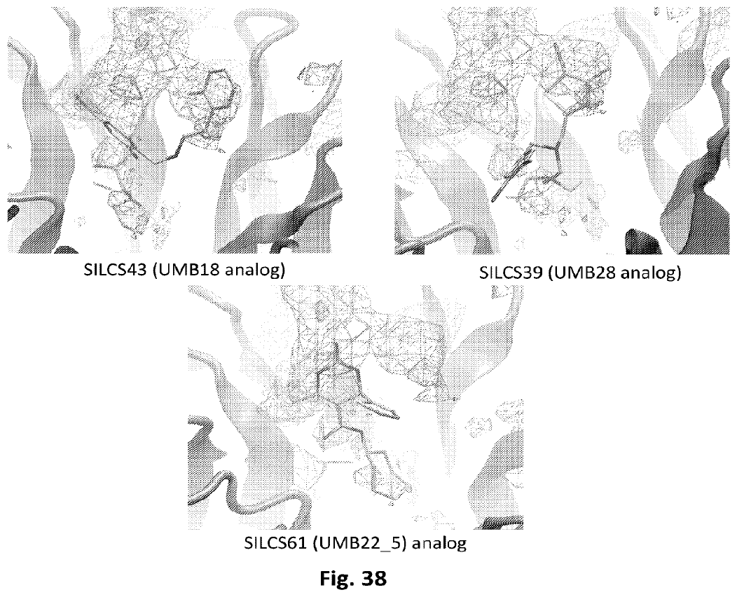

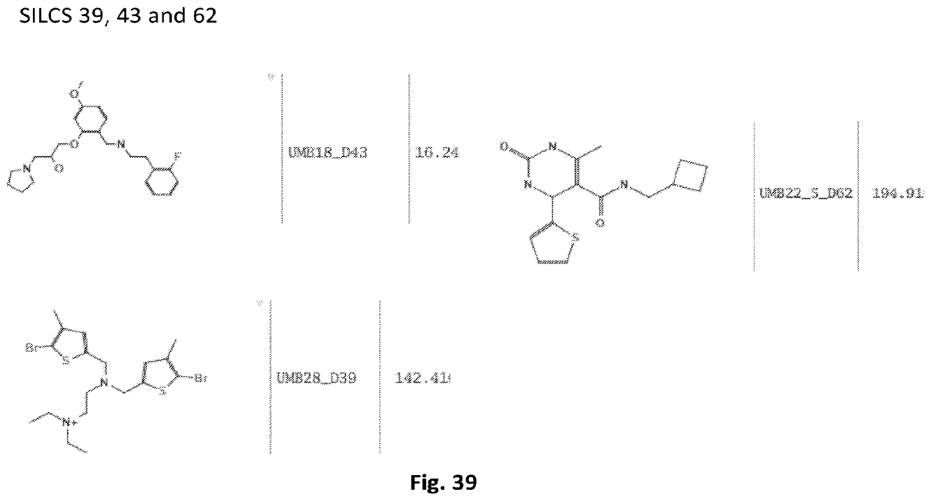

Predicted bound orientations of lead compounds from three of the lead series are shown on FIG. 38. Specifically, SILCS predicted binding orientations to WDR61 of the lead compounds from the UMB18, UMB28 and UMB22_Included are the SILCS Fragmaps in mesh for generic apolar (green), generic hydrogen-bond donor (blue) and acceptor (red), negatively charged (orange) and positively charged (cyan) functional groups at a GFE cutoff of −1.2 kcal/mol or −0.9 kcal/mol for the hydrogen bond donor and acceptor FragMaps. These compounds have been subjected to scaleup synthesis in preparation for PK and in vivo animal testing (FIG. 39).

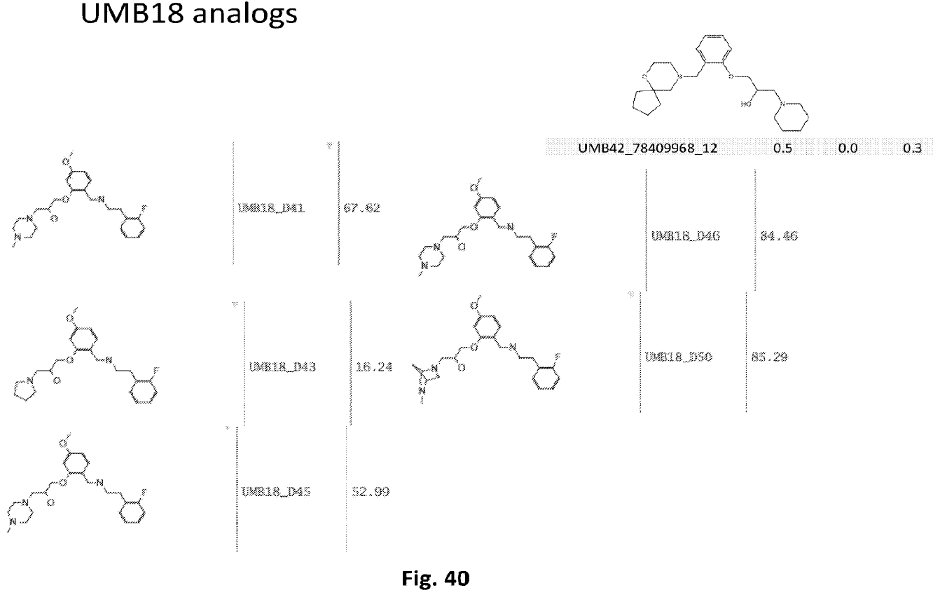

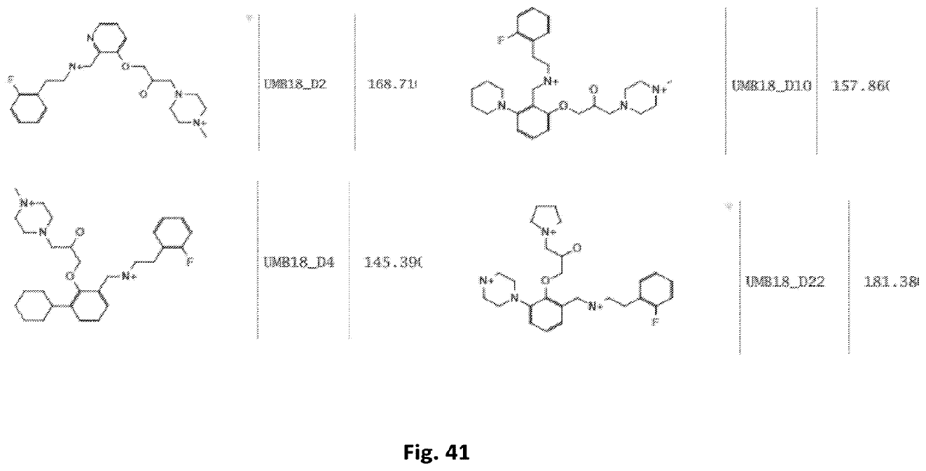

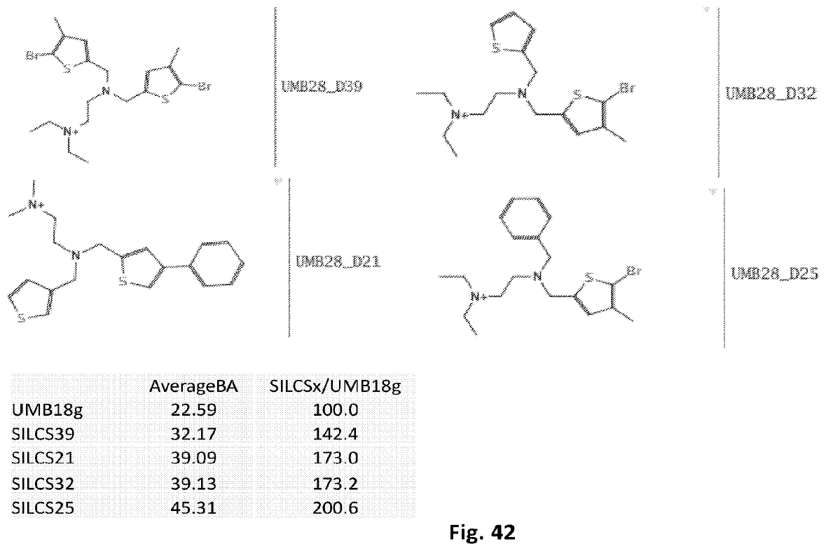

UMB18 analogs are shown in FIGS. 40-42. As shown in FIG. 40, SILCS41 was found to have improved properties over UMB18g. SILCS45 was found to have improved properties over SILCS46, with the R enantiomer is preferred. SILCS43 was found to be the most potent. FIG. 41 shows analogs with a modified central ring. As shown in FIG. 42, four analogs were found to be within 2-fold of UMB18G. UMB18G is a resynthesized version of UMB18. The SARS2 Activity is as follows: UMB18G=100, Improved BA<100. D41 is identical to UMB18G. D45 and D46 are R vs S of UMB18G: D45 was found to be more potent than D46. In a non-limiting embodiment, a positive charge on “west site” may improve properties. In a non-limiting embodiment, a fluorine atom on the “west” substituent may lower activity. UMB18 analog UMB42_78409968_12 with modifications on the “east” site was found to exhibit good activity.

Non-polar substituents at 2 and 3 positions appeared to be more portent, the central amide was not tolerated, and the compounds showed a strong predictive SILCS model.

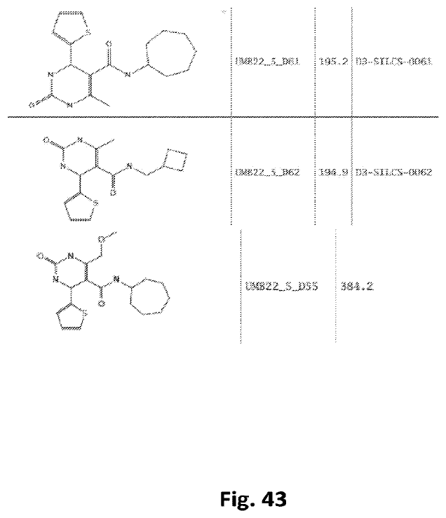

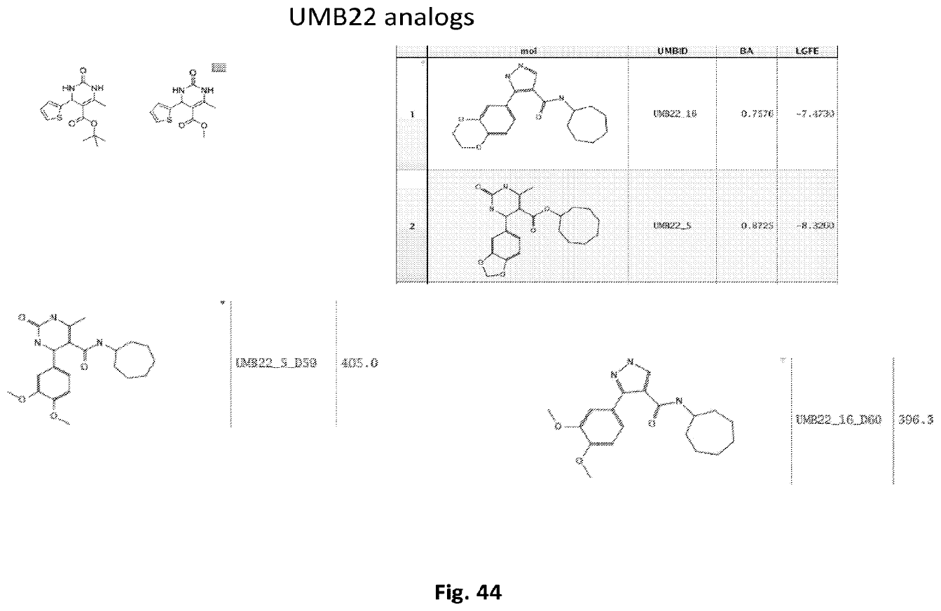

FIGS. 43-44 illustrates non-limiting examples of UMB22 analogs. UMB22 analogs from enamine generally exhibited poor activity, which is associated with polar/charged substituents. UMB22 analogs from Enamine generally had poor activity, which is associated with polar/charged substituents. 17 UMB22_5 analogs were examined: two compounds with activity within 2-fold of UMB18g were identified. The closed ring was found to be important for activity. UMB22_16 analogs were found to have poor activity (e.g. 4-fold worse than UMB18g). An additional ether chain leads to lower activity. As shown in FIG. 44, compounds UMB22_16 and UMB22_5 exhibited improved activity than ring open analogs (this was observed in many UMB22_5 analogs). The thiofuran ring was found to exhibit good activity, but Br substituent (UMB22_5_D53) and CH₂-phenyl on amide were not well tolerated (D51).

### Example 8. Surface Plasmon Resonance (SPR) Experiments

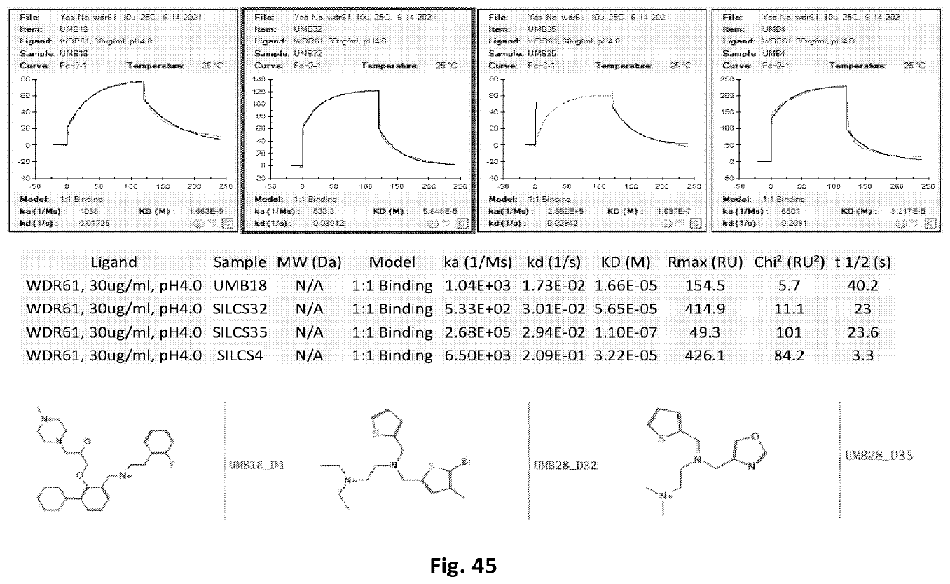

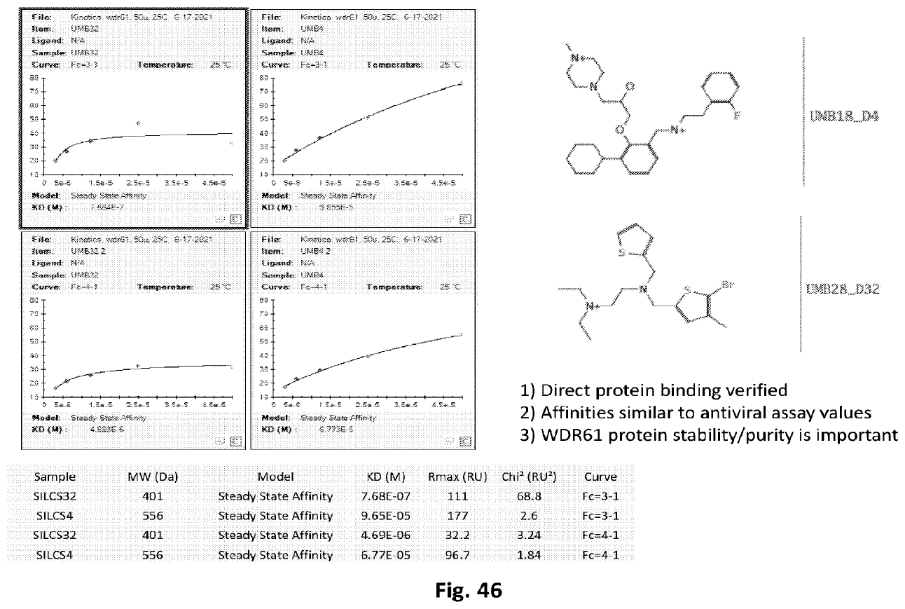

SPR experiments were undertaken on selected compounds with pure WDR61 protein to verify direct binding of the compounds to the protein. As shown in FIG. 45 and FIG. 46, direct binding is occurring. Notably, the binding affinities, based on the Kd values, are in the low micromolar range, consistent with the activity of the compounds in the SARS2 antiviral assay as indicated by the high level of inhibition at 5 and 1 micromolar in those assays.

SPR experiments were performed on 4 compounds, UMB18G, SILCS4, SILCS32 and SILCS35, with pure WDR61 (FIGS. 45 and 46). The SPR traces show that these compounds are binding directly to WDR61. Notable is that the Kd values from the SPR experiments are similar to those from the SARS2 antiviral assays, suggesting the antiviral activity is related to the binding to WDR61.

### REFERENCES

1. MacKerell Jr., A. D., Jo., S., Lakkaraju, S. K., Lind, C., and Yu, W. “Identification and Characterization of Fragment Binding Sites for Allosteric Ligand Design using the Site Identification by Ligand Competitive Saturation Hotspots Approach (SILCS-Hotspots),” Biochimia et Biophysica Acta, 1864: 129519, 2020.
2. Oashi, T., Ringer, A. L., Raman, E. P., and MacKerell, A. D., Jr., “Automated selection of compounds with physicochemical properties to maximize bioavailability and druglikeness,” Journal of Chemical Information and Modeling, 51:148-158, 2011.
3. Ustach, V. D., Lakkaraju, S. K., Jo, S., Yu, W., Jiang, W., and MacKerell, A. D., Jr. “Optimization and Evaluation of the Site-Identification by Ligand Competitive Saturation (SILCS) as a Tool for Target-Based Ligand Optimization,” Journal of Chemical Information and Modeling, 59: 3018-3035, 2019.
4. Weston, S., Baracco, L., Keller, C., Matthews, K., McGrath, M. E., Logue, J., Liang, J., Dyall, J., Holbrook, M. R., Hensley, L. E., Jahrling, P. B., Yu, W., MacKerell, A. D., Jr., and Frieman, M. B., “The SKI complex is a broad-spectrum, host-directed, antiviral drug target for coronaviruses, influenza and filoviruses,” Proceedings of the National Academies of Sciences, USA, 117:30687-30698, 2020.
5. Xu, C., Min, J. “Structure and function of WD40 domain proteins,” (2011) Protein Cell 2: 202-214.

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

1. A compound of formula (I), or a pharmaceutically acceptable salt, solvate, hydrate, cocrystal, or prodrug thereof: 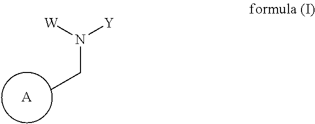
wherein in formula (I):
W is selected from C₁-C₅-alkyl and 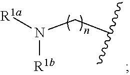
Y is selected from H, 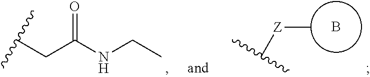
A and B are each independently selected from: 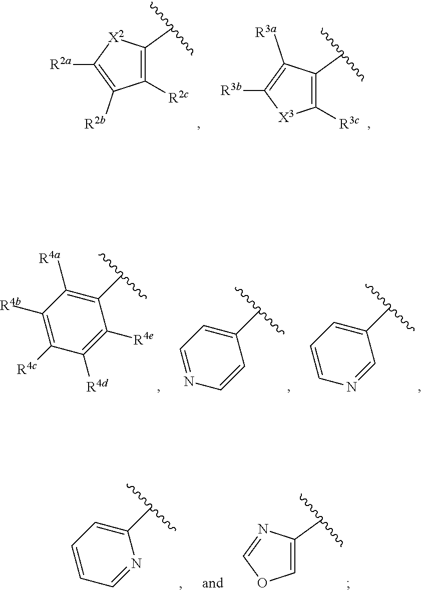
X² and X³ at each occurrence are independently selected from O and S;
Z is selected from —CH₂— and —C(O)—;
R^{1a} and R^{1b} at each occurrence are independently C₁-C₅-alkyl;
R^{2a}, R^{2b}, R^{2c}, R^{3a}, R^{3b}, R^{3c}, R^{4a}, R^{4b}, R^{4c}, R^{4d}, and R^{4e} at each occurrence are independently selected from hydrogen, halo, C₁-C₅-alkyl, trifluoromethyl, —(C₁-C₅-alkyl)-OH, phenyl, thiophenyl, furyl, and —C(O)O—C₁-C₅-alkyl; and
n is an integer selected from 1 to 5.

2. The compound of claim 1, wherein W is C₁-C₅-alkyl.

3. The compound of claim 1, wherein Y is H or

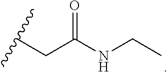 [SMILES: *CC(=O)NCC]

4. The compound of claim 1, wherein the compound of formula (I) is a compound of formula (10), or a pharmaceutically acceptable salt, solvate, hydrate, cocrystal, or prodrug thereof:

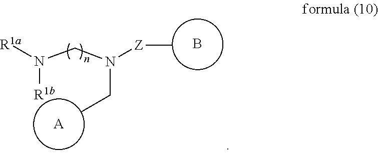

5. The compound of claim 1, wherein A and B are each independently selected from:

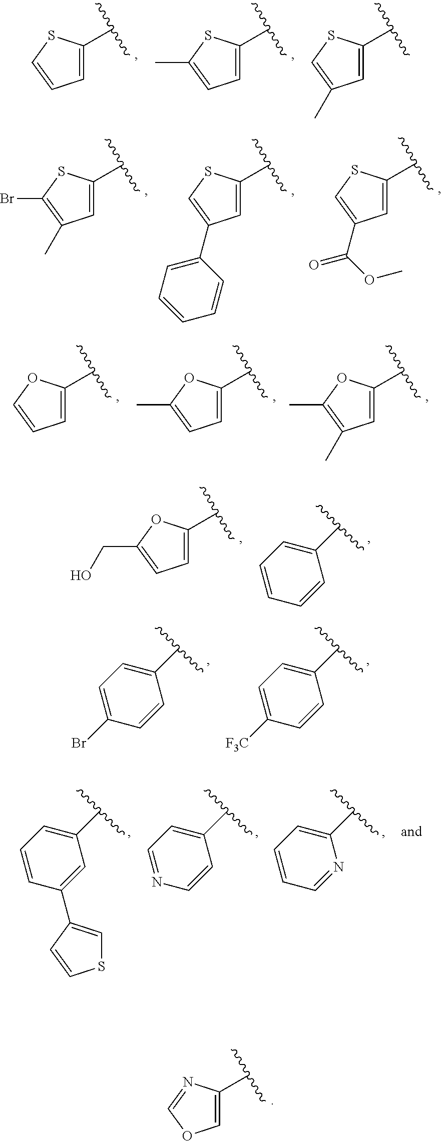

6. The compound of claim 5, wherein Z is —CH₂—.

7. The compound of claim 5, wherein

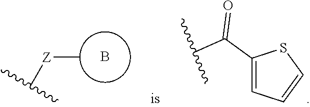

8. The compound of claim 1, wherein 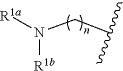
is selected from

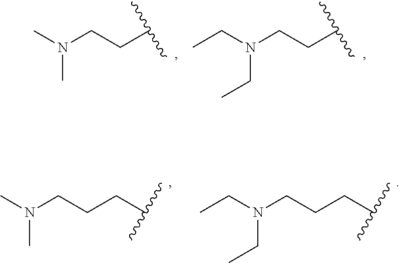

9. The compound of claim 1, wherein the compound of formula (I) is a compound having any one of formulas 1001-1038, or a pharmaceutically acceptable salt, solvate, hydrate, cocrystal, or prodrug thereof:

| Cpd. | |
|---|---|
| # | Structure |
| 1001 | 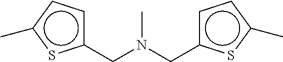 |
| 1002 | 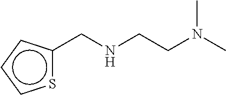 |
| 1003 | 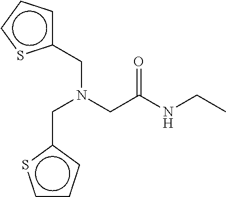 |
| 1004 | 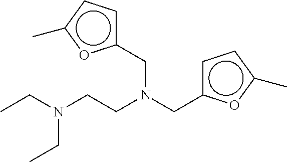 |
| 1005 | 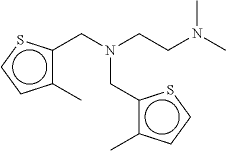 |
| 1006 | 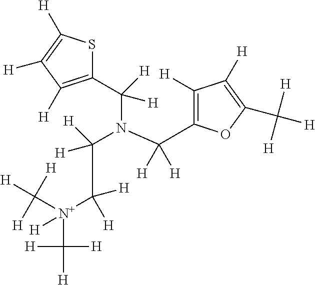 [SMILES: Cc1ccc(CN(CC[NH+](C)C)Cc2cccs2)o1] |
| 1007 | 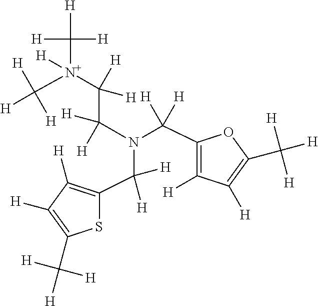 [SMILES: Cc1ccc(CN(CC[NH+](C)C)Cc2ccc(C)s2)o1] |
| 1008 | 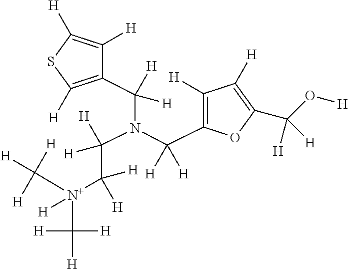 [SMILES: C[NH+](C)CCN(Cc1ccsc1)Cc1ccc(CO)o1] |
| 1009 | 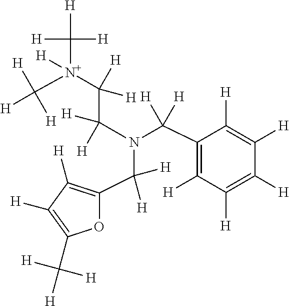 [SMILES: Cc1ccc(CN(CC[NH+](C)C)Cc2ccccc2)o1] |
| 1010 | 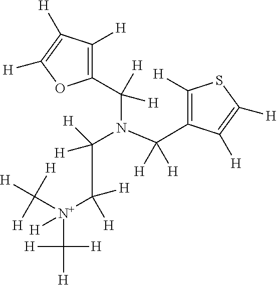 |
| 1011 | 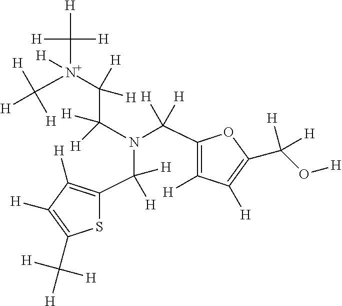 [SMILES: Cc1ccc(CN(CC[NH+](C)C)Cc2ccc(CO)o2)s1] |
| 1012 | 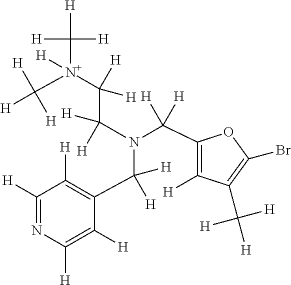 [SMILES: Cc1cc(CN(CC[NH+](C)C)Cc2ccncc2)oc1Br] |
| 1013 | 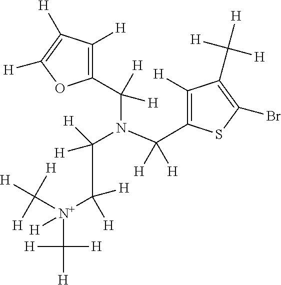 [SMILES: CCN(CC[NH+](C)C)Cc1cc(C)c(Br)s1 ; c1ccoc1] |
| 1014 | 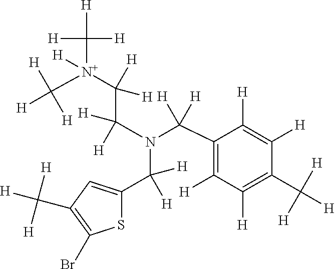 [SMILES: Cc1ccc(CN(CC[NH+](C)C)Cc2cc(C)c(Br)s2)cc1] |
| 1015 | 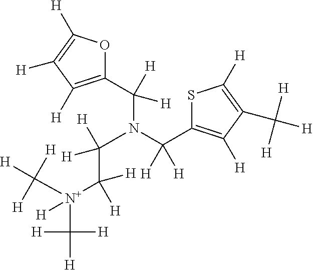 [SMILES: Cc1csc(CN(CC[NH+](C)C)Cc2ccco2)c1] |
| 1016 | 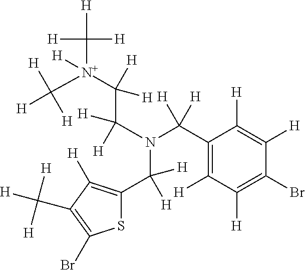 [SMILES: Cc1cc(CN(CC[NH+](C)C)Cc2ccc(Br)cc2)sc1Br] |
| 1017 | 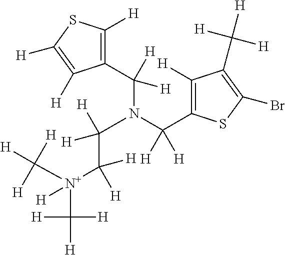 [SMILES: Cc1cc(CN(CC[NH+](C)C)Cc2ccsc2)sc1Br] |
| 1018 | 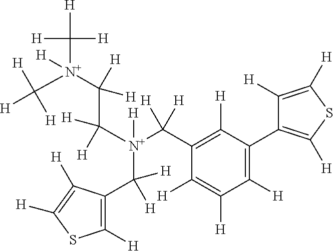 [SMILES: C[NH+](C)CC[NH+](Cc1ccsc1)Cc1cccc(-c2ccsc2)c1] |
| 1019 | 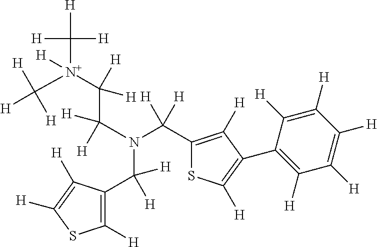 [SMILES: C[NH+](C)CCN(Cc1ccsc1)Cc1cc(-c2ccccc2)cs1] |
| 1020 | 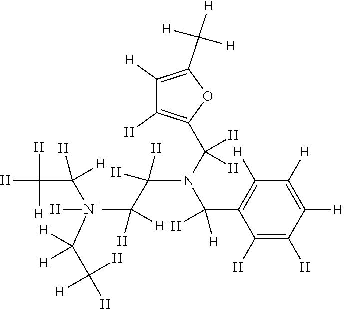 [SMILES: CC[NH+](CC)CCN(Cc1ccccc1)Cc1ccc(C)o1] |
| 1021 | 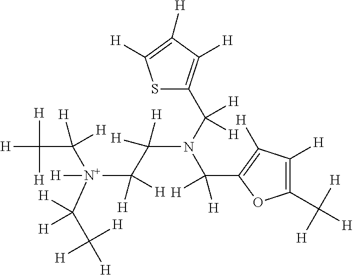 [SMILES: CC[NH+](CC)CCN(Cc1ccc(C)o1)Cc1cccs1] |
| 1022 | 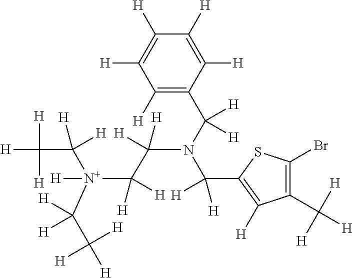 [SMILES: CC[NH+](CC)CCN(Cc1ccccc1)Cc1cc(C)c(Br)s1] |
| 1023 | 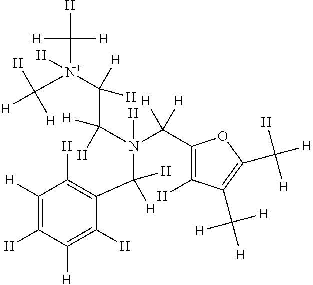 |
| 1024 | 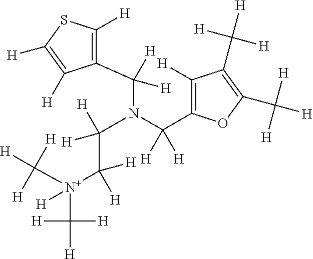 [SMILES: Cc1cc(CN(CC[NH+](C)C)Cc2ccsc2)oc1C] |
| 1025 | 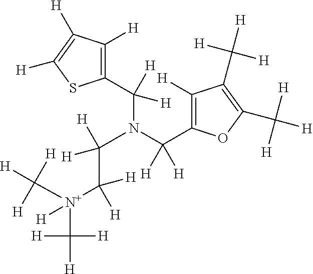 [SMILES: Cc1cc(CN(CC[NH+](C)C)Cc2cccs2)oc1C] |
| 1026 | 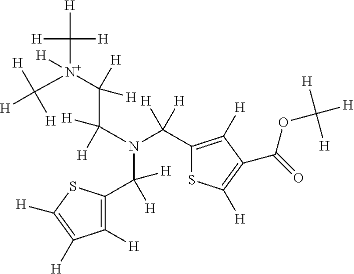 [SMILES: COC(=O)c1csc(CN(CC[NH+](C)C)Cc2cccs2)c1] |
| 1027 | 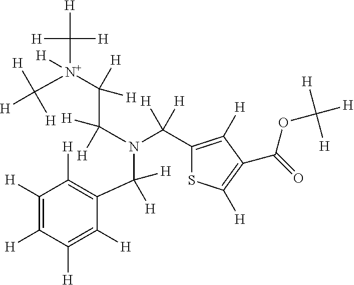 [SMILES: COC(=O)c1csc(CN(CC[NH+](C)C)Cc2ccccc2)c1] |
| 1028 | 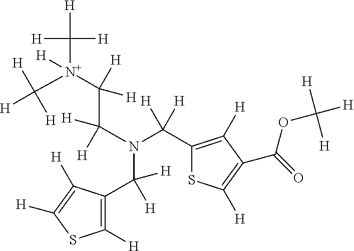 [SMILES: COC(=O)c1csc(CN(CC[NH+](C)C)Cc2ccsc2)c1] |
| 1029 | 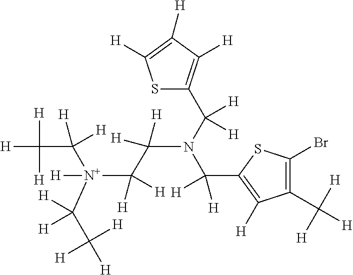 [SMILES: CC[NH+](CC)CCN(Cc1cccs1)Cc1cc(C)c(Br)s1] |
| 1030 | 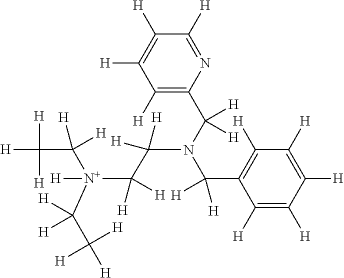 [SMILES: CC[NH+](CC)CCN(Cc1ccccc1)Cc1ccccn1] |
| 131 | 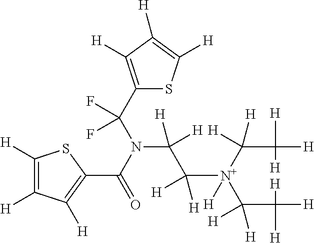 [SMILES: CC[NH+](CC)CCN(C(=O)c1cccs1)C(F)(F)c1cccs1] |
| 1032 | 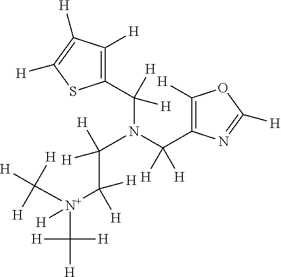 [SMILES: C[NH+](C)CCN(Cc1cocn1)Cc1cccs1] |
| 1033 | 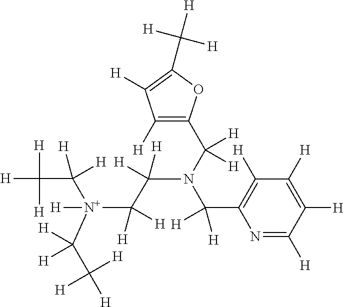 [SMILES: CC[NH+](CC)CCN(Cc1ccccn1)Cc1ccc(C)o1] |
| 1034 | 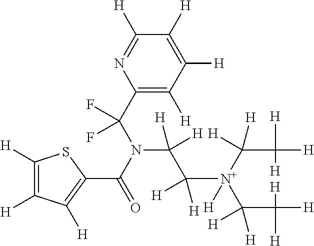 [SMILES: CC[NH+](CC)CCN(C(=O)c1cccs1)C(F)(F)c1ccccn1] |
| 1035 | 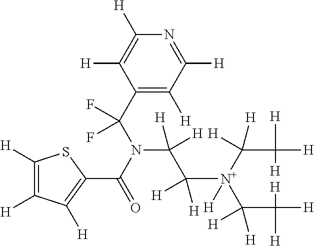 [SMILES: CC[NH+](CC)CCN(C(=O)c1cccs1)C(F)(F)c1ccncc1] |
| 1036 | 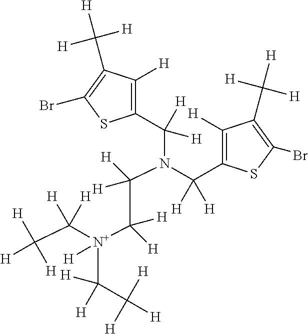 [SMILES: CC[NH+](CC)CCN(Cc1cc(C)c(Br)s1)Cc1cc(C)c(Br)s1] |
| 1037 | 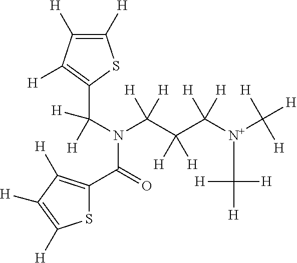 [SMILES: C[NH+](C)CCCN(Cc1cccs1)C(=O)c1cccs1] |
| 1038 | 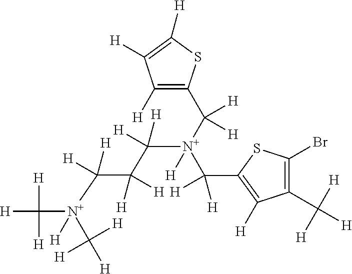 [SMILES: Cc1cc(C[NH+](CCC[NH+](C)C)Cc2cccs2)sc1Br] |

10. A compound of formula (II), or a pharmaceutically acceptable salt, solvate, hydrate, cocrystal, or prodrug thereof: 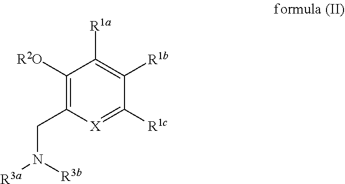
wherein in formula (II):
X is N or CR^{1d};
R^{1a}, R^{1b}, R^{1c}, and R^{1d} are each independently selected from hydrogen, C₁-C₅-alkyl, —OH, C₁-C₅-alkoxy, C₁-C₅-cycloalkyl, 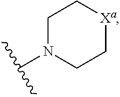
X^{a} is at each occurrence independently selected from —CH₂—, —O—, —NH—, or —N(C₁-C₅-alkyl)-;
R² is selected from hydrogen, C₁-C₅-alkyl, C₁-C₅-alkenyl, —(C₁-C₅-alkyl)-OH, 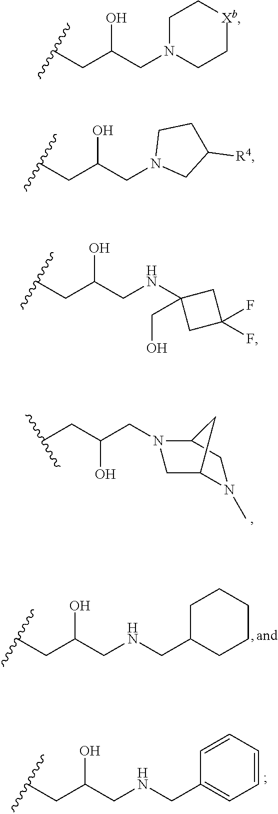
X^{b} is at each occurrence independently selected from —CH₂—, —O—, —NH—, or —N(C₁-C₅-alkyl)-;
R^{3a} and R^{3b} are each independently selected from hydrogen, C₁-C₅-alkyl, —(C₁-C₅-alkyl)-O—(C₁-C₅-alkyl), 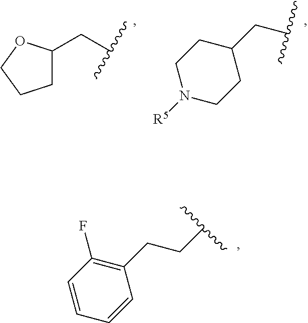
or R^{3a} and R^{3b} are joined together to form 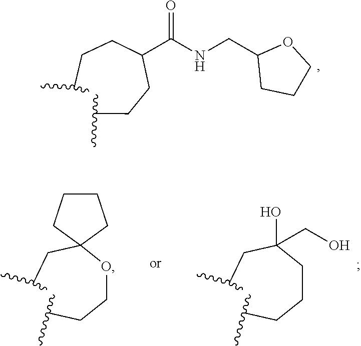 [SMILES: *CCC(CC*)C(=O)NCC1CCCO1 ; *CCOC1(C*)CCCC1 ; *CCCC(O)(C*)CO ; ";"]
R⁴ is at each occurrence independently selected from hydrogen and halo;
R⁵ is at each occurrence independently selected from

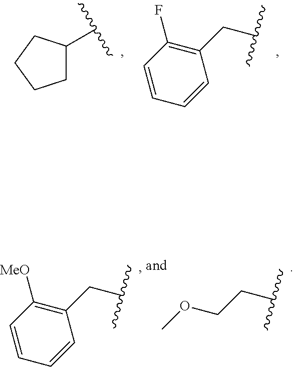

11. The compound of claim 10, wherein the compound of formula (II) is a compound of formula 20, or a pharmaceutically acceptable salt, solvate, hydrate, cocrystal, or prodrug thereof:

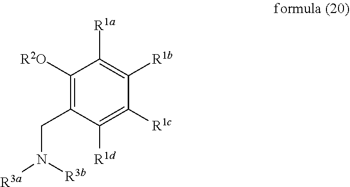

12. The compound of claim 10, wherein three of R^{1a}, R^{1b}, R^{1c}, and R^{1d} are hydrogen.

13. The compound of claim 10, wherein R^{1a} is selected from —OEt,

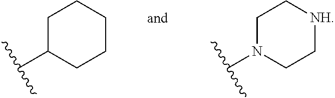 [SMILES: *C1CCCCC1 ; "and" ; *N1CCNCC1]

14. The compound of claim 10, wherein R^{1b} is —OMe.

15. The compound of claim 10, wherein R^{1d} is selected from —OH, —OMe, and

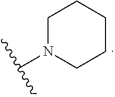 [SMILES: *N1CCCCC1]

16. The compound of claim 10, wherein R² is selected from hydrogen, 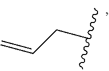 [SMILES: *CC=C]
methyl, ethyl,

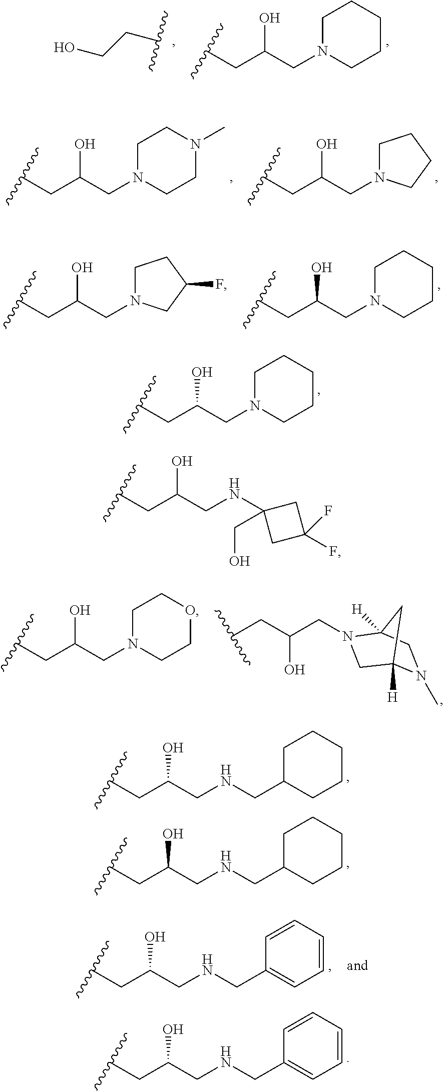

17. The compound of claim 10, wherein R^{3a} and R^{3b} are each independently selected from hydrogen,

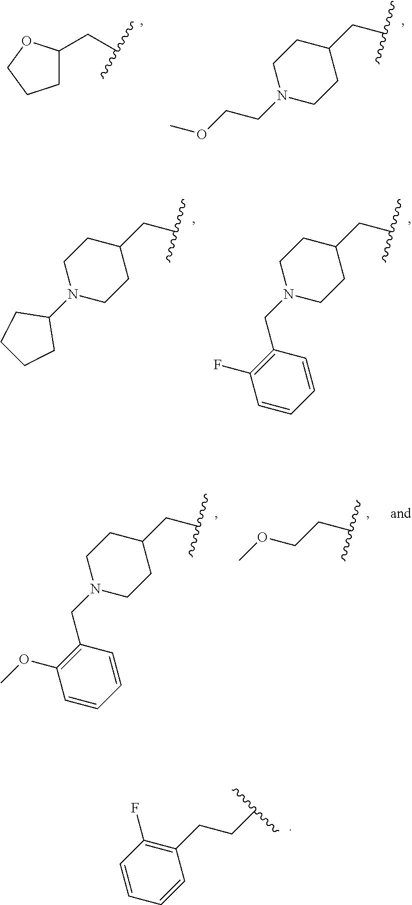

18. The compound of claim 10, wherein R^{3a} and R^{3b} join together to form a ring selected from

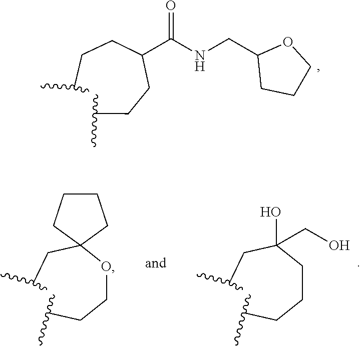 [SMILES: *CCC(CC*)C(=O)NCC1CCCO1 ; *CCOC1(C*)CCCC1 ; *CCCC(O)(C*)CO ; "."]

19. The compound of claim 10, wherein the compound of formula (II) is a compound having any one of formulas 2001-2028, or a pharmaceutically acceptable salt, solvate, hydrate, cocrystal, or prodrug thereof:

| Cpd. | |
|---|---|
| # | Structure |
| 2001 | 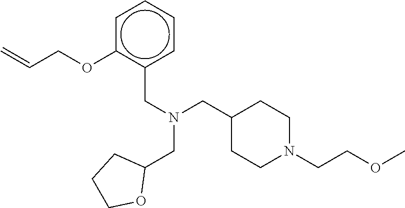 |
| 2002 | 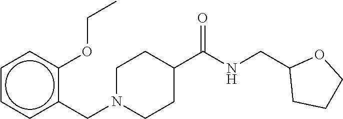 |
| 2003 | 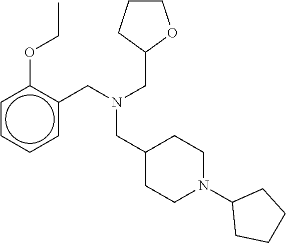 |
| 2004 | 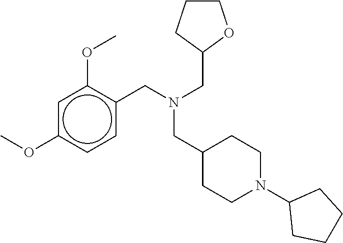 |
| 2005 | 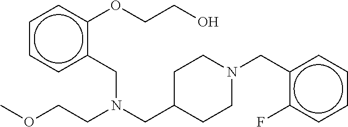 |
| 2006 | 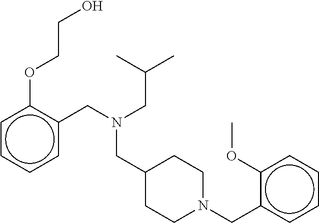 |
| 2007 | 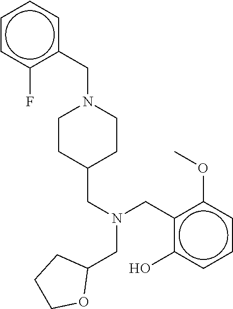 |
| 2008 | 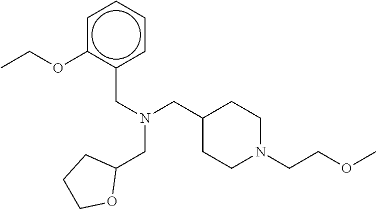 |
| 2009 | 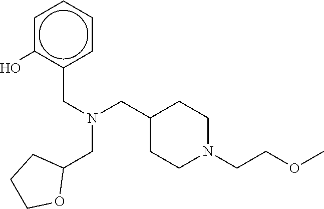 |
| 2010 | 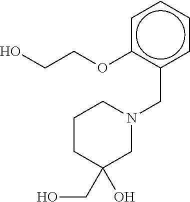 |
| 2011 | 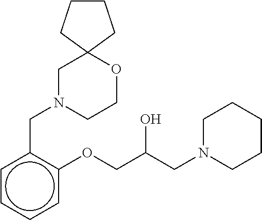 |
| 2012 | 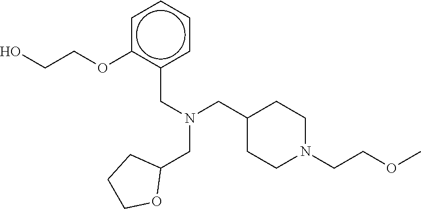 |
| 2013 | 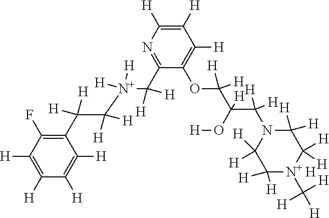 [SMILES: C[NH+]1CCN(CC(O)COc2cccnc2C[NH2+]CCc2ccccc2F)CC1] |
| 2014 | 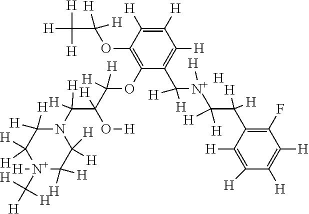 |
| 2015 | 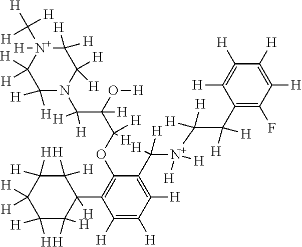 [SMILES: C[NH+]1CCN(CC(O)COc2c(C[NH2+]CCc3ccccc3F)cccc2C2CCCCC2)CC1] |
| 2016 | 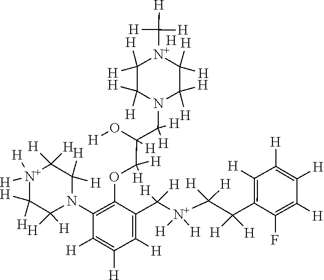 |
| 2017 | 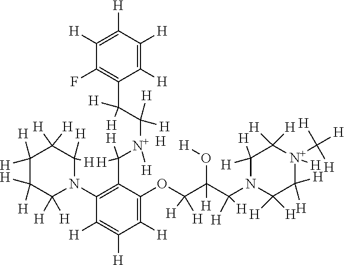 |
| 2018 | 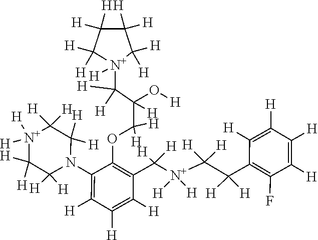 [SMILES: OC(COc1c(C[NH2+]CCc2ccccc2F)cccc1N1CC[NH2+]CC1)C[NH+]1CCCC1] |
| 2019 | 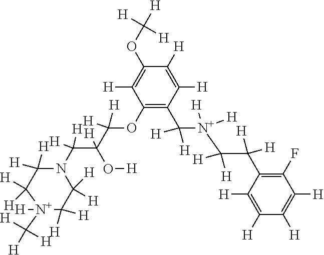 [SMILES: COc1ccc(C[NH2+]CCc2ccccc2F)c(OCC(O)CN2CC[NH+](C)CC2)c1] |
| 2020 | 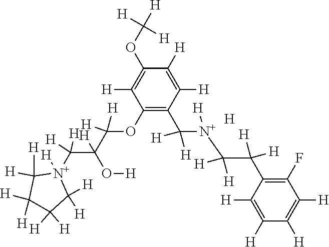 |
| 2021 | 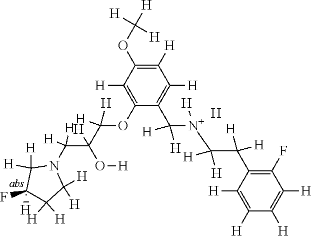 |
| 2022 | 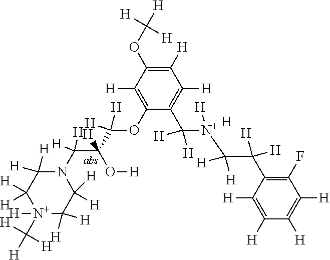 |
| 2023 | 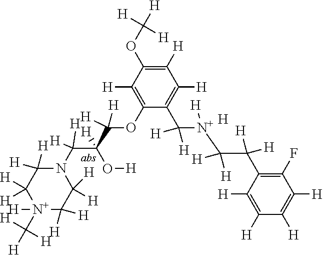 |
| 2024 | 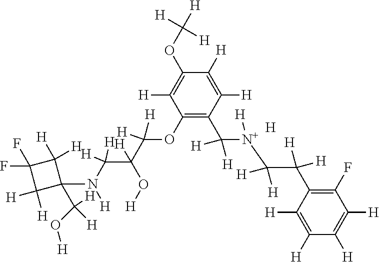 |
| 2025 | 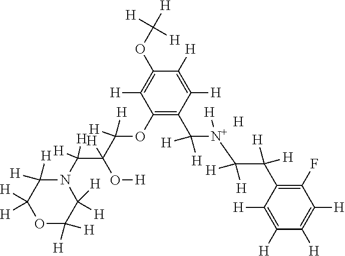 |
| 2026 | 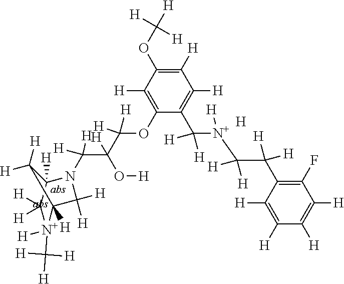 |
| 2027 | 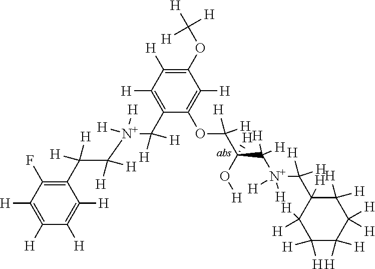 [SMILES: COc1ccc(C[NH2+]CCc2ccccc2F)c(OC[C@H](O)C[NH2+]CC2CCCCC2)c1] |
| 2028 | 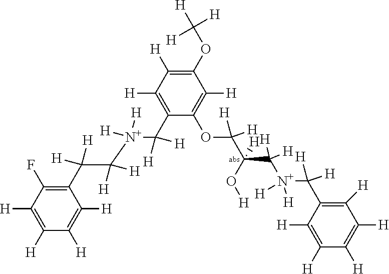 [SMILES: COc1ccc(C[NH2+]CCc2ccccc2F)c(OC[C@H](O)C[NH2+]Cc2ccccc2)c1] |

20. A compound of formula (III), or a pharmaceutically acceptable salt, solvate, hydrate, cocrystal, or prodrug thereof: 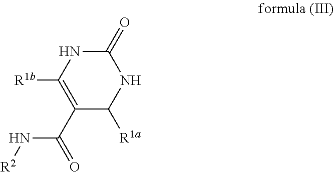
wherein in formula (III):
R^{1a} is selected from 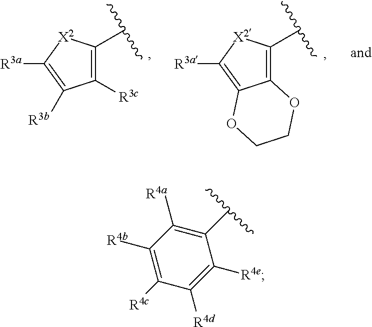
R^{1b} is selected from C₁-C₅-alkyl and —(C₁-C₅-alkyl)-O—(C₁-C₅-alkyl);
R² is selected from C₁-C₁₀-alkyl, C₃-C₁₀-cycloalkyl, —(CH₂)—C₃-C₁₀-cycloalkyl, and —(CH₂)—C₆-C₁₀-aryl;
X² and X^{2′} at each occurrence are independently selected from O and S; and
R^{3a}, R^{3b}, R^{3c}, R^{3a′}, R^{4a}, R^{4b}, R^{4c}, R^{4d}, and R^{4e} at each occurrence are independently selected from hydrogen, halo, C₁-C₅-alkyl, and C₁-C₅-alkoxy.

21. The compound of claim 20, wherein R^{1a} is selected from

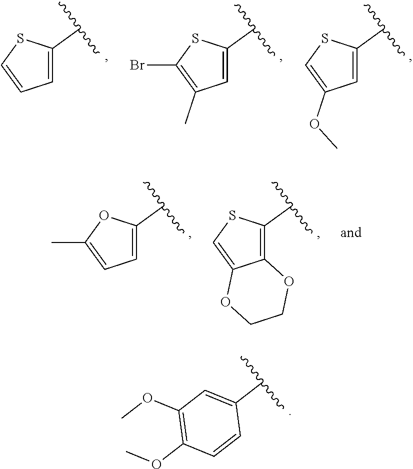

22. The compound of claim 20, wherein R^{1b} is selected from methyl and

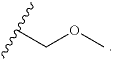 [SMILES: *COC]

23. The compound of claim 20, wherein R² is selected from

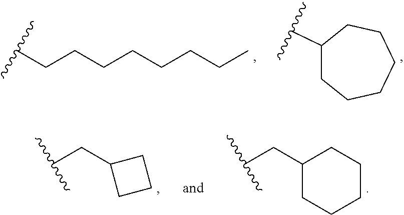

24. The compound of claim 20, wherein the compound of formula (III) is a compound having any one of formulas 3001-3018, or a pharmaceutically acceptable salt, solvate, hydrate, cocrystal, or prodrug thereof:

| Cpd. | |
|---|---|
| # | Structure |
| 3001 | 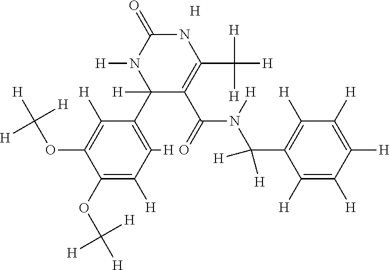 |
| 3002 | 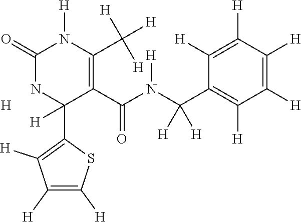 |
| 3003 | 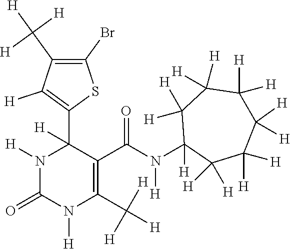 [SMILES: CC1=C(C(=O)NC2CCCCCC2)C(c2cc(C)c(Br)s2)NC(=O)N1] |
| 3004 | 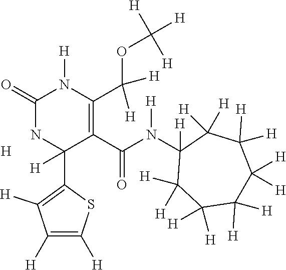 |
| 3005 | 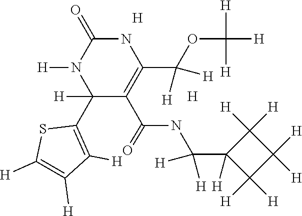 |
| 3006 | 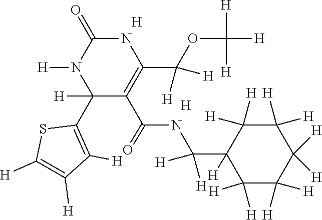 |
| 3007 | 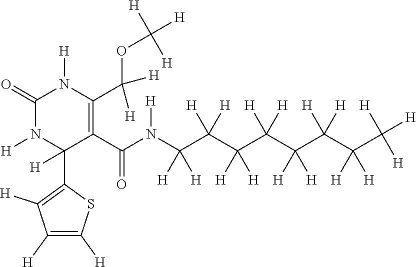 [SMILES: CCCCCCCCNC(=O)C1=C(COC)NC(=O)NC1c1cccs1] |
| 3008 | 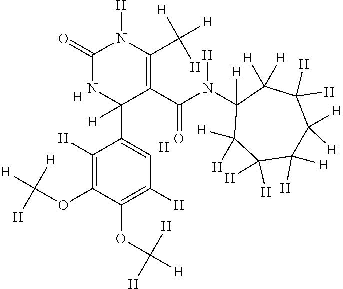 |
| 3009 | 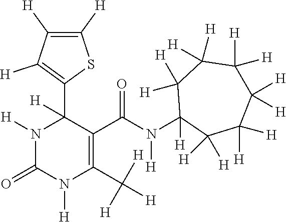 [SMILES: CC1=C(C(=O)NC2CCCCCC2)C(c2cccs2)NC(=O)N1] |
| 3010 | 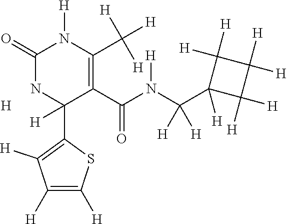 |
| 3011 | 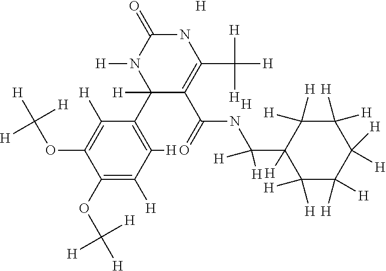 |
| 3012 | 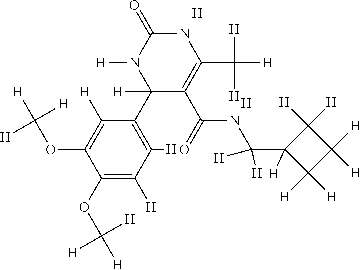 |
| 3013 | 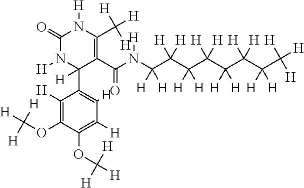 [SMILES: CCCCCCCCNC(=O)C1=C(C)NC(=O)NC1c1ccc(OC)c(OC)c1] |
| 3014 | 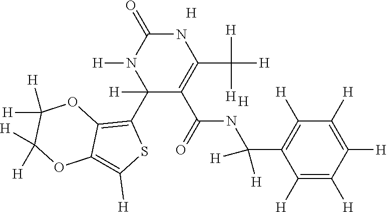 |
| 3015 | 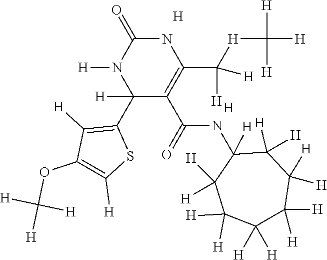 |
| 3016 | 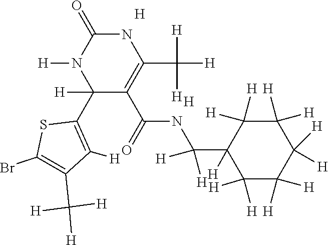 |
| 3017 | 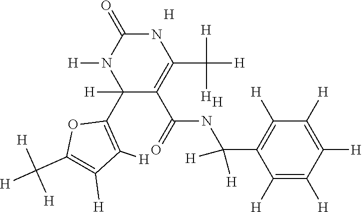 |
| 3018 | 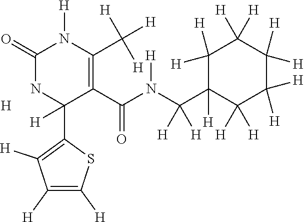 |

25. A compound of formula (IV), or a pharmaceutically acceptable salt, solvate, hydrate, cocrystal, or prodrug thereof:

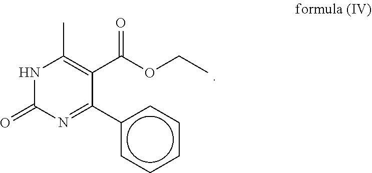

26. A compound of formula (V), or a pharmaceutically acceptable salt, solvate, hydrate, cocrystal, or prodrug thereof: 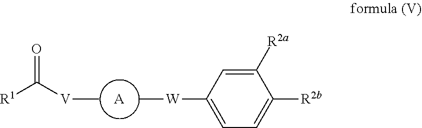
wherein in formula (V):
A is selected from 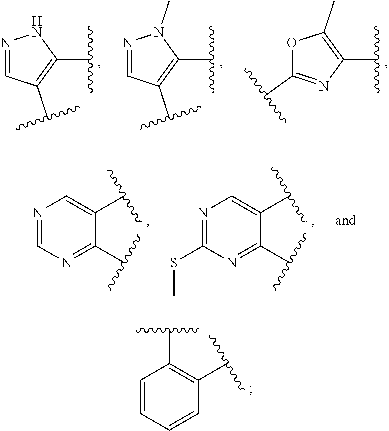
V is selected from a single bond and 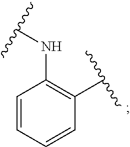 [SMILES: *Nc1ccccc1*]
W is selected from a single bond 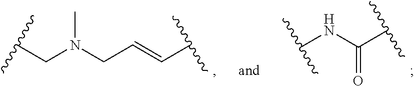
R¹ is selected from 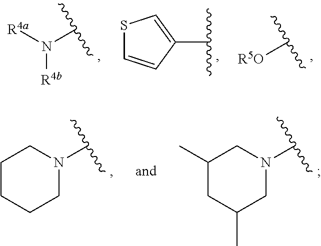
R^{2a} and R^{2b} are each independently selected from hydrogen, —OH, and C₁-C₅-alkoxy, or R^{2a} and R^{2b} are joined to form 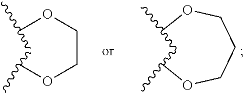
R^{4a} is selected from hydrogen and C₁-C₅-alkyl,
R^{4b} is selected from C₃-C₁₀-cycloalkyl, —(CH₂)—C₃-C₁₀-cycloalkyl, 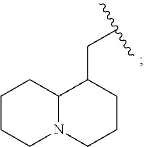 [SMILES: *CC1CCCN2CCCCC12]
and
R⁵ is selected from hydrogen, C₁-C₁₀-alkyl, and

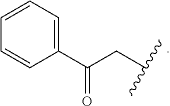 [SMILES: *CC(=O)c1ccccc1]

27. The compound of claim 26, wherein the compound of formula (V) is a compound of formula (50), or pharmaceutically acceptable salt, solvate, hydrate, cocrystal, or prodrug thereof: 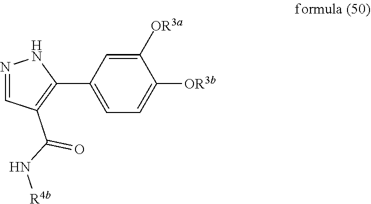
wherein in formula (50):
R^{3a} and R^{3b} are each independently C₁-C₅-alkyl or optionally can be joined to form 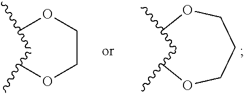
and
R^{4b} is selected from C₃-C₁₀-cycloalkyl and —(CH₂)—C₃-C₁₀-cycloalkyl.

28. The compound of claim 27, wherein R^{3a} and R^{3b} are each methyl.

29. The compound of claim 26, wherein R^{4b} is selected from

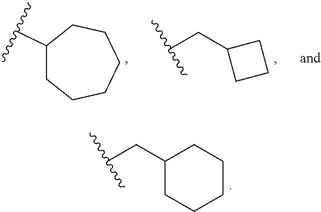 [SMILES: *C1CCCCCC1 ; *CC1CCC1 ; *CC1CCCCC1]

30. The compound of claim 26, wherein the compound of formula (V) is a compound having any one of formulas 5001-5013, or a pharmaceutically acceptable salt, solvate, hydrate, cocrystal, or prodrug thereof:

| Cpd. | |
|---|---|
| # | Structure |
| 5001 | 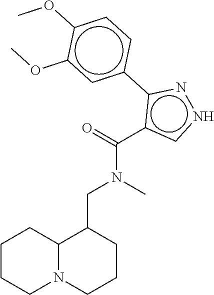 |
| 5002 | 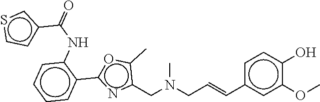 |
| 5003 | 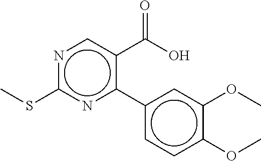 |
| 5004 | 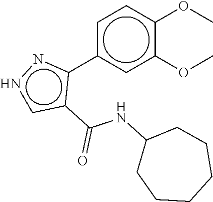 |
| 5005 | 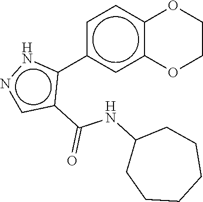 |
| 5006 | 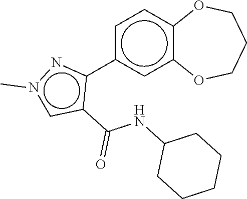 |
| 5007 | 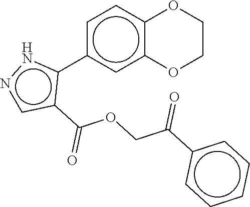 |
| 5008 | 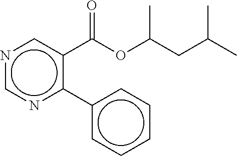 |
| 5009 | 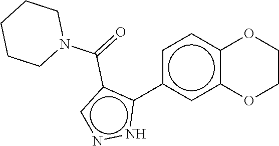 |
| 5010 | 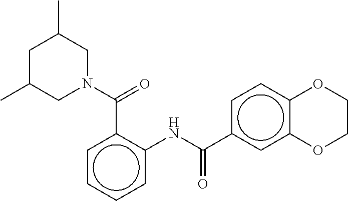 |
| 5011 | 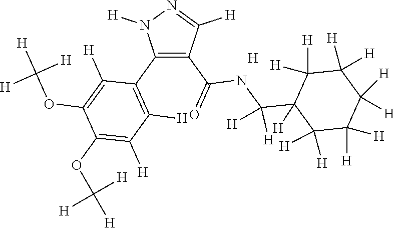 |
| 5012 | 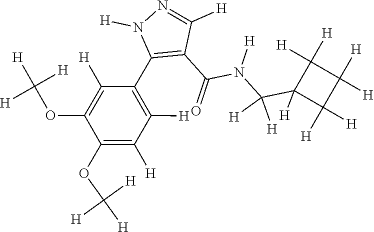 [SMILES: COc1ccc(-c2[nH]ncc2C(=O)NCC2CCC2)cc1OC] |
| 5013 | 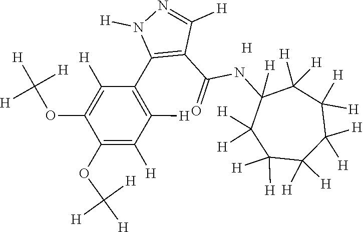 |

31.-57. (canceled)
